(19) United States
(12) Patent Application Publication
(10) Pub. No.: US 2012/0014927 A1
(43) Pub. Date: Jan. 19, 2012

(54) METHODS AND KITS FOR DETERMINING PREDISPOSITION TO CANCER

(75) Inventors: Tamar Rubinek, Or Yehuda (IL); Ido Wolf, Or Yehuda (IL); Eitan Friedman, Tel-Aviv (IL); Bella Kaufman, Kiryat-Ono (IL)

(73) Assignee: Tel HaShomer Medical Research Infrastructure and Services Ltd., Ramat-Gan (IL)

(21) Appl. No.: 13/132,335

(22) PCT Filed: Dec. 3, 2009
(86) PCT No.: PCT/IL09/01147

§ 371 Date: Sep. 21, 2011

Related U.S. Application Data
(60) Provisional application No. 61/119,434, filed on Dec. 3, 2008.

(51) Int. Cl. A61K 35/12; A61P 35/00; C12Q 1/68; C12Q 1/34; G01N 33/573

(52) U.S. Cl. 424/93.7; 435/18; 435/7.1; 435/6.12; 435/6.11

(57) ABSTRACT

Provided are methods and kits for determining predisposition to cancer by determining the presence or absence in a heterozygote form of the KLOTHO functional variant. Also provided are methods of designing treatment and treating cancer based on the increased predisposition to the cancer, and to determine the prognosis of a subject diagnosed with cancer.

### FIELD AND BACKGROUND OF THE INVENTION

The present invention, in some embodiments thereof, relates to methods and kits for determining predisposition to cancer such as breast cancer and ovarian cancer and, more particularly, but not exclusively, to methods of designing treatment regimens, treating and determining prognosis of the cancer.

Klotho protein (GenBank Accession No. NP_{—}004786) is a 1012 amino acid single pass transmembrane protein. Its extracellular domain is composed of two internal repeats, KL1 and KL2, which can be cleaved, shed into the serum and act as a circulating hormone. Biological activities in which Klotho has been implicated are described in Kurosu et al., 2005; Kuro-o et al., 1997; Cha et al., 2008; Yamamoto et al., 2005; Kurosu et al., 2006; Urakawa et al., 2006; Wolf et al., 2008; and WO2008/135993 (PCT Patent Application No. PCT/IL2008/000618).

Arking D E et al., 2002 describe the Klotho functional variant (termed KL-VS) which contains six sequence variants in complete linkage disequilibrium, two of which result in amino acid substitutions F352V and C370S in the Kolotho protein (GenBank Accession No. NP_{—}004786). In addition, while the presence of phenylalanine at a position equivalent to position 352 in the human KLOTHO gene is highly conserved among species it was found that homozygosity for the Klotho functional variant was under-represented in elderly compared to newborns in various populations (Arking D E et al., 2002) and was also identified as an independent risk factor for early-onset coronary artery disease (Arking D E, et al., 2003).

Germ line mutations in BRCA1 (MIM 113705) and BRCA2 (MIM 600185) genes substantially increase lifetime risk of breast and ovarian cancers. Yet, penetrance of deleterious BRCA1 and BRCA2 mutations is incomplete, age-dependent even among carriers of identical mutations (e.g. Ashkenazi Jewish, Iceland population) (Begg C B et al., 2008; Levy-Lahad and Friedman, 2007). Such observations suggest that other genetic and environmental factors may modify cancer risk in BRCA1 and BRCA2 mutation carriers (Antoniou and Easton, 2006). Several genetic variants have been reported as modifiers of cancer risk among these individuals (Antoniou A C et al., 2007; Antoniou A C et al., 2008). Single nucleotide polymorphisms (SNPs) in RAD51, FGFR2 and MAP3K1 have been linked to increased breast cancer risk among BRCA2 mutation carriers only (Antoniou A C et al., 2007) and a SNP in TNRC9 has been linked to increased breast cancer risk among both BRCA1 and BRCA2 mutation carriers.

Additional background art includes WO 01/20031; Lu L., et al., 2008; US 20080261908.

### SUMMARY OF THE INVENTION

According to an aspect of some embodiments of the present invention there is provided a method of determining if an individual is predisposed to cancer, the method comprising determining a presence or an absence of the KLOTHO functional variant in a heterozygote form wherein the presence of the KLOTHO functional variant in the heterozygote form is indicative of an increased predisposition to the cancer, thereby determining if the individual is predisposed to the cancer.

According to an aspect of some embodiments of the present invention there is provided a method of determining a prognosis of cancer in an individual, the method comprising determining a presence or an absence of the KLOTHO functional variant in a heterozygote or homozygous form wherein the presence of the KLOTHO functional variant in the heterozygote or the homozygous form is indicative of a worsen prognosis of the cancer as compared to the absence of the KLOTHO functional variant, thereby determining the prognosis of the cancer in the individual.

According to an aspect of some embodiments of the present invention there is provided a method of designing a treatment regimen to an individual, comprising: (a) determining if the individual is predisposed to cancer according to the method of the invention, and (b) designing a treatment regimen to the individual based on the presence of the predisposition, thereby designing the treatment regimen to the individual.

According to an aspect of some embodiments of the present invention there is provided a method of treating cancer in an individual, comprising: (a) determining if the individual is predisposed to the cancer according to the method of the invention, and (b) treating the cancer based on the presence of the predisposition, thereby treating the cancer in the individual.

According to an aspect of some embodiments of the present invention there is provided a kit for determining if an individual is predisposed to cancer, the kit comprising at least one oligonucleotide or antibody for specifically determining a presence or an absence of the KLOTHO functional variant in a heterozygote form, and at least one oligonucleotide or antibody for specifically determining a presence or an absence of a mutation in the BRCA1 nucleic acid sequence set forth by SEQ ID NO:4 or 27 and/or in the BRCA2 nucleic acid sequence set forth by SEQ ID NO:5 or 28, the mutation in the BRCA1 or in the BRCA2 is associated with the cancer.

According to some embodiments of the invention, the method further comprising determining a presence or an absence of a mutation in the BRCA1 sequence set forth by SEQ ID NO:4 (genomic sequence) or BRCA1 sequence set forth by SEQ ID NO:27 (mRNA sequence) which is associated with the cancer.

According to some embodiments of the invention, the method further comprising selecting the individual as being a carrier of a mutation in the BRCA1 sequence set forth by SEQ ID NO:4 (genomic sequence) or BRCA1 sequence set forth by SEQ ID NO:27 (mRNA sequence) which is associated with the cancer.

According to some embodiments of the invention, the method further comprising determining a presence or an absence of a mutation in the BRCA1 amino acid sequence set forth by SEQ ID NO:29, which is associated with the cancer.

According to some embodiments of the invention, the method further comprising selecting the individual as being a carrier of a mutation in the BRCA2 amino acid sequence set forth by SEQ ID NO:30, which is associated with the cancer.

According to some embodiments of the invention, the method further comprising determining a presence or an absence of a mutation in the BRCA2 sequence set forth by SEQ ID NO:5 (genomic sequence) or BRCA2 sequence set forth by SEQ ID NO:28 (mRNA sequence) which is associated with the cancer.

According to some embodiments of the invention, the method further comprising selecting the individual as being a carrier of a mutation in the BRCA2 sequence set forth by SEQ ID NO:5 or BRCA2 sequence set forth by SEQ ID NO:28 (mRNA sequence) which is associated with the cancer.

According to some embodiments of the invention, the KLOTHO functional variant comprises a valine amino acid residue at position 352 of the Klotho protein set forth by SEQ ID NO:7 or a guanine nucleotide at position 1062 of the KLOTHO transcript set forth by SEQ ID NO:6.

According to some embodiments of the invention, the KLOTHO functional variant is selected from the group consisting of a valine amino acid residue at position 352 of the Klotho protein set forth by SEQ ID NO:7, a guanine nucleotide at position 1062 of the KLOTHO transcript set forth by SEQ ID NO:6, a serine amino acid residue at position 370 of the klotho protein set forth by SEQ ID NO:7, a cytosine nucleotide at position 1117 of the KLOTHO transcript set forth by SEQ ID NO:6, and an adenosine nucleotide at position 1163 of the KLOTHO transcript set forth by SEQ ID NO:6.

According to some embodiments of the invention, the method is effected ex vivo.

According to some embodiments of the invention, the method further comprising informing the individual of the presence of the increased predisposition to the cancer.

According to some embodiments of the invention, the predisposition comprises an increased risk to develop the cancer at a younger age as compared to an individual who is not heterozygote to the KLOTHO functional variant.

According to some embodiments of the invention, the method further comprising recording the increased predisposition to the cancer in the subject's medical file.

According to some embodiments of the invention, the at least one oligonucleotide does not exceed 20 oligonucleotides.

According to some embodiments of the invention, the mutation in the BRCA1 sequence is selected from the group consisting of a BRCA1 185delAG (a deletion of the adenosine and guanine nucleotides at position 2288-2289 of SEQ ID NO:4) and BRCA1 5382insC (an insertion of a cytosine nucleotide at position 69293 of SEQ ID NO:4).

According to some embodiments of the invention, the mutation in the BRCA2 sequence is the BRCA2 6174delT (a deletion of a thymidine nucleotide at position 26050 of SEQ ID NO:5).

According to some embodiments of the invention, the individual exhibits the increased predisposition to the cancer, the method further comprising subjecting the individual to a gold-standard diagnostic test to diagnose the cancer.

According to some embodiments of the invention, the cancer is breast cancer.

According to some embodiments of the invention, the cancer is ovarian cancer.

According to some embodiments of the invention, determining the presence or the absence of the KLOTHO functional variant is performed in a biological sample of the individual.

According to some embodiments of the invention, determining the presence or the absence of the KLOTHO functional variant is effected using a DNA detection method.

According to some embodiments of the invention, determining the presence or the absence of the KLOTHO functional variant is effected using a protein detection method.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic presentation of the genomic region on chromosome 13 [1.2 Mb (megabases) altogether] depicting the markers and polymorphisms in the BRCA2-Klotho region used to reconstruct the haplotype and determine linkage between the two genes. Presented are the 6174delT mutation in the BRCA2 gene, the polymorphic markers D135171, D13S1695 D1351493, and the V/S 352 klotho mutation. The relative distances between the genes and markers are provided in kb (kilobases).

FIG. 2 is a graph depicting the age at breast cancer presentation (diagnosis) among BRCA1 mutation(s) carriers according to KLOTHO genotype. FF, wild type KLOTHO; VF and VV, patients heterozygotes or homozygotes for the KLOTHO functional variant, respectively. The median age at diagnosis was 48 years for FF/BRCA1 genotype, 43 years for FV/BRCA1 genotype and 53 for VV/BRCA1 genotype (p=0.04 for FF/BRCA1 vs. FV/BRCA1, p=0.186 for VV/BRCA1 vs. FF/BRCA1 and p=0.02 for differences between the three groups). “Survival”=being disease free (free of the breast cancer).

FIGS. 3A-L depict the reduced growth inhibitory effect of klotho functional variant in breast cancer cells. MCF-7 (FIGS. 3A-C), T-47D (FIGS. 3D-F) and HCC-1973 (FIGS. 3G-I) cells were transfected with either an empty vector (pEF; FIGS. 3A, D, G), human klotho (pEFhKL, FIGS. 3B, E, H) or human klotho-V expression vector, in which phenylalanine at position 352 has been substituted to valine (pEFhKL-V; FIGS. 3C, F, I) and were grown in G418 for 2 weeks. Colonies were stained with crystal violet and photographed. FIGS. 3J-L: quantification of at least three independent experiments; FIG. 3J-T—47D cells; FIG. 3K—MCF-7 cells; FIG. 3L—HCC-1937 cell. Lane 1 (pEF), lane 2 (pEFhKL), lane 3 (pEFhKL-V). Data are shown as mean±S.D. (*) indicates p<0.05 for the difference between pEFKL and pEFhKL-V;

FIGS. 4A-C are Western blots depicting secretion of klotho functional variant in breast cancer cells. MCF-7 cells were transfected as described for FIGS. 3A-L above and 48 hours after transfection the medium was replaced with serum-free medium and 6 hours later the medium was collected to assess klotho secretion, or cells were washed twice with PBS for the analysis of klotho expression in the cell lysate. Albumin served as a carrier for klotho precipitation in the medium, and thus served also as media loading control. FIG. 4A—klotho expression in the medium (secreted); FIG. 4B—klotho expression in cell lysate; FIG. 4C—albumin expression in medium.

### DESCRIPTION OF SPECIFIC EMBODIMENTS OF THE INVENTION

The present invention, in some embodiments thereof, relates to methods and kits for determining predisposition to cancer and/or prognosis of the cancer by determining the presence or absence of the Klotho functional variant, and to methods of designing treatment regimen and treating the cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that presence of the functional variant of the KLOTHO gene (e.g., SNPs F352V or C370S in SEQ ID NO:7, and/or G1163A in SEQ ID NO:6) in a heterozygote form is highly associated with cancer and thus can be used in determining predisposition to cancer.

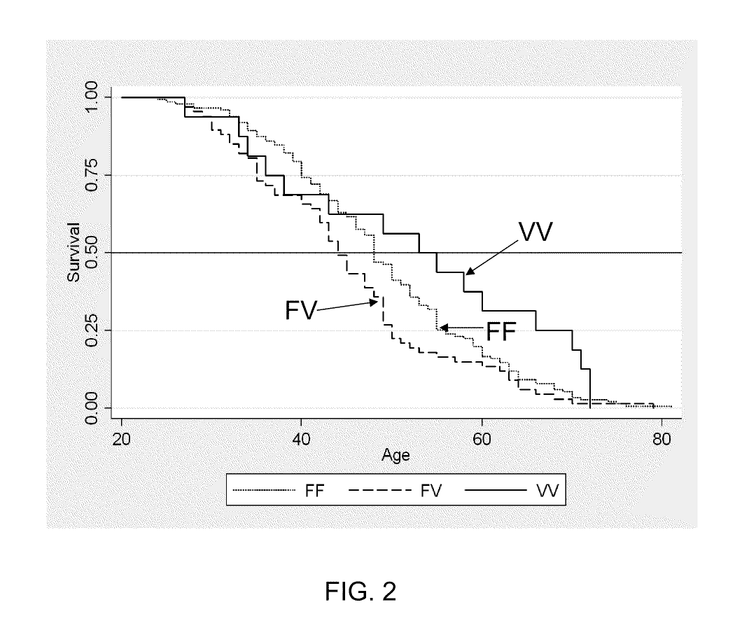

As shown in the Examples section which follows, the KLOTHO functional variant was significantly more frequent among subjects with breast or ovarian cancer than among healthy individuals (not affected by the cancer), which carry a BRCA1 mutation (e.g., the 185delAG and 5382insC mutations) or a BRCA2 mutation (e.g., the 6174delT mutation) (Tables 4 and 5, Example 1). In addition, as shown in FIG. 2 and described in Example 1 of the Examples section which follows, the presence of the KLOTHO functional variant in a heterozygote form among carriers of the BRCA1 mutation(s) was associated with a significantly younger age (5-10 years younger) of breast cancer diagnosis as compared to subjects who are homozygous to the wild-type allele (common allele, e.g., Phenylalanine amino acid residue at position 352 of SEQ ID NO:7) or the functional variant allele (rare allele, e.g., Valine amino acid residue at position 352 of SEQ ID NO:7). Moreover, the present inventors have uncovered that the KLOTHO functional variant is in linkage disequilibrium with the BRCA2 6174delT mutation (Table 6, Example 2) and that the presence of the KLOTHO functional variant in a homozygous or heterozygote form is associated with the presence of cancer (e.g., breast cancer and/or ovarian cancer) among carriers of the BRCA2 6174delT mutation (Table 7, Example 2).

Thus, according to an aspect of some embodiments of the invention there is provided a method of determining if an individual is predisposed to cancer. The method is effected by determining a presence or an absence of the KLOTHO functional variant in a heterozygote form, wherein the presence of the KLOTHO functional variant in the heterozygote form is indicative of an increased predisposition to the cancer, thereby determining if the individual is predisposed to the cancer.

As used herein, the term “individual” or “subject”, which may be interchangeably used herein, includes a mammal, preferably a human being (male or female) at any age.

According to some embodiments of the invention, the term “individual” encompasses an individual who is at risk (i.e., predisposed) of developing cancer.

For example, the individual can be at risk of having cancer if a relative of the individual (e.g., first or second degree relative, such as a parent, a sibling, a grandparent, an aunt, an uncle, or a child) is/was diagnosed with the cancer. In addition, the individual can be at risk of having the cancer based on a genetic predisposition to cancer such as presence of a mutation in the BRCA1 or BRCA2 genes. It should be noted that the risk of developing cancer can be of the same type of cancer or of a related type of cancer. For example, if the relative of the individual has breast cancer, then the individual is at risk of having breast cancer, ovarian cancer, or pancreatic cancer.

According to some embodiments of the invention, the individual is of the Ashkenazi Jewish population.

As used herein, the term “predisposed” when used with respect to cancer refers to an individual who is more susceptible to develop cancer than non-predisposed individuals.

It should be noted that the predisposition is determined when the subject is free of the cancer or not yet diagnosed with the cancer.

The cancer according to some embodiments of the invention can be any solid tumor, non-solid tumor and/or cancer metastases. Examples include, but are not limited to breast cancer, ovarian cancer, pancreatic cancer, stomach cancer, colon cancer and prostate cancer.

According to some embodiments of the invention the cancer is breast cancer.

According to some embodiments of the invention the cancer is ovarian cancer.

The term “KLOTHO” encompasses the nucleic acid sequence [i.e., the KLOTHO genomic sequence (nucleotides 33590571-33640282 of GenBank Accession No. NC_{—}000013.10; SEQ ID NO:20); the KLOTHO mRNA transcript (GenBank Accession No. NM_{—}004795.3 (SEQ ID NO:6)] and/or the amino acid sequence encoding the Klotho protein [GenBank Accession No. NP_{—}004786 (SEQ ID NO:7)].

The phrase “KLOTHO functional variant” refers to the presence of at least one single nucleotide polymorphism (SNP) or several SNPs in the KLOTHO gene/mRNA/protein, which are known to be in complete linkage disequilibrium (Arking D E et al., 2002, Proc Natl Acad Sci 99: 856-861). Examples of SNPs include:

1. “F352V” is a Phe→Val substitution at position 352 of the Klotho protein; position relates to the amino acid sequence set forth in GenBank Accession No. NP_{—}004786 (SEQ ID NO:7), which is encoded by the polymorphism “T1062G” [nucleotide position relates to GenBank Accession No. NM_{—}004795.3 (SEQ ID NO:6)]. This SNP is also known as rs9536314 (SEQ ID NO:1);

2. “C370S” is a Cyt→Ser substitution at position 370 of the Klotho protein; position relates to the amino acid sequence set forth in GenBank Accession No. NP_{—}004786 (SEQ ID NO:7), which is encoded by the polymorphism “G1117C” [nucleotide position relates to GenBank Accession No. NM_{—}004795.3 (SEQ ID NO:6)];

3. “K384K”, silent mutation at position “1163 G→A” (i.e., a substitution of a guanine nucleotide with an adenosine nucleotide at position 1163 of GenBank Accession No. NM_{—}004795.3 (SEQ ID NO:6), which does not affect the encoded Klotho protein);

4. “−79C→T”, a substitution of a cytosine nucleotide with a thymidine nucleotide at position −79 with respect to exon 2 first nucleotide (i.e., 79 nucleotides upstream of the intron1/exon 2 splice site), which corresponds to nucleotide 37255 in SEQ ID NO:20;

5. “−46C→G”, a substitution of a cytosine nucleotide with a guanine nucleotide at position −46 with respect to exon 2 first nucleotide (i.e., 46 nucleotides upstream of the intron1/exon 2 splice site), which corresponds to nucleotide 37288 in SEQ ID NO:20;

6. “+143T→G”, a substitution of a thymidine nucleotide with a guanine nucleotide at position +143 with respect to exon 2 last nucleotide (i.e., 143 nucleotides downstream of the exon 2/intron 2 splice site), which corresponds to nucleotide 37987 in SEQ ID NO:20;

Table 1, hereinbelow, provides the positions of the intron/exon boundaries in the KLOTHO gene and the corresponding nucleotides in the genomic (SEQ ID NO:20) or mRNA (SEQ ID NO:6) sequences.

TABLE 1
Genomic structure and intron/econ boundaries of KLOTHO

| | Genomic | mRNA | Length |
|---|---|---|---|
| EXON | coordinates | coordinates | (nucleotides) |
| Exon 1 | 1-827 | 1-827 | 827 |
| Exon 2 | 37334-37844 | 828-1338 | 511 |
| Exon 3 | 38614-38882 | 1339-1607 | 269 |
| Exon 4 | 44246-45347 | 1608-2709 | 1102 |
| Exon 5 | 47416-49710 | 2710-5004 | 2295 |

Genomic coordinates relate SEQ ID NO: 20 (GenBank Accession No. NC_000013, nucleotides 33590571-33640282); mRNA coordinates relate to SEQ ID NO: 6 (GenBank Accession No. NM_004795.3).

The term “polymorphism” refers to the occurrence of two or more genetically determined variant forms (alleles) of a particular nucleic acid or a particular amino at a frequency where the rarer (or rarest) form could not be maintained by recurrent mutation alone. A single nucleotide polymorphism (SNP) results from a single base difference between related alleles at the same genetic locus.

It should be noted that other SNPs in the KLOTHO gene [e.g., neighboring genotypes (SNPs) or loci] which are in linkage disequilibrium with any of the above described SNPs in the KLOTHO gene (e.g., “F352V”, “C370S”, “1163 G→A”, “−79C→T”, “−46C→G”, and “+143T→G” as described above) can be used to determine the predisposition of the cancer according to the teachings of the invention.

The phrase “linkage disequilibrium” (LD) is used to describe the statistical correlation between two neighboring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) [Devlin B, Risch N. (1995). A comparison of linkage disequilibrium measures for fine-scale mapping. Genomics. 29: 311-322.]. Two loci with a LD value of 1 are the to be in complete LD. At the other extreme, two loci with a LD value of 0 are termed to be in linkage equilibrium. Linkage disequilibrium is calculated following the application of the expectation maximization algorithm (EM) for the estimation of haplotype frequencies [Slatkin M, Excoffier L. (1996). Testing for linkage disequilibrium in genotypic data using the Expectation-Maximization algorithm. Heredity. 76: 377-83.]. According to some embodiments of the invention, LD values according to the present invention for neighboring genotypes (SNPs)/loci are selected above 0.1, preferably, above 0.2, more preferable above 0.5, more preferably, above 0.6, still more preferably, above 0.7, preferably, above 0.8, more preferably above 0.9, ideally about 1.0.

According to some embodiments of the invention, the presence of the KLOTHO functional variant is determined by one or several SNPs of the KLOTHO functional variant (e.g., one or several or all of “F352V”, “C370S”, “1163 G→A”, “−79C→T”, “−46C→G”, and “+143T→G” as described above).

According to some embodiments of the invention, the KLOTHO functional variant comprises a SNP selected from the group consisting of a valine amino acid residue at position 352 of the Klotho protein set forth by SEQ ID NO:7, a guanine nucleotide at position 1062 of the KLOTHO transcript set forth by SEQ ID NO:6, a serine amino acid residue at position 370 of the klotho protein set forth by SEQ ID NO:7, a cytosine nucleotide at position 1117 of the KLOTHO transcript set forth by SEQ ID NO:6, and an adenosine nucleotide at position 1163 of the KLOTHO transcript set forth by SEQ ID NO:6.

According to some embodiments of the invention, the KLOTHO functional variant comprises a valine amino acid residue at position 352 of the Klotho protein set forth by SEQ ID NO:7 or a guanine nucleotide at position 1062 of the KLOTHO transcript set forth by SEQ ID NO:6.

According to some embodiments of the invention, the KLOTHO functional variant comprises a guanine nucleotide at position 1062 of SEQ ID NO:6.

According to some embodiments of the invention, the KLOTHO functional variant comprises a cytosine nucleotide at position 1117 of SEQ ID NO:6.

According to some embodiments of the invention, the KLOTHO functional variant comprises a thymidine nucleotide at position 37255 of SEQ ID NO:20 (“−79C→T”).

According to some embodiments of the invention, the KLOTHO functional variant comprises a guanine nucleotide at position 37288 of SEQ ID NO:20 (“−46C→G”).

According to some embodiments of the invention, the KLOTHO functional variant comprises a guanine nucleotide at position 37987 of SEQ ID NO:20 (“+143T→G”).

As mentioned, the method is effected by determining the presence of the KLOTHO functional variant in the heterozygote form.

The term “heterozygous” as used herein refers to two different alleles of a certain polymorphism. The term “homozygous” as used herein refers to two identical alleles of a certain polymorphism.

The term “absence” as used herein in regard to the genotype of the KLOTHO functional variant describes the negative result of a specific genotype determination test. For example, if the genotype determination test is suitable for the identification of guanine nucleotide at position 1062 of SEQ ID NO:6 and the individual on which the test is performed is homozygote to the thymidine nucleotide at position 1062 of SEQ ID NO:6, then the result of the test will be “absence of genotype”.

It should be note that absence of a genotype in a heterozygote form can be presence of a genotype in a homozygote form. For example, an individual can be heterozygote to the “T1062G” SNP at SEQ ID NO:6 (i.e., presence of genotype in a heterozyote form), homozygote to the “T”-allele at position 1062 of SEQ ID NO:6 (i.e., absence of genotype in a heterozygote form) or homozygote to the “G”-allele at position 1062 of SEQ ID NO:6 (i.e., absence of genotype in a heterozygote form).

According to some embodiments of the invention, the heterozygote form of the KLOTHO functional variant comprises a Klotho protein with a phenylalanine amino acid residue at position 352 of SEQ ID NO:7 and a Klotho protein with a valine amino acid residue at position 352 of SEQ ID NO:7.

According to some embodiments of the invention, the heterozygote form of the KLOTHO functional variant comprises a guanine nucleotide at position 1062 of SEQ ID NO:6 and a thymidine nucleotide at position 1062 of SEQ ID NO:6.

According to some embodiments of the invention, the heterozygote form of the KLOTHO functional variant comprises a Klotho protein with a cysteine amino acid residue at position 370 of SEQ ID NO:7 and a Klotho protein with a serine amino acid residue at position 352 of SEQ ID NO:7.

According to some embodiments of the invention, the heterozygote form of the KLOTHO functional variant comprises cytosine nucleotide at position 1117 of SEQ ID NO:6 and a guanine nucleotide at position 1117 of SEQ ID NO:6.

According to some embodiments of the invention, the heterozygote form of the KLOTHO functional variant comprises a thymidine nucleotide at position 1062 of SEQ ID NO:6 and a guanine nucleotide at position 1062 of the KLOTHO transcript set forth by SEQ ID NO:6.

According to some embodiments of the invention, the heterozygote form of the KLOTHO functional variant comprises a guanine nucleotide at position 1163 of SEQ ID NO:6 and a Klotho gene with an adenosine nucleotide at position 1163 of the KLOTHO transcript set forth by SEQ ID NO:6.

According to some embodiments of the invention, the heterozygote form of the KLOTHO functional variant comprises a cytosine nucleotide at position 37255 of SEQ ID NO:20 and a Klotho gene with a thymidine nucleotide at position 37255 of SEQ ID NO:20 (“−79C/T”).

According to some embodiments of the invention, the heterozygote form of the KLOTHO functional variant comprises a cytosine nucleotide at position 37288 of SEQ ID NO:20 and a guanine nucleotide at position 37288 of SEQ ID NO:20 (“−46C/G”).

According to some embodiments of the invention, the heterozygote form of the KLOTHO functional variant comprises a thymidine nucleotide at position 37987 of SEQ ID NO:20 and a guanine nucleotide at position 37987 of SEQ ID NO:20 (“+143T/G”).

The presence or absence of the genotype can be determined ex vivo (i.e., outside of the subject's body) on a biological sample of the individual. Non-limiting of biological samples which can be used according to the method of some embodiments of the invention include a blood sample (e.g., peripheral blood cells, obtained using a syringe), skin cells (obtained from a skin biopsy), mouth epithelial cells (obtained from a mouth wash), a saliva sample, a seat sample, a bone marrow sample, a muscle sample, a cartilage sample, uterine cells, reproductive fluid cells, a lymph node sample, intestinal mucosal cells present in feces, buccal cells and the like. Such samples can be collected using effusions, scrapes, fine needle aspirates, peripheral blood scrapings, paraffin embedded tissues, frozen sections and the like.

The biological sample can be processed using methods known in the art, and can include a DNA, an RNA or a protein sample. Methods of extracting DNA, RNA or protein samples from biological samples are well known in the art.

Determination of the presence or absence of the KLOTHO functional variant can be performed using a DNA detection method.

One option is to determine a sequence variation in the entire gene sequence of a PCR reaction product. Alternatively, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Following is a non-limiting list of SNPs detection methods which can be used to identify one or more of the SNPs or DNA sequence variations (e.g., a deletion, insertion, duplication, inversion, substitution) described throughout the instant application: Restriction fragment length polymorphism (RFLP) (Gogos et al., Nucl. Acids Res., 18:6807-6817, 1990); Allele specific oligonucleotide (ASO) (Conner et al., Proc. Natl. Acad. Sci., 80:278-282, 1983); Denaturing/Temperature Gradient Gel Electrophoresis (DGGE/TGGE) (Abrams et al., Genomics 7:463-475, 1990; Sheffield et al., Proc. Natl. Acad. Sci., 86:232-236, 1989; and Lerman and Silverstein, Meth. Enzymol., 155:482-501, 1987; Wartell et al., Nucl. Acids Res., 18:2699-2701, 1990; Smith et al., Genomics 3:217-223, 1988; Borrensen et al., Proc. Natl. Acad. Sci. USA 88:8405, 1991; Scholz, et al., Hum. Mol. Genet. 2:2155, 1993); Single-Strand Conformation Polymorphism (SSCP) (Hayashi, PCR Meth. Appl., 1:34-38, 1991; Orita, et al., Genomics 5:874-879, 1989); Dideoxy fingerprinting (ddF) (Liu and Sommer, PCR Methods Appli., 4:97, 1994); Pyrosequencing™ analysis (Pyrosequencing, Inc. Westborough, Mass., USA); Acycloprime™ analysis (Perkin Elmer, Boston, Mass., USA); Reverse dot blot; dynamic allele-specific hybridization (DASH, Howell, W. M. et al., 1999. Dynamic allele-specific hybridization (DASH). Nat. Biotechnol. 17: 87-8); microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5]; the TaqMan system (Holland, P. M. et al., 1991. Detection of specific polymerase chain reaction product by utilizing the 5′→3′ exonuclease activity of Thermus aquaticus DNA polymerase. Proc Natl Acad Sci U S A. 88: 7276-80); various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference; Genetic Bit Analysis (GBA™) which is described by Goelet, P. et al. (PCT Appl. No. 92/15712); peptide nucleic acid (PNA, Ren B, et al., 2004. Nucleic Acids Res. 32: e42) and locked nucleic acids (LNA, Latorra D, et al., 2003. Hum. Mutat. 22: 79-85) probes; Molecular Beacons (Abravaya K, et al., 2003. Clin Chem Lab Med. 41: 468-74); intercalating dye [Germer, S. and Higuchi, R. Single-tube genotyping without oligonucleotide probes. Genome Res. 9:72-78 (1999)]; FRET primers (Solinas A et al., 2001. Nucleic Acids Res. 29: E96); AlphaScreen (Beaudet L, et al., Genome Res. 2001, 11(4): 600-8); SNPstream (Bell P A, et al., 2002. Biotechniques. Suppl.: 70-2, 74, 76-7); Multiplex minisequencing (Curcio M, et al., 2002. Electrophoresis. 23: 1467-72); SnaPshot (Turner D, et al., 2002. Hum Immunol. 63: 508-13); MassEXTEND (Cashman J R, et al., 2001. Drug Metab Dispos. 29: 1629-37); GOOD assay (Sauer S, and Gut IG. 2003. Rapid Commun. Mass. Spectrom. 17: 1265-72); Microarray minisequencing (Liljedahl U, et al., 2003. Pharmacogenetics. 13: 7-17); arrayed primer extension (APEX) (Tonisson N, et al., 2000. Clin. Chem. Lab. Med. 38: 165-70); Microarray primer extension (O'Meara D, et al., 2002. Nucleic Acids Res. 30: e75); Tag arrays (Fan J B, et al., 2000. Genome Res. 10: 853-60); Template-directed incorporation (TDI) (Akula N, et al., 2002. Biotechniques. 32: 1072-8); fluorescence polarization (Hsu T M, et al., 2001. Biotechniques. 31: 560, 562, 564-8); Colorimetric oligonucleotide ligation assay (OLA, Nickerson D A, et al., 1990. Proc. Natl. Acad. Sci. USA. 87: 8923-7); Sequence-coded OLA (Gasparini P, et al., 1999. J. Med. Screen. 6: 67-9); Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72); coded microspheres (Rao K V et al., 2003. Nucleic Acids Res. 31: e66); and MassArray (Leushner J, Chiu N H, 2000. Mol Diagn. 5: 341-80), each of which is hereby incorporated by reference in its entirety.

Non-limiting examples of oligonucleotides which can be used to detect the KLOTHO functional variant include the PCR primers set forth by SEQ ID NOs:2 and 3; SEQ ID NOs:8 and 9 which were used for PCR-RFLP analysis; and the primers used for the 5′ nuclease assay (TaqMan) and set forth by SEQ ID NOs:10 and 11 (for PCR amplification) and SEQ ID NOs: 11 and 12 (as probes).

Determination of the presence or absence of the KLOTHO functional variant can be performed using a variant specific primer(s) [e.g., oligonucleotide(s)] or labeled probe(s) in an RNA detection method such as Northern blot analysis and RT-PCR analysis.

Determination of the presence or absence of the KLOTHO functional variant can be performed using an antibody which specifically binds one protein polymorph (e.g., the V352 polymorph of SEQ ID NO:7 or the S370 polymorph of SEQ ID NO:7) but not the other polymorph (e.g., the F352 or the C370 polymorphs of SEQ ID NO:7, respectively). Such antibodies can be used in various protein detection methods known in the art such as Enzyme linked immunosorbent assay (ELISA); Western blot analysis; Radio-immunoassay (RIA); Fluorescence activated cell sorting (FACS); and Immunohistochemical analysis.

According to the method of some embodiments of the invention, the presence of the KLOTHO functional variant in the heterozygote form is indicative of an increased predisposition to the cancer.

The predisposition to the cancer can be quantified by generating and using Hazard Ratio (HR) values. The HR is used to show the occurrence of an event (risk of cancer) over time. Thus, the HR is an estimate of the ratio of the hazard rate in an individual with a particular genotype (e.g., presence of SNP) versus an individual with the “control” genotype (e.g., absence of SNP). The hazard rate is the probability that if the event in question has not already occurred, it will occur in the next time interval, divided by the length of the interval. The time interval is made very short, so that in effect the hazard rate represents an instantaneous rate. An assumption of proportional hazards regression is that the hazard ratio is constant over time. The HR can be calculated by the Cox regression analysis, log-rank analysis and/or Wilcoxon two-sample test essentially as described in Spruance S. L., et al., 2004, Antimicrobial Agents and Chemotherapy, 48:2787-2792; which is hereby incorporated by reference in it entirety.

It should be noted that since the predisposition to the cancer indicates an increased risk to have the cancer at a certain age, the HR values evaluates the predisposition to the cancer over time.

The predisposition to cancer can be also quantified by generating and using genotype relative risk (GRR) values. The GRR is the increased chance of an individual with a particular genotype to develop the disease. Thus, the GRR of the risk genotype G, with respect to the protective genotype G₀, is the ratio between the risk of an individual carrying genotype G to develop the disease, and the risk of an individual carrying genotype G₀ to develop the disease. The GRR used herein is represented in terms of an appropriate odds ratio (OR) of G versus G₀ in cases and controls. Moreover, computation of GRR of haplotypes is based on a multiplicative model in which the GRR of an homozygote individual is the square of the GRR of an heterozygote individual. For further details see Risch and Merikangas, 1996 [The future of genetic studies of complex human diseases. Science 273: 1516-1517].

Once calculated, the GRR can reflect the increased predisposition risk on an individual with a specific KLOTHO genotype to develop cancer.

As mentioned above and further shown in FIG. 2 and Example 1 of the Examples section which follows, the presence of the KLOTHO functional variant in a heterozygote form was associated with a significantly younger age (e.g., a median of 5-10 years younger) of breast cancer diagnosis as compared to a subjects who is not heterozygote to the KLOTHO functional variant [e.g., homozygous to the wild-type (common allele; e.g., Phenylalanine amino acid residue at position 352 of SEQ ID NO:7) or homozygous to the KLOTHO functional variant (rare allele; e.g., Valine amino acid residue at position 352 of SEQ ID NO:7)].

According to some embodiments of the invention, the predisposition comprises an increased risk to develop the cancer (e.g., to be diagnosed with the cancer) at an age which is at least about 1 year, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, at least about 20 years, at least about 21 years, at least about 22 years, at least about 23 years, at least about 24 years, at least about 25 years, at least about 26 years younger than the age of an individual who is not heterozygote to the KLOTHO functional variant.

According to some embodiments of the invention the individual of which the predisposition is determined is selected from the age of about 1-50 years, e.g., about 10-50 years, e.g., about 10-40 years, e.g., about 20-40 years, e.g., about 20-30 years, e.g., about 20-25 years.

As mentioned above and further described in Examples 1 and 2 of the Examples section which follows, the presence of the KLOTHO functional variant in the heterozygous form was associated with increased risk for cancer among carriers of mutations associated with cancer in the BRCA1 (breast cancer 1, early onset) gene [nucleotides 41196314-41277468 of GenBank Accession No. NC_{—}000017.1); GenBank Accession No. AY273801 (SEQ ID NO:4); BRCA1 mRNA sequence GenBank Accession No. U14680 (SEQ ID NO:27)], or in the BRCA2 (breast cancer 2, early onset) gene [nucleotides 32889617-32973809 of GenBank Accession No. NC_{—}000013.10; GenBank Accession No. AY436640 (SEQ ID NO:5); BRCA1 mRNA sequence GenBank Accession No. NM_{—}000059.1 (SEQ ID NO:28)].

As used herein the phrase “associated with the cancer” refers to a mutation which is known to increase the predisposition of a subject to the cancer based on genetic analysis and association studies.

The term “mutation” (also referred to as a “nucleic acid substitution”) refers to any mutation in the DNA sequence of an individual which can lead to cancer. Non-limiting examples of such nucleic acid changes include a missense mutation (i.e., a mutation which changes an amino acid residue in the protein with another amino acid residue), a nonsense mutation (i.e., a mutation which introduces a stop codon in a protein), a frameshift mutation (i.e., a mutation, usually, deletion or insertion of nucleic acids which changes the reading frame of the protein, and may result in an early termination or in a longer amino acid sequence), a readthrough mutation (i.e., a mutation which results in an elongated protein due to a change in a coding frame or a modified stop codon), a promoter mutation (i.e., a mutation in a promoter sequence, usually 5′ to the transcription start site of a gene, which result in up-regulation or down-regulation of a specific gene product), a regulatory mutation (i.e., a mutation in a region upstream or downstream, or within a gene, which affects the expression of the gene product), a deletion (i.e., a mutation which deletes coding or non-coding nucleic acids in a gene sequence), an insertion (i.e., a mutation which inserts coding or non-coding nucleic acids into a gene sequence), an inversion (i.e., a mutation which results in an inverted coding or non-coding sequence), and a duplication (i.e., a mutation which results in a duplicated coding or non-coding sequence).

It should be noted that once the association between mutations in the BRCA1 or BRCA2 genes was uncovered, hundreds of studies have confirmed such associations and various databases store the information regarding occurrences of mutations in these genes among patients diagnosed with various cancers such as breast cancer, ovarian cancer, pancreatic cancer and stomach cancer, prostate cancer, and colon cancer. A non-limiting example of such a database include the “Breast Cancer Information Core Database” [Hypertext Transfer Protocol://research (dot) nhgri (dot) nih (dot) gov/projects/bic/Member/index.shtm1].

According to some embodiments of the invention, the method further comprising determining a presence or an absence of a mutation in the BRCA1 sequence (SEQ ID NO:4) or in the BRCA1 mRNA sequence (SEQ ID NO:27) which is associated with the cancer.

According to some embodiments of the invention, the method further comprising determining a presence or an absence of a mutation in the BRCA1 amino acid sequence set forth by SEQ ID NO:29, which is associated with the cancer.

Non-limiting examples of mutations in the BRCA1 mRNA (SEQ ID NO:27) or BRCA1 protein (SEQ ID NO:29), which are associated with cancer are described in Table 2, below.

TABLE 2
Mutations in BRCA1 associated with cancer

| Frameshift | Missense | Nonsense |
|---|---|---|
| 185delAG | C61G | R1443X |
| 5382insC | R1347G | E1250X |
| 4184del4 | M1008I | Q563X |
| 3875del4 | R841W | R1835X |
| exon13ins6kb | M1628T | E143X |
| 2800delAA | R496H | E908X |
| 1675delA | Q356R | Y1563X |
| 1294del40 | L246V | Q780X |
| 2576delC | P1637L | K679X |
| 3819del5 | V772A | R1751X |
| 2804delAA | F486L | Q1395X |
| 3600del11 | N550H | R1203X |
| 2594delC | Y179C | Y978X |
| 4154delA | S1512I | W1508X |
| 1135insA | A1708E | Q1200X |
| 5083del19 | S1040N | W321X |
| 3450del4 | R496C | S713X |
| 5296del4 | P1238L | Y1703X |
| 2798del4 | M1652I | Q1408X |
| 3604delA | T826K | W1782X |

Provided are the most frequent mutations in the BRCA1 gene which are associated with breast cancer, ovarian cancer, pancreatic cancer, stomach cancer and colon cancer. The positions of the mutations refer to the BRCA1 mRNA sequence set forth by GenBank Accession No. U14680 (SEQ ID NO: 27) (in case the mutation is designated by nucleotide change, i.e., the frameshift mutations); or to the BRCA1 protein sequence set forth by GenBank Accession No. AAA73985.1 (SEQ ID NO: 29) (in case the mutation is designated by amino acid change, i.e., the missense or nonsense mutations). For further details regarding these mutations see the “Breast Cancer Information Core Database” [Hypertext Transfer Protocol ://research (dot) nhgri (dot) nih (dot) gov/projects/bic/Member/index.shtml].

According to some embodiments of the invention, the mutation in the BRCA1 sequence (SEQ ID NO:4) is selected from the group consisting of a BRCA1 185delAG (a deletion of the adenosine and guanine nucleotides at position 2288-2289 of SEQ ID NO:4) and BRCA1 5382insC (an insertion of a cytosine nucleotide at position 69293 of SEQ ID NO:4).

Non-limiting examples of oligonucleotides which can be used to detect the BRCA1 185delAG mutation include the PCR primers set forth by SEQ ID NOs:21 and 22; Non-limiting examples of oligonucleotides which can be used to detect the BRCA1 5382insC mutation include the PCR primers set forth by SEQ ID NOs:23-24.

According to some embodiments of the invention, the method further comprising determining a presence or an absence of a mutation in the BRCA2 sequence set forth by SEQ ID NO:5 or in the BRCA2 mRNA (SEQ ID NO:28), which is associated with the cancer.

According to some embodiments of the invention, the method further comprising selecting the individual as being a carrier of a mutation in the BRCA2 amino acid sequence set forth by SEQ ID NO:30, which is associated with the cancer.

Non-limiting examples of mutations in the BRCA2 mRNA (SEQ ID NO:28) or in the BRCA2 protein (SEQ ID NO:30), which are associated with cancer are described in Table 3, below.

TABLE 3
Mutations in BRCA2 associated with cancer

| Frameshift | Missense | Nonsense |
|---|---|---|
| 6174delT | I2490T | K3326X |
| 3036del4 | D1420Y | Y1894X |
| 6503delTT | E2856A | R3128X |
| 2041insA | P655R | R2520X |
| 8765delAG | S384F | E1953X |
| 4075delGT | Y42C | S1882X |
| 983del4 | I505T | Y1655X |
| 2157delG | R2108H | Y3098X |
| 5950delCT | I2944F | E49X |
| 8525delC | K2950N | K2013X |
| 886delGT | A2717S | E1308X |
| 9132delC | I3412V | S1955X |
| 4706del4 | D935N | S611X |
| 5578delAA | S326R | Y3308X |
| 802delAT | R2034C | S1970X |
| 5301insA | H2116R | Q2960X |
| 5804del4 | D1902N | R2494X |
| 5164del4 | V2728I | W2586X |
| 9663delGT | H2440R | K1530X |
| 7297delCT | I2285V | S1630X |

Provided are the most frequent mutations in the BRCA2 gene which are associated with breast cancer, ovarian cancer, pancreatic cancer, stomach cancer and colon cancer. The positions of the mutations refer to the BRCA2 mRNA sequence set forth by GenBank Accession No. NM_000059.1 (SEQ ID NO: 28) (in case the mutation is designated by nucleotide change, i.e., the frameshift mutations); or to the BRCA2 protein sequence set forth by GenBank Accession No. NP_000050.1 (SEQ ID NO: 30) (in case the mutation is designated by amino acid change, i.e., the missense or nonsense mutations). For further details regarding these mutations see the “Breast Cancer Information Core Database” [Hypertext Transfer Protocol ://research (dot) nhgri (dot) nih (dot) gov/projects/bic/Member/index.shtml].

According to some embodiments of the invention, the mutation in the BRCA2 sequence is the BRCA2 6174delT (a deletion of a thymidine nucleotide at position 26050 of SEQ ID NO:5).

Non-limiting examples of oligonucleotides which can be used to detect the BRCA2 6174delT mutation include the PCR primers set forth by SEQ ID NOs:25 and 26.

According to some embodiments of the invention, the method further comprising selecting the individual as being a carrier of a mutation in the BRCA1 sequence set forth by SEQ ID NO:4, in the BRCA1 mRNA (SEQ ID NO:27) or in the BRCA1 protein (SEQ ID NO:29), which is associated with the cancer.

According to some embodiments of the invention, the method further comprising selecting the individual as being a carrier of a mutation in the BRCA2 sequence set forth by SEQ ID NO:5, in the BRCA2 mRNA (SEQ ID NO:28) or in the BRCA2 protein (SEQ ID NO:30) which is associated with the cancer.

As used herein the phrase “selecting the individual as being a carrier of a mutation” refers to determining the presence of the KLOTHO functional variant in an individual whose carrier status (i.e., being a carrier or a non-carrier) with respect to a mutation in the BRCA1 or BRCA2 sequence which is associated with cancer (e.g., as listed in Tables 2 and 3 above) is already known from a previous genetic analysis which can be recorded in the subject's medical file/history.

According to an aspect of some embodiments of the invention there is provided a method of determining if an individual is predisposed to cancer. The method is effected by determining in an individual who is a carrier of a BRCA2 mutation, which is associated with the cancer, a presence or an absence of the KLOTHO functional variant in a heterozygote form or a homozygous form, wherein the presence of the KLOTHO functional variant in the heterozygote form or the homozygous form is indicative of an increased predisposition to the cancer, thereby determining if the individual is predisposed to the cancer.

According to some embodiments of the invention, the method further comprising selecting the individual as a female individual.

According to some embodiments of the invention, the method of some embodiments of the invention further comprising recording the increased predisposition to the cancer in the subject's medical file.

Recording the increased predisposition to the cancer in the subject's medical file may assist in the diagnosis of the cancer. Thus, taking into consideration that the subject has an increased predisposition to the cancer, the subject may be advised to check for early signs of the cancer, and be subjected to the gold-standard diagnostic tests for identifying early signs of the cancer at an age which is much younger than recommended for the entire population, or even for individuals who are at risk to develop the cancer based on a family history (having a first or second degree relative diagnosed with the cancer) or based on predisposition to the cancer due to mutations in other genes (e.g., in BRCA1 or BRCA2).

According to some embodiments of the invention, the method further comprising informing the individual of the presence of the increased predisposition to the cancer.

As used herein the phrase “informing the individual” refers advising the individual that based on the increased predisposition (risk) to develop the cancer the individual should take precautions (e.g., being subject to gold-standard diagnostic test, undergo preventing surgeries), which might prevent the cancer or reduce its deleterious effects.

According to some embodiments of the invention, in cases of increased predisposition to the cancer the method further comprising subjecting the individual to a gold-standard diagnostic test and/or to the currently used screening and/or diagnostic tests to diagnose the cancer.

For example, the gold-standard diagnostic test and/or currently used screening and/or diagnostic tests for breast cancer include a physical examination, mammography, an ultrasound scan of the breast and lymph nodes, an X-ray computed tomography (CT) scan of the chest (and optionally the entire body), a magnetic resonance imaging (MRI) of the chest (and optionally the entire body), histological examination of a tissue biopsy (e.g., lymph node, breast tissue), a blood test [e.g., for detection of the CA-15.3, CA125, and/or carcino-embrionic antigen (CEA) markers] and/or a surgical examination of breast and lymph node tissues.

For example, the gold-standard diagnostic test and/or currently used screening and/or diagnostic tests for ovarian cancer include physical examination, an ultrasound scan of the uterus, cervix and ovaries, an X-ray computed tomography (CT) scan, a magnetic resonance imaging (MRI), histological examination of a tissue biopsy which can be obtained in various ways including surgery, a blood test (e.g., for detection of the CA-125 marker).

For example, the gold-standard diagnostic test and/or currently used screening and/or diagnostic tests for pancreatic cancer can be an ultrasound scan of the internal organs including pancreas, liver, stomach, endoscopic ultrasound scan of internal organs (EUS), an X-ray computed tomography (CT) scan, a magnetic resonance imaging (MRI), histological examination of a tissue biopsy of the pancreas and/or liver and/or suspected lesion (e.g. peritoneal), a blood test (e.g., for detection of the CA19.9 marker).

According to some embodiments of the invention, subjecting the individual to the gold-standard diagnostic tests, and/or the currently used screening or diagnostic tests is effected at an earlier age than recommended to an age-matched individual who is not predisposed to the cancer.

According to some embodiments of the invention, based on the increased predisposition to the cancer as determined by the method of the invention, the gold-standard diagnostic tests, and/or the currently used screening or diagnostic tests is performed at least about 1 year, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, at least about 20 years, at least about 21 years, at least about 22 years, at least about 23 years, at least about 24 years, at least about 25 years earlier than recommended for an individual who is not predisposed to the cancer.

Thus, the teachings of the invention enable earlier detection of cancer and thus assist in diagnosing the cancer.

As used herein the term “diagnosing” refers to determining presence or absence of a pathology (e.g., cancer), classifying a pathology or a symptom, determining a severity of the pathology, monitoring pathology progression, forecasting an outcome of a pathology and/or prospects of recovery and screening of a subject for a specific disease.

It should be noted that screening of the subject for a specific disease can be conducted on an asymptomatic individual (devoid of clinical signs of the disease).

The teachings of the invention enable the design of a treatment regimen to treat cancer in the subject.

As used herein the phrase “treatment regimen” refers to a treatment plan that specifies the type of treatment, dosage, schedule and/or duration of a treatment provided to a subject in need thereof (e.g., a subject diagnosed with the cancer). The selected treatment regimen can be an aggressive one which is expected to result in the best clinical outcome (e.g., complete cure of the pathology) or a more moderate one which may relief symptoms of the pathology yet results in incomplete cure of the pathology. The type of treatment can include a surgical intervention [removal of lesion, diseased cells, tissue, or organ], a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

According to some embodiments of the invention, the treatment regimen comprises preventing the growth of the cancerous tumors by removing suspicious lesions, and/or undergoing preventing resection (removal) of an apparently non-diseased tissue such as a breast tissue or an ovary in which the cancerous tumor is expected to grow (based on the predisposition to the cancer).

Thus, the teachings of the invention can be also used to treat a subject who is predisposed to the cancer.

The term “treating” refers to inhibiting, preventing or arresting the development of a pathology (e.g., cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping the pathology from occurring in a subject who may be at risk for the disease, but has not yet been diagnosed as having the disease.

The present inventors have uncovered that expression of the KLOTHO functional variant (e.g., the 352V allele of SEQ ID NO:7) in breast cancer cells is less efficient in suppressing growth and colony formation of the cancerous cells, suggesting a worsened prognosis for cancer patients expressing the KLOTHO functional variant (Example 3 of the Examples section which follows). Moreover, the present inventors have uncovered that secretion of the Klotho functional variant protein from breast cancer cells is reduced as compared the Klotho wild-type protein, thus suggesting a reduced inhibitory effect on growth of cancerous cells (Example 4 of the Examples section which follows). These results suggest the use of the KLOTHO functional variant as a prognostic marker for a worsen (i.e., poor) prognosis among cancer patients.

Thus, according to an aspect of some embodiments of the invention there is provided a method of determining prognosis of cancer in an individual. The method is effected by determining a presence or an absence of the KLOTHO functional variant in a heterozygote or homozygous form wherein the presence of the KLOTHO functional variant in the heterozygote or the homozygous form is indicative of a worsen prognosis of the cancer as compared to the absence of the KLOTHO functional variant, thereby determining the prognosis of the cancer in the individual.

As used herein the phrase “determining prognosis” refers to predicting the outcome of the disease (cancer) and/or the prospects of recovery of the individual being affected by the disease.

For example, a good prognosis of the cancer can be being free of disease recurrence for more than 5 years from diagnosis, e.g., more than 6, 7, 8, 9, 10 or more years from diagnosis. On the other hand, a bad prognosis of the cancer can be a recurrence of the cancer (e.g., of a primary tumor in an additional tissue/area; or presence of cancer metastases) within less than 5 years from diagnosis, e.g., less than 4, 3, 2 or 1 year from diagnosis of the cancer.

As mentioned, the presence of the KLOTHO functional variant in a homozygous or heterozygous form is associated with a worsen prognosis of the disease.

According to some embodiments of the invention, a worsen prognosis is a recurrent of the disease within a shorter time (e.g., 1-5 years less) than expected in a subject having the same type of cancer but who is devoid of the KLOTHO functional variant.

It should be noted that determining the prognosis of the individual having the cancer may also affect the treatment regimen of the individual. For example, if the predicted prognosis is a worsen prognosis, then a more aggressive treatment can be offered to the affected individual.

The agents described hereinabove (e.g., the oligonucleotides, probes, antibodies) for detecting the predisposition to the cancer may be included in a diagnostic kit/article of manufacture preferably along with appropriate instructions for use and labels indicating FDA approval for use in predicting predisposition to cancer and/or the prognosis of the cancer.

The kit comprises at least one oligonucleotide or antibody for specifically determining a presence or an absence of the KLOTHO functional variant in a heterozygote form, and at least one oligonucleotide or antibody for specifically determining a presence or an absence of a mutation in the BRCA1 sequence set forth by SEQ ID NO:4 (BRCA1 genomic sequence), SEQ ID NO:27 (BRCA1 mRNA sequence) or SEQ ID NO:29 (BRCA1 amino acid sequence) and/or in the BRCA2 sequence set forth by SEQ ID NO:5 (BRCA2 genomic sequence), SEQ ID NO:28 (BRCA2 mRNA sequence) or SEQ ID NO:30 (BRCA2 amino acid sequence), the mutation in the BRCA1 or in the BRCA2 is associated with the cancer.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

According to some embodiments of the invention, the at least one oligonucleotide does not exceed 50 oligonucleotides, e.g., does not exceed 40 oligonucleotides, e.g., does not exceed 30 oligonucleotides, e.g., does not exceed 20 oligonucleotides.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

According to some embodiments of the invention, the kit further comprising instructions for use in determining if the individual is predisposed to the cancer, wherein the presence of the KLOTHO functional variant in a heterozygote form indicates of an increased predisposition risk of the individual to the cancer.

The kit can include, for example, at least one container including at least one of the above described diagnostic agents [e.g., KLOTHO oligonucleotides/antibody; BRCA1 oligonucleotides/antibody; BRCA2 oligonucleotides/antibody) and an imaging reagent packed in another container (e.g., enzymes, secondary antibodies, buffers, chromogenic substrates, fluorogenic material). The kit may also include appropriate buffers and preservatives for improving the shelf-life of the kit.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for

Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Subjects and clinical data—Study population included consecutive adult women of Ashkenazi Jewish ancestry who were consulted and tested at the Oncogenetics Unit and the Institute of Oncology of the Sheba Medical Center because of a family history suggestive of an inherited predisposition to breast or ovarian cancers. Women of Ashkenazi Jewish ancestry who were cancer free and had no family history of cancer served as control. All women were genotyped for the presence of three common mutations:

BRCA1 185delAG—a deletion of the “AG” (adenosine and guanine) nucleotides at position 2288-2289 of BRCA1 GenBank Accession No. AY273801 (SEQ ID NO:4); the mutation can be detected by PCR RFLP using the following PCR primers: 185delAG F: 5′-GAAGTTGTCATTTTATAAACCTTT (SEQ ID NO:21) and 185delAG R: 5′-TGACTTACCAGATGGGAGAC (SEQ ID NO:22).

BRCA1 5382insC—an insertion of “C” (cytosine) nucleotide at position 69293 of BRCA1 GenBank Accession No. AY273801 (SEQ ID NO:4)]; the mutation can be detected by PCR RFLP using the following PCR primers: 5382insC F: 5′-CCAAAGCGAGCAAGAGAATCAC (SEQ ID NO:23) and 5382insC R: 5′-GACGGGAATCCAAATTACACAG (SEQ ID NO:24).

BRCA2 6174de1T—a deletion of “T” (thymidine) nucleotide at position 26050 of BRCA2 GenBank Accession No. AY436640 (SEQ ID NO:5)]; the mutation can be detected by PCR RFLP using the following PCR primers: 6174delT F: 5′-TGGGATTTTTAGCACAGCACG (SEQ ID NO:25) and 6174delT R: 5′-CTGGTCTGAATGTTCGTTAC (SEQ ID NO:26).

Two additional cohorts were included in the study; 44 women who were screened at the Women's Cancer Research Institute at Cedars-Sinai cancer center in Los Angeles and found to be carriers of the BRCA2 (6174delT) mutations and 163 women who were screened at the Shaare Zedek Medical Center, Jerusalem, Israel and found to be carriers of a mutation in BRCA1 (185delAG or 5382insC). All participants were interviewed for demographic data and personal and family history of breast and ovarian cancers. Patients carrying more than one mutation (n=4) were excluded from the study. Four subsets of patients were therefore included: (i) women with no personal and family history of breast or ovarian cancer, (ii) women with breast or ovarian cancer non-carriers of the common BRCA1 or BRCA2 mutations, (iii) BRCA1 mutation carriers with or without breast or ovarian cancer and (iv) BRCA2 mutation carriers with or without breast or ovarian cancer. The study was approved by the Ethical Committees of the participating institutions and each participant signed a written informed consent.

The KL-VS variant is characterized by the following SNPs:

1. F352V is a Phe→Val substitution at position 352 of the Klotho protein; position relates to the amino acid sequence set forth in GenBank Accession No. NP_{—}004786 (SEQ ID NO:7), which is encoded by the polymorphism T1062G [nucleotide position relates to GenBank Accession No. NM_{—}004795.3 (SEQ ID NO:6)]. This SNP is also known as rs9536314 (SEQ ID NO:1).

2. C370S is a Cyt→Ser substitution at position 370 of the Klotho protein; position relates to the amino acid sequence set forth in GenBank Accession No. NP_{—}004786 (SEQ ID NO:7), which is encoded by the polymorphism G1117C [nucleotide position relates to GenBank Accession No. NM_{—}004795.3 (SEQ ID NO:6)];

3. K384K, silent mutation at position 1163 G→A (i.e., a substitution of a guanine nucleotide with an adenosine nucleotide at position 1163 of GenBank Accession No. NM_{—}004795.3 (SEQ ID NO:6), which does not affect the encoded

Klotho protein).

4. “−79C→T”, a substitution of a cytosine nucleotide with a thymidine nucleotide at position −79 with respect to exon 2 first nucleotide (i.e., 79 nucleotides upstream of the intron1/exon 2 splice site), which corresponds to nucleotide 37255 in SEQ ID NO:20.

5. “−46C→G”, a substitution of a cytosine nucleotide with a guanine nucleotide at position −46 with respect to exon 2 first nucleotide (i.e., 46 nucleotides upstream of the intron1/exon 2 splice site), which corresponds to nucleotide 37288 in SEQ ID NO:20.

6. “+143T→G”, a substitution of a thymidine nucleotide with a guanine nucleotide at position +143 with respect to exon 2 last nucleotide (i.e., 143 nucleotides downstream of the exon 2/intron 2 splice site), which corresponds to nucleotide 37987 in SEQ ID NO:20.

Genetic analysis—DNA was extracted from peripheral blood samples using the Promega DNA extraction kit (Promega corp., Madison, Wis., USA), according to the manufacturer's instructions and was tested for the three mutations common in the Ashkenazi Jewish population [BRCA1 (185delAG, 5382insC) and BRCA2 (6174delT)] as previously described [Rohlfs E M, Learning W G, Friedman K J,. Clin Chem 1997; 43:24-9].

Genotyping of the KL-VS variant was performed by identifying the presence of the Klotho F352V polymorphism using PCR-RFLP which detects the T1062G SNP in the Klotho mRNA, i.e., a T→G substitution at position 1062 of GenBank Accession No. NM_{—}004795.3 (SEQ ID NO:6). The success rate of the genotyping was 99%.

Restriction assay 1 (Sheba Medical Center)—Using a naturally occurring restriction site to distinguish the wild type allele from the variant allele. DNA was amplified using the following primers: Forward (F): 5′-GCCAAAGTCTGGCATCTCTA-3′ (SEQ ID NO:2) and Reverse (R): 5′-TTCCATGATGAACTTTTTGAGG-3′ (SEQ ID NO:3). PCR reactions were preformed in a 25 μl reaction containing 50-100 ng genomic DNA, PCR buffer (Peqlab, Germany), 2.5 mM MgCl₂, 250 nM dNTPs, 10 pmol of each primer and 1 U DNA Polymerase (Peqlab, Germany). PCR conditions were: denaturation (5 minutes, 94° C.), 35 cycles of denaturation at 94° C. for 30 seconds, annealing at 56° C. for 1 minute, elongation at 72° C. for 30 seconds, and extension (72° C,10 minutes). The resulting PCR product was subjected to restriction endonuclease digestion with 1 U MaeIII (Roche) (55° C., 16 hours) and separated on a 2.8% agarose gel. The KL-VS allele is characterized by additional MaeIII restriction site resulting in fragments of 178, 50, 267 bp, compared to only two fragments of 228 and 267 bp in the wild type allele.

Restriction assay 2 (Shaare Zedek Medical Center)—Introducing a novel restriction site by site directed mutagenesis using the following primers. Forward: 5′-GAAGAATGACCGACCACAG (SEQ ID NO:8) and a mismatched Reverse: 5′-ATGAACTTTTTCTCAGATTCTTTAA (SEQ ID NO:9; underlined nucleotides refer to site of restriction site inserted). PCR reactions were preformed in a 25 μl reaction containing 50-100 ng genomic DNA, PCR buffer (Medox Biotech, India), 2 mM MgCl₂, 200 nM dNTPs, 40 pmol of each primer, 10% DMSO and 0.25 U of SuperTherm DNA Polymerase (Medox Biotech, India). Amplification was carried out as follows: an initial denaturation step of 5 minutes at 94° C., followed by 35 cycles of denaturation at 94° C. for 30 seconds, annealing at 50° C. for 1 minute, elongation at 72° C. for 30 seconds, and a final extension step at 72° C. for 10 minutes. The PCR products were subjected to restriction endonuclease digestion with 10 U DraI (Fermentas, Vilnius, Lithuania) at 37° C. for 16 hours, separated on a 2.8% agarose gel stained with ethidium bromide and visualized. PCR product size 164 bp. The wild type allele is characterized by the artificial addition of DraI restriction site resulting in fragments of 138 and 26 bp.

Validation of the genetic analyses—Each method was validated by direct sequencing of at least 10% of the samples, and 172 samples were also examined using the 5′ nuclease assay. Both sequencing and 5′ nuclease assay fully correlated with the restriction analyses.

5′ nuclease assay (TaqMan)—The KL-VS variant was also genotyped by the 5′ nuclease assay (TaqMan) on the Roche LightCycler 480 Sequence Detection System (Roche applied science). PCR primers were: Forward primer 5′-GAGAAAAAGTTCATCAAAGGAACTGC-3′ (SEQ ID NO:10) and reverse primer 5′-CAATTGGCGGAACTTCATGT-3′ (SEQ ID NO:11). Probes were VIC-5′-CTCTTTCCTTTGGACCCACCTT-3′ (SEQ ID NO:12) and FAM-5′-CTCTTTGCTTTGGACCCACCT-3′ (SEQ ID NO:13). The annealing temperature was 60° C.

Linkage disequilibrium between BRCA2 and klotho—Marker D135171, which is located within the APRIN gene, approximately 280 kb upstream to the BRCA2 and 335 kb downstream to KLOTHO was analyzed in order to determine possible linkage disequilibrium between the two genes. Genomic DNA from each subject was PCR amplified for each marker using specific primer sequences as follows:

For Marker D135171 the following primers were used: Forward primer: 5′-CCTACCATTGACACTCTCAG (SEQ ID NO:14) and Reverse primer:5′-TAGGGCCATCCATTCT (SEQ ID NO:15); PCR product size: 227-241 (bp), GenBank Accession No.Z17151, UniSTS:32702, at Hypertext Transfer Protocol://World Wide Web (dot) ncbi (dot) nlm (dot) nih (dot) gov].

For marker D1351695 the following primers were used: Forward primer: 5′-AGAATCATTGCCCTACTTA (SEQ ID NO:16) and Reverse primer:5′-GATAACTTACCAGCATGTGA (SEQ ID NO:17); PCR product size: 246 (bp).

For marker D1351493 the following primers were used: Forward primer: 5′-ACCTGTTGTATGGCAGCAGT (SEQ ID NO:18) and Reverse primer: 5′-GGTTGACTCTTTCCCCAACT (SEQ ID NO:19); PCR product size: 223-248 (bp).

The forward primer of each pair of primers was labeled with FAM; and following PCR amplification 2 μl of the reaction mixture were mixed with 0.5 μl of the TAMRA 500 internal size standard (Applied Biosystems Inc., Foster City, Calif., USA), and 12 μl of formamide. Samples were read on the ABI Prism 3100 using the GeneScan Software (Applied Biosystems). The GeneScan raw data were analyzed using the Genotyper software to obtain the allele repeat in base pairs. Alleles obtained from the samples were used to construct the haplotype.

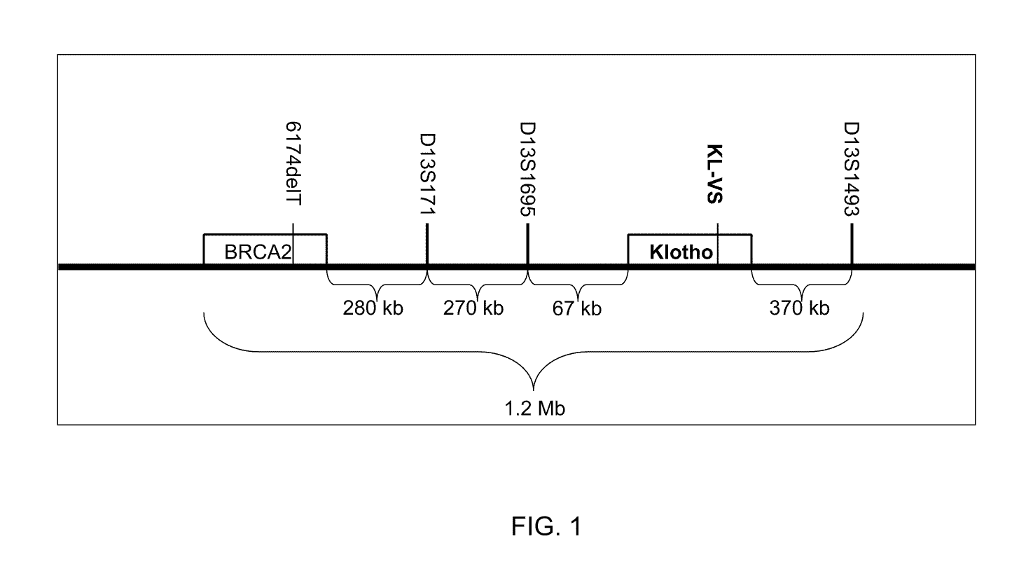

Haplotype analysis—To determine whether there is a linkage between the KLOTHO and BRCA2 genes, haplotype structure of the region spanning the distance between these two genes was constructed using three markers; D135171, D1351695 and D1351493. Markers D135171 and D1351695 are intergenic to the BRCA2 and Klotho, whereas D13S1493 is located downstream to Klotho. The three markers span a region of approximately 1.2 Mb (distance from BRCA2 to D13S1493) (FIG. 1). Nine families were selected for haplotype reconstruction, based on availability of carriers and non carriers of the BRCA2*6174delT mutation. Genomic DNA from each subject was amplified by PCR for each marker. The forward primers of each pair of primers were labeled with FAM. Two microlitres of each PCR product were mixed with 0.5 μl of the TAMRA 500 internal size standard (Applied Biosystems Inc., Foster City, Calif.), and 12 μl of formamide. Samples were read on the ABI Prism 3100 using the GeneScan Software (Applied Biosystems).The GeneScan raw data were analyzed using the Genotyper software to obtain the allele repeat in base pairs. Alleles obtained from the samples were used to construct the haplotype.

Constructs—The mouse klotho expression vector was a generous gift of Y. Nabeshima (Kyoto University, Japan). The human KLOTHO expression vector was constructed by subcloning the full-length cDNA (GenBank Accession No. NM_{—}004795.3; SEQ ID NO:6) isolated from a human kidney cDNA library into the pEF1 expression vector (Invitrogen). The substitutions F352V was introduced by PCR amplification using primers containing the mutation and verified by nucleotide sequencing. The resulting construct encode the wild type 352F Klotho protein (with a phenylalanine amino acid residue at position 352 of the Klotho protein set forth by SEQ ID NO:7) and the mutated Klotho protein 352V (with a valine amino acid residue at position 352 of the Klotho protein set forth by SEQ ID NO:7).

Cells and transfections—Breast cancer cell lines were obtained from the American Type Culture Collection (Manassas, Va.). All transfections used LipofectAMINE 2000 (Invitrogen).

Colony assays—Two days following transfection with the indicated plasmids (i.e., the wild type 352F and the mutant 352V), G418 (750 μg/ml) was added to the culture media; and at day 14, the cells were stained using gentian violet. Untransfected cells were treated similarly, and all died within the 2 weeks of culture in the selection media. Quantification of the results was performed using AlphaImager 2000 (Alpha Innotech, CA).

Klotho secretion—Secretion assay was conducted as previously described (Chen et al., 2007). Briefly, MCF-7 cells grown on six-well plates were transfected with 4 μg of pEF-hKL, pEF-V-hKL or control empty plasmid pEF. Forty eight hours after transfection cells were washed twice with PBS and incubated with serum-free DMEM for 6 hours. Media was collected and centrifuged at 3000×g for 5 minutes to remove detached cells. BSA (10 μg/ml) was added to the conditioned medium as a carrier protein and as a control for precipitation efficiency. The samples were precipitated with 25% TCA and kept at −20° C. for 5 minutes, on ice for at least 1 hour, and centrifuged for 15 minutes at 16,000×g. The protein pellet was washed 3 times with ice cold acetone and centrifuged for 5 minutes at 16,000×g, dried at 100° C. for 10 minutes and dissolved in 2×Laemmli sample buffer at 100° C. for 10 minutes. For cell lysate protein extraction: after media collection, cells were washed twice with ice-cold PBS and lysed with RIPA buffer (50 mM Tris-HCl, pH 7.4, 150 mM NaCl, 1% NP40, 0.25% Na-deoxycholate, 1 mM EDTA, 1 mM NaF) with a protease inhibitor cocktail (Sigma). The cell lysate was centrifuged at 16,000×g for 15 minutes, and the supernatant was collected for SDS-PAGE.

Western blot analysis—Proteins precipitated from media or 50 μg protein extracts were loaded on 10% polyacrylamide gels, separated electrophoretically and blotted from the gel onto nitrocellulose membrane (Schleicher & Schuell Bioscience GmbH, Dassel, Del.). The membranes were then immunoblotted with anti klotho KM2076 antibody (1:2,000, kindly provided by Kyowa Hakko Kogy (Tokyo)) or anti albumin (Dako, Denmark). Band intensities were quantified using Image) software.

Statistical analysis—Unaffected women were censored at age of last follow-up or at the age of the relevant prophylactic surgery, whichever came first. Risks for women with breast or ovarian cancer were censored at age of cancer diagnosis. Women with both breast and ovarian cancers were censored according to the first cancer diagnosis. Difference in klotho allele frequencies between the study groups were calculated by the chi-square test. Unweighted and weighted Cox regression analysis was used to evaluate specific genotype associated hazard ratio (HR) with corresponding 95% confidence intervals (95% CI). The weights were the same as used in the paper of Antoniou et al., 2005. Since the unweighted and weighted methods yielded similar hazard ratio estimates, the results of the unweighted analysis are reported herein. Women diagnosed more than 5 years previous to the genetic test were excluded to avoid survival bias. First-degree relatives of a participant who had already been tested were also excluded from the analysis. Kaplan-Meier survival was used to analyze disease-free survival. P<0.05 was considered statistically significant, and all significance tests were two-tailed. Statistical analyses were done using SPSS software. The survival analysis was performed using STATA 10 SE software.

### Example 1

### Heterozygosity to the KLOTHO Functional Variant is Highly Associated with Increased Risk to Breast or Ovarian Cancer Among BRACA1-Mutation Carriers

Experimental Results

Study population included 1115 women, 909 from the Sheba Medical Center, 162 BRCA1 carriers from the Shaare Zedek Medical Center and 44 BRCA2 carriers from Cedars-Sinai Medical Center. Sixteen patients who were diagnosed with both breast and ovarian cancers were censored according to the first cancer diagnosis (14 presented first with breast cancer and two with ovarian cancer). The patients were divided into four subsets (Table 4): cancer free non-carriers of BRCA1 or BRCA2 mutations (N=109), breast or ovarian cancer patients, non-carriers of BRCA1 or BRCA2 mutations (N=127), BRCA1 mutation carriers (N=631) and BRCA2 mutations carriers (N=248). The mutation carriers were further subdivided into unaffected and breast and ovarian cancer patients.

Similar klotho allele frequencies were noted among non-carriers and BRCA1 mutation carriers regardless of disease status (Table 4). Thus, FF, FV and VV frequencies were 58%, 35% and 7% and 65%, 29% and 6% among non-carriers and carriers, respectively (P=0.16). No statistically significant differences were noted when the analysis was conducted according to disease status within each group or between the two groups (data not shown). Compared to this population of Ashkenazi Jewish women, lower frequencies of the FV and VV variants were previously reported for Bohemian or Baltimore Caucasian (FF-74%, FV-25%, FF-1%) (Arking et al., 2002).

TABLE 4
Distribution of KLOTHO genotypes among unaffected and cancer patients by BRCA1 or BRCA2 mutation status

| | Non-BRCA1/2 carriers | | | | | | | | BRCA1 carriers | | | | | | | |
|---|---|---|---|---|---|---|---|---|---|---|---|---|---|---|---|---|
| | Healt. | | BC* | | OC# | | Overall | | Healt. | | BC | | OC | | Overall | |
| | N = 109 | | N = 94 | | N = 33 | | N = 236 | | N = 290 | | N = 246 | | N = 95 | | N = 631 | |
| Median age† | 51 | | 50 | | 57 | | | | 38 | | 45 | | 51 | | | |
| (years) | | | | | | | | | | | | | | | | |
| FF, N (%) | 61 | (56) | 56 | (60) | 21 | (64) | 138 | (58) | 195 | (67) | 158 | (64) | 60 | (64) | 413 | (65) |
| FV, N (%) | 42 | (38) | 31 | (33) | 9 | (27) | 82 | (35) | 80 | (28) | 72 | (29) | 28 | (29) | 180 | (29) |
| W, N (%) | 6 | (6) | 7 | (7) | 3 | (9) | 16 | (7) | 15 | (5) | 16 | (7) | 7 | (9) | 38 | (6) |

| | BRCA2 carriers | | | | | | | |
|---|---|---|---|---|---|---|---|---|
| | Healt. | | BC | | OC | | Overall | |
| | N = 101 | | N = 116 | | N = 31 | | N = 248 | |
| Median age† | 43 | | 46 | | 62 | | | |
| (years) | | | | | | | | |
| FF, N (%) | 16 | (16) | 9 | (8) | 1 | (3) | 26 | (10) |
| FV, N (%) | 64 | (63) | 82 | (71) | 24 | (77) | 170 | (69) |
| W, N (%) | 21 | (21) | 25 | (21) | 6 | (20) | 52 | (21) |

Table 4: KLOTHO genotypes are as follows: FF = homozygous to the common allele having Phe at position 352 of the Klotho protein (GenBank Accession No. NP_004786; SEQ ID NO: 7); FV = heterozygous to the polymorphism Phe/Val at position 352 of the Klotho protein; VV = homozygous to the rare allele having Val at position 352 of the Klotho protein. “Healt.” = healthy (no cancer);
“*BC” = breast cancer;
#OC = ovarian cancer;
†Age at cancer diagnosis or at counseling for non-cancer patients. N = absolute numbers; (%) Numbers in parenthesis reflect frequency (in percentages) of a certain genotype in the Klotho gene among each of the tested groups, i.e., healthy subjects, BC (breast cancer subjects) and OC (ovarian cancer subjects). P < 0.0001 for the comparison between the frequencies of the klotho alleles among non-BRCA versus BRCA2 carriers.

Heterozygosity but not homozygosity to the klotho functional variant was highly associated with increased risk of breast or ovarian cancer among BRCA1-mutation carriers—BRCA1 mutation carriers included 290 unaffected, 246 breast cancer patients and 95 ovarian cancer patients. Adjusted for age at cancer diagnosis, the presence of klotho FV variant in BRCA1 carriers was associated with increased risk of breast and ovarian cancer (Table 5 hereinbelow, HR 1.4, 95% CI 1.08-1.83, p=0.01). A reduced risk of cancer, with borderline statistical significance, was noted among women with the VV alleles (HR 0.65, 95% CI 0.42-1.02, p=0.06). Analysis according to type of cancer suggested an association between FV genotype and increased risk of breast cancer (HR 1.35, 95% CI 0.99-1.83, p=0.06), and ovarian cancer risk (HR 1.54, 95% CI 0.97-2.45, p=0.07). Analysis of age at breast cancer diagnosis according to FF or FV genotype status among BRCA1 carriers was also conducted (FIG. 2). The median age at diagnosis was 48 years for FF/BRCA1, 43 years for FV/BRCA1 status and 53 for VV/BRCA1 (p=0.04 for FF/BRCA1 vs. FV/BRCA1, p=0.186 for VV/BRCA1 vs. FF/BRCA1 and p=0.02 for differences between the three groups). KLOTHO genotype status was not associated with significant differences in age at diagnosis of ovarian cancer among BRCA1 carriers (data not shown).

TABLE 5
Hazard ratio (HR) and 95% confidence interval (CI) for predicting cancer
among BRCA1 mutation carriers by klotho variant, adjusted for age

| Variable | HR | 95% CI | P value |
|---|---|---|---|
| Overall cancer | | | |
| FF vs FV | 1.4 | 1.08-1.83 | 0.01 |
| FF vs VV | 0.65 | 0.42-1.02 | 0.06 |
| Overall difference between the groups | | | 0.001 |
| Breast cancer | | | |
| FF vs FV | 1.35 | 0.99-1.83 | 0.06 |
| FF vs VV | 0.68 | 0.40-1.16 | 0.15 |
| Overall difference between the groups | | | 0.02 |
| Ovarian cancer | | | |
| FF vs FV | 1.54 | 0.97-2.45 | 0.07 |
| FF vs VV | 0.63 | 0.28-1.41 | 0.26 |

| | |
|---|---|
| Overall difference between the groups | 0.04 |

The analysis was conducted on 236 non-carriers and 576 BRCA1 carriers. 55 BRCA1 carriers in which cancer diagnosis preceded genetic testing by more than five years or belonged to the same family were excluded (in order to avoid bias).
FF = Homozygous for the common allele having Phe amino acid at position 352 of the Klotho protein GenBank Accession No. NP_004786;
FV = heterozygous for the rare allele having Phe/Val at position 352 of the Klotho protein GenBank Accession No. NP_004786;
VV = homozygous for the rare allele having Val at position 352 of the Klotho protein GenBank Accession No. NP_004786;
vs. = versus.
HR = hazard ratio;
CI = confidence interval.

Altogether, these results demonstrate that the presence of heterozygosity to the klotho functional variant (e.g., as determined by the presence of FV variant at position 352 of the klotho protein) is highly associated with increased risk to develop cancer (breast cancer or ovarian cancer) among carrier of the BRCA1 mutations such as the 185delAG and 5382insC mutations in the BRCA1 gene. In addition, the presence of the Klotho FV variant was associated with the diagnosis of breast cancer at a significantly younger age (5 years earlier for subjects who exhibit Klotho-FV genotype and a BRCA1-mutation as compared to the Klotho-FF genotype and the BRCA1-mutation). Thus, presence of the klotho functional variant (e.g., the Phe/Val polymorphism at position 352 of Klotho protein) in a heterozygous form in a subject who is a carrier of a mutation in the BRCA1 gene indicates that the subject has an increased predisposition (risk) to develop breast cancer and/or ovarian cancer as compared to a subject who is a carrier of a mutation in the BRCA1 gene but who exhibits a homozygous genotype at position 352 of the klotho protein (e.g., FF or VV variant). These results suggest the detection of the Klotho functional variant (e.g., FV at position 352 of Klotho protein) in addition to mutation analysis of the BRCA1 gene in a subject in order to predict the risk for breast and/or ovarian cancer in the subject. Once determined, the subject should be informed that he/she exhibits an increased predisposition to develop the cancer and thereby should be advised to undergo intensive follow-up or prevention measures starting at an earlier age (e.g., at least 5-10 years earlier than recommended for the entire age-matched population). The follow-up and measures can be tests and scans such as blood tests, tissue biopsies, detection of presence of lesions by ultrasound, mammography, CT-scans, MRI and the like)

### Example 2

### The KLOTHO Functional Variant is in Linkage Disequilibrium with the 6174delT BRAC2 Mutation

Experimental Results

The klotho functional variant is in linkage disequilibrium with the BRCA2 6174delT mutation—The KLOTHO gene is located on chromosome 13q12, 616 kb upstream of the BRCA2 gene (FIG. 1). Analysis of 248 BRCA2 6174delT mutation carriers revealed significantly higher frequency of the FV and VV phenotypes compared with non-carriers. Thus, FF, FV and VV were detected in 10%, 69% and 21% of the BRCA2 mutation carriers, compared to 58%, 35% and 7% of the non-carriers (Table 4 in Example 1 hereinabove, p<0.0001 for the comparison between the two groups). Analysis of maintenance of the Hardy-Weinberg equilibrium (HWE), revealed a clear deviation from equilibrium in the BRCA2 population (data not shown), suggesting linkage disequilibrium between BRCA2 6174delT mutation and the KLOTHO V allele. The present inventors have assessed possible linkage disequilibrium in all the BRCA2 carriers using the micro-satellite marker D13S171 located between the BRCA2 and the KLOTHO genes at a distance of about ˜350 Kb from each gene. The analysis was consistent with these two genes being in linkage disequilibrium (data not shown). A haplotype of the region between BRCA2*6174delT mutation and KL-VS allele was constructed using nine families with both carriers and non-carriers of BRCA2*6174delT (Table 6, hereinbelow). Linkage between BRCA2*6174delT and the KL-VS variant was noted in eight out of the nine families, thus indicating the existence of linkage disequilibrium between the two alleles.

TABLE 6
Allelic distribution of the markers used in the haplotype
reconstruction in BRCA2*6174delT families.

| D13S1493 | KL-VS | D13S1695 | D13S171 | 6174 ^{†} | ID | |
|---|---|---|---|---|---|---|
| 244, 244 | FV | 326, 332 | 243, 245 | + | 4128 | Family 1 |
| 244, 248 | FV | 326, 332 | 243, 245 | − | 6870 | Family 1 |
| 244, 252 | VV | 326, 332 | 243, 245 | + | 6000 | Family 2 |
| 240, 256 | FF | 326, 328 | 231, 245 | − | 6794 | Family 2 |
| 244, 252 | FV | 328, 332 | 235, 243 | + | 5016 | Family 3 |
| 244, 252 | FV | 324, 332 | 243, 245 | − | 5119 | Family 3 |
| 244, 244 | FV | 328, 328 | 231, 235 | − | 5151 | Family 3 |
| 244, 252 | VV | 328, 332 | 231, 243 | + | 6638 | Family 4 |
| 244, 244 | FV | 328, 328 | 231, 235 | − | 6717 | Family 4 |
| 244, 244 | FV | 326, 328 | 231, 245 | − | 6718 | Family 4 |
| 244, 252 | FV | 328, 332 | 235, 243 | + | 6719 | Family 4 |
| 248, 260 | FF | 326, 334 | 245, 245 | − | 7483 | Family 5 |
| 252, 252 | FV | 332, 336 | 235, 243 | + | 7580 | Family 5 |
| 248, 252 | FV | 330, 332 | 243, 245 | + | 8439 | Family 6 |
| 244, 248 | FV | 312, 328 | 231, 245 | − | 4847 | Family 6 |
| 252, 252 | FV | 332, 334 | 235, 243 | + | 8188 | Family 7 |
| 252, 256 | FV | 326, 326 | 231, 235 | − | 8690 | Family 7 |
| 244, 252 | FF | 326, 328 | 237, 245 | − | 8329 | Family 8 |
| 244, 252 | FV | 328, 332 | 235, 243 | + | 8387 | Family 8 |
| 244, 256 | FF | 326, 328 | 231, 243 | + | 8466 | Family 9 |
| 244, 252 | FF | 326, 332 | 235, 245 | − | 8591 | Family 9 |
| 240, 252 | FF | 326, 328 | 231, 243 | + | 8689 | Family 9 |

^{†} .delT carrier's families6174*2Haplotype analysis in BRCA: 6Table BRCA2 6174delT mutation status: “+” indicates presence and “−” indicates absence of the mutation (as described under “General Materials and Experimental Methods”). KL-VS polymorphosm status: FV = heterozygous to the Phe/Val polymorphism at position 352 of Klotho protein (SEQ ID NO: 7); VV = homozygous to the Val amino acid residue at position 352 of the Klotho protein (SEQ ID NO: 7); FF = homozygous to the Phe amino acid residue at position 352 of the Klotho protein (SEQ ID NO: 7). The polymorphisms in the tested markers D13S1493, D13S1695 and D13S171 are represented by the sizes of PCR products [in base pairs (bp)]; ID = internal sample identification number.

Analysis of KLOTHO variants among BRCA2 carriers (6174delT) revealed higher frequency of the FF variant among unaffected compared to cancer patients (Table 7 hereinbelow, 16% vs 7%, p=0.03). Yet, only 26 BRCA2 carriers, 16 unaffected and 10 cancer patients showed the FF variant and significantly older age was observed among unaffected compared to affected carriers (median age 49 years vs. 43 years, p<0.001).

TABLE 7
Distribution of KLOTHO variants among BRCA2
(6174delT) mutation carriers by disease status

| | Healthy | Breast or ovarian |
|---|---|---|
| | N (%) | cancer N (%) |
| Median age* | 43 | 49 |
| (years) | | |
| FF | 16 (16) | 10 (7) |
| FV/VV | 85 (84) | 137 (93) |

*Age at cancer diagnosis or at counseling for non-cancer patients.
FF = Homozygous for the common allele having Phe amino acid at position 352 of the Klotho protein GenBank Accession No. NP_004786;
FV = heterozygous for the rare allele having Phe/Val at position 352 of the Klotho protein GenBank Accession No. NP_004786;
VV = homozygous for the rare allele having Val at position 352 of the Klotho protein GenBank Accession No. NP_004786;
N = absolute numbers; (%) Numbers in parenthesis reflect frequency (in percentages) of a certain genotype in the Klotho gene among each of the tested groups, i.e., healthy subjects or breast and ovarian cancer -affected subjects.

The tight linkage disequilibrium between the FV variant of the Klotho gene and the BRCA2 6174delT mutation suggests the use of the klotho functional variant for determining predisposition to breast or ovarian cancer.

### Example 3

### The KLOTHO Functional Variant Exhibits Less Colony Suppression Effect on Breast Cancer Cells as Compared to Wild-Type KLOTHO

Experimental Results

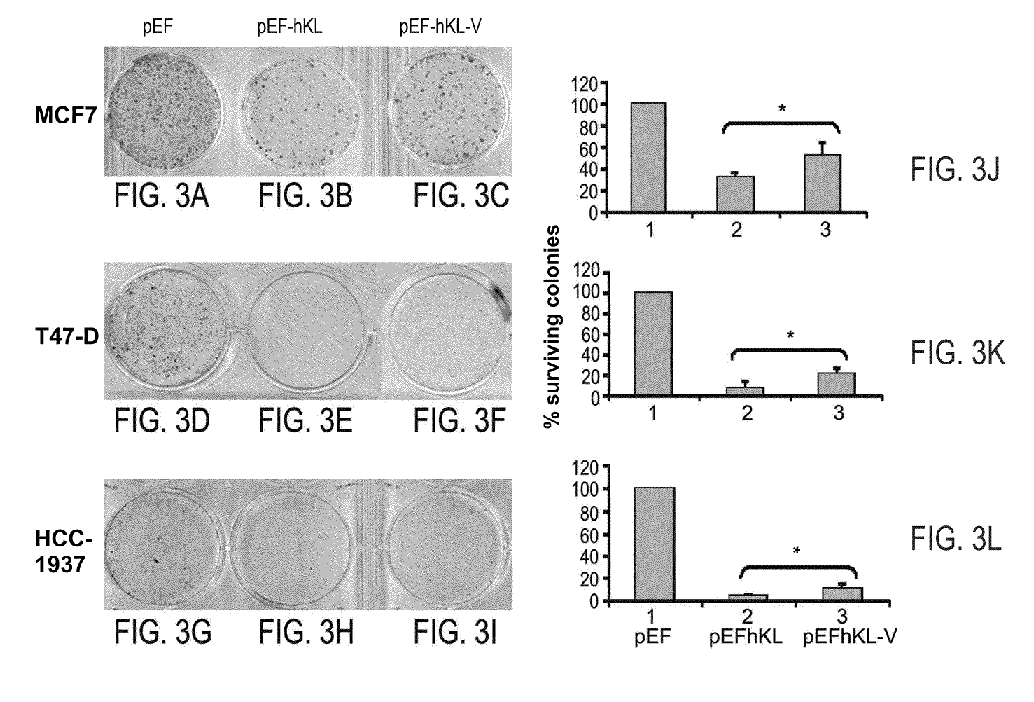

Expression of the klotho variant which comprises Val at position 352 of the Klotho protein is less efficient in growth suppression of cancerous cell lines—Over-expression of klotho in breast cancer cells inhibits clonal growth (Wolf et al., 2008). In order to elucidate whether the klotho variant has reduced ability to inhibit growth of breast cancer cells, a series of colony formation assays was conducted in breast cancer cell lines which contain either wild type (MCF-7, T-47D) or mutated BRCA1 (HCC-1937) (Tomlinson et al., 1998). The cells were transfected with either an empty vector (pEF), wild-type human klotho expression vector (pEFhKL) or human klotho-V expression vector, in which phenylalanine at position 352 has been substituted to valine (pEFhKL-V). Transfected cells were cultured in media containing G418 for two weeks and stained to determine the number of surviving colonies. While expression of wild type klotho reduced the number and size of surviving colonies by up to 95% (FIGS. 3B, E, H, J, K and L), expression of the KL-V variant showed significantly less growth inhibitory activity (about 70% activity, p=0.001; FIGS. 3C, F, I, J, K and L).

These results demonstrate that presence of the Val residue at position 352 of the Klotho protein increases the cancerous phenotype of cells by exhibiting a reduced ability to inhibit cancerous cell proliferation.

### Example 4

### The Presence of the Val Residue at Position 352 of the KLOTHO Protein Reduces Secretion of KLOTHO from Breast Cancer Cells

Experimental Results

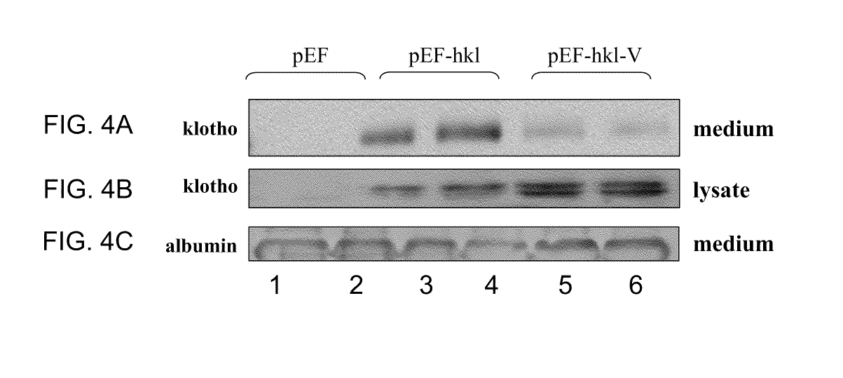

Klotho is a trans-membranal protein which can be cleaved and shed (Chen et al., 2007; Kurosu et al., 2005). Klotho growth inhibitory activities in breast cancer are probably mediated mainly by its secreted form ((Wolf et al., 2008) and unpublished data). It has been shown that in HeLa cells the KL-V variant secretion is reduced compared to wild-type klotho (Arking et al., 2002). In order to examine the effect of the V mutation on the secretion of the protein in breast cancer cells, MCF-7 cells were transfected with wild-type klotho (pEFhKL), klotho-V (pEFhKL-V) or an empty vector as control (pEF), and klotho secretion into the medium was assessed and compared to klotho expression in the cells. While klotho-V expression in the cells was elevated compared to wild-type klotho, the secretion of the Klotho-V was significantly reduced (FIGS. 4A-C). Albumin immunoblot shows that the differences in klotho levels in the medium were not a result of unequal protein precipitation.

Analysis and discussion—The klotho gene was recently identified as a potent tumor suppressor gene in breast and other cancers (Wolf et al., 2008). Germ-line mutations in BRCA1 and BRCA2 substantially increase lifetime risk of breast and ovarian cancers. Yet, penetrance of deleterious BRCA1 and BRCA2 mutations is incomplete even among carriers of identical mutations. The present inventors examined the association between KL-VS and cancer risk among 826 Ashkenazi Jewish women: 236 non-carriers, 340 BRCA1 (185delAG, 5382insC) carriers and 248 BRCA2 (6174delT) carriers. Among BRCA1 carriers, KL-VS was associated with increased breast cancer risk (HR 1.8, 95% CI 1.24-2.61, p=0.002) and younger age at breast cancer diagnosis (53 vs. 45 p<0.0001). The results presented in this study demonstrate that heterozygosity for the KLOTHO V allele (at position 352 of the Klotho protein) is associated with increased breast and ovarian cancers risk, as well as with younger age at diagnosis of breast cancer, in Ashkenazi Jewish women carriers of a BRCA1 mutation [e.g., the 185delAG or 5382insC].

KLOTHO and BRCA2 are located on 13q12. The results of the present study show linkage disequilibrium between BRCA2 6174delT mutation and the KLOTHO V allele. In addition, a significantly lower prevalence of the FF allele among cancer patients as compared to unaffected individuals was shown. Without being bound by any theory, these results suggest a possible role for the klotho functional variant (e.g., the Val allele at position 352 of klotho protein) in the development cancer among Ashkenazi Jewish carriers of the BRCA2 6174delT.

Klotho over-expression specifically reduces colony formation of breast cancer cells (Wolf et al., 2008). The results of the present study show reduced growth inhibitory activity (of breast cancer cells) of the klotho Val variant (position 352) as compared to wild type Phe klotho (position 352). The reduced growth inhibitory activity might be due to decreased secretion of klotho-V to the medium (FIG. 4A). Indeed it has been shown that klotho can be shed from the cells (Chen et al., 2007) suggesting that most of klotho growth inhibitory activities in breast, as well as other cancers are mediated by this secreted part of the protein [Wolf et al., 2008; Wolf et al, unpublished data]. A possible explanation for the clinical observations is decreased shedding of the klotho variant, which halts its growth inhibitory activities.

These data suggest the klotho functional variant KL-VS as a breast cancer risk modifier among BRCA1 mutation and/or BRCA2 carriers.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

Antoniou A C, Goldgar D E, Andrieu N, Chang-Claude J, Brohet R, Matti A et al. (2005). A weighted cohort approach for analyzing factors modifying disease risks in carriers of high-risk susceptibility genes. Genet Epidemiol 29: 1-11.

Antoniou A C, Easton D F. (2006). Models of genetic susceptibility to breast cancer. Oncogene 25: 5898-5905.

Antoniou A C, Sinilnikova O M, Simard J, L'on', M, Dumont M, Neuhausen S L, et al. (2007). RAD51 135G′C modifies breast cancer risk among BRCA2 mutation carriers: results from a combined analysis of 19 studies. Am J Hum Genet 81: 1186-1200.

Antoniou A C, Spurdle A B, Sinilnikova O M, Healey S, Pooley K A, Schmutzler R K, et al. (2008). Common breast cancer-predisposition alleles are associated with breast cancer risk in BRCA1 and BRCA2 mutation carriers. American J Hum Genet 82: 937-948.

Arking D E, Krebsova A, Macek M, Sr., Macek M, Jr., Arking A, Mian I S, et al. (2002). Association of human aging with a functional variant of klotho. Proc Natl Acad Sci 99: 856-861.

Arking D E, Becker D M, Yanek L R, Fallin D, Judge D P, Moy T F, et al. (2003). KLOTHO allele status and the risk of early-onset occult coronary artery disease. Am J Hum Genet 72: 1154-1161.

Arking D E, Atzmon G, Arking A, Barzilai N, Dietz H C. (2005). Association between a functional variant of the KLOTHO gene and high-density lipoprotein cholesterol, blood pressure, stroke, and longevity. Circ Res 96: 412-418.

Begg C B, Haile R W, Borg A, Malone K E, Concannon P, Thomas D C, et al. (2008). Variation of breast cancer risk among BRCA1/2 carriers. JAMA 299: 194-201.

Cha S-K, Ortega B, Kurosu H, Rosenblatt K P, Kuro-o MHuang C-L. (2008). Removal of sialic acid involving Klotho causes cell-surface retention of TRPV5 channel via binding to galectin-1. Proc Natl Acad Sci: 9805-9810.

Chen C-D, Podvin S, Gillespie E, Leeman SEAbraham C R. (2007). Insulin stimulates the cleavage and release of the extracellular domain of Klotho by ADAM10 and ADAM17. Proc Natl Acad Sci 104: 19796-19801.

Hudelist G, Wagner T, Rosner M, Fink-Retter A, Gschwantler-Kaulich D, Czerwenka K, et al. (2007). Intratumoral IGF-I protein expression is selectively upregulated in breast cancer patients with BRCA1/2 mutations. Endocr Relat Cancer 14: 1053-1062.

Imura A, Iwano A, Tohyama O, Tsuji Y, Nozaki K, Hashimoto N, et al. (2004). Secreted Klotho protein in sera and CSF: implication for post-translational cleavage in release of Klotho protein from cell membrane. FEBS Letters 565: 143-147.

Ito S, Kinoshita S, Shiraishi N, Nakagawa S, Sekine S, Fujimori T, et al. (2000). Molecular cloning and expression analyses of mouse [beta]klotho, which encodes a novel Klotho family protein. Mech Develop 98: 115-119.

Kuro-o M, Matsumura Y, Aizawa H, Kawaguchi H, Suga T, Utsugi T, et al. (1997). Mutation of the mouse klotho gene leads to a syndrome resembling ageing. Nature 390: 45-51.

Kurosu H, Yamamoto M, Clark J D, Pastor J V, Nandi A, Gurnani P, et al. (2005). Suppression of aging in mice by the hormone klotho. Science 309: 1829-1833.

Kurosu H, Ogawa Y, Miyoshi M, Yamamoto M, Nandi A, Rosenblatt K P, et al. (2006). Regulation of fibroblast growth factor-23 signaling by klotho. J Biol Chem 281: 6120-6123.

Levy-Lahad E, Friedman E. (2007). Cancer risks among BRCA1 and BRCA2 mutation carriers. Br J Cancer 96: 11-15.

Lu L, Katsaros D, Wiley A, de la Longrais I A, Puopolo M, Yu H. (2008) Klotho expression in epithelial ovarian cancer and its association with insulin-like growth factors and disease progression. Cancer Invest. 26:185-92.

Maor S, Yosepovich A, Papa M Z, Yarden R I, Mayer D, Friedman E, et al. (2007). Elevated insulin-like growth factor-I receptor (IGF-IR) levels in primary breast tumors associated with BRCA1 mutations. Can Lett 257: 236-243.

Matsumura Y, Aizawa H, Shiraki-Lida T, Nagai R, Kuro-o MNabeshima Y-i. (1998). Identification of the human klotho gene and its two transcripts encoding membrane and secreted klotho protein. Biochem Biophys Resh Commun 242: 626-630.

Ohyama Y, Kurabayashi M, Masuda H, Nakamura T, Aihara Y, Kaname T, et al. (1998). Molecular cloning of rat klotho cDNA: markedly decreased expression of klotho by acute inflammatory stress. Biochem Biophys Res Commun 251: 920-925.

Rohlfs E M, Learning W G, Friedman K J, Couch F J, Weber BLSilverman L M. (1997). Direct detection of mutations in the breast and ovarian cancer susceptibility gene BRCA1 by PCR-mediated site-directed mutagenesis. Clin Chem 43: 24-29.

Shiraki-Lida T, Aizawa H, Matsumura Y, Sekine S, Lida A, Anazawa H, et al. (1998). Structure of the mouse klotho gene and its two transcripts encoding membrane and secreted protein. FEBS Letters 424: 6-10.

Tomlinson G E, Chen T T-L, Stastny V A, Virmani A K, Spillman M A, Tonk V, et al. (1998). Characterization of a breast cancer cell line derived from a germ-line BRCA1 mutation carrier. Cancer Res 58: 3237-3242.

Urakawa I, Yamazaki Y, Shimada T, Iijima K, Hasegawa H, Okawa K, et al. (2006). Klotho converts canonical FGF receptor into a specific receptor for FGF23. Nature 444: 770-774.

Wolf I, Levanon-Cohen S, Bose S, Ligumsky H, Sredni B, Kanety H, et al. (2008). Klotho: a tumor suppressor and a modulator of the IGF-1 and FGF pathways in human breast cancer. Oncogene: 27: 7094-7105.

Wolf I, Sadetzki S, Catane R, Karasik A, Kaufman B. (2005). Diabetes mellitus and breast cancer. Lancet Oncol 6: 103-111.

Yamamoto M, Clark J D, Pastor J V, Gurnani P, Nandi A, Kurosu H, et al. (2005). Regulation of oxidative stress by the anti-aging hormone klotho. J Biol Chem 280: 38029-38034.

Yee D. (2006). Targeting insulin-like growth factor pathways. Br J Cancer 94: 465-468.

1. A method of determining if an individual is predisposed to cancer, the method comprising determining a presence or an absence of the KLOTHO functional variant in a heterozygote form wherein said presence of the KLOTHO functional variant in said heterozygote form is indicative of an increased predisposition to the cancer, thereby determining if the individual is predisposed to the cancer.

2. A method of determining a prognosis of cancer in an individual, the method comprising determining a presence or an absence of the KLOTHO functional variant in a heterozygote or homozygous form wherein said presence of the KLOTHO functional variant in said heterozygote or said homozygous form is indicative of a worsen prognosis of the cancer as compared to said absence of the KLOTHO functional variant, thereby determining the prognosis of the cancer in the individual.

3. A method of designing a treatment regimen to an individual, comprising:
(a) determining if the individual is predisposed to cancer according to the method of claim 1, and
(b) designing a treatment regimen to the individual based on said presence of said predisposition,
thereby designing the treatment regimen to the individual.

4. A method of treating cancer in an individual, comprising,
(a) determining if the individual is predisposed to the cancer according to the method of claim 1, and
(b) treating the cancer based on said presence of said predisposition, thereby treating the cancer in the individual.

5. A kit for determining if an individual is predisposed to cancer, the kit comprising at least one oligonucleotide or antibody for specifically determining a presence or an absence of the KLOTHO functional variant in a heterozygote form, and at least one oligonucleotide or antibody for specifically determining a presence or an absence of a mutation in the BRCA1 sequence set forth by SEQ ID NO:4 and/or in the BRCA2 sequence set forth by SEQ ID NO:5, said mutation in the BRCA1 or in the BRCA2 is associated with the cancer.

6. The method of claim 1, further comprising determining a presence or an absence of a mutation in the BRCA1 sequence set forth by SEQ ID NO:4 which is associated with the cancer.

7. The method of claim 1, further comprising selecting the individual as being a carrier of a mutation in the BRCA1 sequence set forth by SEQ ID NO:4 which is associated with the cancer.

8. The method of claim 1, further comprising determining a presence or an absence of a mutation in the BRCA2 sequence set forth by SEQ ID NO:5 which is associated with the cancer.

9. The method of claim 1, further comprising selecting the individual as being a carrier of a mutation in the BRCA2 sequence set forth by SEQ ID NO:5 which is associated with the cancer.

10. The method of claim 1, wherein the KLOTHO functional variant comprises a valine amino acid residue at position 352 of the Klotho protein set forth by SEQ ID NO:7 or a guanine nucleotide at position 1062 of the KLOTHO transcript set forth by SEQ ID NO:6.

11. The method of claim 1, wherein the KLOTHO functional variant is selected from the group consisting of a valine amino acid residue at position 352 of the Klotho protein set forth by SEQ ID NO:7, a guanine nucleotide at position 1062 of the KLOTHO transcript set forth by SEQ ID NO:6, a serine amino acid residue at position 370 of the klotho protein set forth by SEQ ID NO:7, a cytosine nucleotide at position 1117 of the KLOTHO transcript set forth by SEQ ID NO:6, and an adenosine nucleotide at position 1163 of the KLOTHO transcript set forth by SEQ ID NO:6.

12. The method of claim 1, wherein the method is effected ex vivo.

13. The method of claim 1, further comprising informing the individual of said presence of said increased predisposition to the cancer.

14. The method of claim 1, wherein said predisposition comprises an increased risk to develop the cancer at a younger age as compared to an individual who is not heterozygote to said KLOTHO functional variant.

15. The method of claim 1, further comprising recording said increased predisposition to the cancer in the subject's medical file.

16. The kit of claim 5, wherein said at least one oligonucleotide does not exceed 20 oligonucleotides.

17. The method of claim 6 wherein said mutation in the BRCA1 sequence is selected from the group consisting of a BRCA1 185delAG (a deletion of the adenosine and guanine nucleotides at position 2288-2289 of SEQ ID NO:4) and BRCA1 5382insC (an insertion of a cytosine nucleotide at position 69293 of SEQ ID NO:4).

18. The method of claim 8 wherein said mutation in the BRCA2 sequence is the BRCA2 6174delT (a deletion of a thymidine nucleotide at position 26050 of SEQ ID NO:5).

19. The method of claim 1, wherein when the individual exhibits said increased predisposition to the cancer, the method further comprising subjecting the individual to a gold-standard diagnostic test to diagnose the cancer.

20. The method of claim 1, wherein the cancer is breast cancer.

21. The method of claim 1, wherein the cancer is ovarian cancer.

22. The method of claim 1, wherein said determining said presence or said absence of the KLOTHO functional variant is performed in a biological sample of the individual.

23. The method of claim 1, wherein said determining said presence or said absence of the KLOTHO functional variant is effected using a DNA detection method.

24. The method of claim 1, wherein said determining said presence or said absence of the KLOTHO functional variant is effected using a protein detection method.


SEQUENCE LISTING (30 sequences)

SEQ ID NO: 1 - DNA, 52 bp, Homo sapiens
  misc_feature: rs9536314
ataacctttcatctattctgcctgatnttactgaatctgagaaaaagttcat

SEQ ID NO: 2 - DNA, 20 bp, Artificial sequence
  Single strand DNA oligonucleotide
gccaaagtctggcatctcta

SEQ ID NO: 3 - DNA, 22 bp, Artificial sequence
  Single strand DNA oligonucleotide
ttccatgatgaactttttgagg

SEQ ID NO: 4 - DNA, 82900 bp, Homo sapiens
  misc_feature (2288)..(2289): AG can be deleted
attggctcttaccacttgtccctcaaaacgaccaccccattgactggtggcgattgcgtcgacggagacggggcaaaagcaagctgaacccgaaaaataacaaacactggggctgaggggtggaactacgagtgcgcagacatgggccagagcgcatttcccctgccccaggcaaattcggcgctcactgcgtccccgcaggccactgaccttacaagactacttgccccagactcctggggctggatgggaattgtagtctccctaaagagttgtacgtatctttttaaggcctagtttctgctttcaaaatacgaaaacataacaacactccagtccataactgttgacaagtacaagcgcgcacaggtctccaatctatccactggatttccgtgagaattgtgcccgctctggtattggatgttcctctccataagactacagtttctaaggaacactgtggcgaagacctttcattccgcaacgcatgctggaaataattatttccctccacccccccaacaatccttattacttatatttaccgaaactggagacctccattagggcggaaagagtgggggattgggacctcttcttacgactgctttggacaataggtagcgattctgaccttcgtacagcaattactgtgatgcaataagccgcaactggaagagtagaggctagagggcaggcactttatggcaaactcaggtagaattcttcctcttccgtctctttccttttacgtcatccgggggcagactgggtggccaatccagagccccgagagacgcttggctctttctgtccctcccatcctctgattgtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaaggtagtagagtcccgggaaagggacagggggcccaagtgatgctctggggtactggcgtgggagagtggatttccgaagctgacagatgggtattctttgacggggggtaggggcggaacctgagaggcgtaaggcgttgtgaaccctggggaggggggcagtttgtaggtcgcgagggaagcgctgaggatcaggaagggggcactgagtgtccgtgggggaatcctcgtgataggaactggaatatgccttgagggggacactatgtctttaaaaacgtcggctggtcatgaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctggtatgtatgagaggatgggaccttgtggaagaagaggtgccaggaatatgtctgggaaggggaggagacaggattttgtgggagggagaacttaagaactggatccatttgcgccattgagaaagcgcaagagggaagtagaggagcgtcagtagtaacagatgctgccggcagggatgtgcttgaggaggatccagagatgagagcaggtcactgggaaaggttaggggcggggaggccttgattggtgttggtttggtcgttgttgattttggttttatgcaagaaaaagaaaacaaccagaaacattggagaaagctaaggctaccaccacctacccggtcagtcactcctctgtagctttctctttcttggagaaaggaaaagacccaaggggttggcagcaatatgtgaaaaaattcagaatttatgttgtctaattacaaaaagcaacttctagaatctttaaaaataaaggacgttgtcattagttctttggtttgtattattctaaaaccttccaaatcttaaatttactttattttaaaatgataaaatgaagttgtcattttataaaccttttaaaaagatatatatatatgtttttctaatgtgttaaagttcattggaacagaaagaaatggatttatctgctcttcgcgttgaagaagtacaaaatgtcattaatgctatgcagaaaatcttagagtgtcccatctggtaagtcagcacaagagtgtattaatttgggattcctatgattatctcctatgcaaatgaacagaattgaccttacatactagggaagaaaagacatgtctagtaagattaggctattgtaattgctgattttcttaactgaagaactttaaaaatatagaaaatgattccttgttctccatccactctgcctctcccactcctctccttttcaacacaaatcctgtggtccgggaaagacagggactctgtcttgattggttctgcactggggcaggaatctagtttagattaactggcattttggcttttcttccagctctaaaacaagctccatcacttgaaatggcaaaataaaatcatggatgaggccgagggcggtggcttatgcctgtaatcccagcactttgggaggccaaggtggtaggatcacgaggtcaggagatcgagaccatcctggccaacatggtgaaaccccctctccactaaaaatacaaaaattagctgggcgtagtggcatgtgcctgtaatcccagctactcaggaggctgaggcaggagaatcacttgaaccaggaggcagatgttgctgtgagccaatatggcaccactgaactccagcgacagagctaaactccatctcaaaaaaaaaaaaaaaaaaaaaaaaacatggatgatcggtgtcgttgagaggataggtatttggaagaacctttgtttgaaactggctctgtacatacaatgaaattacatacttatttacatacaatgaaatgcagaggttttttttttatataggatctctgtcgagaggctggagtgcagtggtgctatcacagctcactgcagcctcaacctcgtcaggctcaagcaatcctcccacctcagcctccagagtagcagggacgataggtgtgcaccaccatgcccagctaatttttgtatttttttttctttttttgagatggagtcttgctctgttgcccaggctggagtgcagtggcgcgatctcagctcactgcaaactctgcctcccgggttcatgccattcttctgcctgagcctcctgaatagctgggactacaagcacccactaccacgcccggctaattttttgtatttttttttcttttttagtagaggcgggatttcaccgtgttagccaggatagtcttgatctcctgaccttgtgatccacccgcctgggcctcccaaagtgctaggattacaggcataagccactgcgtccagccattcttgtatttttctgttgtagagatagggttttgctatgttggccatgctggtctcaaactcctgacctcaagtgatctaccctcccttggcctctcaaggtgctgggattacaggcctgagccattgcacccagccatggtctaaaaatcttgattgaaataccaccttttcatttccagacacccctatttaaaattaccacacccccagcacacactttatcttctattcctgctgcttctccataacactgattactagctgacattctatgtaatgtatccattttttatctctagtcccacagaatgtaaactccaggatgggatttttgttttgtttacatacatctgtatgttcagtagttagaacggtacttgggacctagttgccactcaataaacatttgtcaaataaataataaactaaactaaattagttctttaatttttttaaatatggtgatggttagtagtgagtaacattcaaaaaataagttgaaaagttgtaccattgcctcttacccacaataaaaaagggtaaattcttttctgctttatgaaagttgtttttcatatttgaagtcaagttaatcagattaaggaaaatgtatgttgtgttttcagagcgatacaagatttataaataaccatcctctcccttgcccttcaacattatagctaaacaaaaataagaggaaaacaggattcacaatttatcaatttattgaaaatcagagccagagaagcaggaaatgacattgtaggaaaaaactgcttttgaaaaagcacaaaacttactcatgacaatcagtgatcaggaaaatcctcaatagtgtggcatttggatacatttatgtttcatttccatgggagagagtcataaaaataggatgttctttctcattctggcaaattaaaccatcaattaaaaactcagatacataaaaattaaagatgtaagaatgaaaatgctaaattgttattttcaatcaactattatgttttctagcttttcattgcttttttctgtttcctgttaagattaatttctttttttttttttttttttttttttgagacagactttggctcttgttgcccaggctggagtgcagtggcacaatctcggctcactacaacctccacctcccgggttcaagcaattctgctgcctcagcctccggagtacctgggattgcaggcatgtgccatcacaccagctaattttgtatttttagtagagacagggtttctccatattggtcaggttggtctcgaactcctgacctcaggtgatcctcctgccttggcctccgaaagtgctgggattacaggcgtgagccaccgctcccagactttttgttttgttttgttttgtttttttgagacacggtctcgctctgctgcctaggctggagtgcagtggcacgatcttggctcactgccagctccgactcccgggttcaggccattctcctgcctcagcctcccgagtagctgggactacaggcgcccaccactatgcccggctaattttttgtatttttagtagagacggggtttcaccatgttagccaagatggtctcgatctcctgaccttgtgatccacccgcctcagccttccaaagtgctgggattacagtcctgagccactgcgcccggcctggacctttttttttcggggtggggggttggagtctggctctgtcgcccaggctggagtgcagtggcgccatcttggctcactgcaacctccgcctgccaggttcaagttcaagcgcttctcctgcctcagcctcctgagtagctgggattataggcgcacgccaccgtggccggctaattttgtatttttagtagagatagggtttcatcacgttggtcaggctggtcttgaagtcctgatctcgtgatccacccgcctcggccttccaaagtgctggcgtgagccactgcgcctggcttaagattaatttttgtttgttttgtttttgagacggagtctcgctctttcacccaggccggagtgcagtggcgccatctcggctcactgcaagctccgcctcccgggttcacgccattctcctgcctcggccccccaagtagctgggactacaggcgtccaccaccacgcccggctaattttttgtatttttagtagagacggggtttcaccgtgttagccaggatggtctccacttcctgacctcgtgatccgcccacctcggcctcccaaagtgctgggattacaggcgtgagccaccgcgcccggccttaagattaatttttatggtgttttacattcatttgtatggaaagttctaggatagggatcatatttcacttccttttaatatagtacagtatagcacaatttgcagttatgtcttaatatgtgatcaggaatgatcatgactggaaacagtgttatttgtggtagctatagggtaggtaaggttttcagcctgttttaggtttcttgaactaaaattccttctgctgtcttctaagtcaatattggcagctatttctgacaattggtagttctttgtaactttttacctatgactataacatttttgactttcagaagaatttgctaaaatgtgttccccggtgggttgttgtttttcaacctaaacctagctgctttttccagtcacttatccgtattggaagctcaaaatgcaaatatacagtaggcctaaaatattgcctggtttgaaaagtgtttaaaatatttgaatcatttttatagtaaacatttactctcatcaggacctagaaggggaacattttaattttttttcttttcccttttcacagtcttccttcaacattcattacctttttacatatcggagttttcatctgttcaaagtttgtgtttacagtgtgtttatatagtttagattataattaccatactgaaatataattgtttcagaattgagtcagtggtgagaatgaaagccatctggtatgataactgaatccaatttttcttttacggagaatttctttgaaatgtagcttatctcagaaatagggatttagtaaccaatcagagttttctttgtcaaggttgtttttctttttaaagtcacatttggtcccagtaataataccaatgttggtacaagttatctcaggttgtgaagcatttttcccaagtcatctcaggttgtgaagcattttcccaagtagcatttaattttattcttgcaatagcccaaggagtctggcagggtgaatggcaagagaaggaaacaggttcaggtagagtggttagcccaaggtggctctgcttatatacacaactggtagtagaaacccagcctcctgacttagttcattgtttttcttttcactgccctgtgctatgtcaaaaaccccatgattacaagagttgtattacaacccttcacaataaggttactgtccacaagcttttcttgtgatccttttcttttttttttttctttttttgagatggattctctgtcacccaggctggcccgccttggcctcccaaaatgctgggattacagcgtgagccaccgcacctggcccttgtgatccttttctaaaaagttaaatatttaaggaaaaaaccacattcttgtcacactgccaggttagtcgttctttgatatcttgcctggactttatccaaaaaatccgtttcaaaaattcacatttagagctaagtgtagtggctcacgcctgtaatcccggtcgaggcagatggatcacttgaggtcaggacttcaagaccagcctgggcaatatggtgaaaccccttccctaccaaaaatacaaaaaaattagccgggtgtggcagcacgcgcctgtagtcccagctacttggaatgctgaggcacaagaatcacttcaatccgagaggcagaggttgcagtgagccaagaccacaccactgcactccagcctgagcagcagagtgagtgagactccatctccaaaaaaaaaaaaaaaggttcacattcagaagaaagctaaaggccgggtatagtagctcacacctgtaatcccagcactttgggaagccgaagcaggaagattgcttgatgccaggcattcaagaccagcatgggcatcatagtgagatcctgtctctacaaaaattaattaacattaaaaattaaaaagatggctggcatggtggctcactcctgtaatcccagtactttgggaggccaaggcatggtggtgcatgcctttagtcccagctactcgggaggctgaggcaggagaatcacttgaattcaggaggcggaggttacagagagccgagatggtgccactgcactccagcctgggcgacagaacgagactctgtctgaaaaaaaaaaagaaaattaaaaagaccagaataaagctaaagatttaaaatagcctataggttcctaccagaagttaccagctacctctctgatagtctttccctacaatatcctcctggattattacattttagcaccttgacctatctgatgtcctgcatacacaggcatggtcctgctcagggtttgccttctctgctccctctttcttggaatgctcttcccctaattgttgcatagtgtgtttctttacattattaagctatcctctagtctcacctcagtgaaacctttcctgactccccccatgtacatctcacccccacatagatattgaactacctgtttccccttaccctgcttaatttttctctttaatgcacttattcccatgtattctttaattccgtatcaactgtctaccacactagaatatgagctctatgagagcaggctttattttgtaaactgctacatttctatctcctagaatagtacttgaatatagtagtagatacttaataaacacttgttatattagtataataaatgaactaatctcaggaatgccttggttttgtggatagacaggtagggatgggaacttgggtgatgtattttctgaagtttttatttttaagcttattattattttgagatggagtccagctctgtcgcccaggttggagtacagtggcgcgatcttggctcactgcaacgtgcacttccccggttcaagcgattctcctgccttagcctcccaagtagctgggattacaggcgcatgccaccatgcccagttagttttggtatttttagtagagacagcgtttcactgtgttggccaggctggtctcgaaatcctgacctcatgatccgcccgcctcggcctcccaagtgctgggattacaagcatgagcccccgtgtctggccttattttcttttttttgagacagagtcttcctctgtcacctaggctggagtgcagtggcacgatattggctcactctgcaacctccacctccaggattcaagtgatccttctaccttagtctccaaagtagctgagaccacaggcatgcgccaccacgcccggctaatttttgtatttttagcgtagacagggtttcaccatattgtccaggatgatctggaactcctgagctcaggtgatccacccacctcagcctcccaaagtgctaggattacaggcatgaggcaccatgcccggccttaagcttatcattttctaaatttcctttagtgagtacttattacactgtttttacaaagtaatcacaaaccaaacatcatgcctcttctgaagtgatctaataagagtacacagtaccatctgtaaagtgttcttgccagaaagttgaacctgaatgattaagcctgtaagtctagtttataggaaataaggctagaggaacaagttaaacctcaccatagggttatacaatcagcaaaatccagaatgggggaaactccacaggtcaaatgacctaattttaaaaataaatgacaagggagaaaaagtaagagacacctatagatcagaagacacttggggctgggcatggtggctcacacctgtaatcccagcactttgggaggccaaggcaggcggatcacctgaggtcaggagttcaagaccagccggccaacatggtgaaaccccaactctactaaaaatacgaaaaatcagccgggcgtggtggcgcacgcctgtagttccaactacctgggaggctgaggcaggagaatcacttgaacttgggaggcagaggttgcagtgagccgagatcgcaccattgcatgccagtctgggctacaaaagcaaaaccccatctcaaaaaaaagaagacacttgggtttgggtgtgttggctcatgcctgtaaaccccgtgctgggaggattgcttgagcccaggagttcaaggctgcagtgaggtatgtttgcaccactgcactccagcctaggtgacagagtgtgaccttatcttaaaagtaataataattaaaataatctggggtaggggtggatatgggtgaaacagcttggccatgagttgatggttgttggaccagggtgatggtccatatagttcattttattattttatttacttgaaattttgaaatacttgaaattttccatattaagttaaaaaggcatttacagtaaacaaaaaaaaagttctaggaaggaattcaaaagaaatataagcagaaaattttgtctttatggagcttaaagatgagatgtgcacccacagtgatagtgcagaaaaatatatcactggaaatgaattcgtacgaactattatcaactaatcttttaaatgctgatgatagtatagagtattgaagggatcaatataattctgttttgatatctgaaagctcactgaaggtaaggatcgtattctctgctgtattctcagttcctgacacagcagacatttaataaatattgaacgaacttgaggccttatgttgactcagtcataacagctcaaagttgaacttattcactaagaatagctttatttttaaataaattattgagcctcatttattttctttttctccccccctaccctgctagtctggagttgatcaaggaacctgtctccacaaagtgtgaccacatattttgcaagtaagtttgaatgtgttatgtggctccattattagcttttgtttttgtccttcataacccaggaaacacctaactttatagaagctttactttcttcaattaagtgagaacgaaaaatccaactccatttcattctttctcagagagtatatagttatcaaaagttggttgtaatcatagttcctggtaaagttttgacatatattatctttttttttttttttgagacaaagtctcgctctgtcgcccaggctggagtgcagtggcatgatcttggctcactgcaacctccgccccccgagttcaagcgattcttctacctcagcctcccaggtagctgggactacaggcacccgccaccatgcttggctaatttttgtacttttagtagagataaggtttcaccatattggccaggctggtctcgaactcctgaccttgtgatccacctgcctcggcctcccaaagttctgggattacaggcgtgagccaccacacccgactgacatatattatctattaggatgtaacatcattttgaacagtgttttgtattttttgtgtccatcagtgaaagcaaactgcaagcagttttgaaataagcacattgtgtttgagccttcccagtttctcctttctgttcatttctgcatatccttatgcattcccccttctaagggtcagtgtttgcccgctttgtaatcattgtgaagacaggaaaggacctgataccagtttctatttaggccaaaattcatttatagcagtgattcaagttatatttacgtatttgatgatcttgtcttttgaaatgaaaatgtttgtttcttaataaaagaatttcagaaaaagtagagtaggtaatttagtagaacaagtgggctttctccttttctttatgttaagctatggctcacatcttaccttaaatgtcaactaatttgtttttaagtatttatgtacctggtacataacctggtaccaggtacaaactatgtacttggtaaaaagtttattagcacaaaaaggtatatgatgcaaagtatacttccctcttaccctacaacccctgcctccctgttccctccccagacaaccacaatgatcaatttcttatgtatcctttgaggaatttttaaattccagagttcttaacttggggtttatgaatagtctttatgaatttcctagaattatatttaaattgtattcaaaactatggccatgtacatttttctgggaagatagtccataattttcatctgagtgagctaagatcatgccactgcattccagcctgggcgacaagagggagactcaaaaaaaaaaaaaaaaagtcccagtatttactacagagagctaaagattaacctttaaagccctggggctttcaatttatctggatgagaatctttctggaatgaactgtatgttttcttgtcagcttgagtaacaaatgctgaacatactatactattattacagggactcaagggcccagtgtggtagctcctgcccataaccccagcactttgggaggccaaggcaggaagatcacttaaggccaggagttcgaagctgtagtgagctatgatcacaccactgcactccagcctagatgacagagtgagaccctgtctttttttttttttgagatggtgtttcactctattgcccaggctggagtgcagtggtgtgatctcggctcactgcaacctccacctcctgggttcaagcgattctcctgcctcagcctcttgagtagctgggattacaggcatctgccaccacacccagctaatttttgtatttttagtcgagacagggttttcaccatgttggccaggctgctctcaaactcctgacttcagctaccttggccttaaaaagtgttgggattacaggtgtaagccaccgcgcctggctgaccctgtctcttaacaaaaaaagagagattaagttatgaatatagttgctttgagaacttgtggaagaaggaaattataggcttataggcagagataataatacgagcaaatgtacaaataaaagaaaatagaggacgggcgcggtggctcacgcctataataccagcactttgggaggtcgaggtgggcggatcacgaggtcaggaaattaagaccatcctggccaaaatggcgaaacactgtctctactaaaacacacaaaaaactagcctggcatggtggcacgtacctgtagtcccagctacttggtaggctgcggcaggggtatcacttgaacctgggaggcagaggttgccctgagccgagatcatgccaatgcactccagcctgacaacagagtgagactctgtctgaaaaaaaagaaaagaaaagaaaatacatccaggaaaaataagctaactttgcatatgtgtataggagttgtgttagaaaaggaagaagccctcaaagatgggaagccatttgcaagaaagagaaggtccaagaggaggcagaagggattggaaatagaaaaaggatgtaagaaagagttgattattactcataaacagtaatgaaggaaaaggagagtaattctacaggaagatgctgaggtgctttgagcccagtgaagttggaggtaaagacagctgttgaggccgggcacggtggctcacgcctctaatcctagcacttttggagcccaaggcaggtggatcacctgaggtcaggagctcaagaccagcctgaccaacatagagaaaccccatctctactaaaaatacaaaattagacgggcgtggaggcgcatgcctgtaatcccagttacttgggaggctgaggcaggagaatcacttgaacctgggaggcggaggttgcagtgagccgagattgcgccattgcactccagcctgggcgacaagagtgaaaactgtctcaaaaaaaaaaaacaacaaaaaacagctgttgagattgagaggattagagttggcaactggagaagagtgagaagcttggtttcaagcttgtgatagtcaggattgtgatagtcaggaaagaaccagtcataaagatatatgtgtgtgtatacatataaatatgttatatatatgtgtgtgtgtgacacatatatatttttgtttgtttctttgagacagtgtctccctctgacacccaggctggagttcagtggtgtgatcatagttcacttttaccttgcaatctgggttcaagcaatctctcatctcagcccctcaagtagctaggactacaggtacatggcatttgcccagctaatttttaagtttcttgtagagatgggccagccatattttaaattgtgttttgaatgttatattagaattaaaagtccaaagccgggtgtggtggctcacgcctgtaatcccagcactttgggaggctgaggtgggcggatcacgaggtcaggagttcgagaccagcctggccaatatggtaacaccatctctactaaaaatacaaaaattagctgggtatgggggcacatgcctgtagtcccagctactcaggaggctgaggcagaggaacctcttgaacccaggaggcagaggctgcagtgagttgagatcgtgccactgtactctagcctgggcgacagagcaagattccgtctcaaaaaaaaaaaaagtccagtataatgcccatgtgatagatcgactttttcatgaaatctcttctgtaatatcaatataatctgaataacactttgatctatatgatgagaaagctgggagcctgggagcgatacccccatgcttttgttgtattaattgtattttctacggataaactctaattgctaaaaataaaacaactttattgacccaagcaagcctaaagttctgaaatcttttttttatttttgtttgtttgtttgtttgtttttgtttgttttgttttgagacggagtctcgctctgtcgcccaggctggagtgcggtggtgcagtctcggctcactgcaagctccacctcccgggttcacaccattctcctgcctcagcctcccaagtagctgggactacagacgcctgccaccacgcccagctaatttttttgtatttttagtagagaaagggtttcaccgtgttagccaggatggtctcgatctcctgacctcgtgatctgcccgccttggcctccctaagttctgggattacaagtgtgagccaccacgcccggctgtttttttttgttttgttttgagacggagtctcactgtgttgcccagactggagtgcagtggcatgatctcagctcactgccacctccatctcctgggttcaagcaaatctcctgcctcagcctcccgagtagctgggactacaggcatgtgccaccacacctggctaatttttgtatttttagtagagacggggtttcactatgttggccaggctggtccaaaactcctgacctcaggtgatctgctcgccttggcctcccacagtgccaggattacaggcatgagccaccttgcccagccagttctgaaatcttttatgaagcctataaaaaaagataataataccaatctagaaaatatttcttaaggcagtcatgcattagtttgaactttccaaacaaaaaaatgcaatgtgtaatacttttttttttttttttgagatggagtcttgttctgttgcccaggctggagtgcagtggtacaatctcggctcactgcagcctctgcctctctggttcaagtgattctcctgcctcagcctcccaagtagctgggattacaggcgtgcaccaccatgcatggctaatttttgtatttttagtagagacagggtttcaccatgttgacaaggctgatctcgaactcctgacctcaggtgatccgcccacctcagcctcccaaagtgctgagattacaggcattagccaccacgcccagccttttattttagtagagaccatgtttcaccatgttgaccaagctggtcttgagctgacctcaagtgatccgcccacctccacctcccaaaatggtgggattataggcatgagccaccgcacccagcctgtaatacttttttgaagatctagaaccacattgttcaaagagatagaatgtgagcaataaatgtaacttaaatttttcaacagctactttttttttttttttttgagacagggtcttactctgttgtcccagctggagtacagtggtgcgatcatgaggcttactgttgccttgacctcctaggctcaagcgatcctatcacctcagtctcccaagtagctgggactgtaagtgcacaccaccatatccagctaaattttgtgttttctgtagagacggggtttcgccatgtttcccaggctggtcttgaactttgggcttaacccgtctgcccacctaggcatcccaaagtgctaggattacaggtgtgagtcatcatgcctggccagtattttagttagctctgtcttttcaagtcatatacaagttcattttcttttaagtttagttaacaacctttatacatgtattctttttctagcataaagaaagattcgaggccgggtgcggtggctcacgcctgtaatcccagcactttgggaggctgagatgggcacatcacgaggtcaggagatcgagaccatcctggctaacatggtgaaaccccgcctctactaaaattacaaaaagttagccaggcgtggtagcgggcacctgtagtcccagctactcaggaggctgaggcaggagaatggcgtgaacccaggaggcagagcttgcagtgagcagagattgtgccactgcactccagcctgagagacagagcgagactccgtctaaaaaaaaaaaaaaagattcgaatccttatcttggttgatttttgcgtatctagttccactgaattatttatataattgtatagactacagcacgagacagcttagcttgtcactctactgtactatattctgcagtactatcataagggaatttcctccctacccctgctctgaattgttcaattgtactatttgctggagtaatgcttgatgccttcttgatccattatactagagtatatgtagtatttgtagattctgaaggagtgggagcctctattctgagttttaaaggtacttatgtacagtggaggtagctttttgacagcctcatcttccaaactatagagtcattgttttgttgagtgcaatatggtacttgaagcatctatatcggcgaagaaggacccaagtctccttgaccttacctacctacattcactttctctggtaggaagattgtgggtgcctctctccagacttagtttccatgtcaaaaaagaaaaaaggaagattgtgggctttgctacaatccaattctggatccaatataaccttcattgcttaattactgtgtgatctgggacaagcctctactctataaaaatgaagataaggccaggcttgatggctcatgcctgtaatcccagcacgttgggatgccaaggcaggaggatcacttgaggtcaggagttcgagaccagactgggcaatatagtgaaaccacatctgtacaaaaataaagatagaaagtagcccagcgcaatggctcacacctgtaatcccagcactttgggaggctgaagcaggcgatcacttgaggtcgggagttcaagactgtagacagatagataggtaggtagatagataggtaggtagatagatagagatatagatatagttggggtttttttgttttgttttgttttgtttttgagatggagtttcgctcttgttgcccaggctggagtgcaatggcgcgatctcagtttactgcaacctccgcctcccgggttcaagagattctcctgcctcagcctcctgagtagccaggattacaggcatatgccaccatgcccggctaatttttgtatttttagtagagacagggtttctccgtgttggtcaggctggtcttgaactcctgacctctcccaaagtgttgggattacaggcgtgagccaccgctcctggccttttttttttttttttttttttttttgagacagagtcttcctctgttgcccagggtggagtgcagtggcactcttctcagctcattgcaacctctgccatcctgggttccagtgattctcatgcctcagcctcccaagtagctgggactcaggcgtgtgcccaccacgcctggctaattttgttgtatttttagtagagacagggtttcaccatgttagccaggctggtctcaaactccaggcctcaagtgatctgcctgcctcagcctcctgggattgcagacatgagccactgcacccggccaagagagggtaataaatgttaaattacctggctagtaaaaaatattctctaagtgtcttttctcacaattcccaatgccttttttttttttttggcacaatctcactctgttgcccaggctggaatgcaatggtgcaatattggctcactgtaacccccgcctcacaggttcaacttattctcatgcctcagcctcccgagtaactgggactacagtgcaccaccaccacacccagctaatttttgaatatttagtagagacagggtttcaccatgttggccaggctggtcttgaactcctggcctcaagtgattcacccaccccgcaagtgctgggattacaggtgtggaccaccgtgcacagccctagtgactttttttttagccccttaatcttttctttcctgggtctcttcattgtcagtgtctgctatttactccctacctagtcacccccttcaccagtatattatgtcctttatgttttattttgcaggatcttattttgcttttctattgaatcccctccatctagaatagtactagacatagtaaatattggttgtatgagtgaatcgctgcttttaattatcatcaccattgctctctctacttctggtctatgatccactttgagttaacttttgttatttggtgtgagataggagtataatttcattcttttacatgtggttatacttttgtctcaacactgtttgttaaaaacacaaaaagtattattttcccatttaatcatctttggcctgggcacggtggctcatgcctgtaatcccagcactctggaaggccaaggcagatggatcaatttgaggccaggagttcaagactagccaacatggtgaaactaaaaatacaaaaaattagctgggtatggtggtgcatgtctgtaatcccagctactcgggaggctgaggcacgagaattgcttgagcctaggaggtggaggttgtagtgagctgagattgtgtcactaccctccagcctgggtgatagagtgagtctgtctcaaaaaaaaaaaaaaaaaattaagaaaataaaaatcgtcggccaggcatggtggctcacacctgtaatcccagcactttgggaggcagaggcgggcagatcacgaggtcaggagatggagaccatcctggctaacatggtgaaaccccgtctctactaaaaataaaaaaattagccgggcatggtgctgggcgcctgtagtcccagctgctcgggaggctgaggcaggagaatggcgtgaacccaggaggtggagcttgcagtgagccgagatcgtgccactgcactccagcctgggagacagagcgagactccgtctcaaaaaaaaaaaaaaaaaaaattgtcttggtatttattattgttgaaaatcgcttgatcacagatgtatgtatgagtttatttctgtactgtcaattccattttattgatgtatgtgtctattcttatgctattaccacactttcttgattactatagctttgtggtgaggtgttgagattttaaactaattataagcatcttacatgaactacttaccgtttatatttgattatgcagcatgaaataattatgaatatatcattaaatatgccatattaacttttattaagttttatgtgatcataacagtaagccatatgcatgtaagttcagttttcatagatcattgcttatgtagtttaggtttttgcttatgcagcatccaaaaacaattaggaaactattgcttgtaattcacctgccattactttttaaatggctcttaagggcagttgtgagattatcttttcatggctatttgccttttgagtattctttctacaaaaggaagtaaattaaattgttctttctttctttataatttatagattttgcatgctgaaacttctcaaccagaagaaagggccttcacagtgtcctttatgtaagaatgatataaccaaaaggtatataatttggtaatgatgctaggttggaagcaaccacagtaggaaaaagtagaaattatttaataacatagcgttcctataaaaccattcatcagaaaaatttataaaagagtttttagcacacagtaaattatttccaaagttattttcctgaaagttttatgggacatctgccttatacaggtattagaaacttactgcctttctctaatgcttctagtgtaaaaacttgcagacttatgtaaagtagggctgtatcgccgtgcccccattgtctgttaatcttgtttttatatttttgattgtgtttccttttctttttttttttttttttaagacagggtcctgctctgtcactgaggctggagtgcagtggcgtgatctcggctcactgtagcctctgtctcccagcctcttcctgccttagcctcccaaatagctgggactacaggcacacgctaccatgcccggccaatttttgtattttttgtagagatgaggttttaccatgttgcccaggctggtaactcctgagctcaggtgatctgcccacctcggcctcccaaagtgctggggttcacaggtgtgtgtttatttctatctaattatttacacaaacacaatgtatttatatattgtgtatctcttctgctacaatgtaaattctatgagagtagtaattttgtctgtctcaacactgtttttcctaagtttggtacatagtaggcactcagatgcttaaaggaatgaatgaattgtgctttaattccactttactaaacccaaatctccctttggacattgttatctatgtgttttcaaagaagtataatcataatttgacagaaatccttgagaggcagaactaagtgagggattgggcagggttcagatgttaagaacagtaagctcagcagggtgtgattgctcatgcctataaccctagcactctaggaggctgaggtgggatgattgcttgaggccaggagtttgaaatcagcctgggcaacatagtgagaccccatcactaccaacaaaataaataaataaatgtacatggtggcatatgcccatagtcctagctacttgggaggctatagtgggaggatagcttgagtacagaagtctgaggctgcagtgagctatgattgtggcactgcatgctagcctgggcaatagagcaagaccctgtctctaaattaaacaaaaaaaaaagtactctagttttctatgcaatgcattatatctgctgtggatttagggcagtattatatcagataattttaggcatttggtaggcttaaatgaatgacaaaaagttactaaatcactgccatcacacggtttatacagatgtcaatgatgtattgattatagaggttttctactgttgctgcatcttatttttatttgtttacatgtcttttcttattttagtgtccttaaaaggttgataatcacttgctgagtgtgtttctcaaacaatttaatttcaggagcctacaagaaagtacgagatttagtcaacttgttgaagagctattgaaaatcatttgtgcttttcagcttgacacaggtttggagtgtaagtgttgaatatcccaagaatgacactcaagtgctgtccatgaaaactcaggaagtttgcacaattactttctatgacgtggtgataagaccttttagtctaggttaattttagttctgtatctgtaatctatttttaaaaaattactcccactggtctcacaccttattttatcaatcgtaaggtgcacatttttcacatcttaacatctctgaaattgggaacattttactattgagggtgtgtcatttgtttaatttgtgtgctttctttcttagtgatacagaaaataatagtgcaacttacattgttggtgtcttagctttagtgaaatacagtattgataggcaaatttcttagtgttaaggtagaaaacaaggactctaaataactttgatggtctgtgtatttgtttttgtttcctaggagtaaaatttccagttgattttttaaaatttgatttttaaaaaaaatcacaggtaaccttaatgcattgtcttaacacaacaaagagcatacatagggtttctcttggtttctttgattataattcatacatttttctctaactgcaaacataatgttttcccttgtattttacagatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggtaaaaccatttgttttcttcttcttcttcttcttcttttcttttttttttcttttttttttttgagatggagtcttgctctgtggcccaggctagaagcagtcctcctgccttagcccccttagtagctgggattacaggcacgcgccaccatgccaggctaatttttgtatttttagtagagacggggtttcatcatgttggccaggctggtctcgaactcctaacctcaggtgatccacccacctcggctccccaaattgctgggattacaggtgtgagccactgtgcccggccggtaaaaccattttcatttattctggcaacatctctttattgagcattgtgaatatgttagtgaatgtgctagatgctcatagatttatataaaaagttagtgaagaaggaaagatggtatattaagtggttagacaagtgttctaatcagttagagttcagagaaggtcagggtacctgatataatcaagagagagaccttacagccaggtgaggtgaatgtacctataatcccagctacttaggaggctgaaatgggaggatcacttgagtccaggtttgagaccagcccaggcaacatagcaagatccccatcagatacaccaaaaagacagatttcttttttttttttttttttgagacagagtctcgctctgtcgcccaggctggagcgcagtgacacgatgtcagctcactgcaacctccgcctcccaggttcaagtgattctcctgcctcagcctcctgagtagttgggactacaggggtacgacaccagacctggctaatttttgtaattttagtagagtcggggtttcaccatattggtcaggctggtctcgaactcctgacctcaggtgatccaccctccttggcctcccagagtgctgggattacaggcgtgagccaccaagcccggccaaaaaagagagctcttataggcccttccttgctttggagctttatctgctctgtgatgcttatctaaaatagccataaggtcactgatatttttaagcatttggaaattacttcagctgggtgccatggctcatgcctataatcccaaccctttgggaggctgaggtaggaggtcctttgagcccagcttgggcaacacagtgagacactgtctctgcaattaaaaaaaaaaaaaaagtagctgggtgccgtggctcacgcctgtaattccagcactaggaggcttgaggattgcctgagctcaggagttcaagaccagtttgggcaacatagcaagtccttgtctatattaaaagtttttttaaattatctgggcatggtggtgtgtgcctgtagtcccagctacttgggaagctgagacagaaggatcacttgagtccaggagatgtagactacagtgagctatgatcactccactgcacttcagcgtgggcggcaaagcaagatctagttgcaaaaaaaaaaagaactggctgggtgcggcggctaacacctgcaatcccagcaccttgggaggctgaggccagtggatcatgaggtcaggagattgagaccaccctggccaacatggtgaaacccggtctctactaaaaatacaaaaattagctgggtgtggtggcacgtgcctgtaatcccagctactccagaggctgaggatggagaatcacttgaacctgagagtcggaggttgcagtgagccgagattgcgccactgcactccagcctggcgacagagcgagactccgtctcaaaaaaaaaaaaaaaaaagcttcacgcctgtaatcccagcactttgggaggccgagtcaagtggatcacgaggtgtggagatcaagactatcctggctcacatggtgaaagcccgtctctactaaaaacacagaaaaattagctgagcgtgatggcggactcctgtagtcccagctactcgggaggctgaggcaggagaatagcatgaacccgggaggtggagcttgcagtgagccgagatcccgccactgcgatccagcctgggcgacagagtgagactctgtctcaaaaaaaaaacaaaaaaacttagctgggcgtggtggtatgcacctgtggtcctagctacttgggaggctgaggctggagcattgctttaacatagagagtcaaggctgcagttgagctatgactgtgccactggactccagcgcaggtgactgagaccctatcttttaaaaaaagggaaaattacttgaacttaaaaggtgtaattgttaaagaaaatgtagtgatttgctctgttgttacttatatgtgcatgaatgatggagatcttaaaaagtaatcattctggggctgggcgtagtagcttgcacctgtaatcccagcacttcgggaggctgaggcaggcagataatttgaggtcaggagtttgagaccagcctggccaacatggtgaaacccatctctactaaaaatacaaaaattagctgggtgtggtggcacgtacctgtaatcccagctactcgggaggcggaggcacaagaattgcttgaacctaggacgcggaggttgcagcgagccaagatcgcgccactgcactccagcctgggccgtagagtgagactctgtctcaaaaaagaaaaaaaagtaattgttctagctgggcgcagtggctcttgcctgtaatcccagcactttgggaggccaaggcgggtggatctcgagtcctagagttcaagaccagcctaggcaatgtggtgaaaccccatcgctacaaaaaatacaaaaattagccaggcatggtggcgtgcgcatgtagtcccagctccttgggaggctgaggtgggaggatcacttgaacccaggagacagaggttgcagtgaaccgagatcacgccaccacgctccagcctgggcaacagaacaagactctgtctaaaaaaatacaaataaaataaaagtagttctcacagtaccagcattcatttttcaaaagatatagagctaaaaaggaaggaaaaaaaaagtaatgttgggcttttaaatactcgttcctatactaaatgttcttaggagtgctggggttttattgtcatcatttatcctttttaaaaatgttattggccaggcacggtggctcatggctgtaatcccagcactttgggaggccgaggcaggcagatcacctgaggtcaggagtgtgagaccagcctggccaacatggcgaaacctgtctctactaaaaatacaaaaattaactaggcgtggtggtgtacgcctgtagtcccagctactcgggaggctgaggcaggagaatcaactgaaccagggaggtggaggttgcagtgtgccgagatcacgccactgcactctagcctggcaacagagcaagattctgtctcaaaaaaaaaaaacatatatacacatatatcccaaagtgctgggattacatatatatatatatatatatatcatatctatatatatatatatgtaatatatatgttatatatatattacatatatatatgttatatatatgttatatatatataatatatatatgttatatatatgttatatatatatatacacacacacacacatatatatgtatatatatatacacacacacacacatattagccaggcatagttgcacacgcttgtagacccagctactcaggaggctgaggcaggagaatctcttgaacttaggaggcggaggttgcagtgagctgagattgcgccactgcactccagcctgggtgacagagcaggactctgtacaccccccaaaacaaaaaaaaaagttatcagatgtgattggaatgtatatcaagtatcagcttcaaaatatgctatattaatacttcaaaaattacacaaataatacataatcaggtttgaaaaatttaagacaacagaaaaaaaaattcaaatcacacatatcccacacattttattattactactactattattttgtagagactgggtctcactctgttgcttatgctggtcttgaactcctggcctcaagcagtcctgctccagcctcccaaagtgctgggattataggcatgagctaccgctcccagccccagacattttagtgtgtaaattcctgggcattttttccaggcatcatacatgttagctgactgatgatggtcaatttattttgtccatggtgtcaagtttctcttcaggaggaaaagcacagaactggccaacaattgcttgactgttctttaccatactgtttagcaggaaaccagtctcagtgtccaactctctaaccttggaactgtgagaactctgaggacaaagcagcggatacaacctcaaaagacgtctgtctacattgaattgggtaagggtctcaggttttttaagtatttaataataattgctggattccttatcttatagttttgccaaaaatcttggtcataatttgtatttgtggtaggcagctttgggaagtgaattttatgagccctatggtgagttataaaaaatgtaaaagacgcagttcccaccttgaagaatcttactttaaaaagggagcaaaagaggccaggcatggtggctcacacctgtaatcccagcactttgggaggccaaagtgggtggatcacctgaggtcgggagttcgagaccagcctagccaacatggagaaactctgtctgtaccaaaaaataaaaaattagccaggtgtggtggcacataactgtaatcccagctactcgggaggctgaggcaggagaatcacttgaacccgggaggtggaggttgcggtgaaccgagatcgcaccattgcactccagcctgggcaaaaatagcgaaactccatctaaaaaaaaaaaagagagcaaaagaaagaatatctggttttaaatatgtgtaaatatgttttggaaagatggagagtagcaataagaaaaaacatgatggattgctacagtatttagttccaagataaattgtactagatgaggaagccttttaagaagagctgaattgccaggcgcagtgctcacgcctgtaatcccagcactttggaaggccgaggtgggcggatcacctgaggtcgggagttcaagaccagcctgaccaacatggagaaaccccatctctactaaaaaaaaaaaaaaaaaaattagccggggtggtggcttatgcctgaaatcccagctactcaggaggctgaggcaggagaatcgcttgaacccaggaagcagaggttgcagtgagccaagatcgcaccattgcactccagcctaggcaacaagagtgaaactccatctcaaaaaaaaaaaaaaagagctgaatcttggctgggcaggatggctcgtgcctgtaatcctaacgctttggaagaccgaggcagaaggattggttgagtccacgagtttaagaccagcctggccaacataggggaaccctgtctctatttttaaaataataatacatttttggccggtgcggtggctcatgcctgtaatcccaatactttgggaggctgaggcaggtagatcacctgaggtcagagttcgagaccagcctggataacctggtgaaacccctctttactaaaaatacaaaaaaaaaaaaaaattagctgggtgtggtagcacatgcttgtaatcccagctacttgggaggctgaggcaggagaatcgcttgaaccagggaggcggaggttacaatgagccaacactacaccactgcactccagcctgggcaatagagtgagactgcatctcaaaaaaataataatttttaaaaataataaatttttttaagcttataaaaagaaaagttgaggccagcatagtagctcacatctgtaatctcagcagtggcagaggattgcttgaagccaggagtttgagaccagcctgggcaacatagcaagacctcatctctacaaaaaaatttcttttttaaattagctgggtgtggtggtgtgcatctgtagtcccagctactcaggaggcagaggtgagtggatacattgaacccaggagtttgaggctgtagtgagctatgatcatgccactgcactccaacctgggtgacagagcaagacctccaaaaaaaaaaaaaaaagagctgctgagctcagaattcaaactgggctctcaaattggattttcttttagaatatatttataattaaaaaggatagccatcttttgagctcccaggcaccaccatctatttatcataacacttactgttttccccccttatgatcataaattcctagacaacaggcattgtaaaaatagttatagtagttgatatttaggagcacttaactatattccaggcactattgtgcttttcttgtataactcattagatgcttgtcagacctctgagattgttcctattatacttattttacagatgagaaaattaaggcacagagaagttatgaaatttttccaaggtattaaacctagtaagtggctgagccatgattcaaacctaggaagttagatgtcagagcctgtgctttttttttgtttttgtttttgttttcagtagaaacgggggtctcactttgttggccaggctggtcttgaactcctaacctcaaataatccacccatctcggcctcctcaagtgctgggattacaggtgagagccactgtgcctggcgaagcccatgcctttaaccacttctctgtattacatactagcttaactagcattgtacctgccacagtagatgctcagtaaatatttctagttgaatatctgtttttcaacaagtacatttttttaacccttttaattaagaaaacttttattgatttattttttggggggaaattttttaggatctgattcttctgaagataccgttaataaggcaacttattgcaggtgagtcaaagagaacctttgtctatgaagctggtattttcctatttagttaatattaaggattgatgtttctctctttttaaaaatattttaacttttattttaggttcagggatgtatgtgcagtttgttatataggtaaacacacgacttgggatttggtgtatagatttttttcatcatccgggtactaagcataccccacagttttttgtttgctttctttctgaatttctccctcttcccaccttcctccctcaagtaggctggtgtttctccagactagaatcatggtattggaagaaaccttagagatcatctagtttagttctctcattttatagtggaggaaataccctttttgtttgttggatttagttattagcactgtccaaaggaatttaggataacagtagaactctgcacatgcttgcttctagcagattgttctctaagttcctcatatacagtaatattgacacagcagtaattgtgactgatgaaaatgttcaaggacttcattttcaactctttctttcctctgttccttatttccacatatctctcaagctttgtctgtatgttatataataaactacaagcaaccccaactatgttacctaccttccttaggaattattgcttgacccaggtttttttttttttttttttggagacggggtcttgccctgttgccaggatggagtgtagtggcgccatctcggctcactgcaatctccaactccctggttcaagcgattctcctgtctcaatctcacgagtagctgggactacaggtatacaccaccacgcccggttaattgaccattccatttctttctttctctcttttttttttttttttttgagacagagtcttgctctgttgcccaggctggagtacagaggtgtgatctcacctctccgcaacgtctgcctcccaggttgaagccatactcctgcctcagcctctctagtagctgggactacaggcgcgcgccaccacacccggctaatttttgtatttttagtagagatggggtttcaccatgttggccaggctggtcttgaactcatgacctcaagtggtccacccgcctcagcctcccaaagtgctggaattacaggcttgagccaccgtgcccagcaaccatttcatttcaactagaagtttctaaaggagagagcagctttcactaactaaataagattggtcagctttctgtaatcgaaagagctaaaatgtttgatcttggtcatttgacagttctgcatacatgtaactagtgtttcttattaggactctgtcttttccctatagtgtgggagatcaagaattgttacaaatcacccctcaaggaaccagggatgaaatcagtttggattctgcaaaaaagggtaatggcaaagtttgccaacttaacaggcactgaaaagagagtgggtagatacagtactgtaattagattattctgaagaccatttgggacctttacaacccacaaaatctcttggcagagttagagtatcattctctgtcaaatgtcgtggtatggtctgatagatttaaatggtactagactaatgtacctataataagaccttctgtaactgattgttgccctttcgtttttttttttgtttgtttgtttgtttttttttgagatggggtctcactctgttgcccaggctggagtgcagtgatgcaatcttggctcactgcaacctccacctccaaggctcaagctatcctcccacttcagcctcctgagtagctgggactacaggcgcatgccaccacacccggttaattttttgtggttttatagagatggggtttcaccatgttaccgaggctggtctcaaactcctggactcaagcagtctgcccacttcagcctcccaaagtgctgcagttacaggcttgagccactgtgcctggcctgccctttacttttaattggtgtatttgtgtttcatcttttacctactggtttttaaatatagggagtggtaagtctgtagatagaacagagtattaagtagacttaatggccagtaatctttagagtacatcagaaccagttttctgatggccaatctgcttttaattcactcttagacgttagagaaataggaggtgtggtttctgcatagggaaaattctgaaattaaaaatttaatggatcctaagtggaaataatctaggtaaataggaattaaatgaaagagtatgagctacatcttcagtatacttggtagtttatgaggttagtttctctaatatagccagttggttgatttccacctccaaggtgtatgaagtatgtatttttttaatgacaattcagtttttgagtaccttgttattttttgtatattttcagctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtattggaaccaggtttttgtgtttgccccagtctatttatagaagtgagctaaatgtttatgcttttggggagcacattttacaaatttccaagtatagttaaaggaactgcttcttaaacttgaaacatgttcctcctaaggtgcttttcatagaaaaaagtccttcacacagctaggacgtcatctttgactgaatgagctttaacatcctaattactggtggacttacttctggtttcattttataaaagcaaatccaggtgtcccaaagcaaggaatttaatcattttgtgtgacatgaaagtaaatccagtcctgccaatgagaagaaaaagacacagcaagttgcagcgtttatagtctgcttttacatctgaacctctgtttttgttatttaaggtgaagcagcatctgggtgtgagagtgaaacaagcgtctctgaagactgctcagggctatcctctcagagtgacattttaaccactcaggtaaaaagcgtgtgtgtgtgtgcacatgcgtgtgtgtggtgtcctttgcattcagtagtatgtatcccacattcttaggtttgctgacatcatctctttgaattaatggcacaattgtttgtggttcattgtctccttaaattagactgtaagcaccttgatggaactcatactaccttttatttcacacacacgcacacgcgcacgcagcgacacagcctacacatacactgcctagctcattgtagcatactaaatactgattttaatgaataagctaaaccttcgaaacccatttgctaatcccagcactttgggaggccaaggtgggtggatcacctcaggtcagaagtttgagaccagcctggccaacatggtgaaaccccacatctactaaaaatacaaaaattagctgggcgtggtggccaatgccttgtaatcccagctattctggaggctgagacaggagaatcgcctgaacctgggaggcggaggttgcactgagctgggattgtaccactgcactccagcctgggtgacaaagtgagactccatctcaaaaacaaacaaacaaaaacacatcatttcccctatagcaaaaacatgacggcacttactgtatcaagagaggtgagaaaaaggagccacagcaggatgattcaagggactctgcatagctccattttaagaatatgcctactgcaggtcagagaaggtaagcaaactgcctaaggccacacagccaggtacagaactctcaccaatattattgccagcaatcgcaattttggtgtttattcttggtaccaagttggagactatagggttctcttcctaatagagaccatctagcctttcactgttttgtggatacttctttctcttcttctttttttttttcccttttaaaatctagttatttttttctttttggtttctttgacacagggtctcttactctgttacccaggctggaatggagtagtgcagtcatggttcactgtagctttgacttcctgggctcaagcgatcctcctacctcagcttcccgagtagctgggaccacaggcgcccaccaacacctccagctaatttttaagtttttactagagacaacatctcactatgttgcccaggctggtctcaaaatcctgggctcaagtgatcccacctcagcctcccaaaatgctgggattacaggtgtgtgcaaccacgcctggcctattttttttttaattgctcataaatcatcttttttctttaaaaaaaagaaagatgggaggctaaagcaggagaatcacttgaacccaggaggcggaggttgcagtgagctgagatcatgctgctgctctccagcctgggcaacaagagtgaaactccatctcaaaaaaaaaaaaaaagaaagtacacaattttactttctggacctaatggtcaaggccaataatttggtcacctatgaaataaataaaagctttaccatatatatgaccatttgataatgtaatatgaaatgtttatgtactaaaggcagaatagtctagaaaaaacattctgtatcacaacgtctaaaaatgaatatcatcttcatcatagaaccaggctctttctcctaatttttttttttgagatggagttttgctctgtcacccaggctggaatgcagtggcacaattttggctcactgcaaccttcagctcccaggttcaggatcaagtgattttcgtgcttcagccttctaagtagctgggattacaggtgactgccaccacacccagctcatttttttgtatttttttagtagagagagggtttcaccatgttggccaggctcgtctcgaactcctgacctcaaataatccacccgtctcagcctcccaaagtgctgagattacaggcgtgagccaccaggcctggcctcctaatttttatttgtagaagtggcaccaaaattttccaagttctcatgcaaaaattcaggctcatctcagtttatttttttcatttatttatctcccactaaattgacaacttctaataattaggttggttctttgtattcccagcacagggttctatgcagaatacacacacagcagttgctggcaataatattggtgagagttctgtactgggctatgtgatcttagacagtttgcttatgttctctgacctgccgtaggcacattcttaaaatgaagctgttcagaccccctcgattcatcctgctgtggcttctttttcccacctaaatcttaaatacccttttagctgctagtaagtgaatgatgtttttttatgaactttctgaagtcagattagatgaagttgagaaaagcctgatattcttataaagttatatatgtgcatcatagaaaacttagaaaatacagataaacaaaaatcatccatggacgaaccttgaagacattgtgttaactgaaataaaccggacaccaaaggacacatgttatatgcttccacttatatgagatacctagaatagttacatttggttactctgggtacattgcctatagataagccttgctccacaaggagcagttaaaaaaaaaaaaaagataaattcataggatggaaggtagaatagtggttactagggacttggggagggggaaatggggagttactgtttgatgagtgcagatttcagtttgggatgatgaaaaagttctggagatagatagtggcaatggtaacacaacagtgtgaaaataatgccactgaactgtacacttaaaatgattaaaatgataagttaattgtaatttgtgttatccagaaatggttagcaatttattggtgtatattcttttagtattcctgtgtgtgcacaggggtgcttgtatatactttatctttaaaatatatccaggaagctaggcacagtggcttacacctgtaatcccagcactttgggagggtgaggcaggaagattgcctgagccccggaggtcaaggctgcagtgagttgtgatcacgctactgcactctgttctgggcaacccctgtctgggaaaaaaaaaaaaattagtgaggcttagtggtgcacacctgtagtctcagctacttgagtggctggggtaggattgcttgatcccagcaagttgaggccgtggtgagccatgatggtgccactgcactccatcctgggtgatatggtgagaccctgtctcaaaaacaagaaatccagataattctgtgcattataatctagcttttactggatcattaaaattcttttttctttttttttttttttttctgagatggagtttcactcttgttgcccaggctggagtgcagtggtgtgaccttggctcaccgcatcctctgcctcccgggttcatgcgattctcctgcctcagcctcccgagtagctgggattacaggcatgtgccaccatgcccagctaactttgtatttttagtagagacagggtttctccatgttgaccaggctggtctcaaactcctggcctcaagtgatccacccacctcggcctcccaaagtgctggggttacaggcgtgagccaccgcactcagcctgggtcgttaaaattcttaagtgacttcatttttaattactatatgggattctatctttccagtgtatcatgatttatttgacctattgctgaatgttggaggtttcagggtaagaggcacagtttgctattatgtacatcactatagtggcatcctgatagctaaatatttgcctacatccctgattatttccttagtctaaattactgggactaggattttggtgtttgatacatgttactaaattgttttttagaaagattaaaccagtttatgctcttccagcccctgtggtatatgatagttcccattttcctgtaccttgccaacactgggtgatatccagttttaaaatctaaatcttgcattgctatgagaactacaattagagaaggcttatcttctactgcccattctctgtacagagcaaatccctctagacctgaagccccttggagttgtcaagaaacctttgagatgactccccactctgtatctgagctgtcaccagtattctccacttcttcaggattgccatggcaactaaattgatgaaaagatttaggaggccttttctctctttgcaattcctatgatcctttttgaatgtgggtttgggactctgtcaatatacccatcatctaattctgtccattgtgttttaaagtttaaggttgcaatttctgattacatctgccttagccatactgtattatatttgacattcaatatacaatgtccttgtttttctgtatttctaatcttattcccagagatgtgtctatttgttcaggattcattttgcaacgtgtttttactaagcatctacccaaaaccgttgaagtcagatttcaggctgtcttacgtctaaagtagcacaggcaggaaaaactattgaagtgggatttttttttccctttttgtactgaaccgagaaaaagtatatagatgatagagaattcctaatttggtatcattgatatctgggtttttgtttgtttttacagaagactgattaactatacttatttattaatttatcttctcattaataaacacttgctgagtgcttactgtctgctaggcattagggagacaaatatgattaagggaagcttcctcctatcaaggtcatgtgttccatttgggtatactaatgcattagcaatgtaaatcaagtagtgagagatcatctgttcccgataggagatggattattggtggggacttctgtgtgtgtgtgtgtgtgtgtgtgtgtgtgtgtgtgtgtatgtatgtgataaaataaatataggaaatgttaattatagattctaagtagtagatatataaacactcattgcaaagttgcttcaagttttctgtatatttgaaaatattcacaacatgtcgacaaaactagcatgataaagccactatttgtgctaagacttcagcttgtatctggattaggcttattatgtagtagtaggaacattagaaatagttttaactcattaaatacacatgttttatgggaaggttttatatatatatttatatgtaatgaatgtgaacaaacaagggtcagatatacactctgcttccctccagaccagttccggctgctctgctgcacatttcaggagtcttattagaattagccacattctgcccacttgcccttacttctcatatttcacaactcctcctggtggggactttaaggagacattcaaactaggccttgaaagatgagaatttttccaagtggaaaaagaggagtggcagcaagtaaggtaaaggtacagagtcatggaattcccaggaaacgtaaagttgtcatgtgttataggaaaacaacttgtgtgaggggtgttgggagaaatgagagataataccagggtataaagggccttttgaatgctatgttgaggaattttatcctaatggcagtaatgactaacaattatatagtgttcaaaaagtataaatcagcagtggtataccactaagggtttttttcttttctttttttttttgagacagagttttgctctgttgcccaggctggagtgccgtggcacgatctcagctcactgcacttccgccacctgggttcaagtgattcttctgcctcagccagtgtttcactgtgatggccaggatggagcactaagggtctttatggaagaaaaagacatgataaacaaggcttttagggaacttctacagtaatgtagctgtattaaaagtagagatcagagcagcatagtagaagtagaaggctagagctaattgaaggagcacttcagaattagaatcaagaagtcttagaaacctattggttttattctccctaatgtatttggccacttacctgctggggaatttgtctaagttataaaaaataattcctttgggaaacccaaaggaaagttatctattaataattaccccactactttttctgatttatgtaatggccacgtagaggttagatgtgatggttgtgacagtagtgactaatacagcctgtgaagcattttggtcagatatctatgtgctttcattccaggttgactgaggcaagactttggctagggtttgatcagtgatgtaactactcacgagtaccacgtggtggcaatggcattgctgcagaccttggcagcaaagcagtgttagagtagcagtagaaacctttgtgaagctaggaatacattttctggtcataaaaacctcctgaaaattgtgaactcagtgtagcaggagaaagaagatggcttgtttttagtaaagggcaaagtcatttttaaggatcagaagaagaaacggagagtgaaacaatgtgttcctgccctactcccccactggactttttggcaaccattgctgttccttctaaaagtgatttttaaacatgtatattttgaagccaggcacagtgactcacgtctgtaatcccagcactttgggaggccgaggcgggcagatcacctgaggtcaggagttcaagaccagcctggccaacatggtgaaaccccgtctctactaaaaatacaaaaattaggccaggtgtggtggctcacgcctgtaatcccagcactttgggaggccgaggcgggaggatcatgtggtcaggagatccagaccatcctggctaacacggtgaaacaccatttctactaaaaatacaaaaaattagctgggcatggtggcgggcgcctgtaatcccagctactcaggaggctgaagcagaagaatggcttgaacctgggaggcggagcttgcagtgaaccaagattgcgccactgcactccagcctgggcaacaaagtgagactccgtctcaaaaaaaaaaaaaaaaattagtcgggcatggtaacaggtgcctgtaatcccagctacttgagaggctgaggcagggagaattgcttgaaccaggtaggcggaggttgcagtgagccaagatcgcaccactgcactccagcctggggcaacagagcaagactgtctcaaaaaaaataaataaataaaataaattcttaagaaggatattttggaaaactccttacatacctaaattctttgtttatcaaatacttggacttagcacactcttctttgaaatggaccaataaacaacaggagcccataagcaaaaagaactcattattttaaaaacagtaactatccttacaggctttctcagggctctttctgttggatccttccctctcacaggtccttgctaatgatctctaggtggacacattctagatgagatgtccctgtctagaatggcagcaccatgagggctatatcctcagtactaggacagcgcctggtgcttaatagatagtaaatagttgtctaattaactgagcaaacagatagattcatgaattagctttttgctttttctgttagaaactaaaggttcaggtcaggcacaatggcgcatgtctctaatcccagcactttgggaggccgaggcgggctgatcacttgaggtcaggagttcaagaccagcctggccaacatagtaaaaccctgtttctacaaaaattaccaaaattagccgggcgtcttggcaagcacctgtaatgccagctacttgagaggctgaggtgggagaatcgcttgaacctgggaggaagaggttgcagtgagccgagatggtgccaacctgggtgacagagggagacttaaaaaaaaaaagaaagaaagaaagaaaagaaactaaaggttcaaagaatcccagaaaaggaagagtcctcacaagccagtaatctaggcaggattactgatagtatttttatatttgttgtatttttataaaatgccatagatagagggcttttttcaacattacatcagtctaaaaatcacacatttttatatgaactaacctaaatgtctgatgaatctcacaacaccaagtctttgaaatgtgcccatataaataaaatgttaacagattcatgctaattttaaatatcgatagtgtttaaatgccttaattattttttcactccctagctttaaaagaaaataaccaacttcaaaaggacatcacaataacatcaagtctatttgggggaatttgaggattttttccctcactaacatcatttggaaataatttcatgggcattaattgcatgaatgtggttagattaaaaggtgttcagctagaacttgtagttccatactaggtgatttcaattcctgtgctaaaattaatttgtatgatatattttcatttaatggaaagcttctcaaagtatttcattttcttggtgccatttatcgtttttgaagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaaggtgtgtattgttggccaaacactgatatcttaagcaaaattctttccttcccctttatctccttctgaagagtaaggacctagctccaacattttatgatccttgctcagcacatgggtaattatggagccttggttcttgtccctgctcacaactaatataccagtcagagggacccaaggcagtcattcatgttgtcatctgagtacctacaacaagtagatgctatggggagcccatggaagatacatggtatacaacatagctcttgctctattggaagctaagtggaatgggagaaattggtgacaggcaaccccataatttcagaaagctatgaaaaagtactcagacatattccttataacactggtgtcacatcacaaagacctatttaatgtgcttctgatttatagggagagacatcctatacttcaggaactgcactttgatccacagaaagcctagtgatgtagagctcctgttagttcaaaaggaaaagaaaagaacaacacagaaagcctaattatgcaatagagtcaagtgctttatagcaatgttacagttatcaaaaaaaatccagatggacctctgagaggatgccattggagtaaccaggcagatgcagttgatcagagctgacttcctataagaagtgagcactgagctgaggaataatggcataaatgaaggaaagtgagatggaaatttgagtttttaattggaaagacaatacatcaggcagatttttaaataggggcaaacaaacagacacataggagatgctaggcatggggtccccactaggatgctgcttagaaacatgcaggggtggtgagtactcccaaagtacacttcattcctagctcagtgattcttatctgagtgttaaagttccttcttcagcaccccgttccacagtccaactgggaactttaagacctttcttggagtctttctaggaactcaagtctgctacttatacagaacagtggctttggtccccagttgtgccttgcagtatttttgtgttcaggaagaaacagtagctcttggataaagaagctagctagaaactctgttgctatggcagtgcttcaaaatgtatttccttaaatgctttctttgtaactatcttcatttagttcatctctcagataatgagagatcagagtcccatccccagtataatactcttctttagggtactttcaccatcttcagtctaaacacagactagactttcaattataatgtgtaagatttaaaatgttattattgtgtgactttgaatatctgtgtaaatctactatctcctctttggtatatacgtgtgtttatttttttctggagatctgtaactgaaatgcttaatttctgaattgttttggatatcacaacttaataccaacataagttttgagcctttttctccctaaatctggtgtgagtctaactgaaactcaaatgaactttttaaaaataattttttcttttctttaattttttttttaagtagagacagggacgcactgttaactaggctggtcttgaactcctgatcttgagccatcctccccgacctgagcctcaccttatagagagggtcttgctctgttgcccaagctggagggcagtggcataatcacagctcactgcagcctctcgacctcctcaagcgatcctcctgccttagcctcccaagtagctgggactataggcgtccaccaccatacccagctaatttttttttttattttttgtagagacaaggtctccctatgttgcccaagttggtctcaaactcctggactcaagcagtcctctcacctcagcctcccaaagtgctggggttacaggtgtgagccatggcacctggccagaacttctagtaaaaagaatattgttgccgggtacggtggctcacgcctgtaaccccagcactttgggaggccaaggcaggcgaatcacctgaggtcgggagctcgagaccagcctgaccaacatggagaaaccacatctctactaaaactacaaaaaattagccgggcgtggtggcacatgcctgtaatcccagctacttgggagctacggtgcctggcctagtttattatttcttaatatctgttgtcttccagtgtcttccttaattcttcacaataccctgtacaatgcttagcacacagtgggcagtctgtaagtttattaaatgtttggtgtggcccatacttcctatccacaaagaatgtaacatgttaagacatctagatgagggaatgatttaagaggaactacaataatattctgaaacttggactctggatctctgcatttagactttcctaaaccagccagcaagtagatcatcatgtcacaaggcttaggttgggcttgctgttcagagaatgaattaaggattaaggagaaaaaaaagcagaaaggttttgctctgtttttcaggttctattgagttgttaacttctaacaagttatcttatttgcttcattgcatgaggcccattgtagtaagaagaggaatttatatgctaaatgttctggtgatagaatgacttttctttttttttacagtccaaaggtcttttttttttttttttaacacctattatgccatgaattcatagggaataggttccagctgctcaggctccttcccattggttctcacaaagtgtgcttctctgggtggagcaggctggtgcttcagttgaacccacgtacctttctctttggcttctttctttttctgatcattttccttcacgcgtttcaggaagctgtcttggctcttagagtgtttaatgtgctcaatacgcacattaattctcttggcaagaatcttgcccttaacttgtttacagcgatgccaacagcatgctgggtcacgttgtagacttttccagttttgccatggtaacacttgtggggcattcctttttgaacagtacccgttcccttgatgtctacaatttcacctttcttacagattcgcatatacatggccaaaggaacaactccatgttttctaaaaggcctagagaacatatatcaggtgcctctcctctttccctttgtgttcgtcattttggcaaattactgaaagatggtggttctggccaaaaggaggaatgactttttaatagctgtgtttgtatctgagccttccctctgcctttcattttttttgttttgttttgttttgtttttgtttgagatgaagtttcacttttgttgcccaggctggagtgcaatggtgtgatttcggctcattacaatgtccgcctcagcctcctgggtagctgggattacaggcacccgccaccacgcccagctaatttttgtatttttagtagagacagggtttcaccatgttggccgggctggtctcaaactcctgacctcaggtgatctgtccacctcggcctctcaaagtgctgggattgtaggcgtgagccacatcacctggccacttttttaactctttccaatggttaattccgtttgatatggttccttggaacttgcacattaccctttatcaattatcaccctgtattgggggtggggaggatgatacctctcttcatagttagatcctacttactttcaacagagttcttaacaatcctagaaactcacaggtccagaaaagacaagcataaaggaaactataaataatgcatttgaagactaactcaggaaatcaatgattatttccccccaggctacccagtgtcttaaaaaaacagtttaattaatacaatcttttgtttcaattttctacctatatttatggcttttagcttttctaataaaagctcaaaatgaattacagtcatcagtgactttttaatgaatagaagacttttgcaatttttaactatttgtttttacttattaaatatttccgccttggccaggcatggtggctcacgcctataatcccagcactgtgagatgccaaggcaggaggatcacttgagtttaagagttctagaccaggctgggtatggtggctcatgcctataatcccagcactttgtgaggccaaggttggcggatcacctgaggtcaggagtttaagaccagcctggccaacatggtaaaaccccatctctacaaaaaatacaaaaattagccaaggggtggtggtgggcacctataatcccatcttcttgggaggctaaggcaggagaatcgcttgaacctggaggcagaggttgcagtgagccgagatcatgccactgtattccagcctgggtaacagagcaagactctgtctcaaaaaaaaaaaaaagtttgaaaccagcctggtcaacacagcaagacacccatctcgttgaaaaataacggtcgggcgcagtggctcacgcctgtaatcccatcactttgggaggccgaggcaggcagatcacctgaggtcgggagttcgagaccagcgtgaccaacatggagaaaccccatctctactaaaaatacaaaattagttgggcgaggtggtgcatacctgtaatcccaactacttgggaggctgaggcaggagaacagcttgaacctgggaggcagagaggttgtggtgagccaagatcatgccattgcactgcagcctgggcaacaagagcaaaactccatctcaaaaaaaataaataaataaaaataaataaataagtacttctgcctttaagccacttcctagaaggcagtggcacaaagtgatacatttggaggagtaaatatattacaaaatgaattaggctgggcgcagtggctcatgtctgtaatccccgcactttgggaggccaaggcgggtggatcacttgaggtcaggagttcgagactagcctgatcaacagggtaaaatcccatctctactaaaaataccaaaaaaactagctgggcgtggtggcaggcacctgtaatgtcagctactaggaaggctgaggcaggagaatcgcttgaacccaggaggtggaggttgcagtgagccaagattgcaccattgcacttcagcttgggcaacagagtgagactccgtctcaaaaaaaaaaaaagaactaacatgccagaactttgccttcagtatgttttgtgatttttcccttcttgtgccatttcatcattagttccatgtattatttaagatttcttatcaaccagcaccttgggatttttttgtgtatgtgttggtttagggggtttatttgtttttttcttttttttcggtaattgaaaatgtgaagcaaaatgtcacctgttttttctttcatgtctgacactcatgtcttgtttacccccgacatgcagaagctgaaatccccatttcatacagtcttcaatgtggaggcagtagggatggagaaaataatgtactttgtgctctccggtactctttctttcctattgtctgaggggatttgggcataatttattttgctgcagagataaaaatttgttatatatattttttatcattcagggccaaggaatatagattttttttttcagccttgtctcagctgggtgtctttatttactctgtcttaaagtgttccttttattatcattattattttttaatcattgaattccatttggtgctagcatctgtctgttgcattgcttgtgtttataaaattctgcctgatatacttgtttaaaaaccaatttgtgtatcatagattgatgcttttgaaaaaaatcagtattctaacctgaattatcactatcagaacaaagcagtaaagtagatttgttttctcattccatttaaagcagtattaacttcacagaaaagtagtgaataccctataagccagaatccagaaggcctttctgctgacaagtttgaggtgtctgcagatagttctaccagtaaaaataaagaaccaggagtggaaaggtaagaaacatcaatgtaaagatgctgtggtatctgacatctttatttatattgaactctgattgttaatttttttcaccatactttctccagttttttgcatacaggcatttatacacttttattgctctaggatacttcttttgtttaatcctatataggttttttgaacctataacataagctacaacatgagaaatgtgcggttagatagatatgtcccttctgaaggtcagaaaaaaatataatggaggtaaaacctgaacaagcttggaaactgatggtagacttcttcaaggcagcccttgccctaattaaaattcttgtctttctagaaaaagtctagctgttgatttaccacagaaaataataataataattactattattattattttttgagacagggtcgccctgtgtcacctagattgcagtggtgcagtcatggctcactgcatcctccgtttttcaggctcaagcaatcctcccaccttagcctcctgagtagctgggtccacaagcatgcgccacccacacccactaagtttttgtatttttggtagagatggagttttaccttgttgcccaggctggtctcaaattcctggactcaagtagtccgcccgccttgccctcccaaagccagaaaacatttagaatatctttcagagatgtgtatttacaccactattaacacagggctgtatagcagtccagtactggactatgtagtccagtactattcttttccttactggagggccaggcgtggtggcaggtgcctgtaatcccagctactcaggaggctgaggcaggagaattgcttgaacctgggaggcagaggttgcagtgagctgggaccgtgccattgcactccagcctgggcgacagagcaagactccgtctcaaaacaaaaaaaaaaagagagagagagcagtaattcaggtctcacccatcttcaatccagggggcctagccttagtatttgacccatagtaagcacccaataattgtttaaattaattaacctctgaggccctttaaatctgttgataagtatcttattttgcaaagtcctaagcacttggaagagcagaggaactatttactgggtgtgtatgcttttctaacaatattttatagctggcttttgtttttagaatgaatttgaacattgaaaaggcaggcaatagggatgattctgtgaattctgctaaaactgagtagaaagaatgagtgtagagatgtcgacattgatcaactttctatcttcataagagatctgattctaacatatccatttagactcaagtagaatattgtgtatagagtgagtggcagtgagtaatttggtaaaaatttgctgacctgcttttattctttcctcctttctttcttcctttccttccttccttccttccttccgtcctttcctttcctttccctcccttccttccttctttccttctttctttcctttctttcctttcttcctttctttccttcctcccttccttttcttttctttctttcctttccttttctttcctttctttcctttcctttctttcttgacagagtcttgctctgtcactcaggctggagtgcagtggcgtgatctcggctcactgcaacctctgtctcccaggttcaagcaattttcctgcctcagcctcccgagtagctgagattacaggcgccagccaccacacccagctactgacctgcttttaaacagctgggagatatggtgcctcagaccaacccaaccccatgttatatgtcaaccctgacatattggcaggcaacatgaatccagacttctaggctgtcttgcgggctcttttttgccagtcatttctgatctctctgacatgagctgtttcatttatgctttggctgcccagcaagtatgatttgtcctttcacaattggtggcgatggttttctccttccatttatctttctaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctaggtaatatttcatctgctgtattggaacaaacactttgattttactctgaatcctacataaagatattctggttaaccaacttttagatgtactagtctatcatggacacttttgttatacttaattaagcccactttagaaaaatagctcaagtgttaatcaaggtttacttgaaaattattgaaactgttaatccatctatattttaattaatggtttaactaatgattttgaggatgagggagtcttggtgtactctaaatgtattatttcaggccaggcatagtggctcacgcctgtaatcccagtactccaggaggccgaggcaggtggatcagctgaggtcaggagttcaagacctgtctggccaacatggtgaaaccctgtctctactaaaaatacaaaaaaattaactgggtgtgctagtgcatgcccgtaatcctagctactctggaggctgaggcagcagaatcacttgaacccgggaggcggaggttgcggtgagccaagatcacaccactgcactccagtctgggtgacagagcaagactccatctcaaaaaatatatatatatatatatacacacatatattttatttcaactgttagacaagagtccaaaggccaaagaataaagttttaggccagtcctttattagaaaatgagtcaaatcccaaagcaagtttttttatgagttaatgaatataaatgactacatattttatgccttaaaaatcacttttaatgaatggtgttttatggcttgtaaatcagagttttaatcagtaaagaaagtttttaatcctcaaaaacacgttatcataaaagacactgtttggcatcaaatgtggtatttggccatgttcattagggtcattttaggaatctcatacattctacttagctatgcttaattcctgataccatggcattttctgaaatgtttcaaggatgacatctctgctgtttttaatttggtaatgatatctgctgatttattaagtgaaaaaagtaatggtgtcattaccttggatgaagaaacaaaaataaagcatttgccacatttttcaactttgttttcctttcttacaaaattgctataagctcattgcccccaaattggacaatatagggaataaaaaagataatttggggtggggttagacacgggtcttgttatgttgccgaggctggtctctaactcctggcctcatgcaatcttcctaccttggcctcccaaagtgctgggattataggtgtgagccacttcaccaagctgagatgccacctcttaaaagagagaataaggacagattacagccactgctcatgcctgtaatgtcagtactttgggaggccaaggtgggagaattgctcgaggccaagagttcaagaccagcctgggcaatgtagcgagacctgatctctatgaaaaggggggtgggggggaaaactagctggggccaggcgtggtgggtggcttacgcctgtaatcccagcactttgggaggccgaggcgggcagatcacctgagggcaggagttcaggaccaacctgaccaatatggagaaaccctgtctctactaaaaatacaaaattagccaggcttggtggcttatgcctgtagtcccagctactcgggaggctgaggcaggagaatcgcttgaacctgggaggcagaggtttcagtgagctgagatcgcgccattgcactctagcctgggcaacaagaatgaaactccatctcaaaaaaaaaaaaaatcagctggaaggtggcaaacacctgtggtcccagctactcaggaggctgagacaggaagatcacttgagtccaggaggtcaaggctgcaggtgagccatgtttgtgccactgcactgcagcctggatgacagaccgagacccttctcaaaaaaaaaatttttcccggtatttttttttgggggggggtttaattcttgttgcccaggctggggtgaattggggaattttgggttaagggaaccttcggcttcctgggttggggggtttttcctgtttaggcttccccagtagctgggattacaggcatgcaccaccacgcccggctaattttttgtatttttagtagagacagggtttctccatgttggtcagactggtctcgacctcttgacctcaggtgatccgcccaccttggcctcccaaagtgttgggattacaggcctgagccaccgcacccggcctgtactcttattctttaataataaaatatttctgtgtttctttagtcattttacataaacttttatttatttatttatttttatttatttatttttttgagacggagtctcgctctgttgcccaggctggaatgcaatggctcaatctcagctcactgcaagctctgcctcccgggtacacgccattcccctgcctcagcctccctagtagccgggactacaggcgcccgccaccacgcccagctaatttttttttttgtattttcagtagagacagggtttcactgtgttagccaggatggtcttgatctcctgacctcgtgatccacccgtctcggcctcccaaagtgctgggattacaggtgtgagccaccgtgctcggcccataaacttttatttttaaaataatgtcatgataaataatattgcttaggtgtctttaatatattagtaacatttctgttttattgtacatcaacatttatattcaaattaatgggtgaagagtactccattggactaggtatatcgtaatttaatctcctattattggacaactacattgtttctaaaattatactattacaagcattgttacagtgaacattcctatgactaaacctttgcatatatcttttatctccctaggatatatttctaaaactagcattgttgactgaaagtgtaaatacgtgttaaggtgtttgctacataatgccatatttcctttttaggaaactaagctactttggatttccaccaacactgtattcatgtacccatttttctcttaacctaactttattggtctttttaattcttaacagagaccagaactttgtaattcaacattcatcgttgtgtaaattaaacttctcccattcctttcagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttgtgagtgtatccatatgtatctccctaatgactaagacttaacaacattctggaaagagttttatgtaggtattgtcaattaataacctagaggaagaaatctagaaaacaatcacagttctgtgtaatttaatttcgattactaatttctgaaaatttagatctagataaagctatagtgtggattattttatgtatatttacttgagaaaataattattaaatattagtggaaaagctatactttgggtatgatataggactttcgaattggaattttcctttctatctgtaaaagcaagtaggtatagttttattccccagaaggcatctttttctcccccttgtctcacatgggtgaatttaccagcatatttaactaaattcagactggttccaaatgtactgccagatagtagcatttctctagtgtttgttttcatcctggcttgtaagaatgccctgccacttctgccctgcaatatcccttgctattaggattttggcatcaccttgggtccttaatgccagaaatgggaattgcttcatactgtggaaaaatacccattaaaatattaagaccagtaaaacctcgtttctgcttgggctatttgtggatttcagacatcctgagaagtttaccacccctgtaattaattgtcattgtcatcacttcataataaaaataattgcatggccgggcatggtggctcaagcctgtaatcccagcactttgggaggctgaggtggtcagatcacctaaggtcaggagatcaagaccagcctgaccaacatgaagaaaccccatctttactaaaaatacacaattagccgggcgtggtggcgcatgcctataatcccagctactcaggaggctgaggcaggagaattgcttgaacccgggaggcggaggttgcggtgagccgagattgcaccattgcactccagcctgggcaacaagagcgaaactctgtctcaataataagaagaagaattgcgtgaatatttctttaaaactatgatgagataacataccagattatcaaatggattcagtagtgggtgtgccatttattgcacactgagagatgaccaagtcattctgaaatatctttattaatatatccttcctaggatttttcatcctaacttctccataggtagttacttagcataacatctctgtggccagatgtatcccactactaaaagggcaaagtaagctgtggctgccctggtagatacaatgagtaagtgcacagtgatggctataaatgttttcatctcataatcccatgtccagaccagcaatttgctctgaaagctcttacctgtgtctgtttcaatggctcttgatcacttgcctgcacgtccagaattccttatttattcattgaaaattagcgttctttatccctttgttttgcaagttcagctttttagagatggctaaaatggtctaatctttcttggcaaaggcaattctgagctgcagattagactacaagtggcttgggtacatgttgtctttaaacaagcgaagaggaaaactttgagctctattcagacttggtgaagtgtggtaaatttatgatgaaagctactgactgtattacacatgattaattctgaagcccatattaagatgatcttttcagcagttcagcattgctcttctaactgaacagtttcaaggctgggatttcagcaattaatcagttcagaattgctaatgatcctggcggagggtggtagcaaaagggggaggatgtcattagcttctctagcctgccttttttcagtgccctgtggcagtatggagtgaggcaacatgaaagaaagatggcctgaccttcatggcagtattgtgcaacacgtaaatactggtgtgagtggctgtggctatggctagtaaatgatggcccttggtaaacaaagttatttatcagacaatacctaccagctaggtcaactgtgcccataattgatctggttaatttcttttgctgcctattgatttttatttggttgatagataatagctagaggactctaaatttctttggggaagaacatgaaccccttctaagccttcttacgagagaattgatcgcttttgcactgacctttagtaacatcctgatttcagtgttttgtaactatcagagggttgagtcttggttttaagccatgtatatctgtagcataactttctgtgtaggctagttacctctcagcttataaagtgtaggctgataaatttatagtacagtagagtgtcactatgcaaagaaacgatcttagggaatcgaatgatatctgctattaaagcaaaattaatatatattttttctttttacttttttttttttttaaagacatgaaatctcactgtattgcccaggctggtcttggtctcagactcttgagctcaagcagtcctcccacctcagcttcccaaagtgctgggattataggcatgagctgccgtgtctggcccagtatatattttttaagttttaagttttgtggtacgtagtaggtttataatattattttgaatccttagttgtaattttatgtctgctgatgtgtacataatttttattaaactatttatttgagacttcaggtatcttttttttttttttgagacggagtctcgcactctcgcccaggctagagtgcagtggcgccatctcggcttactgcaagctctgcttcctgggttcacgccattctcctgcctcagcctcctgagtagctgagactacaggtgcccgccaccacgcctggctaattttttgtatttttagtagagacagggtttcaccgtgttagccaggatggtctcgatctcctgaccttgtgatctgcccgcctcagcctcccaaagtgctgagattacaggcgtgagccaccgcgcccagccgagacttcaggtgtcttagaattttttaaatgtaccctttctgagaaaaacagagacttaaagctaggataactggtattctattttttttttttttttttttttttacctccagcctgggtgacagagcaagactctgtctaaaaaaaaaaaaaaaaaaattcactttaaatagttccaggacacgtgtagaacgtgcaggattgctacataggtaaacatatgccatggtggaataactagtattctgagctgtgtgctagaggtaactcatgataatggaatatttgatttaatttcagatgctcgtgtacaagtttgccagaaaacaccacatcactttaactaatctaattactgaagagactactcatgttgttatgaaaacaggtataccaagaacctttacagaataccttgcatctgctgcataaaaccacatgaggcgaggcacggtggcgcatgcctgtaatcgcagcactttgggaggccgaggcgggcagatcacgagattaggagatcgagaccatcctggccagcatggtgaaaccccgtctctactaaaaaataaaaaaattagctgggtgtggtcgcgtgcgcctgtagtcccagctactcgtgaggctgaggcaggagaatcacttgaaccggggagatggaggttgcagtgagccgagatcatgccactgcattccagcctggcgacagagcaaggctccgtctcaaaaaaaaaaaaaaaaaacgtgaaaaaataagaatatttgttgagcatagcatggatgatagtcttctaatagtcaatcaattactttatgaaagacaaataatagttttgctgcttccttacctccttttgttttgggttaagatttggagtgtgggccaggcacggtggctcacacctgtaatctcagcactttgggaggccgaggcgggtggatcacctgaggtcaggagttcgagaccagcctggccaacgtgttgaaaccccgtctctactaaaaatataaaaattaggtgggcgtggtggcaggcacctgtaatcccagctactcaggaggctgaggcagcagaatcgcttgaacccaggaggtggaggttgcagtgacccaagatcgcaccattgcactccagcctggggacaagagcgagattcttgtctcaaaaaaaaaaaaaaaaaaaaaaggtttggagggtggtgagctgagatagtcaactattaactcctatctacctgctgggactacactggtgaggtggagcctaagtcctaaaacaacaagtgaggcagctggacgcggtggctcgcatcagtaatcccagcactttgggagcctgaggcgggcagatcacaaggtcaggagttcgagaccagcctggccaatatggtaaaacccagtctctactaaaaatacataaattggctgggcgtggtggtgtgcacctgtaatcccagctacttgggaggctgacacagaagaattgcttgaactctggaggctgaggttgcagtcagctgagatcctgccactgcactccagcctggcgacagagtgagactctgtctcaacaacaacaaaagaaagaacaagtgaggcaaaacctggagaccccagcttcatgtaacacctagtttgagtattgttgagagtttttcaggaaaaaagtctgataacagctccgagatagtcttaacatatgaaaaagcaaaaaagggaggagacagatcatttgtcctatacctttctcttttaaggttttaattataacttgtgtaatacaggagacctctgggtgtttttagttgactataaactaaatctgagtacacatttcagggctgctaaaaatgcttatttgaaactgggccgtattaacacaagcagaggctctggagcaagtgaagtacagatccagagccccactgtattctccaatggagtgattgcctgaaagatgatgtcagttttaagcaccgtgcttggtttttaacatggtcactgacaaattggagagtgtttatccagaggtagatggtaaagatacataaaagtaacttgaaatactgtcttttgaagaagaaatgagaagatttaaggaaataagacactgtcttcaagtatctgaagaaccgttacccggaagagaactgttatctggaacaggattaagactcactcatggggctccagaaagcagacgagtgcatggaggacgcagaagatgcagattgtgtggctcaactctaaaatctttctaacaaaattagttctctggatgtgttccagttcacttgatgatgattcttttgtttttgtttttgtttttgaggtgtagtttttcactcttgttgcccaggctgctggagtgcaatggcacgatcttggcttgctgcaacctccccctcccgggttcaagcgattctcctgcctcagcctcccgagtagctgggattacaggaatgcaccaccatacctaattttgtatttttagtagagacagggtttttccatgtcagtcaggctggtcttggactcccgacctcaggtgatccacctacctcggcctcccaaagtgctgggattacaggtgtgagccatcgcgcctagcctatgatgattcttttcacagagatacaggcacttaaggagaggatctaaaccccttggacacattgccgttgaacttctaagatcttaggtttccacttactcatgaaaattataccacagggtcagagggtagtgttcattggagccaggtgccagaacaagttattacaaactactattttagagaaaaatgtcattaaagtttaagataccttaagctataggtttgcatcaaagttaatgaaaggtaaaaagatgccaagcgtggtggctcaggcctgtaatcccagcgctttggggggccaaggcgggcagatcacgaggtcaggagatcgagaccatcctggctaacacggtgaaaccccatctctagtaaaaatacaaaaaattagccgggcatggtggcgggcatctgtagtcccagctactcaggaggctgaggcaggagaatggcatgaacccaggaggcagagcttgccgtgagctgagatccagccactgcactccagcctggctgacagagcaagactgcatctcaaaaaaaaaaaaaaaaaaaaatgcaaatcaaatctaaagtagttcagtctttaaactcaaagccaatacatttgctttgaactacaaatgaactgaagtttttaagtgtaataaatgttactaaatcggcttttgtagcagttaaacaaaaaacttcaaaaattgtaaggattctgtgagggagcatggctgctgctgctgctgctgcttgcagatagcctgctgtgtttaggatttagttaaatacatttctcctgtttaaaactaaatggtctttccttagtttgcttagttcttcagaagggcctttgaaacactgggaaataaacaagtgattctttagctactgctttctgaaatacttatataaaagctctgcactgtattctcccatccctctcaggggaatattagagggttaggactccccaggtagacattctaggggtgaaaatttgtcattacattgacatttcagatttaggttttcaacaatactgttttcttctttcacatattgccatctagtaatatagatgttctccgtccacattaatcaaaactattgacatggataattcctaattccttgaacactataatggagatctatagctagccttggcgtctagaagatgggtgttgagaagagggagtggacagatatttcctctggtcttaacttcatatcagcctcccctagacttccaaatatccatacctgctggttataattagtggtgttttcagcctctgattctgtcaccaggggttttagaatcataaatccagattgatcttgggagtgtaaaaaactgaggctctttagcttcttaggacagcacttcctgattttgttttcaacttctaatcctttgagtgtttttcattctgcagatgctgagtttgtgtgtgaacggacactgaaatattttctaggaattgcgggaggaaaatgggtagttagctatttctgtaagtataatactatttctcccctcctccctttaacacctcagaattgcatttttacacctaacgtttaacacctaaggtttttgctgatgctgagtctgagttaccaaaaggtctttaattgtaatactaaactacttttatctttaatatcactttgttcagataagctggtgatgctgggaaaatgggtctcttttataactaataggacctaatctgctcctagcaatgttagcatatgagctagggatttatttaatagtcggcaggaatccatgtgcagcaggcaaacttataatgtttaaattaaacatcaactctgtctccagaaggaaactgctgctacaagccttattaaagggctgtggctttagagggaaggacctctcctctgtcattcttcctgtgctcttttgtgaatcgctgacctctctatctccgtgaaaagagcacgttcttctgctgtatgtaacctgtcttttctatgatctctttaggggtgacccagtctattaaagaaagaaaaatgctgaatgaggtaagtacttgatgttacaaactaaccagagatattcattcagtcatatagttaaaaatgtatttgcttccttccatcaatgcaccactttccttaacaatgcacaaattttccatgataatgaggatcatcaagaattatgcaggcctgcactgtggctcatacctataatcccagcgctttgggaggctgaggcgcttggatcacctgatgtcgggagttcaagaccagcctgaccaacatggagaaaccccgtttctactaaaaatacaaaattagccgggcttggtggcacttgcctgtaattccagctactcgggaggctgaggcaggagaatcacttgaacctgggaggcgggggttgcagtgagctgagatcgcatcattgcactctaacctgggcaacaagagcaaaactccatcaaaagaaaaaaaaaatcgggtgcagtggctcatgcctgtaatcctaacactgtgggaggccaagacaggcagattgcctgagctcaggagttcgagatcagcctgggcaacatggtgaaaccctgtctctactaaaatacaaaaaattactcagcgtggtggcatgcgcctttagttccagctactcaggaggctgaggcaggagaatctcttgaacccgggaggtggaggttgcaatgagccaagatcgtgccactgcactccaacctggcaacagagcgagactccgtcttaaaaaaaaaaaaaattttgcagcgcaaaccaggatatcctctgttctcatttgttctagatttcaaaagaaacagtcctttctttggggaaaagagaaaggaaaaggagttttataaaaggaaagaaaagattcataagaacaagaagtgggcccacttgcatatacctttgtagaaaactgttcactgttgttgaagaaaagctcttcatattaatatgcagtccagatgcagtggctcacacttataatctcagccctttgggaggctgagacaggaagattacttgaggccaggagtttgaaaccagcctgggcaacatagtgagactctgtctccacaaaatttttttttaattagccgggcatggcagtgtgcttctgtagtcttagctactgaggaagctaagccagaagaatcacttgagcccaggagttcaaggctgcagtgagctatgatcataccattgcactcttgcacttgcacagagcaagaccctgtctcttaaaaaaaaaaaagtgtgtgtgtgcatatgcatatatacatatatatacatgcaaatgtatctgtttataattcagattgcttcaaaaagatgttgcactttatgatactgagaacagtgagaagtaaataagatagagtgtaggaggaggaataatttcagaacagccatctgagaacttctgtgacaacagatcaggcaaaatgaaatgtgaaagtaattttataggccaggcgtggtggctcatgcctataatcccagcactttgagtggccaaggcaggtggatcacttgaggtcaggagttcgagaccagcctggtcaacatggtgaaaccttgtctctactaaaaacacaaaaaaattagtcgagcgtggtggcatgtgcctgtaatcctagctgctggggaggctgaggcaggagaatcacttgaacccgggaggcggaggttgcagtgagcctagattgcaccactgcactccagcctgtgagacagaatgagaccctgtcttaaaaaaaaaaaaaaagtaattttataaactattgtgcacaattcgatgtattcataattaattaaatgattatttttgttggttttaacttttattcagtggctatttattgggagcctactgtgttctgggcactaggaatgcaacagtaaataagactaactaagtccctggtaggattcaggttctgtcgaggggagatacacaataaagatgaatttaagataacaataaatgctatggagaaatatacagaacagtggaatagtattagctgtcaaaggttgttgattactttcgtttaaggaggccagggaaagcctttctgaaaaaattgagctgagacctaaataacaagaaataattgtccttgaaaaatgaagggaatgcatcttataggcagaggaatagcaaacataaaggtcttgaggtaataatgagtgtggttttttgatttctgtattttggtttttttgagatggtgtctccctctatcccccaggctggagtgcagtggcacaatcttggctcactgcaaactctgtctcctgggttcaagcaattctcctgccttggcctcctgagtagctggtattacaggcacgcgtgctaccacacccgactagtttttatttttagtagagatggggttttaccacgttggtcaggctggtctcaaactcctgaactcaagtgatccaaccacctcaacctcccaaagtgctgggatcacaggcgtgagccaccatgcccggccagagcttggtttattttttaaaagataggccaatgttggtcgtgtgtggtggctcgtgcctataatcccagcactttgggaagccaaggcaggcaaatcacttgaggtcaggagttcgagaccagcctggccaacatggtgaaaccccatctctactaaaaatacaaaaaactagcatggtgtggtggtgtgtgcctgtaatcccagtgcctgtaatcccagctactccagaggctgaggcaggagaatcacttgaaccgaaaggtaggagttacagtgagccaagatcgcatcactgcactccagcctgaacgacagagcaagactcctgtctcaagaaataataatgataaaaggttcgggcacagtggctcacacctgtaattccagcactctaggaggccgaggcaggcagatcccctgaggtcaggagtttgagaccagcctggccaacgtggcaaaaccccatctctactaaaaaatgcaaaaattagctgggcacggctgggtgtggtggctcattcctgtaatcccagcactttgggaggtcaaggcggacagatcactgaggtagaaaccctgtctctactaaaaatacaaaaatttgcccagcgtggtggcgcgtgcctctaatcccagctacacgggaggctgagacaagagaatcacttcatcaacccgggaggtggaggttgtggtgagctgagatcgcaccattgcactccagcctgggcaacaagagtgaaactccatctcaaaaacaaaaaaaaattagctgggaatggtggcatgtgcctgtaatcacagctacttgggaggctggggcaggagaatcgcttgaacccaggaggcggagattgcagtgagctgagattgcgccactgcactccaggctgggcgaaagagcaagactccgtctcaaaaataataataataataataataggccagtgtagctggagtaatttgcaaattatgtgtggaggcagagattacacaaggaatgggagaaggtcatagatgagggccagatcacatagtatttggtggtaaggaattcagattttatccttgtggtaattggtggtgtggagatggttaaaaacaaggttggtttgggatgggtttgaagagaggacttgctaatggattaaatttggaggataaggtaaagagaaattgaaggagtgacacttgggttttggcttgaacaatagatcttgttagtaatattaaattagatgaagaaggcatggtagggaatatgggggagtgggaaaggcaggaagcaggaatggaaccaggaactctgttttagatgtgagaatttgttgttgttgttgttgttgttgttgttgttgttgttgttgttgtgacagcatctcgttctgttgcccaggctagagtgcatggagtgcggtagcacgatctcagctcactccaacctccgcctcccggttcaagtgattttcctgcctcagcctcccgagtagctgggattacaggcacctgccacaatgcctggctaatacttgtatttttagtagagatggggttttaccatgttggccaggctggtcttaaactcctgacctcaggtaatccacccacctcggcctcccaaagtgctgggattgcaggtgtgagccactgtgcccggccagatgcatgaattttgagatgtatactagacttctggatagagaagttaagtaggcagttggacacattgtatgaagctcaggggtacaaggaggactatgaacatgggagtcttctgacaaatttatcactagactcctcattcaagtaactaggaaatgtcagatattcttcccctagtaatagccagtggttatactcttgcctttagttttcttcacaatactcttggcaacacataaggccttccctacaatctgagtttcagtcagaattgtttctgagcgttcttcctcaaatttctccccagtctcattattctttattctcatgtccatgaccagtcataatagtaattatgaaaaacctctaactttctttagtgcattgaatgtatattttatcattttggttgtgttaactgtaaatctctcagtggaaatctgaaaagcctttatttccttagatgataatatacaattgatttaggagatagggaatttttcagttacctttataacagcacagtattagcagtctaatctaaatgctaagtgaatgttttgagaggagatagatgttgaaaattaaaatacattaagtcccagtgaggtgaaaagccgattgttaagttctgcacacaaaagatttgcttcagtgaattgatttcaacagctgagatcctagtcatttcacctggtctaccaaaaagaatgattttacttgcttttggtcaaatctctgcccagcaattctttttctttctttcttttttttgttttatgtgtgtgtgtgtgtgtgtttttttttagcagagtctcactttgtcacccaggcgggagtgtggtggtatgatcacagttcactgcagcctccaactcctgggctcaagtgatcctccagcttcagcttttcaagaaattgggactgcaggcacatgcaactatgcctggctgaggttttatgtatcttttttctagagaaggggtctcactgtgttgcccagctgggtctccagctcctggtctcaagctgtcctcctgcctcagcctcccaaagtgccaaagtgctagggttataggtgtgagccattggtgcccagctactgcctgcctggcaattctgaatgccttaaattttttttttttttttttttttttttgagacagagtttcactctgtcacccaggctggagtgcagtggcatgatcgtggctcacagcaacctctgcctcctggattccagcaattctcatgcctcagcttcccgagtagctgggactacaggtgcatgccaccacgcccagctaatttttggtttttttgtttgtttgtttgtttgttttgagacggagtctcgctcagttgcccaggctggagtgcagtggcgtgatctccgctcactgcaagctccgcctcccgggttcacgccattctcctgcctcagcctcccgagtagctgggactacaggcgcctgccactacacccggctaatttttttgtattttaagtagagacggggtttcaccgtgttagccaggatggtctcgatctcctgacctcgtgatccgcctgtctcggcctcccaaagtcctgggattacaggcgtgagccaccacacccggcctaatttttttttttttaattttatttttaattttttgagatgcgagatggagtctcgctctgttacccaggctggagtgcagtggcaccatctcagctcactgcaacctccacctcctgcattcaaaagattctcctgcctcagcctcccaagtagctgggattacaggtgcctgccaccacgcccaactaattttttgtatttttagtagagatgaggtttcaccatgttggtcagactggtgtcgaactcctgacctcaagtgatctgcctgcctcagtctcccaaagtgctaggattacaggggtgagccactgcgcctggcctgaatgccttaaatatgacgtgtctgctccacttccattgaaggaagcttctctttctcttatcctgatgggttgtgtttggtttctttcagcatgattttgaagtcagaggagatgtggtcaatggaagaaaccaccaaggtnccaaagcgagcaagagaatcccaggacagaaaggtaaagctccctccctcaagttgacaaaaatctcaccccaccactctgtattccactcccctttgcagagatgggccgcttcattttgtaagacttattacatacatacacagtgctagatactttcacacaggttcttttttcactcttccatcccaaccacataaataagtattgtctctactttatgaatgataaaactaagagatttagagaggctgtgtaatttggattcccgtctcgggttcagatcttagctgataagtggaagagctgggactttaagcagatgagaatctaaagactttgctcttttcacttcactggggtgtctttctctctctctctcttgctctctctctctctttttttttttcccaagacggagtctcactccattgcccaggccagagtgcagtggtgcgatctcagctcactgaaaactcatcttgcccaggctggtcttgaacccctgaccttgtgatcctcccgccttggcctccccaagtgctgggataggcgtgagccaccgtgcccagccaataatagctaaaatttatataatgttcactgggccaggcacagcggctcgttcctgttatcccagcactttgggaagctgaggcaggcagatcgcttgagccaaggagttcgataccagcctgggcaacatggcaaaaccccatctctaccaaaaaaaatatacaaaaattagccaggcgtggtggcatgtacttgtagttccagctactcggaaggctgagttgagagtatctcttgagcccaagaagaggggactacagtgaacggagattgcgccactgcactccagcctagacgacagacagaagatctcaaaagaaaaaaaaaaaaaaaagatcactttatgctgggactgctctaaaggcccaaccatgttttaactaattaacaattttatgacaactctatgagctatgtactgtaattatgcctatattacagatgtgaaaattgaggctcagagaggttgaataagttgctcaaagtcacacaggtaataagtgatggaactagaagttgaactcaggaagtctagctccaagtctaaattctttgttaatttatttttcgggccagagtcttactctgtcacccaggctggagtgcagtgccactatctctgctcactgcaaccttcacctcccaagttcaaaccttgttcaattcttgtgccttggcctcccaagtggctaggattacaggcatgtgccacaacaactagctaattttttgtctgattctgttggccagtctggagtgcagtggcgcaatctcagctcactgcagtctccagctcccaggttcaagtgattctcgtgccttagcctcccaaatagctgggattacaggcacgtgccaccacaccgagatagttttttgtatttttaatagaaacaaggtttcaacatgttggccaggctggtctcaaattccagacctcagatcatctgcccgcctcaggctcccaaagtgctgggattacaggcatgagccactgcacccggccttaatttttatatttttattagagatggggttttgccatgttggccaggctggccttgatctcctggcctccagtgatccacccgccttggcttcccaaagtgctgggattacaagcatgagccactgcacccggcctccaattctaaactcttaacaacaatactatagtttcttgaaaagttgttgaaggcttcacggagggaaaaaaaatggagcattctaacaactttgcagatgagacccaagaagactcaatgactttctcctgatcatattgtagcagatgacttagccagaactctgacttcctcacagggagaaagtctgcaagatttcacacttacctgtcaggcctgagctggctgctttctcagctccctaagtgctatgttcccagtctgcttttcttcctttttcaagtgtgcactaccaggcatttcagaacatcccaggctggtcgcggtggctcacacctgtgatcccagcactttgggagcccaaggcgggtggatcacctgaggtcaggagttcgagaccagcctggccaacatggtgaaaccccatctctactaaaaatacaaaagttaactgggcgtggtggtaggcacctgtaatcctagctcaggattactcgggaggctgaggctagagaatcggttgaacccaggaggcggaggttgcagtgagccaagattgcgccactgcactctagcctggggacaagagggagacttcatctcaaaaaaaaaaaaaaaatcccagctgggcacagcggctcacttctgtaatcccagcactttaggaggccaaggcaggaggatcacttgagcccaggagttcaagactagcctgggcaacatagtaagaccctgtctctacaaaaaaatttaaaaattaattgggtgtcgtagcacactcttgtattcccagctactcaggaggctgaggtgagaagaatgcttgagtctgggaggtcgaggctgcagtgagccatgatggtgctactgcactccagcctggccaacattgtgagaccttgtctcaaaacaaaacaaaacatccttctactgagcactttctgtccctttatagaaacttaagagggaaccagtagaggtaatttcctaaggaaaactgctttgggacatgatcacaaatgaagcctggagttttgaactgctgaggtcagcctgtttttaccttctgagcctatcaagtaattgttccagatgccaagaaaagctgctggccttatttctgcttctgcctttaccacaggggagcgccatgtgagccagtcctctgtttttcctccactgtatgctaggcagtattagcaccagattcttcccctctttaaaaagaaattctagtgctttggattttttcctccatgcagaatagcaatgatggaaagtatgtggtcaaagtaatgacattctgaaaatactaaatgtcaccatagtatttttctctggaagagaaatgtatatgtagaggtgaaacttcaaatttctttttttttttttttaagacgaagctttgctcttcttgcccaggctgaagtacaatggcgtgatcttggctcaccgcaatctctgcctccagggttcaagtgattctcctgcctcagcctcctaagtagctaggattacaggcatgtgccaccacgcccagctgattttgtatttttagtagagatggggtttctccatgttggtcacgctggtcttgaactcccgaccccaagtgatccacccacctcggcctcccaaagtgctaggattacaggccaccgcgcccggcctgaaacttcaaatttctttttttttttgagacagagtctcgctatgtcacccaggctggagtgcagtggcgccgtctcggctcactaccagctccactccacctcctgggttcacaccattctcctgcctcagcctcccaagtagctgggactacaggtgcccgccaccatgcccagctaattttttgtatttttagtagagacgggttttcactgtgttagacgggatggtctccatctcttgacctcgtgatccgcctgcctcagcctcccaaagtgctgggattacaggcgtgagccactacgccaagcccgaaacttcaaatttcttatctcataactaggcatcctcatcactgagtgttagcctggatataaacattcctaatcttttgtacttttcatgtcagcatttggctccacttggctgcctggggagaacttctagcattatgagcatgcaggtcctatcaacaggttgggggtgcggtttattcatacaggtagtgagagtggcacagatggatgctgtcccttaaaacaaacagacttgtctttgggagcctgaggcgggtggatcatgaggtcaggagttcaagaccagcctggccaacatagtgaaaccccgtttctactaaaaatacaaaaaattagccgggtgtggtggtgtgcacctgtaatcccagctactagggaggctgaggcaggagaatcacttgaacccaggaggtggaggttgcagtgagccgagatggcaccattgcactccagcccaggcgacagtgcaagactgcgtctcaaaaaaaaaaaaaaaacacacagacttgtcctactgccatttcttttcactctggcggtaaagtaagagtgtgtgtgtgtgtgtgtgtgtgtgtgtgtgtgtgtgtgtgtgtgtgtgtttctgtccgtctgtctgtcaagggggagggtgaccactttctaaaaggccatccgtgtatttttagcttcctgatttttttctctatcgcagtctctttgaagccaggtgaattttaggccttggcaattttctttttattgcaatgggaaggtcaagacactgagagtcacccaaaacatatccatccaaaatgatacaattttagggtttatttttaagtgatacccaagttatttgctaagaacctatgccagtgtgtttatgagaatttgcactgtcccacactgttgccaccagccacatgggactgtttaaatttaaattttacaaattagccagtcatggtggtgtgcacttgtagtcccagctacttaggaggctgaggcaagaggattgcttgagcccagaagttcaatactgcagcaagctatgatcgtgccactgtactccagcctgagtgacagaatgagacctcatctcttcaaaaaaaaagaaaaaaattaaaatatgaagtttagttcttcattcaccctaaccacatctccagtgctcaataactatatgtgactcatggctaccttattagcatagatatagaacattgtgactatcacagaaagttgttttgaacagtgttgccaagccctgtaagtggaagaggcagtgcagtgtgatctgtgtcttcaggaaaccaggtagtcagactagttcaatgaggagaggcagaacctggcttcacttctagattaaaaactgcttaggtggcctaaagatacaatggccattctcagagtagtgagaaggaaggaacagatgtttagggggctagaagaaagtcagagagggccgggcgcagtggcttatgcctgtaatcccagcactttggaaggccaagacaggcagatcacgaggtcaggagttcgagaccagcctgaccaacatggtgaaaccctgtctctactaaaaatacaaaaattagccgggcgtggtggtgcgcgcctgtaatcccagctactcaggaggctaaggcaggagaatcgcttgaaccctggaggcagaggttgcagtgagcccagatcgcaccactacgctccagcctaggtgacagagagagactccgtctcaaaaaaaaaaaaaaaagtcagaggagacaaggagcatgtacacctaaaatcaacatagacccctctgttgatggggtcatagtgagtacttgaggtaccaagtctggataaacatcaaacttcagccaataactttgagtttctagccatccaagcctcttattaaacatacagaaggaccttttttcccttgcatctaacaagttaaagcacctgcagagatcattagggaggagccttggcctgattggtgacaaaagtgagatgctcagtccttgaatgacaaagaatgcctgtagagtgcaggtcaactacatatgcacttcaagaagatcttctgaaatccagtagtgttctggacattggactgcttgtccctgggaagtagcagcagaaatcatcaggtggtgaacagaagaaaaagaaaagctcttcctttttgaaagtctgttttttgaataaaagccaatattcttttataactagattttccttctctccattcccctgtccctctctcttcctctcttcttccagatcttcagggggctagaaatctgttgctatgggcccttcaccaacatgcccacaggtaagagcctgggagaaccccagagttccagcaccagcctttgtcttacatagtggagtattataagcaagatcccacgatgggggttcctcagattgctgaaatgttctagaggctattctatttctctaccactctccaaacaaaacagcacctaaatgttatcctatggcaaaaaaaaactataccttgtcccccttctcaagagcatgaaggtggttaatagttaggattcagtatgttatgtgttcagatggcgttgagctgctgttagtgccaacatgttagtgagaaaatatctttggataggtaaaaatcaaggaggagttctcctcttcctaaaccatcttaatttacttacatagaagaaagcacagcagctggcccaccacggacgggcccagagcaggggaagattctcggtgaacatttctttttttttttctttttttttgaggtcgagtctctgttgcccaggccagagtgcaatggcgcgatctcggctcactgcaacctccacctcccgggttcaagtgattctcctgcctcagcctcccaaatagctgagactacaggcgtgtgccaccacgcccgactaattttttgtattttttttttagtagagacggggtttcaccgtgttagccaatatggtcccgatctcctgacctcgtgatccacccgcctcagcctcccaaagtgctaggattacaggcatgagccactgtgcccagccctctccatgaacattttctaattaaacttgacacttaatacaatgttatgcttaggactgctataaagcttacctctggagttgcgcagcacaaaggccttggtgtgtgtataaatttggtttgttcttttcacagcaaaagctacccacctttgcctcctgtgcctgcttctgcccagggacttaggtcctcttacaccttagagaaaggccttagcatctggtcacaggcagatgagtgacagcaagaaaacctggctgcaatgtaattttgtttccatcctctttattagttatcaattggatttttatgaaatttccaagttccactcaaggatttctcagtgtttttttactttggtatagtggaaaccagggttgccagaaagtattattttgggggtgagttagtcaaccttcgttcagtcagacagacaggagcacctcagcaattcccagaaacgggctgatgggaaagagcaacatacatgaatgtcttgaagaacacagccaacagagcccattgggcagttctgattttccaggtacacagcatctccacagtctcttctgatttttattcccctgagtatatggattccagctcagcatgtagcctttccctgctgagtctctaaccaggataacatgtatttttttgactggatgaattatcttcccatctcttgacatttacagtaattaccaccaagtatggtattttcagtggccgtgattatcagttaccaacacagaattaggatgaagggaggaagggagggaaggaaggtgggtgttttttcacacagtgtcttagccagcaatttagcaaattaatggaaattagatctttgatttttttttctttcaagcattttatttgagagactatcaaaccttataccaagtggccttatggagactgataaccagagtacatggcatatcagtggcaaattgacttaaaatccatacccctactattttaagaccattgtcctttggagcagagagacagactctcccattgagaggtcttgctataagccttcatccggagagtgtagggtagagggcctgggttaagtatgcagattactgcagtgattttacatctaaatgtccattttagatcaactggaatggatggtacagctgtgtggtgcttctgtggtgaaggagctttcatcattcacccttggcacagtaagtattgggtgccctgtcagagagggaggacacaatattctctcctgtgagcaagactggcacctgtcagtccctatggatgcccctactgtagcctcagaagtcttctctgcccacatacctgtgccaaaagactccatctgtaagggatgggtaaggatttgagaactgcacatattaaatatactgagggaagactttttccctctaactctttttcccatatgtccctccccctcctctctgtgactgccccagcatactgtgtttcaacaaatcatcaagaaatgatgggctggaggctgggcatggtggctcatgtctgtaatcccagcactttgggaggccgaggcaggtggatcacttgtcaggagtttgagaccagcctggccaacatggtgaaaccccatctgtactaaaaaaaaaaaaacaaaaagtagccaggcctggtggagcatgcctgtaatgccagctatttgggaagttgaggtgtgagcatcgcttgaacgtgggaggcagaggttgcagtgagccaagattgcaccactgcactccagactgggtgacagagtgagactttgtctaaaaaaaaaaaaaaagagagagagagaaaagctaggtgcggtggctcacgcctgtaatcccagcactttgggaggctgaggtgggcagatcacgaggtcaagagatcgagaccatcctggccaaccaacatggcgaaaccccgtctctactaaaaatacaaaaattagctgggcgtaggggcgcacgcctgtagtcccagctacttgaggggctggggcaggagaatcgcttgaaccccggaggcggaggttgcagtgagccaagatcgcgccactgcactccagcccgggcgacagagcgagactccgtctcaaaaaaaaagagagagagagaaatgatgggctgggccagtgccccacccctgtaatcacaacactgggaggccaaggtgggagaatcgcttgagcctgggagctgaagaccagcctgggcaatacagtaggacctcatgtctacaaaaaaattattaaaaattagccaaggctgggtgcggtggctcatgcctataatcccgggggtgaagttgagcccaggagtttgagaccagcctgggcaacatggcaaaaccctgtctctaccaaaaatacaaaaaaattagccaggggtggtggtacgtgtctgtagttccagctacttaggaggctgagatggaaggattgcttgagcccaggaggcagaggtggcagtgagctgagatcacaccactgcactccagcctgggtgacagagcaagaccctgtctcaaaaacaaacaaaaaaaatgatgaagtgacagttccagtagtcctactttgacactttgaatgctctttccttcctggggatccagggtgtccacccaattgtggttgtgcagccagatgcctggacagaggacaatggcttccatggtaaggtgcctgcatgtacctgtgctatatggggtccttttgcatgggtttggtttatcactcattacctggtgcttgagtagcacagttcttggcacattttaaatatttgttgaatgaatggctaaaatgtctttttgatgtttttattgttatttgttttatattgtaaaagtaatacatgaactgtttccatggggtgggagtaagatatgaatgttcatcacaaaaacataaatcaaggccgggcatggtggctcatgcctataattccagcactttgggaggtcaagatggaggtcaaggtgggagcctagaagttcgagaccagcctgggcaacataaggagacttcatctgtacaacaaatttaaaaagtagctgggtgtggtggcagatgcctgtagtcgcagctacttgggaagctgaggtgggaggatcacttgagctcaggaggttgatgcttcagtgagccacgatcacaccactgtactccagcctgggcgacagagcgagaccgtgtctcaaaaagaaaaaagaaagtataaatttacacaaaaacaataaaataatcccagtaattccaccacttggagatgatcaccataaaactccaccaggcatatgtgcgtatatatacacgtgtattttataaaatgtgatcataattacactgttttgcttttttccttaagatattacatacatttttccacatcgttaaattacagtgctgttttcctggtggctttcctttaacagattgaagttcatgttaatacagttgccagaggctgtgggctttcactgtcaccaggagtcactcctagggcctcttcagagcaaggccttatgtcctgaagcattgccttttttttttttttttgaggtggagtctcactctgtcacttagcaggctggagtgcagtggcccagtcttggctcactgcaacctccgcctcctgggtttaaatgattctcctgcctcagcctcagggcggatcacctgacatcaggagtttgagaccagcctggccaatatggcgaaaccccatctctactaaaaatactaaaaaaaattagccaggcatggtggcacgcacttgtagtcccagctacttgggagactgaggcaggagaatcgcttgaacccaggatgttgaggttgcagtgagctgagatcacaccatcacaatccagcctgagtgacagagtgagactccatctgaaaaaaaagaaaaaacaattagcctggcatggtggcaggcacctgtaatccctgctacttgggaggctgaggcaggagaattgcttgaacccgggaggtggaggttgcagtgagctgagatcgtgccattgcattccaggctgagcaacaagagcaagactccgtctcaaaaaaaaaaaaaaaaaaaaaaaaaggccaggtgcagtggctcacgcctgtaatcccagcactttgggaggccaaggtgggtggatcacctgaggtcaggagttccagagcagcctggccaacattgtgaaacccccgtctctactaaaaatacaaaaattagctgggtgtgatggcatgtgcctgtaattccagctactcaggaggcagagacaggagaattgcttgaacccaggaggcggaggttgaatgagccgagattgcgccatcacactctagcctcggcgacagagcaagactccgtctcaaaaaaaaaaaaaaaaaaattagcttctacctcattaatcctaagaactcatacaaccaggaccctggagtcgattgattagagcctagtccaggagaatgaattgacactaatctctgcttgtgttctctgtctccagcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaccatgaatgactgttcttgagacttaggccagccgactttctcagagccttttcactgtgcttcagtctcccactctgtaaaatgggggtaatgatagtatctacctcctaggatttattgaggcagcttaaataccttttgtatttcctgttgctgccaaaacaaattgttgcaaggtcagaagtctgaggtggctcaactgtttctttgtttcaggtttcatgaggccaaaataaaggtgttcgcagggcgtgttcccttctagaggctctgggtccttgcagttctaggactaagatccctgtttcccactggctgttggctgggcatcattctcagcttcttgaggctccccacattcctaggctcctggcctgtctgcctccatcttcaaaaccagcaatgggtggtcaagtttttctcacactgaatcttgctgactactgtatctttctaactcctgccagagacatttctctgtttctaagggctcaagtgattagattgcacccacttggtaatccaaagtgatcttcatatcttaaggcccatagccttaattatagctgcaaagtcccttcgcagcagtacctagattactgttggaatgaataaccagaagacagcaatcaagggaggacatctttagaattctgcctaccacttgtatttaacatgcttaatccacagatgacactctctaccattatttcctggtcctcacactgctcagagattggaatcctttttaagcaaagagaatgaagtcatcacatagttcagtcctgctgtatttgctggaaacagtgaag

SEQ ID NO: 5 - DNA, 86101 bp, Homo sapiens
  misc_feature (26050)..(26050): T can be deleted
aggcctccttctacaactaaggagatctgattatcaaactaaaatggtccctaaggaaccaagcggagcccaccctttttctaatccaaggttgcttctgtttactgctcaagcaccttctggaagcagcaaggcccccatgggagcaactctcactgaatccatttgaaggttttgtaggtcttacaacaaaccctattcagccttgtattaggcatgttacagaaccaacgaattcggagatgaagtcaggtcttccagttcagcctgcgaggaagacaggtgatccgaatcctaagaatgcaaaagatgggccgggtgtggtggctcatgcctgtaatcccagcgctttgggaggccgaggcaggcagatcacctgaggtcgggaggttgagaccagactgaccaacaacggagaaaccccgtctctacttaaaaatgcaaagttagccgtgcgtggtggcccatgcctgtattcccagctactcgggaggctgaggcaggagaaccacttgatccctggaggcggaagttgcggtgagcggagattgcgccattgcacaccagcccgggccacaagagcgaaactccgtctcaaaaaaaaaagcaaaagatactaccaagccctgcggagcaaggtacctcacacttcatgagcgagttaagatgggtttcacaatttttcaagcaaggaaacgggctcggaggtcttgaacacctgctacccaatagcagaacagctactggaactaaaatcctctgatttcaaataacagccccgcccactaccactaagtgaagtcatccacaaccacacaccgaccactctaagcttttgtaagatcggctcgctttggggaacaggtcttgagagaacatcccttttaaggtcagaacaaaggtatttcataggtcccaggtcgtgtcccgagggcgcccacccaaacatgagctggagcaaaaagaaagggatgggggacttggagtaggcataggggcggcccctccaagcagggtggcctgggactcttaagggtcagcgagaagagaacacacactccagctcccgctttattcggtcagatactgacggttgggatgcctgacaaggaatttcctttcgccacactgagaaatacccgcagcggcccacccaggcctgacttccgggtggtgcgtgtgctgcgtgtcgcgtcacggcgtcacgtggccagcgcgggcttgtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcgggttagtggtggtggtagtgggttgggacgagcgcgtcttccgcagtcccagtccagcgtggcgggggagcgcctcacgccccgggtcgctgccgcggcttcttgcccttttgtctctgccaacccccacccatgcctgagagaaaggtccttgcccgaaggcaaattttcgccaagcaaattcgagccccgccccttccctgggtctccatttcccgcctccggcccggcctttgggctccgccttcagctcaagacttaacttccctcccagctgtcccagatgacgccatctgaaatttcttggaaacacgatcactttaacggaatattgctgttttggggaagtgttttacagctgctgggcacgctgtatttgccttacttaagcccctggtaattgctgtattccgaagacatgctgatgggaattaccaggcggcgttggtctctaactggagccctctgtccccactagccacgcgtcactggttagcgtgattgaaactaaatcgtatgaaaatcctcttctctagtcgcactagccacgtttcgagtgcttaatgtggctagtggcaccggtttggacagcacagctgtaaaatgttcccatcctcacagtaagctgttaccgttccaggagatgggactgaattagaattcaaacaaattttccagcgcttctgagttttacctcagtcacataataaggaatgcatccctgtgtaagtgcattttggtcttctgttttgcagacttatttaccaagcattggaggaatatcgtaggtaaaaatgcctattggatccaaagagaggccaacattttttgaaatttttaagacacgctgcaacaaagcaggtattgacaaattttatataactttataaattacaccgagaaagtgttttctaaaaaatgcttgctaaaaacccagtacgtcacagtgttgcttagaaccataaactgttccttatgtgtgtataaatccagttaacaacataatcatcgtttgcaggttaaccacatgataaatatagaacgtctagtggataaagaggaaactggccccttgactagcagtaggaacaattactaacaaatcagaagcattaatgttactttatggcagaagttgtccaactttttggtttcagtactccttatactcttaaaaatgatctaggacccccggagtgcttttgtttatgtagcttaccatattagaaatttaaaactaagaatttaaggctgggcgtggtggctcacgcctgtaatcccagcactttgggaggccgaggtgggcggatcacttgaggccagaagtttgagaccagcctggccaacatggtgaaaccctatctctactaaaaatacaaaaaatgtgctgcgtgtggtggtgcgtgcctgtaatcccagctacacgggaggtggaggcaggagaatcgcttgaaccctggaggcagaggttgcagtgagccaagatcatgccactgcactctagcctgggccacatagcatgactctgtctcaaaacaaacaaacaaacaaaaaactaagaatttaaagttaatttacttaaaaataatgaaagctaacccattgcatattatcacaacattcttaggaaaaataactttttgaaaacaagtgagtggaatagtttttacatttttgcagttctctttaatgtctggctaaatagagatagctggattcacttatctgtgtctaatctgttattttggtagaagtatgtgaaaaaaaattaacctcacgttgaaaaaaggaatattttaatagttttcagttactttttggtatttttccttgtactttgcatagatttttcaaagatctaatagatataccataggtctttcccatgtcgcaacatcatgcagtgattatttggaagatagtggtgttctgaattatacaaagtttccaaatattgataaattgcattaaactattttaaaaatctcattcattaataccaccatggatgtcagaaaagtcttttaagattgggtagaaatgagccactggaaattctaattttcatttgaaagttcacattttgtcattgacaacaaactgttttccttgcagcaacaagatcacttcattgatttgtgagaaaatgtctaccaaattatttaagttgaaataactttgtcagctgttctttcaagtaaaaatgacttttcattgaaaaaattgcttgttcagatcacagctcaacatgagtgcttttctaggcagtattgtacttcagtatgcagaagtgctttatgtatgcttcctattttgtcagagattattaaaagaagtgctaaagcattgagcttcgaaattaatttttactgcttcattaggacattcttacattaaactggcattattattattattatttttaacaaggacactcagtggtaaggaatataatggctactagtattagtttggtgccactgccataactcatgcaaatgtgccagcagttttacccagcatcatctttgcactgttgatacaaatgtcaacatcatgaaaaagggaaatgattccatagcgttattatgaaagtagttttgaactgtaatggtagaggatgaatagctcacaatacaaatttgtcatttccctttaagagagaattcccattttatgtgagagtccacatgttcctcatacccatagtttgccacatcttgagtactcttcagaattatttgaattttttgaattttatctgtggaatgtatttttttttttttcttttttgagacacagtcttgctctgttgcccaggctggaatgcagtggcgtgatctcggctcactgcaaccaccgcctcctgggttcaagtgattctcctgtggcagcctccggagtagctgggactacaggcgtgtgccaccacgcttggctaattttttgtgtttttagtaaagatggggtttcaacgtgttagcaaggttggtctcgatctgacctcgtgatctgctcgcctcagcctcccaaagtgttgggattacaggcgtgagcccccgcacctggccgaattttatcgtggaatgtattcttaatgtgaatagtttttgattccgaaccatgaataataagaaaataaataaaatttaaatgaaaataaaagctaatatatacagcttttaataatatagttaaatgccatcttgtaacttttgtgaactcttgttacacctttctatagattcgcaagagaatggattaatgatcttgtttaattaatatgccttaacaaaagtaatccatagtcaagatcttaagcatttttttccttatgatctttaactgttctgggtcacaaatttgtctgtcactggttaaaactaaggtgggattttttttttaaatagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggtaagtaatgcaatatggtagactggggagaactacaaactaggaatttaggcaaacctgtgttaaaatcttagctcattcattaattgtgtcatgctgggcaaatcagtctctctggcctctttttcctcactcgaaaaatggagacgatgaaaataatgtctcataggtttggattaaattaaataatgtaggtacttagtaaatgttctctttcatccctcctttgataaatttgccaactgagatttgctgaattacgtctttcttatgccaaaaaaacctaggacttgttttgatgttaattaaactaaactatatttctgcaggctatcacagaggacagagattattttaccgatatactataagtatcatgatttggaaggagtttccctggcgtaggtgccgcatgtttctaagcaattatgtaataagattatatattcagtcattcaaataattattacctacttgacataagtaatgaactttcccttttcttcagagtgttaatctctagtaaggggaataaagagtacacagataaagtatagtgtaaggttgaatgtagtatgtgctaagagaaaaatataaaaaagtataatgagagttgagaagaaagagcaaatagtattgggcaaagttaggcaattattcctttgagctaaaccttgaaggataggtgagagattaagaaatttgaagatgtggtagagtgataatgttctaggcagagggaacaacatgaggaagaatatgtagtgtgttcaggaaatagcaagtaattcaggttggctttggttgttttgtgtctgaaagggaccaatagacaaggcaaaaaggcagactaaaggcaggcattgaatgccaagctaaagaaattgaatttgtttggttggttggtgagcagagaaatcacatgcaaatttcatcatgctacttattgtgtcaaaccttagatcacctccctttgtccttatagcaaaatctaaacttgatatggctttcaagttcctttgtgatcaggcccctgatttacactcttggctcagcttgccatattcatcctctcacctatcttcatttgccattcattcctactgaatttcttttcgttaccaaaaccacaatgctctctggctctttattaaacatattgttacctctacccacaacctactttttccctactttttgtctagctaatttgcgtgctcgtctttcagatcttggcttatttctgcttctgagaaatacttcctgtctgccctcgttgagcttctagtgaaggagacatacataagcaattatagtgtgatacatgctttgaaagaaattcatggctatagggagtgcatatacaaagggaatataggtaatgggcaaatatttacatgtatgttattggataccaaatggtatacataggattcagtaaatatttgtagagtgagtattagtattatttgctttagaaagcctaatgatcaaacagcagtctttggagataacgtttttcaaaatgtcatgtctgtgccattagaatcttctagactgctcattgaaaggacagattccaggccccactctgaatctcttaatttataatttttggaaatgatgcccatgagtctacattttaaactacctgaatgatccctatagaaagagaaaactggaggtaggaagatcagttaggggatgtgtaatggtctaggtgatagagacaagtgcctgaattacagtaataacagtgaaagtaaatatggaacataaaactataggaccttgcagtagtctagatatggaggattcaaaaaaaggaacaaatgacagggcaaagcatatgcagaacacagtagtaacagtcatagaaatggataagggagtcatccattctgcaaatacttactgcttacttgtgtctggcaacctgctcggcattaaggatacaaatatgaataagatgtcctttgacctctaagtactcagtctcgtaagcacgtcttgtaagcacatcttggttgcttccataaaaataaatacactagtgtgatatgttataagagcatgtaccaagtgcatgaaaagtgagcagccatctctggttggtcagaaaaagctccataaagcagtttttgctgaatcttgaaagatatacctaaggtcaaatggttaattctttaatcataacctgctagaattgatctataaccaaggaaggatagtaaggaattaataaggccactctcaactcactgcaaaggagttaactttttgaaggctgtaatacataaatctgctgactagtctcttgagaccttttgcttttacgtttactttagattcagtattgaaaagtaagagtaatggacttaagctgtgtttttcaacctgttttgttcagttctaacatgtaatattttttaaaaaattattcctaaagttctatgaggaattgtgctgtttctgcctctcagcagtccttccttttgcattaaatcataggcatttctgttaccattcttcagcttattaatgagatcctcaggttatttgggaaatgtttatttggtaattaactctttttcacctagttcatttttttaacttttttttttaaatagccgagtttcttttcattgctgaactaaaatggatgtgttattattagctgaactccttagtttactttagagttcaccctttgtatggttctatggattttgacaaattgtataatgtcgtatatctgccattatggcattatacagaataattttgctgccctaaaaatctcccgagttccacctgctcacccatccctcctcctgagcccctggcagccactgatctttttactgtctgtatagttttgccttttccagaatgtcatgtagttggaatcatacagaatatagcattttcagactggcttctttcacttagcaatatgccgagaccagctcgattgtagagaccctaacccagcggcactagaggaattaaaggcacacagaaatatagcggtgtggagtgggaaatcaggggtctcacagccttttgacagcaagccagtgataagcattgtttctatagattatagattaactgaaagtattccttaggggaaataaagggctgggccgaagtaaagggatgggtctggctagttatctgcagcaggagaatgtccttaaggcacaggtcgctcatgatagtttgtggtttaagaacgcctttaagcggttttctgccccgggtgggccaggtgttccttgccctcattccggtaaacccacaagcttccagcgtgggtgtcatggccatcacgaacatgtcacagtgctgcagagattttgtttatggccagttttggggccagttcccaacagcaatatgtgtttaaggttcttccatgtcttttaatgatttcatgctgaataatattccatcgtattgatgtaccacagcttgtttatccattcatctattgaaggacatcttgattgcttccaaattttggcaattatgaataaagctggtataaatattcacatacaggtttgtgtgtgaatatattttcaactcattttggttcacaccaaagagcacgattgtgggatcatatagtaagagtatgtttagttttatgagaaactacaagctttcttccaaagtagctgttgcattttgtattcccaccagcagtgaatgagagttcttgttgctcacatcctcaccagcatttggtgtgtcagtgttttgaattctagccattctaacaagtgtgtagtggtacctcattgtttgttttatttaattttttttttttttttttggagatgaaatctcgctttgtcgcccaggctggagtgcagtggcgtgatcttggctcactgcaagctccgcctcccaggttcacgccattctcctgccttagcctcctgagtagctgggactacaggcacccgccaccacacctggctgatttttttgtatttttagtagagacggggtttcactgtgttagccaggatggtcttgatctcctgacctcgtgatccgctcgcctcggcctcccaaagtgctgggattacaggcgtgagccatcatgcccggcctgttttattttttaaagtcaattttctttcaagaattagctactttttagtatctttaattaaaaatctcattagagaaggaggttggatattttgttgaagtggggtttttaagttacacatccatttgctttattagtgattatgtctagtccatgttaacttgaaaaatgagactataatgagacattttatttaggctgctacaaacagttttaaatttggtcttcactttattttagtaacattgatagagcttatttttcccaaaagctaagttagagattataggaccaaccgaagcaactattttctaagagtaataataagtgactcaggtgccaaatttgtagttaccatcaactattggaaccatatgagtacttaatgccctggagagtcaaatataatctactctaatacagaaaatagaaatattgaaaaactgtaaattggatttcatattgttaaagccacctatagctttagaaactctgaacattattttcttagaaaatggatgtgttcaataagaatagaaattatgtattactgtctgcaactcactttgtctaattatatccaatttattcatccagtcaatatttcaggagtgactaatataccagacatttttgtagttgctagggatacagtgacaaataagacaaaatctctacctcagattgctcacagcctagtagggggaaaaagaacagtgtatgatcaaactcttcagggaacacataggggggcaaacacttaatcttaccttagggatcactacagttttctggaggaggtagtttctaaatggaagcctgaaagagttgttccaggtcaagaaaagcaaagaaggggaaacagcttgtacaaagtcctagaggttaaagaaaacattctttcaggatatgcaaatggttgggtatgggtaaaaagtagactgtaaaagaatggcatcataaaaattaagtaaattgtcacataaatatatatatttcttatgtacccacaaaaattaaaaatgaagaaattaagtaaattgtgaaaggccttcatactatggagtttgacttgatcttgaaaagtaagatcttgaaaggtttttagcacaagtgatattgtcagatctggtacattggtaggttttcagtaaatgtcttcccttactccttttttctctttccttctgcttttgtttaaagcgacaagatgttgctcttttcccaggctggaatacagtggcatgatcatagctcaagctcctgggctcaagtgatcctcccgcctcagcctctcaagtagctaggactacaggcatatcaccacaccagcgttttctttgtagaggcagagtctcactctgttgctcaggcaggtgttgaactcctgcctcaagcaatcctcccacctcagcctcccagagccctcaaattataagccactgtgctcggggcatcctttttggggggtaatcagcaaactgaaaaacctcttcttacaactccctatacattctcattcccagtatagaggagactttttgtttttaaacacttccaaagaatgcaaatttataatccagagtatatacattctcactgaattattgtactgtttcaggaaggaatgttcccaatagtagacataaaagtcttcgcacagtgaaaactaaaatggatcaagcagatgatgtttcctgtccacttctaaattcttgtcttagtgaaaggtatgatgaagctattatattaaaatatttaaatgaaacattttcctacatatatttgttctataaagatgaatctgatttttatgctaatattttggctaagagcctggtagaagatcttacatttttaaataatcttttaggttgagtcctttaatagaatagtttttacattagaaacatgtaagttgttgttcttgtgatgttgaattggctggttttctgtatattctgtgattttttaagtaacaaaaataacagtggtgaaaagcagtaagtcagtccttgaattatcaatttaaaataaattgtgtacttttcatctttggagagaatatgatttactttacaaattttttttttggttttttttttttttgagatggagtctctgtcacccaggctgtagtgcagtggtgcgatctcagctcactgcaagctccgcctcccgggttcacgccattctcctgcctcagcctcccaagtagctgggactacaggcgcccgccaccatgcccggctaattttttgtatttttagtagagacggggtttcactgtgttagctaggatggtctcgatttcctgacctcgtgatccgcccgcctcagcctcccagactgctgggattacaggcgtgaaccactgtgcccggcctactttacaaaatttttgagtttaaaatacacggtttccagcagctgaaatttgtgagtacatatgtgttggcattttaaacatcacttgatgattatttaatgcttcatgagagatttactttttaaaatgtaatataaaatatctaaaagtagtattccaacaatttatatgaatgagaatcttcttttaaaaataagataaactagtttttgccagttttttaaaataacctaagggatttgctttgttttattttagtcctgttgttctacaatgtacacatgtaacaccacaaagagataagtcaggtatgattaaaaacaatgctttttattcttagaatactagaaatgttaataaaaataaaacttaacaattttcccctttttttacccccagtggtatgtgggagtttgtttcatacaccaaagtttgtgaaggtaaatattctacctggtttatttttatgacttagtaattgagaatttgacaatagcgttatacctttgccctgagatttacaaatctgtacctagcattctgcctcatacaggcaattcagtaaacgttaagtgaaataaagagtgaatgaaaaaataatatccttaatgatcagggcatttctataaaaaataaactattttctttcctcccagggtcgtcagacaccaaaacatatttctgaaagtctaggagctgaggtggatcctgatatgtcttggtcaagttctttagctacaccacccacccttagttctactgtgctcataggtaataatagcaaatgtgtatttacaagaaagagcagatgaggttgataattgtcatctctaatacttctgttaaaaggaaatatgaaaagaaaatattagataatgtctttgataagtgtgttagtaactgacaataattttattctattaagtgtagattggaataaatacaaatacatttagtggtagtccagtggtgtcaagcattatgttttagtacgatgtgattaacgtagaatagcttacaaatattcctttactggcctatataagcgtttaagaggcagtatttggtgtgactgaattctttttacaaatgattgtggtaattggggcattaaagcagcattaaataagcttttgttttctctacttaaatgtgttctaaggtctgtattgccagtagtactgaattgaggtcttaaattccacaagtgtaattacacaactatgtgataaactgcaatatttatccattcattaaactgtaaactctttgcagtctcaccacagtttctcttactaggatctagaaatatttcctattgtaggctggttgcagtggctcacgcctgtaatcccaacactttgggaggctgagaagggtggatcacgtgaggccaggagtttgagagcagcctgtacaacgtggtgaaaccctgtctctactaaaaataaaaaaattggccaggtgtggtaacacacacctgtaatcccagctacctgggggctgaggcatgggaattgcttgaacctgggaggcagagggtgcagtgagccgagattgtgccactgcactccagcctgggtgacagggaggctgaggtgggaggatcacgaggtcaggagatcgagaccatcctggctaacgtggtgaaaccctgtctctattaaaatagaaaaaattagctgggcgtggtggcagacacctgtagtcccagctactcaggaggctgaggcaggagaatggcatgaacccgggaggcggatcttgtagtgatctgagatcatcacgccactgcactccagcctgggcaacagagcaagactctgtctcaaaaaaaaaaaaaaatcctgttataaaactacttaaaaatctctgagtagctgagatttggctaatcatgacttagtatttgaaaagttgtgactattttttttttttttaattgagacaaggttcttctctgttgcccaggctggagtgcagtggcaccgtcgcagttcactgcagcctcaacctcccaggctcaattaatctttcttcctcttagccttccaagtatctgggactacaggtaccatgccaccagtatactaccagtcctggctaattttttttgtattttttgtagagatgggtcccgccatgttgcccacacttgtctcaaattcctgagctcaagcagccaccacacccacctgtgaccattcttttttatttttatgagataataaacatacaagtttaaagaaatgtctgtacataaatgtgattatagtacaaacaagtatttggaagttcatctaaacaaatgcatcacagtttataggcaaaacatgaaagattggataataatgggaaaaaaagtaaatattcaccaacattctttctcttttttctttttcgttttttttttttttgaggcggagtcttgcccttttgcccaggctggagtacagtggcaccatctcggctcacagcaacctctgccttctgggttcaggcgattctcctgccttagactcccgagtagctgggattacaggcacccaccaccacgcctgactaatttttgtatttttagtggagaggaggtttcactgggttggccagggtggtcttgaactcctgacctcaagtgattcgtttgtctcagccacattttttttgtctaagaagatactgggccagatcattgtttctcaaattgcagattatgacctgttcatagttgtgaaacttattttgtgagtcgtatatgctctttttaaatgaaatgaaaattctgagtacatcacatgtagttagggtttagaaaataaaaaatacaatatatctagttaaatttggacttcaggtaaacagcgaataattttgagatatacttaacactaaaaaattattcattgtttatctgaaattcaaatttaatgaggtgtcctgtattttatccagaagtcctacacacagtaaagtttgttttgtaaaacttttttacttaacctttgtgtgcccatgtgtgtgtgcagtcataaagtgtgtgtgtgtgtgtgtgtatttaaaaaactaggttgtactcaaagcctgagcttaatttattcccaaaccagtattacattttgtttattctagcaaaatagcattctgttttgattcctctttagctgggagtaagttaaccctattctgttgcttagatgaaataatatggataaaatcattttgaaaatatgtatttaatatatagtatgcctttaggctgtagtgttgtctaaatgaatgctaaagtctccaagctttagcttttaagtcataacctcacagcatcatctgactttccaactcattgtggacagtattaccataaagtaatgatcaccaagccatatcttaccaccttgtgagtagtactaaggaagtaagtatagtttattcactgtgttgattgacctttctaattactatacttaagtacttgaatcaattcattttgtttcaaatgtgtcatgtaatcaaatagtagatgtgctttttgatgtctgacaaaaaataagtttttgcattctagtgataatatacaatacacataaatttttatcttacagtcagaaatgaagaagcatctgaaactgtatttcctcatgatactactgctgtaagtaaatatgacattgattagactgttgaaattgctaacaattttggaatgccttgttaaattatttatcttacatttttaatttcctaatctgtaatttatctaagcctttgagaaagtctctaaacctggtcctatatgtgattttaacttcctgtgaaactctgctgtctctctgttaaagttgcatatatacaatatataccgtagtcccctattcatggggtatacattccaatatcccccagtgaatgcttgaaaccttagatagtaccgaaccctatatatatatattaaaaatgtgtagtatttatatatatatatacctataatctttttttctataagcacataccctgtgataaagtttaattcataaattaggcacagtaagagattaacaagaactaataataaaataggacaattataacaaaataccgtaataaaagttatgtgaatgttgtctctctgtctcaaaatatcttattgttctgtactcatgtggcagcagcttcatcagcagatgtggcctctccagtaatttttatatttttcagtccaaacctattcttgaatctgtgtaaccaaccatcccttacttgcagtaaatggcttggtgtcattcatttcaggggatcccttactgaagttttcgtttaggctctatgctttctggcgtaatatgtagctgtcaatcaaaacaacctgttcatgttttctacccacaaatgtaataccttttctacttctatggtgcactgtgtggccacaacatttgcagtttgaggtgtgacagcaaaaccagctcatatgtctttctccttcacaatctcacagatagatttgttcttaccatagatgtcgcagtacaatttttttcctttccttaagtcgagaactttcactgtttcaattaaaggaagcactttatggcttctttttggcatatttgaattgccagcatcattatacttgtgctttggggccattgttaagtaaaataagggtgacttgaacacaagcactgtggtaccacaatagccgatctgataaccaagacaactactaagtgactaataggtgggtaccatatacagcctggatacgctggacaaagggatgattcatgtcccaagtgggatggagcaagatggtgcaagtttttttttctccatttccattttcctttcctaagatttccacatcctagtggtgcaagatttcatcacactactcaggatgacacacaatttaaaacttactaattgcttacttctggaattttccattaaaaatttttggacctaggttgattgcagataactgaaatcaccaaaagtgaaaccatggataaggggggactactactatatgtgcattgagagtttttatactagtgattttaaactataatttttgcagaatgtgaaaagctatttttccaatcatgatgaaagtctgaagaaaaatgatagatttatcgcttctgtgacagacagtgaaaacacaaatcaaagagaagctgcaagtcatggtaagtcctctgtttagttgaactacaggtttttttgttgttgttgttttgattttttttttttgaggtggagtcttgctctgtcacccgtgatctcagtttaccgcaacctctgcctcccgtgctcaagcgatcctgcctcagcttgccaagtagctgagattacaagcatgcaccaccatgcccaactattgtatttttagtagagatggcatttcaccatgttggccaggctggtctcaaatggtcgtgagccaccatgcccagcctgaactactctttttaattggcaccattgaaggattgctcctcttttcttaaagagaaaatatattacctttcctttcttgactactgaagtagtattttatctcaaagtattgagagtagaaactaacttggtgtgcctgtgatcccagctactcaggaggctgaggtgggaggatcgcttaagcccaggcggtcaaggttgcagtgagctgtgtgtgtgccactgcactcccacctgggcaacagagtgagaccgtgtctcaatggaaaaaaagagaaactaatttgatttcgatgacagtatttaaatactgtgtaagacagtactatttaatatgtggttgtgacacaaaaacaaagcctattgaaaattttcagagacaataagatatataattaacaaaatctgagctttttttttttctaattagaaagtaaatgtggtttagatataccatagtttacctaatcaggtcatggaatattgcatttttcttagtatgtgtgtatgtctgtataactgtgtaggatttgatatctgtttttgtctgtgtggtatcatgtacgtatgtatatgcatatgtaaaatcagatttacccttgttatagggccacagaattgatttggaacatctgttttgataggtcttagaatatttaattgtatatatagtaagattaggtgagttttaattgtgtagaactgctaaagaaaggtttttagggattgttgtatgaataaaaggctttaggttcattggaatcaggggaatcaggctttactagaagaacaggagaaggggtgactgaccgaaaaataaaatgccaagtactcagaataaccctttaaatactgatatgtaatatttagcacattctacataaactgtttctatgagaaaggttgtgagaataatataaattatatggcttataaaatattaatgtgcttctgttttatactttaacaggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtacctctgtctttttttttttgtaaatagtacatatagttttatagatgacgattccttctgtgtttttttctgctttttaaaatcttcatatcttatatttaatcttaggcatcatctgtatacatgattgtttaggtctttaattaccagtgtttagaatcaggtcactcaaacatggtagataagtttgcatagtttgtgtatatccatcactcttgagacagttttattttaagttccggggtacatgtgcaggatgtgcaggtttgttacataagtaaacgtatgccatgttggtttgctgcacctgtcaacccttcacctgagtattaagcccagcatgcattagctatttttcctggtgctctccttccccccacacacccccacctcctgacagaccctagtgtgtgttgttcccctccctgtgtccgtgtgttctcattgttcagctcccacttatgagtgagaacatgtgatgtttagttttctgttcctgcattagtttgcttaggataatggcttccagctccatctgtgtccctgcaaaggacgtgatcttgttcctttttatggctgcatggtattccatggtgtatagttccacattttatttatccagtctatcattgatgggcatttgggttgattccatgtctgtgctattgtgaatagtgctgcagtgaatgtacaggtggatgtatctttataatacaatgatttatcttcctttgggtatataccccgtaatgggattgctgagtcagatggtatttttggttctaggtctttgaggaattgccacactgtcttccacaacggttgaactaatttacattccagccaacaacttgagacagtttttgactcataaacattcagagcttggctagctaattcctgctttaatttaaaaagtgtttattatatgcaaattggacaactcatataaatatgtggtgctacttactatgtattttctctaaagcatgttaaaaaaataggctagatatagtggctcatgcctgtaatcttagcactttgggaggctaaggcaggaggatcacttatggtcaggagtttaagaacaccctgggcaacatagcgagaccccatctctacaaaaaatttaaaatacccaggcatggtggcatgcttctgatgttgtagctactcaggatgctcagacaggaggatcacttgagcccaagtgactgaggctgcagtgaaccaaaattgtaccagtgcactccagcctgggccacaaaatgagaccttgtccctgaaaaaaaaaaaagaaaaaaaaaatttaaatagaggaaatactagctaagtttaatgtaggccagttctaaaataatgatttattgctgctgttgttacataattttcttaaatattttaaagattgcatactgttactgctctatttctgcatctccgtggtgtaactctgtcctctttgttgttgcaacagttcacttagcaactaaactgtatgtttacaaagtgattttatctccctatgagaagactttagtgaatagctcagtgaatagtagagttggtgagaccacagtacagaactgtttgaagtttgggttaaatttttagaggaaaatgtttgatactatgcatatcatagttaaagccaatgaaaaagctaatataggccaggcgcagtggctcacgcctataatcccagcactttgggaggccaaggcaggcagatcacttaaggtcaagagttcaagaccagcctggccaacatggtaaaaccccatctctatgaaaaaaaacaaaaattatccagatgtggtggcatgtgcctgtaatcccagctactcgggacgctaaggcaggagaatcacttgaacctgggagatggaggttgcaatgagctgagatcacgccactgcactccagcctgggtgacagaacgagactccatctcaaaaaaaaaaaaaaaaaaagctaatacatgtgatcactgatgaaatgcaattaagaactggttagtagaaaattcagagggtcaagaaatttaacagagcagttgaactcatttgcctttatcgttgagattagatcatctttcaggctgttagtatatggaccctgtttttaaaaattgtggttttgtttttttcaatgtgaaagaattaagaaaattgttacttttctaattccttttctgtgccttgcttttctgttcacaccagtattaacagcaatgaaattttttcaattttattttccaataaaaattactttgagttttttttatggtagctagctacttccttgacctagatactaattttgattgagttggtaactattattaaaaaaacaacttaggtctaatttatcttgagctaaaaaatgtaataactgaaaaatagagcatatttaggattctttctgctttaaatttgacattcagttattttcatgtaatttgtgttttgagcactaccttttaattaatttatttatttttattttttagagactgtctcattctgttacctagtctggagtgcactagtgtgatctcagctcaccgtagcctcaccctcctgggctcaagcagtccttgcacctcaccctcctgagtaactggcaccacaggcatacaccaccacacccagctaatttttatttttcatagagtcatggtctcactatgttgcccaggctagtctcgaactcctgggctcaagcagtcttcctgcctcagcctcccaaaagtgctgagattacaggcatgagccactgtgcccaaacactacctttttaacttagtgaaaaatatttagtgaatgtgattgatggtactttaattttgtcactttgtgtttttatgtttaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggtaagtgttcatttttacctttcgtgttgccaatcactatttttaaagtgtttattcagtagacttggtatgctaacaattaagagtgttataaactatgtcttttcagccatttttgtgtagtcagtttgggggagtatggtttgatatacagatacacagattcagtattcgtatacagatttgatatcttggtatacagattcgatatctctgaatctgtataccaagaaatcatgttttaagggtctcaatatattttcaaaaagattattagtataataattgagaaattactgttaaaaagttttgagtttctctagaaaatttgaaactcttaacaaaacctgcataatactaacttaactgttttcatatacatagcaagttcagactctgacttatatgaactttaaaagttggtttccgggaggccgaggcgggcggatcacgaggtcaggagatcgagaccatcccggctaaaacggtgaaaccccgtctctactaaaaaaatacaaaaaattagccgggcgtagtggcgggcgcctgtagtcccagctacttgggaggctgaggcaggagaatggcgtgaacctgggaggcggagcttgcagtgagccgagatcccgccactgcactccagcctgggcgacagagcgagactccgtctcaaaaaaaaaaaaaaagttggtttccgattataccatttactgggtaatatatactacttagttacactacttacatagcttcagtttccttatctataaaatgcaaataacacctcccatgagggctgggcgtggcgctcatgcctgtaatcccagcactttgggaggccgaggtgggtggatcacctgaggtcaggagtttgagaccagcctgaccaacatggtgaaaccccatctttactaaaaatacaaaaaattagccaagcgtggtggcgcgcacctataatcccaactactccagaagctgaggcaggagaatcacctgaacctgggaggtggagggtgcagtgagctgacatcacaccactgctctccagcctgggcaacagagcgagactgtctcaaaaaaaaaaaaaaaaaagtgtatttaaagcacttagcagtgaacttgacatatagtaggcagagagcattcagtaagtgttggcttgctccctttttttcatttaggaagtgatctaaaaacagtattgttagtaaatggtatcttgatcttaatgttatgtggactattttaacttcccttttaaatgtatatatatctaacaacttagttcaactacagtcatgtgtcatttgacagggatatatgttctgagaaatagattgttagatttcatcattgtgggaacatcatagagtatacttacacaaacctaggtggtatagcctactatatacctaggctgtatggtatagcttattgctcctaggctgcaaacctatacagcatgttactgtcctgaatactctaggcagttttaacacagtggcaagcatttgtgtatgtgaacatagaaaaggtacagtaaaaatacggtattaaaatcttatggggctgggctcagtggctcatgcctgtaatcccagcactttgggaggctgaggcaggcggatcacctgaggtcaggagtttgagaccagcctggccaacatggtaaaaccttgtctctactaaaaatataaaaattagctgggcatggtggtggcacacgcctgtaatcccagctactagggaagttgaagcaggagaatcacttgaaccctggaggcagagatttcagtgagccaagatcgcaccactgcactcctgcctgggcgacagagcaagactccatctgaaaaaaaaaaaaaatcttatgggaccactattaaagtcttataggatgaccattgcatatgtggtctattgttgaccaaaatgtcattatgtggcaaatgactgcattaggttaaccttatacatacctatattaggtatgtatttggttttgtttttttgtgtgtgtttttttctattagtgtatctgactggtaataatcttaaataattgaatctgtttgttagttgcaattaaagcaaatgccaaaactccaacatttcagtggataatcttaaataactagttcctttttaaaaaacctataaactcataaaaatattttagttattagaactcttcctgtctagaccccatgtattacagagagacaccgaagttagtctcctcattcaaaaagtgccttttgcccctaagtcattctggtggatacagatttacttaatcaagtgttgtccaggtcacattcaatataggatttactttatggacaaagtagtacgtttatagtacttaaactatttgctgtcctttagtgtgaaattctgaggtatatatgcttaaagatatttgtaattcttttgtggaaaataatggctttatttatagcaacccattctgttcttgtgcatactgaagtatattgactttccacctagggaaaaaaaaaacaataactcagacttgtaaatgctttcaacggtgttactacttaatttccctcatttctgtaacatataagtgtataacttagtcagcttctggttactggaacagtacaggtcactgttaaacaattaaaccacttttataataatctaacacctcctaaagccttgcatggacatttttacttattaaattatacaaatttattccctgtaataaagcatcaaaaagcaaagtacctgttatatattatctcagcatgacatggaaatgcctaccttgaattatggtttaatcttaccctcttagcctctgtagaatttttaaataagaattgtttctattactagtactttaatgtaatttgataattgtaaaaagcctcttaactctaattcaaggacctacataataaattactccttcagttaatggctgcccccgtgctgaaaaaaaaaaaaaaaaagagagaaaaagtttatttgaagaaattttgttaggccttattgccagtaaacctagagttatatttagtgtcagtttttcaaaaagtagcttatctgtggtatctggtagcatctgtttatcctatttaggatttatcctgtttagaccctgttaaatagtggtgttttaaagtggtcaaaacagaacaaaaatgtaattgacattgaagactgactttactctttcaaacattaggtcactatttgttgtaagtatttttgtttaacatttaaagagtcaatactttagctttaaaaaaatggtctatagacttttgagaaataaaactgatattatttgccttaaaaacatatatgaaatatttctttttaggagaaccctcaatcaaaagaaacttattaaatgaatttgacaggataatagaaaatcaagaaaaatccttaaaggcttcaaaaagcactccagatggtaaaattagctttttatttatatctgttctccctctataggtatggtatataatattctgacctcaggtgatccacctgcctctcaaagtgctgggattacagacatgagccactgtgcctaatcaaggacctctttatactcttaaaaattactgaggacctaaaagagcatttgtttatgtggaatatatctattgatatttaccatattagaaatgtaaattgattaatgttaaaattagtaatattatgcgttggtcatttggaagatatgagttcactgagttatgcggatcttccgaaagttgacagttttattatgcagtattaaacaatcactttcattgatgccattaccgatcagaaaagtttaagtagtagaaagctgtcaagcttacagagccagatacaagcttcccaaaaattctgattttcatctaaaagcttgaatttttccccggcaataagtattgtcacttatttttcttgtaggtgacaagcttattttcattcatttttgaaaagatgtctgccgaatacccaagtctgaataactatagtttgttggttattctttcaagtaaaaggtatttcatgaaaaaatagctagtatagctcacaactcaatcatttaagtgtgttttcttgagaaacgcactgaagtatgcaagcataatataccaacagtacaaatatcaacagtgaaaaggacatacataacattttactaataagacagttttgacagcttggattccctaaaatggttgtagatacctaacaggattccactgatcatttcttgagaatcattgtcctataatatatacataataatctaaatttacaatatcagtattaactactgacaataaaactactaaggaaaatgtaagaattgtttgcagtttttgtccttagagtatataggttgagtatccctatctgaaatgcttgggaccaggactatttcagatttcagattttttcagattttgaaatgcttgcatatacaatacataatgagatatctggggataggactcaagtctaaacacgaaatttatttaagtttcataaacaccttatacatataacttaaatgtaattttatacaatattttaaataatttttgcataagacaatttaaattgtgatccatcacatgaggtcagatgtggaattttctactggcctcatgttggcactcaaaaagtttcagatttgtgaccattttggattttcagattagggatactcaacccatatattattaagaatgtttagtcaaaatactgtgttcaaatgtcactcaaaataattcttccggatgtggttaccaatttgataattaggttacattcctttttttccatttgttttcaattttaggatttgtcttttcttatttaattttacatttgaataaataaaacattacatagttcattcatcagaactacaaaaaggtatacttagagtttttattcacccacctcttgcttactataggtaatcttttttagtgtttttttttcaggattctgtttaataaaaataagcaaatacatgtatatactcattaccctttcttactcaaaagatacagtatatacaccattttgcaccttgtttattggttgttgtttacttaagaattatttggagatgactccttaatgagtatatagagatcgtcctcattcttttttgtggttacatagtagttgatcatctggctgtgtcagtgtttcctagtttatttaaccaatttccaactagtggacttattgaagatttaattaggttccagttacatactgagaatgaacaatatctaaagcttagcttttaaaccttcataagactaaattttaaatttggtatttgcatcagaaattagctaacacctttgagttatgatggttaacatcaactgactaaatttatgctgatttctgttgtatgcttgtactgtgagttatttggtgcatagtcattatcaatttgtgaatcaatttattttcatagttaacatttattgagcatccgttacattcactgaaaattgtaaagcctataattgtctcaaattttttgtgtatttacagtaacatggatattctcttagattttaactaatatgtaatataaaataattgtttcctaggcacaataaaagatcgaagattgtttatgcatcatgtttctttagagccgattacctgtgtaccctttcggtaagacatgtttaaatttttctaaattctaatacagtatgagaaaagtctcgtttttataaatgaacatttctaaaaataatgacactaacgttaagaagttaacacttcccgttttataaaatttataaaatttataaaatactttggtagtattttatagtgctgttcatatcattattttattttttaattttatgacagctttgtaaagtagacagattttattctaattttatggatgaagtactaaggttgagaggaattaaggaaattgctccgaatcagttaacaaaaagattgcagatattaaaaatatccttttatctctcctctctaaacctttaaaaaagtactaagatagtttttttaatgtataattcccaaggacaatgatgagaagaaacaacaaaagtttggaagccaaaaacataaaggatttagtaagcatgagaaagctaaaacctgacactagagcaaacagagatgctttcccctaaaaaacctgaaaaagattcaaattggcagcaacaggtacttctgaaggtgaagtagaaaataggaagattagttgaaattctttttaagaaacatctatatttcctcccccactgcaaataggcggttatccttcttctgccaggaaatcagaaggttgttcttgaaaaagatgaattgagaggattctgaattgaaggtgggctggagggaggggacaccaggcacaattgagggaaagatactaaaatgaaagatcagatacaaatctgtatgtcaagcagtgagacctagctccttcccacacttggttcccaaatgcaggccctctaggcatgagactggaagatttttttttcctagggaatatgcctgacccaatagaaaagaccaaaaaatactgacagttgaggatactcagatgaaacagtatagccagtcaccagaccaggaagttaactgttgacatgcacagagcttccaggaagctacttagtgcttcacttttaaataagaaaagatagtcaaagataactagtcattggaagaaagctactatgaaacatagtcaccaaagtacaaaatccatagcagaaaggaacctagaggaaatcgactatgaaaacttcataaaaacctactaatattctcaggtaagaaaagaaaaaatggccgtaaaataagaacaagttgctataaaaagctcttagaaattaaaaatatgatagcacaaataaattaactcagtagaaataatggaagaatcatgaaagttcccagaatacagaataaaatgaaaaaaggtatgaaaagtcaattctgtggatctatcatctgaaaatacagagtttgagaaggaaggcacagaagagaaatgaagaaagaaattttaaaataaatacataattttaaaagttctactagtactgaaggacatgagtttccttaattaaaagggcccactgagtgagcacacaagtaaaaatgacccacagtaaggcacatccttgtgaatttttagaataatagaggcagacaggaaccttaaattcattagaggaccaagaagttaggtttcaaattgtttcaagccataatagtatgaattctcttattatcaacaatggaatctagaagactgtagatcttatataatacagagaagtgccttcaaaatactgagagaaaatgatttccaacctagaatctgaattaagtgtgagggtagacatttttcagatgtgaagtactaaaagatctcttgtgcgcttttctcaggaaactaaccaaaacaaatgcatacaccaagaaggaggaaggtataggacttaagaaataagaattcaacatagaagagaggcaaagggagctttcaggatgatattgaagggagatcccagagtagctgtgttgctaagtctagaaaggcagctagactactttggaactgaagaagataagagactttggaagagtttgccttcaagataaaaataaagcagtacctgcatgttttaatgtattaggaaacttcttagtaaagatggtgaattgaggccaggcacagtggcttacacctgtaatccagcacattgggaggctgaggtgggtagatcacttgaggccaggagttcgagactagcctggccaacatggtaaaatcccatctctactgaaaatacaaaaattagccaggcgtggtggcacacgcctgtaatcccagctactccagaggctgaggcacaagaaccgcttgaacctttgaggtggaggttgtggtaaaattgcaccactgcacttcagcctgggtgacagagtgagactctgtctcaaaaaaaaaaaaaaaaaaaaaaagatggtgaattgaacatactcatatcctttctttgccttccaaacttttaccaaaacatcattgaagaaacttacacacacacaaaaaaaaaacaaggaaaataggaaataacaaagtaactaaatttctcaaagcatgcagaaggaaactgaatgaaagctggtggtggggacagcagagaaccaacgattttacactcaggtctcaaaagactaggaattggtggcttcatttcttatctttagaattgggtggtgcagaaggagggagccaaaatggaataagttgaaattatgtttaagaagcaatactcgccgggtacggtggctcacatggaggctgaggcgggtgaatcacctgaggtcaggaattcgagaccagcctggctaacatggtgaaaccccatctctgttaaaaatgcaaaaattagctcggcatggtagcatgcccctgtaatccagctactcaggaggctgaggtgggagaactgcttgaacccaggaggtggaggctgcagtgagccaagattgcgccactgcactccagcctggacgacagagcaagaccccacatcaaaaaaaaaaaaaaaaaagcagcagcagcaatactcatgaagctgggcaactgtctcctgcccgctctatgaaaagaaccagaggcttattctccagagaggatacagtagaaggtgaacacactaggcacagttgaaggcagaagcaactacttgaaagcaagaagaagttaatatatgcatattgaatgttgggatctcccctcaccaagcccttttccaccactcagcttccagaacatagacagctaagttttcactagtggaagtttccatttaatcaagctactgtgtagcttgcagtcaacaagttctatctttgtaccaagtgcttcaaaacagccttttggtccctcactcttaactataaacagacatccaaagattatgagacatcagaaaaagcaaaaataaaataaccaaaaaacacattaatgaaaacaacttagaagaaacattattcaaggagaagaaaaaatgtttttttaaaaactataatttgtgaacagaatgaaaagaggtttatatatatagctaagagtttagatgtgaataaacagtaagtacatagaaaataagcagattttaaaaattaactcaagagaaagcaaaagttgtaaaggaagtacactatttatatactacccattaatggccgggtgtggtggttcacgcctgtaatcccagcactttgggaggccgaggcgggtggatcacaaggtcaggagatcgagaccatcctggctaacatggtgaaaccccatctctactaaaaataacaaaacaaaattagccagacgtagtggtgggcgcctgtagtcccagctacttgggaggctgaggcaggagaatggcatcaacccaggaggcggagctttcagtgagccgagattgcaccactgcactccagcctgggcgagagagcgagactccgtctcaaaaaaaaaacaacaaaataaaaaaataaaataaaatatactgcctattaatactacatatactttatactgacttagccgtaatgtaaatgttgaacattgatagtgagaggtgaagctggctgggcttctgggtcgtgtggggacttggagaacttttctgtccggctaaaggattgtaaacacaccaatcagcgctctgtgtctagctaaaggtttgtaaacgcaccggtcagcactctgtgtctagctaaaggtttgtaaatgcaccaatcagcactctgtaaaatagaccaatcagcaggacgtgggcggggccaaataagggaataaaagctggccacctgagccagccccagcagccgctcggctccacttccatgccatggaatctttgttttttcactctttgcaatgaatcttgctgctgctcactctttagtgagcactacctttatgagctgtaacactcaccacgaaggtctgcggcttcactcctgaagtcagcaagaccacgaacccaccaggaagaagaaacaaccctgtacgtgccatctttgagagctgtaacactcactgggaaggtctgcggcttcactcctgaagtcagcaagaccacaaacccaccagaaagaagaaactctggacacatctgaacatcaggaagaacaaactcgggacacactatctttaagaactgtaacaccatgagggtccacagcttcattcttgaagtcagcaagaccaagaacccaccagaaggaaccaattccggacacagtagaattaaatacgtaatttaggaagatgaaaggcaagagtgtgtgtgtagtaaggtagaagctgtgttgacagagctgaattttcattttctgtaggggtacttcaagagaaaaagtcaagaagaaacatgtcacttagacatataaatatgataaaatcatctaaaactgtttaaagtagttgcaaaatcttttctagctgataaatttttaagcctaaaaatatcattgaaattattttaatgttacattttattttattttattttatttatttatttattttgatacagagtctcactctgtcgcccaggctggagtacagtggcacgatcttggctcactgcaacctctgcctcctaggttcaagcgactctcctgcttcagcctcccaagtagccgggattacaggcgcgtgccaccatgcccggctaattttttgtatttttactagagaaggggtttcaccgtgttagccaggatggtctggatctcctgacctcgtgatccgcccaccttggcctcccaaggtgctgggattacagacgtgagccactgtaccaggcctaatgttaccttttcaaaaacacctgattgtggaattgttgaagtcactgagttgtatttctggaatgtgttttttagcaggctgcacatacacatatgtagaaagccaggtgatttttttttcatttcttttttttttatcaaaaacagttgtattaaataagaaaggaaatacgtatttacccgtgtattaccttaatttatgtgtaaaatgggagaatagtttaatgtatttaacaaacaaacatttgttaaagtacctgctcaaactacctaatatatactatagtgaaagatataaggataaataagtctaactcagattgctagcctgggaaccagacatgaaaacaagaattataatgtaatataaattctagaatagatgtaaaaagtgatctaagaacatagaaaaattatcagctaatcacatgactgctcaatgggaaaagtacttcagacagaatgtaaagaatgcttggttaaagatggcattccaaatcttggaatttggttgggggacagagggaaacaaaaagaaatggggaggttaggaccaaataggaagcttcctgtatgtcatttctgataagttgaaacctaggtaggtgataggctgtctttggaagtttctaacaagaggaacaaaataagattggtgttttagaagtataccaaagcaaaactgttgcaaggagattagtaaatacaggtcttaacctagcagaggaggtagagggtagagaatgattgagatagaaattcagtagatttggccagatagtgataagttgagactggcaaattatttccacttagatttaaatatagatatcttgagcataacctacaaggcaaactccttatactaaaaatattctgaatatttaaaaagaaaggattaaaagatcaatcaatagaagtttggggacagaaggtttattcattcttgtgcattagatctcatctagatcacctgtttgaagaaatcattccagcaattatcttgtctctctcctgcatggatttttttcctaatagattgttctcatcaccctaagcagttgttgtacatctctcatcttaaaaagaacagcctttcttaagtaatctcaacagtccattttcttctcttaaagcccaactcattagaattgtccctcctcttttcacttatctctttgagtactctcctgaacccagtctagtcagtcctttcagtagaactggtccccctgcttacctccctactcctcaatacacagtgaattctcaacaaagaagccgggggatccttttaaacataagacagattatgtcatttctttactcagaactattccgtggtgtgccatctcagagtagagactaaaagccacttgtcatggtgtacagattcttcatgatctggctgctttgctatttttccagtctgaccttctaatgttccccttgctctccttgctccaggcacacttgtgtctaggccaatcgacatatttgtttgtctgtttccttccactgaaatatacatgcaaaaacaaaattttgttaccgtgttccccagcaaaacaatgtctggcacctggtaggaattcattaaatagttgatggatgggcgaacggataactaaaggaacaacttcaagttccaggtatccagggtttggtaaaaggaaatctggggttttcaacaagatatcaagtattaggaagaccacgtatgctgagaaagatgatcacttttggacatgttgagtttgaaatgagtgtgaaacatcaaggtacagatgtctgatgctatatgtagtgtaaaatgtaggaacaaccctaggagaaaaatcgggcatgaggataaaggatattttcattgttaggtgataatttaagcaatggaaatgactcacattagcaagggaaagtgtctaaggaagacatccagttttggagactttttttgaggaatcaggaagaggtaaaaccagtaaaagatgaaagaggtacagtgatggtgagaattttaaaagaaggaaaatgtaaactgtcatagctattaggaaagttgagtagaatgagtttgcgcgcatcccacatgcatctgggaggtcattaacaactttattgagaacagtttctgtagagtagtgggagaaatgagagtttattgagtagaaattgaggaagtgaaaatagctacattacctattgaagaaggttgactgtggagtgtaacagtgagtattagcttgaggcagagataaaggtgagtgagaaaataagagtttcaaaggtaggcaagatttttgggctaaataaaaagggcactttaaaaaaggtataaataggtagaagagagaaaagggagcgaggtgggataattgaaagaggggatctcctgtggagactgaggtattaggcggagtagagagttcaggtgaagatgtgaaggtgagagaagaggatgggtagacatttccctggtgaaggaggtaaggagtactatgatggaattagaggggacacactgagagggtccacacttgacagactctcttctattatgtgttatgtgaggtagattgtaaagtcaaaggctagccttgaaaaatgtgatattgttttggaatggcaaccatggtgaatacaaaacagttaccagaatagtatcaccatgtagcaaatgagggtctgcaacaaaggcatattcctaaatatttatatgtgtactagtcaataaacttatatattttctccccattgcagcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttaggtattgtatgacaatttgtgtgatgaatttttgcctttcagttagatatttccgttgttaaataatgtcctgatggttttccccctttggtggtggtaattttaaagccctttttaatgttttagattttctaaatccaaagattaggtttaaattattctaatgtttctttcaaagataacttcttgtggacttgttaaaaaaaattagacacacaatctaggactgctgttactggaatatattttctatcatgctactaattttctttttaaaatgtgataaaaatagggccgggcgtggtggctcatgcctgtaatcccagaactttgggagactaaggcgggcggatcacctgaggtcaggagttcaagaccagcctggccaacatagtgaaaccctgtctctactaaaaatacaaaataaataaataaataaataaatagctgagcgtggtggcaggcacctgtaatcccagctgcttgggaggctgaggcaggagaatcgtttgaacccgggaggcagaggttgcagtgagccgagatcgcgccattgcactccagcctgggcaacaagagtgaaaaactctgtctcaaaaagagataaaaatagtaaagatattcatatttatacagctttacaagttgaaacatcctttcatttatgaagaattaaaaggggtaccctttttagagaaaaggagagcatgtaaacttcgaggaaattgatatgtataattttataaaacagggcttgcgcttttttttttttgagacagagtttcgctcttgttgcccaggctggagtgcaatggtgcaacctcggctcaccgcaacctcctcctcccgagttcaagtgattctcctgcctcagcctgctgaatagctgggattacaggcatgtgccaccacacctggctacttttgtgttttttttacttttatatattttttttttgtttagtagagacagggtttctccattttggtcaggctggtcttgaactcccgacctcagatgatctgcccgcctcagcctcccaaagtgctgggattacaggcgtgagccactgtgcctggccaggggttgtgctttttaaatttcaattttatttttgctaagtatttattctttgatagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacaggtatgtgtttgtctacaatactgatggcttttatgacagagtgtaattttatttcattaactagtatctacaaatggctttgtttaaagaatgaacacattagtgcaggaatggatgaatgaaatcatcatattttctaattagcctgcagtggcagcctctggccccttgctaggcctgcctcatcctactaaagtgatctgtgcttccaaattactacttcttttcccccttcaaatctttcttattttgtcattgtaaatgctctcagctaggtgttaaagtagtcttactgatattcaaatgtgaataactgatagccctgaaccttctatgagctatttatattttccaaagaggattctccttaagccaatattatctaggtagaattttaggcaatggagaggtgaaaataatattgatgacattaatagctaactttgagcattttctaggtgtaagatgctcttctaagcacttcacatgcattaggtatatcttgcttaatcctcacagtcaccttgaaagaaaggcactgttactttgtttccattttgcaaatgagagaactgaagcatagagagggttaagtaactgccccaaagtcacttaactagtaagtggaagtgctatgattccaaagcaaagagtctgactccagagtcaaactctgaacaaacaaaaagacactttgggttagatatcctggggtgaaagcaagcactttgaaagtaagccaagcctgtgtacagatctgaccacctgaggtcacattccctaaaatacttaaacttctcccttttgtttcccatctaagtttttgaacttaagagattttgtaaaacatcacatttttttatcctcacagtaccttcctatggcagatttagcaggaggcgtataaacggggtggaaaaggtacagcagactgtggaatgtatggatcatttatattacattaaaatttttagtttctagtaaataacttaaatgtttttgtagtgaagattctagtagttaatgaaaatttttggtaaattcagttttggtttgttataattgtttttattgtgtgatacatgtttactttaaattgtttttcttttttgtgtgtgtttattttgtgtagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttataggtactctatgcaaaaagattgtgtgttaacttttatgtattccctcatccctctttcttctcttaactgtctctcgaactaaaaagttggctagaaatcaaatttttatgcatttaattgttttaagtgcattatggttaagcattctgtagaagtcttttgaaaagtgctgtttgtcctggggtttaatgaactggattttcttgatttgggacatttttcttaggcatttataaatatagcccaatttataaagttaaatttggccgggtacagtggctcatgcctgtaatcccagcactttgggaggccgaggcgggtagatcacctgaggtcaggagttcgagaccagcctggccaacgtggcgaaaccccatctctactaaaagtacaagaactatctgggcgtggtggcaggcacctgtaatcccggctactctggaggctgaggcaggagaatcgcttgaacctgggaggcagaggttgcagtgagccaagattgagccactgcactccaggctgggcgataagagtgagactccatctcaaaaaaaaaaaaaagaaaaaagttaaatttgagggccagacatggtggctcatgcctgtaatcccagcactttgggaggctgaggtgggcagatctcttgagcacaggagtttgagaccagcctgggcaacatggtgaaaacccatctctacaaacaaattaaaaaattagcccagccaggcgcggtggctcacgcctgtagtcccaacacttcggaaggccaagatgggccaattacctgagggtcaggagttcgagaccagcctggccaacatggtgaaaccccagctctactaaaaatacaaaaattagccaggtatggtgatgcatgcccgtaatcccagctactcgggaggctgaggcaggagaatcatttgagcccagtaggtggaggttgcagtgagccaagatcacgccactgcactccagcctgggcaacagagcaagaccctatttcaaaaaaggccaggtgcggtggctcacacctataatcccagcactttgggaggttgaggtgggcagatcacctgaggtcaggaatttgaaaccagcctggccaacatggcaaaaccccatctctactaaaaatacaaaaattagctggacgtggtggcacgcgcctgcaatcccacttacttgggaggccgaggcaggaaaatcgtttgaacccgggaggcggaggttgcagtgagccaaaattgcaccactgcactgcagcctgggcaacagtgagactccatctcaagaaaaaaaaaaaaaaaaaaaaagaaagttaaatttgaaatggccttatggtagattcctcccccgacacacacttacttcatgttttctttcattatatattttaatggatacaaaatataaataaacactaaaagttaaacagaaatatttgaatatcaataatgccaaataactagaaaatctcagagctctaaaacagcaacaatttagaaactatataacctctttttattgtagtttttacagaaacataatttaaagctttttgttatcagagatatattacattatgccagtggcaaaagatgggatttatttcctcagcatccttatctttaaatttctgtacatctttccaaaatttatagctttggaaaagtgataaaactttttttcctgaattttgttttaacttttaaaaacagaaatattgtttacatcttgcgtatcttatataacaaacatctgcttatagattccagtaagaaaagttggttaaacggttgtattattttctcgtactaaatagactgcataaggtagaagttaagaatgattgccctgtagtctaagtgggaatgtggaggctttcgttagttttttctgataattcagcaaatctctattgagcacttgctatgtgccaggtactattctgggtactagggataataaaggaaaacaaaaaagtccctgccctgatgagtcatacattctatgtggaaggcatagaaaatattgaaatataagtgaattgtgtagtatgttagaagaagatacatactataaagatagataaagttggaaaggtggcagagaaagttgggcaaggagatgcgatttttaatctaataagtagttaggaaggcttcactgagtcagctacatttgataaatgacctaaagtaaaaggagggagcataggactatcctagcaaaagacccccagcctctaagaggggagcatgcttgaagtatttgaggaacaggaagttagtgcaactggagtagagtgggcaggagaagagtagtagtagatgagatacaaaaggaagacctcatagaccttcgtaagacccttaccttttactctgcacgatttttactgaataaaccactggaaggcttaaagggtaacatgatctgacttttttttgagacagtctcactctattgctcaggctggagtgcagtggtgcaatctcggctcactgcagcctccacctcccaggttcaagcgattctcctgcctcagcctcccgtgtagctgggactacaggcgcgcaccaccgtgcccggctgatttttgtgtttttagtagagacagggtttcaccatgttggccaggttggtctcaaactcctgacctcaggatccaccctcctcggcctcccaaagtgctggaattacaggtgtgagccactgtgtccagcctgatctgacttatttttgaaaaaataattctggctgtttgttgaggagaggggcaaagatggacacacagagaccacttaagctattgcagaaatacatgtgagaggtggttggttggaccagggaagtggcagtggaattggtggaaagcagttggactctggggtattttgaaagtggcactattaggagttgctcaaggattagatataaaacgtgagagaggaggagaataagaatggctgtgaagattttggccttagcagctggaaggatagagttgtatgtaactactagaattgagaagaccaaagatggagggaaatggagagtttggttttggacatttgaagcttgagatgtaatagtagacagccaagtggagatgttaggtaggcagttggatatggaagtctatacacaggtaaagtataggccaaataaatcaattcacaagtcatcggcatatagatggacttgaaggccatgaaaaagagactgagaaagagcagccagaaagttaggaataaatgcagaatggggtgttgcattccaaatgaagatggaattttagggaatagaaaatgaccagctgtggaagctgcttctaataggtaaagtaagatgaggactgagattggccgctggatttagcactgcagaagacattactaatgttattaaaaatagctcaatagattggtggagtgatatcctgattagaatgcaattaaagagcagttgaagaggaggaattggagacacagaatacagtcgattcttttgggagttgccaaaaagaagcagagagagggacattgcttggagaggaagtaggatcaaagagtcttagttttggcttgttttaaggtagaaaaaaaccgtttcttatgctgattcacattgttcagtagagagggaaaaaattgatgatgcaggagagagaggaggcatttcctgatcgttggccttgtaggcagcaaggggtaggagctagtgcacaaatggtagaagagggcaaagtgtaaggatgcagatgcttggaagagggcaaagtgtagggatacagatgctgtgagtggatagatatgagggtgggagcttatggaagttctcttttgattacttctgttgtcttagtgctaagagtatgaatgagaaaggaggagttagagatttgagaacagaggggacaggatcaaagagagcaccaagactaagaaaggcagttctcaggcgtgatttctaaaaaaatctctttcataagaaaaataatctaaaatataattatttaaaatcaaggatctcatttttcaggaacaaatatgagttgaaatcattctgttgactgttaagtggaattttttgttttgtttttatattttgagatagggtctcactcttgtccaggctggagtgcagtggcactatcatggctcactgcagcctcaacctcctgggctcaagcaatcctcccaccacagcctcctaagtagctgggaccacagatgtgagctaccactcttggctgattttttttattattttttgtagagatgtgggggtctcactatgttgcctaggctggtctcaaacttctggcctcaagcaatcctcctgcctcagcttcccaaaatgctgggagtataggcatgagccaccatgctcagcaatgaagtttttatcagtatgatactttgatacatgtcaaataattttctgaaattatattgtagatcatatgaactcataaaaacttaatgatcttgaacaatgtagtttttgtacagagaatagttgtagttgttgaattcagtatcatcctatgtggtttttatgataatattctacctttatttgttcagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacaggcaagtttaaagcattacattacgtaatcatatacggcagtatggttaaggtttctgtgtagtctgtgacttccatgtcaaaatgttgcacaagccagttgtcagtgacagttgccatcccacactgctgttctcctgtcatccctagcccccatttaagagagatcacacattcatgcattgcttgcttccctctttccccaccccctccttaacctcttgatgtatgagaagaatatgagttactaatttgatccactatttggggattgctaataaagcatttttgcattttattttttgctttttaaaaataattgatattttaacaatatgaaacaatatattcctagctacaaaatttttaattctcagtatttcttagataaattcagtttttattctcagttattcagtgacttgtttaaacagtggaattctagagtcacacttcctaaaatatgcatttttgttttcacttttagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaaggtaaattaatttgcactcttggtaaaaatcagtcattgattcagttaaattctagaagttttacatttaaattttaaatgcttactaaggatgctcaatttcttagatgtactgataattttagtataaaaagcatattcttcagacagttaaagtttttgtgcagtttttgggaggtccagagatctttcttgagcttaaataatgcatttccaattaaaaagcaaaataaatttgcaccatttgattttggtatctgtagcttgctgccctcttgttctcatagctttgctttgatcagatccctattccactctggattagagaattacattttagtacttttcaaatatgtaatagatacactttttatctctatgtagattttaaactacataacaggactctttgtcatattgaatggtctgcagtattgctatctgaaattaccgataatattgtacattcagattcacttaagaggtaaccttgcagagaatttacttctgtggtattctggatcactctaaagagaatgttttataaattaaacatttttaaggtaaagatatattttgtttggcattagttccatgttggattgattgctttttactgaaagcattccatcaagctgaaacagtcttttgttttatgttgcttagaacatcaagcttgcagtggctttcattttcttgtttttgtttttttttaaatcaaatcaatgcatgtgcataatttggaaactcaagtaatataagacatataaccaaaaaaaagcagttgctagcacaaccctccccatcctcattcatggtcccaagaggcaatcgtttccagtctgtttgactattttttagcttgtactgctgtcttctttctctcttcagtttagacagcaaccgttaacttcctgctatggaaaatgaggatttcattgtcttactccaacactgctacatatttctctaccctcttccccatcttccctctactaatacagcatgatttttagtgaaatctgtattcagtgtatacattataatgactgaaaaatattttttgcaatttaatacctaatcgacttgtgatcacatttcctttctttacagcttactgctttccatgaagttattaactgtccttttcatttctcttaattttctgtgtccttacacaggagctataaaaatcctgcccaatatgttaaaatttctccattggttccatttctgtttcttggagaacccctggacctcttcatatgcttactccattctgaagtgctcattttctatgtgtgccatacagctctcattctgtaaattattcttacctcttttctgtgttagaaaccccatttcctggtttccatgtcatctttctagagtttttcccccaattttaagggcccacaactctcagaggcttgttgggaaaaggagcatgcaagctttttgatatcttagggtaaggttggctggatatagaattctaggttgaaataatttcttcagaatttttaagacatttcatttatcttctttagcatagctcctgctaattgcagtgcattcttttttttttttttttttttttttttttgagacaacgtctcactgtgttacataggctggagtgtttcattttgtacagatgaggtctccctgtgttgcctaggctggtcttgaacttctgggctcaaacggtcctcacgctttagcctccgagagtgctgggattacagacatgaaccactgtacctggcctgcggtgctgtttttatcttcagtcattcattatgtgctctggttttaagaagttaagattgttttgtctttatttttatttatttatttatttttgagacaaagtctcgctcttgttgcccaggctggagtgcaatggcgtgacctctgctcactgcaacctccgcctctcgggttcaagtgattctcctgtctcagcctcccgagtagctgggattacaggtacctgccaccacacccagccaaattttgtatttttagtagagacagggtttcaccatattggccaggctggtctcatcctgacctcaggtgatccacctgcctcagcctcccaaagtgttgggattacgggcatgagccaccacgcccagcctgttttgttgttatttaaatttcacagtaatgtactttggtgtctttttttttttttttttttttaacttttttttttcctttaattcttggcccagatgcttactggcacttactctagaaacacatagtccttcattgagttctggaaaattttctttaagtggttcttttataatttccctccatttttttctctagtttttttctggaactcctgatgtttggacactaggcctcctatattgttcctataattttcttgtcttttctcctactttctatttgattgtctttttttattctaacttctgggatggtttttaatttttttcttctaattctttattaatatgccatgttttatagatttgtctttaaatatttttacataattttgtaacaaaatacaaaaaaagcaattcctaaaaatttagaaagtcaaatgaaagcaaagaacttaagtgtgttttcaattagagcataatcataccaagaaagtatttcaagtaacttaaaaaatgttttatgtccctagtggtatataccccaagaacaacaatagcaacaacaactataaaatgaaacaaaatcttaagctattgttagtaatcatattgctggtggtagtgttggtattcctattctgaagttatagtggatgtgaagtatgttgtatgtgtatactcttttacgtatatttgttgtatgtgagtaattatatgattatagagaacagggatctttttatcagagaaaggtgcagatgtgagattgaagtaaaagaaaacttgtggttctgcatttgtattggaaatatcattatgaactcgagatctatattatctttaaaaaatacatgctggctgggcacagtggctcacacctataatcccagcactttgagaggccaaggtgaatggatcacttgaggtcaggagttcaagaccagcctggccaacatggtgaaaccccgtctctactaaaaatataaaacttagccaggcatgatggcatgctcctgtagtcccagctactgtggaggctgaggtgggagaatcacttgaacctgggacgtagaggtaggttgcagtgagccaaggtcgcaccactgcactccagcctgggcgacaaagtgagaccctgtctcaaagaaaaaaaaaaatatgcttattggtttcatctatcaaaaagaacaaaaaaaaaaaaaaaaggaataatccagtagtagtgagtaaccctagctctcagactgttttctaagtactgttttctcttaaaaggatgcaaggtgtcttgaagaaatggctgattccagattcagaggaggaaatatatgaatctggaaagtcttgacataccagaaaaaaactaagcatcaaacactactagtgtcatgtcaaaaggacttaggagtgtaatagaatagattcttactgatcacagataaaataatttgagcatcagaaaggataataacaggctgggcacactggcccacacctgtaatcccaagattttgggaggccgaggcaggcagatcacttggggtcaggagtttgagaccagcctggccaacatggtgaaaccccttctctactaaaaaatacaaaaattaggtaggcctgggggcgggtgtctgtaatcccagctatttgggaggctgaggcagggagaattgcttgaacccaggaagcggaggttgcagtgagccgagattgtgccactgcattctagcctgggtgacagagcgagactccatctcaaagaaaaaaaaaaggataataacagcaaaaaattgaaattcataacaaatgataacttctattctcattgttttaaaaactaaagcccaggcacagtgtctcacgcgtgtaattgcagtactttgggaggctgaggtgggcatactcaggagtatgagaccagcctgggcaacatgacaaaaccccatctctacaaaaaatacaaaaattaaccaggtgtggtggcatgtgcctatagtcccagctacttgagaggccgtggtgggaggatgacctgagcccaggaggcagaggttgcagcgagttgagatcgtgctactgcacttcagcctgggtgacagagccagagccagacccagcctcagaaaaacaacaaaaactaggtaaaaggagaaaaaaatcaagcatttatctttcttctcttatatgatctatattttgggaccctcaagtagatgaggggaagtttctgtttataaaaatgtttcaacaaataaggaataatagaattaaaatataaccatttcgcaaccctcaaattagggttgtcttttcttctaattcttttttttttttttttttttttttttttttttttttgagatggagtctcgcactgtcgcccaggctggagtgcagtggcacgatcttggctcactgcaacctctgcctcctggattcaagcgattctcctgcctcagcctcctaagtagctgggattataggcacccaccaccacacctggctaatttttttgtatttttagtagagacagggcttcactatgttggctacgctggtgttgaactcctgacctcgtgatctgcccgccttggcctcccaaagtgctgggattacaggcttgagcctgtaaatccagaaaaggattacagcccttttcttctagttcttaaagtgaatttgtgtctaccaacataattttcaagagacctttattattccctaaatgtttttcttttttttgtgacattcagttcttgtttcatagatacagtatcttctcatctttctaaagctaatgattaatgttttattttcattttcgttttttctgctccctgcattttttttttttaatgaaaacctttgtctatttgagtctctctggttagagactttcctcaatggtgatcattcactgatccagaagttgtatgtgaggtgaggcttgtcagctcatagggtagtaatgtagtgatttagtttttaactaggagaccctcaaatatcagtgactgttctgagagctgagcagagtaaggaaattaatagggagactcattgtcagtgtaagaattttatttcagtttgttgtttgttgtttgttttttgtttttttgttttttttttggttttttttgttttttttttagatggagtcttgctctgttgcccaggctagagtgcagtggtgcgatctcggctcactgcaacctccacctcccaggttcgaggaattctcctgcctcagcctcccgagtagctgggactacaggcgcctgccaccacccctggctaattttttatttttagtagagacggggtttcaccatcttggccaggctggtctccaactcctgaccttgtgatccacccgcctcggcctcccaaagtgttgggattacaggcgtgagccacagcgcccggcccagttttgttttcaataatagcatctcacccccaccctggctgcgtctgctcagtattccagagtccacgatatgtatggttcagccctccagaaaataaagtctcctgcatttttttttttaatgctgcagcaagaatagggtcctgggttcttttttagtatgagggagagacagccacctggctacttgggataggaaagaaaatctggtgtttcaactacgtttgtacaaaatgtcaaccatttcttcctattttcagccccaccatatgctcctgccttcacaggtacctgttgcctccaatttctgagtgttttcctttaattattttgtttcatgttaactccttacaacaagtttggtggctgaataaccttgggcaagttgtgtagtttctcatatactttagttttatcgttgtctgtaaaatggagatgagtcttcagattattgtgaagataatttgtttgtttgttttttggagacggaggcacgctctgtcccccaggctggagtgcagtggcacaatctcggctcactgcaccctctgcctcccaggttcaagcagttctcctgcctcagcctcccgagtagctgggattacaggcgcccaccacctcacctggctacttttttggtttttagtagagacggggtttcaccatgttggcccgggtggtctcgaactcctgacctcaggtgatacgcctgcctcggcctcccaaagtgttgggattacaggcatgagccaccttgcctggccaaagattaattgttaatatacataaagcgcttaacaccatgccaggtaccttagtaagtgttcgatgaatatttgctttttgtattagccataatcattctcaggctgctttgtcatttacttgttccacaaattcttagcttccaaaattttggtgatacctcatttcctattctctctagttgcctttgtccatgtagattttttgaggaagcttgggtaaataagtgtattttaaactattatgtttaaatcgaagttccttttatctgttttctaatagaaacatttaaatagcattaagaacttgtagcagtataaacaatatgtttgagaagtactatattgtgaaaatattttcacttttatacagttttttacttatttactgtcttactaatcttcctaagactttttaaagtgaatatttttaaggcagttctagaagaatgaaaactcttatgatatctgtaatagaattgaatacatatttaactactaaatcaatatatttattaatttgtccagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacaggtatgatgtattcttgaaacttaccatatatttctttcttttgatacaattaatttgtttgtttgtttgagatggagtttcggtctcttgcccaggctggagtgcaatggcgtgatcttggttcactgcagcctccacctcccgggttcaagtgattctcctgcctcagcctctcaagtagctgagccaccacacctggctaattttgtatttttggtagagaaggggtttcatcatgttggtcaggctgatctcgaactcctgacctcaggtgatccactaatctcagcctcccaaagttctgggattacagatgtgagccactgtgcctggcctgatacaattaacttgaatgttatatatgtgacttttttggtgtgtgtaacacattattacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaaggtaaaattagttatatggtacacattgttatttctaatatgagaacaaagtcttagagactttgaatttaacatttttaatgagtaaattgtttttattttgagtagtaaattgactttattttttagtatctagggtattcttttttggtgttagacaaagaatagcaacaagggacagaaatatcaggtctaagccatttgtaatatttttcctgaattcttacctatatgatgtggcttttgcatttttgtcatggtagttattagctttcatgtgttattatgcctggaactaggacctattgtggtgtcaattttaatattaaaaatcatggtgttttgatgtttatatgacataaattttattttttcgtatctcccttttgttgttgctgaagattttatgtttttctgcatttcctcatgatttatatagatgtaacatgttctataggacatgtaatttacatgtcctatagaactataagttacatgtcctatagaacttacagttctatagttatctgcagaaatattgctccttatgctttatttgcttaaaattatcactagatcatactattttcataaataaatgaatatgaaatcattcacaggcatacctcagagatactgtggatttgattctagaccaccgcaataaagcaaatattacagtagagcaaatcacacgaatattttggtttcccagagcatacaaaagtaatgtttacactatagcataatctcttaaatgtgtagtagcattgtgtctaaaaaaaacaatgcacataccttaatacactttattgctaaaaaatgccaatgatcatctgagccttcagtgagttgtaatatttttgctggtggaggatctttcctcaatgttgatgcctgctgagtgatcagagtggtagttggtgaaggttggggcagttgtggcaatttcttaaaataagacaatggcatttgcaacattgattggcttttcctttcatgaaagatttctctgtagcatgcaatgctgtttgatagcattttatccatggtagaactgctttcataattggagtcaattctatcaaactctgctttatcagaatattatgtaatattctaaatcctttgttgtcatttcaacaatattcacagcaccttcgccaggactagattccctctcaagaaactactttctttgcttatccataagaagcagctctgtattaatctgttcccacactgctataaagaatacctgagactgggtaatttctaaaggaaagaagcttaattgacttacagttccacatggctgaggaggcctcaggaaacttacaatcatggcggaaggcaaaggcaaagcaagtacccttttcataaggtggcagaagagagagtgcaggggaaactgccacttgtaagccatcagatctcataagaactccctcactagcacaagaatagcatgggggaaaccacccccatgatccaatcacctcccaccaggtctctccctcaacacatggggattacaatttgagatgagatttgggtagggacacagagccaaaccatatcattctgccctggaccctcccaaatctcgtgtccttttcacatttcaaaaccaatcatgccttcctaacagtctccgaaagtcttaactaattccagcattaactcaaaagttcaagtccaaagtcttcatctgagacaagacaaatctcttccacctatgagcctgtaaaatcaaaagcaaattctttacttccaagatacaatgggggtacaggcattgggtaaatgttcccatctcaagtgggagaaattggccaaaacaaaggggccacagtccccatgcaagtccgaaacctagccaggcaatcaatgaatcttttttttttttttgagacagggtcttgctctgttgtccaggctagagtgcagtggagagatactggctcactgcaacctccgcctcctgtttcaagcaattctcatgcctcagcctcccaagtagctgggattacaggtgtgcaccaccatgcccagctaatttttgtatttttagtagagatggggtttcaccatgttggccaggctagtcttaaaactcctagcctcgctgggtggggtgtctcattcctgtaatcccagcactttgggaggctgaagtgggcagatcacaaggtcaggagttgaagaccagcctggccaacatggtgaaaccctgtttctatgaaaaattcaaaaattagctgggcgtggtggcacgcgtctgtaatcccagctattccagaggctgaggcaggagaattgcttgaatccaggaggcagagattgcagtaagccaaaatcacaccactgcactctagcctgggcaacaaagcaagactctgtctcaaaaaaataaataaaaaaaaataataaaaataactcctagcctcaagtgagccactgcacccaggccagtcattaaagctctaaaatctcctttgactccatgtctcacatccagggcatgctgatataaggggtgggctcccacggccttgggcagctctgccctttggctcttcagtctacagcccctgcagctgctttcatgggctgccattgagtgacagctgcacagtgcaggctgtcagtgggggatgatggccctcttctcacagctccactaggcagttccccactgaggactgtgggaggtggctccaaccccgtatttccctcacagtttccctcccggtttccctcctgtactgcactaacagaggttctctatgagggctctgcccccacagcagacttgtgtgtggacatcctggcacttccatacatcctctgaaatctaggcagaggctcccacagctgaactcttgtcttctacatacccacaggcccagcatcacatggaggccaccaaggcttagggcttgcaccctctcaagcaatggcctgatctgtaccttggccctttttatcaatggctggagctggagcaagtgggacacaggttgccatgtcccatggctgcacagagcagtggggccttgggcccagcccacaaaaccatttttccctcctaggcctcaaggcctgtgatagaagggtctactgtggagatctctgacatgccttggagacactgtctccattgccttggctattaacgtttgtttccttgttacttatgcaaacttctgcagctggcttgaattctttcccgggaaatggatttttcttttctacttcatggttaggctgcagattttccaaacttgcatgctctgcttcccttttaaatataggttccaatttcaaaccatctctttgtgaacatgtatgactgtgtttctagaaaaagccacatcacaccttgaacactctgctgcttagaaatttcttccacaggataccctaaatcatctctctgaagttcaacattccacggatctctagcgcaggggcaaaatgccactagtctctttgctctaaagcatagcaagagtgacctttgctccagttcccaataagatcctcatctccatctgagaccacctcaggctggacttcactggccacatcactgtcagaattttggtcaaaaccattcaacaagtctctagggagttccagactttcccacatcttcctgtcttcttctgagccctccaaactcttccaatctctgcctgttgtctagttccaaagtcacttccacattttcaggttatcggtatagcagtcccccactcctggtaacaattatctgtattagttcatcctcatactgctataaaaaatacctgagactgggtaatttgtaaaggaaaaaggtttaattgattcacagttccacatggctggggaggcctcaggaaacttacaatcatagtggaagatgaaggagaagcaagtaccttcttcacaaggtggcaggaaagagagtacagggagaactctcacttttaagccatcagatcttgtgagaactccctcactatcatggaacagcatgggagaaactgcccccgtgatccagttacctcccaccaggtccgtccctcgacacgtgaggattaccgttcaagatgagatttgggtgaggacacagagccaaatcgtatgaagctccttatcatttaagttttattgtgacattgcagtaattcagtcacatcttcaggctttacttctaattctagttctcttgctattttcaccatatatgcagttacttcctccactgaagtcttgaaccccccaaagtcatccatgagggttggaatcaacttctggtaaacttgttaatgttgatattttgacttcccatgaaacactagtgttcttaatggtatttagaatggtgaatcctttctggaaggttttcaattcactttacccatatctatcagagaaatcactatggcagctattctttataagacatgtttcttttttttttttttgagatggagtttcgcacttgttgcccaggctggagtgcaatggcgtgatcttggctcacagcaacctctgcctcccaggttcaagcaattctcctgcctcagcctccggagtagctaggattacaggcatgtgccaccacgcccggctaattttgtatttttagtagagacggggtttcaccatgttggtcaggctggtcacgaactccggacctcaggtgatccacccgcctcggcctcccaaagtgctgggattacaggtgtaagccaccgtacccggcaagacatgtttcttaaataataagacttgaaagttgaaatgactccatgatccatgggccacaaaatggatattgtgttaggagtcatgaaaataacattaatcactctatacatctctgtcagagctctgtgacagagacatgaagtgagcacatactgttgcgaaaatggcgccagtaggcttgctcaacatagtttccacaaaccttcaatttgtgtttttaaaaaaatgcagtatctatgaaactcagtgaagtgaaatacattaaaacaaatatacctatgttaactcacatattactgtaattaaactctgtatgactttttttttttttaaacatgagtacactggtttcaaaatttcctggaaaacttatagcaggccaggtgtcatgggtcacatctgtaatcccaacagtttgggagtccaaggtggtggatcacttgaggtcaggagtttgagaccagcctggccaatatggtgaaactccgtctctaccaaaaatgcaaaaattaaccgggcatgttggatgtgcctctaatcccagctactcgggaggctgaggcaggagaaccacttgaacccaggagacagaggttgcaatgagccgagatcacaccactgcactcccagattgggtgacagagtgagaccctgtctcaaaaaaaaaaaaaagaaaaaacttttagcagttatatagtttcttatctttaaatctcccttctttgggtgttttatgcttggttctttagttttagttgcttttgaatttacagtttagtgaattaataatccttttgttttcttagaaaacacaacaaaaccatatttaccatcacgtgcactaacaagacagcaagttcgtgctttgcaagatggtgcagagctttatgaagcagtgaagaatgcagcagacccagcttaccttgaggtgagagagtaagaggacatataatgaggcttgatgattattcaaggtgagaagctgttttagactctctggccatcacaggaaggagtatgttgaaatgctgcatttctcaaaagggatgtgtacatttctgggattttcagtgatgtgccagacgagtgtggtggtatgttttcaactatataccgagtagaggatgggagggttctagaattttatatattaattaaatttggtttaaaatgcaggcaaaacttgttttatttttgtccctcctgtactctgaagcaaaaaaacttttttatttttaagataaaacaaatatcttcaaagtaatggcttagtttccatgttcttagctgtttctcaagtccttcctggagtgtacttgataatcctctaccctaagggtacttgggtagaaatgtttccgaagcactaaactgttagaagtagcataggctttagaatcgtggcactctcattttattagcaaagtaaatgacaataaaatagctggccaggcgcggtggctcacgcctataatcccagcactttgggagaccgaggcagaaggatcacctgaggtcgggagttcgtgaccagcctggccaacgtggtgaaaccccgtccctattaaaaatacaaaattagccaggcgtggtgcacatgcctgtaatcccagctgttcgggaggctcaggcaggagaatcgcttgaatccaggaggcagaggttgcagtgagccaaggtcatgccattgcactccagcctaggcaacaagagcaaaactccgtctcaaaaaaaaaaaaaaaaagctattaaatgggcgtaaaatgttgttttaggatcaaataaataatctatataaaagttccatataaatgttagttactattattagaacataattttatatattaaactacctcctaaatttttagacaggtagatagctaaaaaaaaattcaaattctaagattagtttgttagggaggaaggagcaaatattttaccaaaactacttgtttttaattgattaatttcattcacttgatgacttagtaaatcttgtgaatatagccttaaatttcttaaatagtgggactacaaaataaacaatatttcatcagtaatgtaagcagtgctatactgagtagaattccctcctgttccgaaatgttacaatttgggttctccctgtgagaagtgagtccggttttaaaacctgtgagtatacttgctgcaggtctgaaaatgaaggctttatgattctttcttgaaaaattatttgcctctatcttttataatattatttgttgaagcttgtgcattctatgaatcatcatgaagatagctttaatttcatccacaaaatttaacaatatttttttgtctggacataagggggcagaataagagttggagtagggccttgcccagccactctgtaactggacaagtgatgtatttatttcttaggacctcatttccaccttctatcaagggaaaacctaagagtaggttatctttagggttctaagtgcctatgagtctatgagatttgactttattaaagttatctttgtaattctttgaggagaacgtaggcatccatttttaaaacagtcctgttagaatttgttttcagtaacaatgttgaatgatggccttttgaaatcaggttttacaacaaaattgtttaaacactgcctgcatatttagaatctctatacctatattaagatacagagattggatagtctcccttttcagtatagataatctcccttcccggtagagattaggatattaagatgtataatatcctaaagtgtagcagcagtctggtatgttacatgtctaaattccatttcctattttatttgtttattgatttatctgtttatttttgttacactgggtaagattcccaagaggtacaagtagaaatttgctaaagtgagtaggacagaagtgtagaggcaaacataaaagtatgtttagtacatatctgttttaaattgtatctactatttcaaagttaatggaattatactcctggggctaagaatgagggttctagggccaacctctactacctatgtggcttgtgcaaattagttgtcccctttgtgcctcagttttacctacaacacagaaacaatgatattacctaccccatggactgttgtgaagattaaatgaattagtacatttactacacatagatctatttctcaaaataatgagcattcagatattagccatctgtaatgtagttggtgatgattatgattattagagtacatttataattggaggatcatttttgccgtagggaaatagaattattaatagtttgaggcacctgagaatattatgtgagaaactgattacattaaccacacccttaagatgagctctaattttgttgtatttgtcctgtttaaagccatctagttacaatagatggaacttttttgttctgattgctttttattccaatatcttaaatggtcacagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcaggtaagtatgtaaatgctttgtttttatcagttttattaacttaaaaaatgaccttactaacaaaatgattataaatccagataaagtataaagttagtttatatcagagaagcaaaatccactactaatgcccacaaagagataatataaaagaggatctgtatttattttgaaacaaacatttaaatgataatcacttcttccattgcatctttctcatctttctccaaacagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtacaaacctttcattgtaatttttcagttttgataagtgcttgttagtttatggaatctccatatgttgaatttttgttttgttttctgtaggtttcagatgaaattttatttcagatttaccagccacgggagccccttcacttcagcaaatttttagatccagactttcagccatcttgttctgaggtggacctaataggatttgtcgtttctgttgtgaaaaaaacaggtaatgcacaatatagttaattttttttattgattcttttaaaaaacattgtcttttaaaatctcttatgattagttggagctaccagttggcaaatttgctagctaactagtgatctgaaagtaagcctctttgaacctctgatttttcatgaaaagcaattctctcaattctatattatttcaagggtaacaagttacatcctagtctgtgtacttaattttatagaaattgtccttaattttattttctgcaatttatgttttcttactatttctggtgtatgtgtttatcccattgtgatgttatattggtgtcctcaatttatttccttagccatacactctacttttcattgtacagggctatttattatctcagagtcaagcttttttttttttttttttttccccgagatggagtctcactctgttgcccaggctggagtgcagtggcgcaatctcagcccactgcaagttctgcctcccaggttcacaccattctcctgcctcagcctcccgagtagctgggactacatatacccgccaccgagcctggccaattttttgtatttttagtagagtcggggtttcaccgtgttaaccaggatagtctaaatctcctgacctcgtgatctaccagcctcggcctcccaaagtgctgggattacaggcgtgagccaccgtgcctggccagagtcaagcttttattttattgaatatatggtcttactaagttcaatagcatgaatctgttgtgaagaattcaagaattttcttctatttgttgagttttgttttcttaggagttttgctctttctcttttgctgtgttttctccttattttttaaatgtgtttgtgtttggtgagttatgttttagtgcttcgtaggtttttctttgactatattatattagtaagccacattgttcccatgccattttatttcatcttggtcatatttggatgactcttttcacacattttattgttattatagaaggtggataacttttgttcatttaattcatcaatatttatttaatgactgttatgtgctagacagtgttttaagtgctgggtacatagcgattaacaaaacagataagaatccctaccctcatagagcttacattatgaggttgggggagggagattacaaacaaagaaataagtaatatacatgtgtatagtttttttagtgctcagaaaaaaaattaagtgggtaagggggtaatgtcagagaagagagagggatgtaattttagattgagaggtgaggagagagacctccctggaaagctgacatttgagtgaagcttgaaggaattgagggagtgaggtgaggcatgtggccatctggggaaagctttccaggcaattacaaaggccgcagtacagcaggatcatgcctagtgtgccgtgaagcattggcagagaccagagagtgagaagtaacatccagggacagaggcagtgaagagccaggtcgtgtgggggtccttgtgtggactgtaacttcctgtgatgacaggaagtcacaggaaaattccaggtagagggacactgtctgacaggttttcacagaatcattcaggccactgtgttgagaataggctgtagggggcacaagagtacaaacaagccatttggaggctctttcaagcacttaggcaaaagatgatgaaccaaacaaaagcaatggaagtggtgagaagcagtcagattcttgttgtattttgaaggtagggggacggtgcaggatggtctgaacattgggaaaaatggaattgccacttagaaggaaagactgcaagaaaagcaagtatgtggggaagttcaggagctcagttttagacagttaagttttagatgcttattaggcatctaagtagaaatgtctacttgatggttacataggaatctgttcagaggaatggctggatatgaatttgggagtctttactacaaatttttttgtatttttagtagagacggggtttcaccgtgttagccagaatggtctcgatctcctgacctcgtgatccacccaccttggcctcccaaagtgctgggattacaggcatgagccactgcacccggccagtcatacaggggacatttaaagccgtgagactggatgaggtcactgtgggcatgggagtagatagagacgggaagagatccaagacctgattgaagccttttatacttagaagcagggaaatataaatgtaaatataggaatcagtaaaagaaacagaggaatggccagagaggttggaggaatactggagtgaggtatgctgaaagccaagagaaaaaaagaattgtcgagtagtgagagtgattaagtctgccaaatgctatttcatagaattgataatgaagtgaggaccaagaattgatcattggctttaacaccgtggaggagcactttcagtggactgaaggtggggcaaaggaaatggagggaaaggaggaatgatagtgaatataggcatttcaaggatttttgctttaagagaagaagagaaatgaatcagtagccagaaggggaatcaggatcaagagaacatttgctttttcagttgaaagtgctaatagcatactgatgagatactgtatgctgatgagaaagatccaataaagaaggtaaaatgcaagatggaagcaaaacaggaacagctgtggggcactgttctcagatactgtgtggtatggtatctagaggctctgttgaaattggccttagctagcaggagagactgttcatctgtaatcacaggaaaaaagtaaagtacgtaggtatagataccaatggaagagttgatatacaagaggaaacttgtggcagacctcttttgattgctctatttcctcgctgaaacagggcacaaaatcatcagctgagagtcagaatgaagaaaagggggccaggcgcggtggctaacgcctgtaatccagcactttgggaggccaaggtgggtgtaatccagcactttgggaggccaaggtgggtggatcacgaggtcaggagatggagacaatcctggataacacggtgaaaccccgtctctactaaaaatacaaaaaattagccgggtgtggtggcgggcgcctgcagtctcagctactcgggaggcagaggcaggagaatggcgtgaacctgggaggtggagcttgcagtgagctgagatcacaccactgcactccagactgggagattccgtctcaaaaaagaaaaagagaaagaaaagggtgttgaaggtttgagagaagaggaaaggcatgaaatcattatctaagaaagtggtagagtaaatggactaagtaaacacatcatgactgccagggcccactggaggtttaatgttcatgaatatattgttgttgtgtgatattttttcaaccgtgttcagctctgatggtgtgggcatgaagtagttggaaagtagaatttaaccaggtctgtagtttagctgggtaagtaatgcaaagcaagaagggcaaagaatttgggggtatatgcaaaaggaggatttaaataattgaccttggacacaatgcagagcaaagaagagacattagaagacgtggatcaatgaacaggagataagaaaagctgattgtaggtcacggtgggtttgagttagggttttagagggagtgaactgggcagatcaaaggtaggtggttgaagaaggaggtacttcaaattgagattctgggggaaatggagttattggaaataaaagtcttgggtatgtccattgcagtgagttaccagtggaaaatagaggacatgatcatttaggaagaaaacaaggaacttgggagcaacagagtattggaaggattgcctgtgaggatactgaaatttccaggaaacatgaccatcgtgatgacagaatgacagtgagttatgagttaaaatcttcaaggaatgaaaggcaatgagtgagccagggtcaatggatgcctgtagcaaggaacagtaatgaatgacagtctgataacacgaggttcaaaactgagtgtttttagagtgggaggagcagcaatgaagcatgaggaagacatctgcctcatctcagcctccagtagcacaaggtctgcaggggcaactgtacaggcagacaagaaccaggtttgttgcaacaagatggcaatgagagcacctgcaggaaagggtgacggtagtggagatcttactgagttccagaggccccattgaaaggattcgaggagatgaagaggtaggaggagatggtgcccagaaaggccacatccaaagcctggaagcggaatccagggaatttggcatgactgagagcctgtgctgccttttttaaatgttttaatttttgtgtgtttatagcaggtgtatatatttatgggggagcctgtgcttgttatggggactgacacagatcagctcttggccccaaggcaaggtgtgtgggagaagaaaaagtgaggaggcctagatgtcagaggagtccggctaaaccactgcagaactgctgcctaattcacagcaaccatgagtaaaaatgctgatgatcatcaggtcaaggatagtctggagcagttaagatgttactttacatgggaggtatcaattaaagatgatgaaatgcctgatttggaaaacagagtcttggaccagattgggtttctagactaaatacagtgtgggaatacacaatacacaacctactagcctatgtgaaacacccgaaaggccagaatgaggaagtgctggagaacttgaaagaagctgaagacttaatccagaaagaagatgccaatcagatttgagaagcctggtaacctggggcaactttgcctgggtgtattaccacatgggcagactggcagaaacccagacttacctggacaaggtagagaacatttgcaagaagttttcaagtcctttctgtcacagaatggaatgtccagagatggactgtgaggaagaacgggccttgctggaatgtggagggaagaattatgaacaggccaaggcctgctttgaaaaggatctggcagtggctgctgaaaaccctgaactcaacactgggtatgaaatcaccgcctgtcgcctggatggctttaaattagcaacgggggatcacaagtcattttctttgcctaccctaaggcaggctgtcaggctaaatgtagatgatagatatagtaaggttcttcttgccctgaagctttgggatgaaggacaggaagctgaaggagaaaagtactttgaaggagctctggccaatacgtccatgcagacctttgtctttggatatgtctttggatatgtcttctcttcttactgaagagaagactttgtggatgaagctcttgagctcttaaaaacctccttgcaggcaactcccacttctgccttcctgcatcaccatatagggctttgtgacaggacacaaaagatccaaatgaaggaagctaccaacaagcagcctagagggcagaacagagaaaaggtagacaaaatgataagatcacctgtatttcatcttcaatctgctgtggaacaaaagcccacatttgaggttgcctatatagaactggcaggaaagtatatagaagcaggcaatcacagaaaagctgaagacacttttcaaaaagtgttgtgcatgaaaccagtggtagaagaaataatgcaagacatatatttgcactatggtagatttcaggaatttcaaaagaaatctgatgtcagtgcaattatccgttatttaaaagcaataaaaatagaaaaaggcataattttcaagggataaaagtgtcaattctttggagaaactggttttaaggaaacttcagagaaatgcgttagacctggaaagcttaagcctccttaggtttctgtacaaattgaaaggaaacatgaatgaagctctagagtactgtgagtgggccctgagactggctgctgagttttagaactctgtgggacagggtccttaggcactcaaatattgaccactttcatatttcatttgattttctgttaacgtgctaatcaaggctgaggtgagtggatcacctgaggtcaggagttcaaaaccagtctggccaaagttgcgaaaccgcttctctactaaaaatacaaaaattagccaggggtggtggcacacacctgtaatcccagctactcaggaggctgaggcaggagaattgcttgaacctgggaagcggaggttgcagtgaaatgagatcgcaccactgcactccagcctgggccacagaacgagactgcatctcaaaataaagaaaaaatgataatcatcttttctgcttgctgttttcagaaacatattatgtaattcactgtaatgatgtaattattgaacagttactcaaatctgataaaatattagttgcattcaataattaatgaatcaatgtgtgtgtgcatgtaagaaaaaccatttgtatgaagcagaaaaaaaatgttaggtagatttgtaggaaacaaaatactggacttacactaaaataaagtgagaaggtcggtgccatgacccagtgtaattcttgactctctatttatccaagattaatttctcccccccttagttacagtgtgagggctggatatttgagtttctattcatgatacagtatgtatccctggaagatgtctctggttctagatcatttttcccagttcagtgagtaacgccgtgtatttatttgctataacaaagaaccatagactgggggcttaaacaacagaaatttgttttctcacagttctggaggctggaaatccaagatcaaagtgttggcagggttaattccttctgaggctgtgagaatctgttcattgcctctctctttgcttctggtgatttgctggcattttttggcattctttggcttgtagatgtctcacctccacctttgcttcattgtaaggtggtggtctcccaatgtacatgtctgtgtctaaatttccccatatattaggacaccagtcattatattagagcctgcccatatgtgtgggtggaggattacccaggtgccgaggcaagagactgaaggcacaaactatttcagtataataaagaaaatagttagattaaggatagtcataatacaaattagatatagagatgatcatggacaattagcaatcattataaaccttaatcattagcttttagtattattctttgctgcattactaatataacctaggaataactggcgggtatagggtgaggtgctgaagggacattgtgagaagtgacctagaaggcaagaggtgagccttctgtcacgcccgcataagggccacttgagggctccttggtcaagcggtaacggcagtgtctgggaagacacccgttacttagcagaccgcaaaagggagtctcatttccttggaggagtcagggaacactctgctccaccagcttcttgtggaagcctggatattacgcaggcctgcccgcagtcatccggaggcctaaatcccctccctgtggtgctgtgcttcagtggtcacactccttgtccacttttatgcttctcccgtactcttggttcctctttgaagttcgtagtagatagcggtagaaggaatagtgaaagtcttgaagtctttgatctttcttataagtgcagagaagaaaacgctgacgtatgctgccttccctctctgtttcggctacctaaaaggaaagggccccctatcctgtaatcacgtgaattgcttcaccttttcaatcacttagaagattcaccctccttaccatgcccccttgtcttgtatgcaataaatatcagcaagcccagccgttcgggccgctaccggtctcccgcgtcttgatggtagtggtcccctgggtccagctgttttctctttatctctttgtcttgtgtctttatttcttacaatctcttgtctccgcacacgagaacacccgctaagccgcatagggctggaccctacacatatgacctcatctttttttttttttttgagttagggtctcactctgttgcccaggctggagtgcagtggtgtgatctcagctcactgcagcctctgcctccccagctcaagcaattattgtgccttagcctcccgagtagctgggacgacaggcacgtgccaccacacacggctaattttttgtattttaatagagctggggtttcaccatgttgcccagggtggtcttgaactcctgagctcaggcattctgcttgcctcagcatcacaaagtgctgggattataggcttgagccactatgcccagcgtgtcttaggatttttcaactttataatggtctgaaaacaaaacatgagtaccttacaactattctttttcacttccagtacagtatgcaataaattacatgagatattcaacacttttttttttaaaaacaggccttgcgttagatgattttgctcaactgtaggctaatataagtgttctgaacacatttaagataggctaggctaacctctatgctcagtaggttaaatgtattaaatgcattttctttttttttttttgagattgaaagtcacatttattgccacgtgaaggaaagccaattaagtcaaattgattaatgtttgcagagtatatctgtttcgatccttttgtttttaatctacctttgtcgaatttaaagtgagtttcctataaggaacaaatagtcgggttataaatttttatccactctttcaatctctgtccattgattggatatttacatcacttacaatgaaggtaattattgatatgccagcattcaactttgtcattttattatttgttttctatttgtttctcctgcttttcattcctccatttctcttttcttgccttcctgaaggttacttaaatatattttaggattccatcttgatttatttttagtgttttcaggcatttttgtattatttttgtagttgtttctctaggtgttacaacatacatgtatgacttacagcagtctactagtatcaatattctaccactctaagtgaagtgtgagaattttgctttcattttgattttggagggaaagggattggatagtgaccatatacagacctttttcttccctacttttaaatactattttcttggatatcatagtattattatttttgttttagtcatcaaacataatttataaactgcatgaggaaatgatagcttatcgtatatgtccatttctcaaactctttccattgtttcttcctccatcctattgtcctcagtcctgatgctctgagagtcttcctttaatcttctcttttctgtttaaacaatttcttttaggcaatttttgagttagagaaaattcttttagttttctctcatctcagaatattttatttctcctttattccttgagaattgtttcaggatatagaatttgtagttgataattctttctttcagtacttgaaaaatattgtgccacttccctctggcctctggttttagatgagaaatctgtcatcattccaattgatgtgtccctatttaaatgcattttcaactaacaacgggtttattgtgacataaccccatcataagttgtcctcgactgtgtttccagataaggctgcattcacaggtactgggaattagtcacaatgtatcctgtaacatcctggaacctttccttatgtcagtcagtcctggtcacctgtttggtccctggcagttttttctcatttctgttcagaagaagaaaaaggtaaagattcttcagatttaggaataacagaattctcaaaaatgtcattttaccagtttagttggggaaggagattagactctgagccttcctgtgcttagttctatccataatcttctgtttcttaatcaccagattttaaattttatatcatgatattccacgtttgtagattttgggtgaatgtttcctttttaaaatttaatttgtttttaagtgttaaagttctattagaaagtttcaaggagccaccttaaaccagaagtctttaacacccagatctagtcatgatgcctcccagcttaaaatcttcagtggtcccctgtcccttagaaaatgaaacccagactcttttgtatacccccaaaaccattcaacttcctgccttaaaccaccaccatacacacgtcagctaaactctaattttaggctgggcgcagtagctaatgcttgtaatcccaggactttgggaggctgaggcggctcgcaaggctaggagtttgggacaagtctgggcaacagtgagattctgtctctataaacataaaaactaaatttaaaaaaaaaactactttaaaacataataataaaacaaactgtacaagttcatattcctgatgcatgataacatttaatgcttcctacctttaaaaatcttctttgttttgcatagatgctcttttgttctccccctcttgatgaatttgtcctattcattattcagctcaagtattacctccctagtcagatgcttacagtctgggttagatcctccttcctccagcttttgaagcattttgttggtttggctgtttcagcacttgtcacatggtccatgatcctacctgtacacttctgaataagccacctactagcctcagtaatacaatgatctcttcaaggacagggactctgtttggatttctacttccacttctgacataatgggttgaagcaggggtcagttgttgaaaactccaggtatgcagcatctcatacggtcctcatagtcacactgttcatttgctattatcatcccactttacagatggggaaactggagctcagagaggttaagtagcctgctcagggtcacaatgctataaattgatttgaacttatactttcaggccctaaagctcttaggttctttcttatacttcatgctgacaaaacaaaagcaaactcaacatttgagagttgggcttaaaacagggacatctgtatttttaatctaatgctttgttactgtattacagaaacactgtgatatataatgagttaattaaacgagaacctttcttaggttgggaaagatttgttttggggaaagcctgtttccctgggaaacagacttacagatttcatgtaggagattagaaaatgcccttaggaacagcacttgtgaggaagcaaatgcagcaggattgggcagaagaagaagaaaggttcagttagtcccacagggacttctggagctgggtggcccttcagagtcttgctagcctgaggcaagagccagtccttgcatactggctcctccagggaggggcatagccttaagcaagacaatgcctttcgacagagggcaagtccaggagggaactcagaggtgagttgtcaatagctagccctccccagaagctggaggatcacatgccttggtcctgaaggggatctgcacccgcaccatggcatccactccaggtggaatcggtgacagtggttttataaattgctaagtcctttattgtttcagtgacattttctaaaaggaattaaaagctttgaagaattctatgcaagttcagagtaggccagaccacaaagctgcaaaataagtttcttcttttccttttgtcttgtgaagatttacctagtgtaggattctaaaagtgttacactaaaaattcttccagttgttatcagttgtatgataattaaagaaataaggcttccatttcttttgatggaatttgatccagacatggaaacctccacttacgtaaaagtgcaatgaggaaactacactgtttactttttatttttctgtattctaaatgtttttcaataaacatgtattttatatttggggaaaacaagatatatttaaaaaggacatttgggagaaggacagtactatcatcaagttttgaatataattagaggcaagttccaaaattagaaaatggaacatgcttgagaaacttataatttctttaaaagcagtgtttaaaattaagatccattaaataaatttgaaatgaaaagaaagaaaagatgaaccatcatttgcaatagaataaacaaatccttatctgagaatgacatttccattccctgttatttacaacggatacataattctcagagtaacaaaaacaataaagtataaagagtagaattaaatgcttagtgcctcttccaactccagctttcctagcctcagttctatcctggtttatcccaggatagaatggatagcacctacccatctttcactttagaattttataactcttggctaggcacagtggctcatgcctgtaatcccagcactttgggaggccaaagcaggcagatcacttgaggccaggagttcaagaccagcctggccaacatggcttgaacccgggaggcgggggctgcggtgacccaagattgcaccactgcaccctagcctaggccacagagtgagactgcgtctcaaaaaaaaaaaaaaaaaattataactctttattttgaactaatttcagacctttaaacaagttgcaaaaatcgtggaatttccaaatattcctcacctagcttcccctaatgttaacatcttgcataaccgtagtacaattagaatcagaaagttaatagtggtataattattacccatactgtagatgtatttgaaaaattgtttgagtttaaggtattttaccctgtttccccttttttgttctgggatcccaaattgcatttagtcatttttcccctgtattttctaccagtctttaatacttcctgtcttctttttcatgatcattatgcttttgaatagactgataatgatcattatgtttttgaatagactgatcaattattttgtagcattcccctcaatttgagttcgtctgatgttttctcatgactaggatgaagttatgcatttctggcaagactaccactgaagtgatgatgtgtctttctcagtacatcatatcaaggggttaatgatactgatcttaatcacttgattaaggtgataatctgctgggtttctcctctgtacaataacttcctttttctttgtagttaataaatatcttgagggagattctttgagactgaatcctatttcacatcaaactagcattcatcagttgattttgtttgcaacaatgattactgtggtatttgcctaattatgatttttctccctttctttccttctacattaattggaattctataaggaaaagctgtgccctttccaccaatgtatttatttggttatttatatcagtatggactcaagaacatttattttattctgcaggttaaaatttggtaccttcattattttattgtttaactttttttttttagctttgaccattaggagctttctcatatggactcgtgtgttctttcaaccagctttcttcactttttgaacacttccttattttttgacatcacaagatgttccaagttcatcttatatgttccctgccctagtcttggaatcagccatttctcttggcttcttttttttagtagaaaatggtgtttagatccaagatctggttgctagataggctcattactttactgtggaggcatcagtactgccaggccctctcagcagacagaggtgggaaatgtttgtcttccattttggattccctcatgtctagttggatttgtttgttggttggtttattgggtatgtgaagtatcactgtggttctagggagtcagacctgtacaaaaagatatacttcatgtcacttcctccttatccttgcagtcccaatcctaatcctcctttttcccccactcacaccctgttcctttagtttctgagtttatccttcctgatttcttttgctcaaatgaacagatacatgtgtattttcttatattcccttcttccttataagaaggggaacatactctagcctttcttttatgcttttcttttttaactcaacactgtcctggagatcagttcatagaaatcgtcctcactctttttttacagctacgtggtacaccattgtttggatgtaccacagtttatccaactctatcctgtatatgagctaataaaagttgcttccaatattttataattataatgtttcagtgagtaaccttgttcataggtgttttcatgaactttatgttcatgtgtattttactattattagaggtctatcttcagagaggagtacaagaaatgggattactgggtgcaaaggtaaatggatatgtgtctttgctaggtattgccaaatttatctccagaaatcttgcacaaatctgtactcctgttagcaatgtgtgcgtatacctgcttccacatgacctcagtaaaagaatgtgttgtcatattggtattgaaattttagcactgtaagcaacaggtcattttggaaaacctgagctttcgccaaattcagctattttgatttgcttttattattagcatataccaaaataaataggcatattagagtttcctttcttgcatcttaaaattcatctaacacatctataataacattcttttcttttttttccattctaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgaggtaaggttacttttcagcatcaccacacattttggtatttttctattttgacagtccagtatcaaggaaatagcttttatacaaattggatagttgaggtagtatgtgaggtaaagtttaatcatatattaattgcccatgaacctcaggagatgggggaatggggaaatgacagcaactagaaagagaagaatgacttgaagggaaatgagttaggagaaattgtgagaaggatgttcagaaatgcagactttgtaagcaaactggaaattggttacaagaataatatgagttatctgtggtttgcagcagtcagcagtgtgattagttaataatatagagactacaggtttacatttaaactccatatctagtgttttatacagattatatttctttgacttgatttaatcccagataagagacactgatattattttccctagatcatgtatgcattttctgcttaaatctatatatacattatataatattagctggtgtttattgagtgtttactatgtgtcagaccttgttctaagcttctcatttaattctcccacaaccttatgaggtagggaactgtttttctattttatcagtgagaaacaggttaaatggcttgccttaggtcaaatgccaagttagtaaaactaggattcatacttaggccatcgaatgcagaacccagactaggaactgctatgcaatgctgcttcccagtaaaatttgagatttcatgagttggtaactagtgaagaatacacaaaaaataagcctcttaattctgtagtttaatatttgaaatgtgtgttattcagaatttatataaaaatatattttaaaagcattagagtagctgtataaagaaagctgtgttgacattttacctagagactctatgcataatgaataacactctgctatatctagtttctaaattaggggtgggagttgtattcattatttagttcccatacagcatatctactgtttacaccccacattttctttttttctttctttcttttttttttttttttttttttttttttagagacagagtcttgctttgtcacccccaggctggagtacaatagcacaatctcggatcactgcagtgtctgcctcctgggctcaagcgattctcgtgccttagcctcccaagtagctgggactacaggtgcgtgccaccacgcccagctaatttttgtatttttagtagagacatggtttcaccatgttgggcagtctggtctcgaactcttggcctcaagtgatctgcccaccttggcctccccaagtactgggtttacaggcatgagccaccgagcccgcattttctctgagacgtcttcaaaggcagtttactaatcctgctgaagagacaactgtcatttacacagcattttaaagttttacaaaatactgtcatgaattaggttaaaccatatgaaattgctgatatttgaccagttgtgatttctcaagcaacagtttcatgtagtttaacctataaatcatttcaattaattcttggaacagacgtgaggtaggtgaggcaattctttcttttctctaaccaaagaagtacctttatagatgtgagatgattcccagctattaagtagtaaatagagctaggacttgagccccaatcttccagcttcaatccagatcatatgacagcttgctgattaaactagatgacagagaagatctctttccttcagatacacatactttttctctgttcccctctccctatcagctagattcccctaaatcactgatactggttttgtaattttgcatcggcatgtttgacaattggtatcacatttagggtttttcattcttttttggtccaaacttttcatttctgcttttaaaggaaatacttttggaaacataaatatgtgggtttgcaatttataaagcagcttttccacttattttcttagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctggtaagttaatgtaaactcaaggaatattataagaagtatatatggaggccatcgtatattctgttgtatacctagtaaacatggtaaaatgtaattaaacttaattagaaaatgtggttgttatgtggctcctgtaagtatagttatttagaaattttatttattgaagcaagatatgaaactctgggtgcacactttccaaacaggtgctttcatttacatgtgattgaaaagtgttttttgccatttatttcactgttccatacaattagggttgtttctaagctgtttgtaagctgtttctaagctatttaagtggttaaatcacagtagatgcaaatcaagctaaagtctttaacattggctaatggctgattcttaaatagctaatacttgctaagggtatctatattaactcatttaatcctcataacaaccctatgagataaaacctaagtcctcacttaacattgtcaataggtttttggaaactgattttaagggaagtgatgtataacaaaaccatttttttttctcatcactgttctaacaaaatgatgttgaagatttaaatgacattgctcaaagacctgctatacattgtttgacttaaagtcacagtttccgagaacctatcaattatgttaagtgaggacttgactctattatcctgattttgtagatgaggagactgtggcatagagagaggttaagcaattgcctaataaggtcacaaagctagaaaagtaggtattagaacccagatagtgtgtgttctcaagatggctttaaaatatttatctttgtttaatctgttaataataaaaaacaaaagattaaagcataagtgacgtcccctacctccttttttatcttttactgtgattattcttcatcttccttccttttcatgtcattttatatgttcttatgtaaaattactttcatctagaataggaataatgtgaactgaaatcacctaacctattaggagttaggggagggagactgtgtgtaatatttgcgtgcttaaatattttcaatgaaaagttactttgatttagttttttatgttactacataattatgataggctacgttttcatttttttatcagatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacaaagaaaaaaaaaaggggaaaagaaaatcttttaaatctttggatttgatcactacaagtattattttacaagtgaaataaacataccattttcttttagattgtgtcattaaatggaatgaggtctcttagtacagttattttgatgcagataattccttttagtttagctactattttaggggattttttttagaggtaactcactatgaaatagttctccttaatgcaaatatgttggttctgctatagttccatcctgttcaaaagtcaggatgaatatgaagagtggtgtttccttttgagcaattcttcatccttaagtcagcatgattataagaaaaatagaaccctcagtgtaactctaattcctttttactattccagtgtgatctctgaaattaaattacttcaactaaaaattcaaatactttaaatcagaagatttcatagttaatttattttttttttcaacaaaatggtcatccaaactcaaacttgagaaaatatcttgctttcaaattggcactgattctgcctgctttatttttagcgctatcacaggacccagagcctatgcccttttaaacttaccacaaaagcagaagattaattcaatttaagatgatactctcatttgttacgtcctttttttttttttttggagatggagtcttgctttgtcgcccatgctggagtgcagtggcatgatcctggctcactgcagcctccacttcccgggttcaagtaattctcccacctcaagcctccctagtagctgggattacagggacgcaccaccatgcccagctaatttttgcatttttagtagagactgggttttaccatgttggccaagctggtctcaaactcctgatgtcaggtgatccatctgcctcagcctcccaaagtgctgggattataggcgtgagccactgtgcccggccaatatttgttactttcttaggtttaatagagaaaagggataaaacatttctaactgggagttaatagcatggagaaggtcttaaatcagatgttttaatgccttaaatgtctgtataatatcatgttttcaaatctaattataaatacgtttaaagccaagaataaatcttttaaaaaattgacttgtttccttccataactctgagccatgatt

SEQ ID NO: 6 - DNA, 5012 bp, Homo sapiens
  misc_feature (1062)..(1062): T1062G single nucleotide polymorphism
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttcttctgcacccacttctaaatttaatgtttttctggaagtagtaattgcaagagttcgaatagaaagttatgtaccaagtaaccatttctcagctgccataataatgcctagtggcttcccctctgtcaaatctagtttcctatggaaaagaagatggcagatacaggagagacgacagagggtcctaggctggaatgttcctttcgaaagcaatgcttctatcaaatactagtattaatttatgtatctggttaatgacatacttggagagcaaattatggaaatgtgtattttatatgatttttgaggtcctgtctaaaccctgtgtccctgagggatctgtctcactggcatcttgttgagggccttgcacataggaaacttttgataagtatctgcggaaaaacaaacatgaatcctgtgatattgggctcttcaggaagcataaagcaattgtgaaatacagtataccgcagtggctctaggtggaggaaaggaggaaaaagtgcttattatgtgcaacattatgattaatctgattatacaccatttttgagcagatcttggaatgaatgacatgacctttccctagagaataaggatgaaataatcactcattctatgaacagtgacactactttctattctttagctgtactgtaatttctttgagttgatagttttacaaattcttaataggttcaaaagcaatctggtctgaataacactggatttgtttctgtgatctctgaggtctattttatgtttttgctgctacttctgtggaagtagctttgaactagttttactttgaactttcacgctgaaacatgctagtgatatctagaaagggctaattaggtctcatcctttaatgccccttaaataagtcttgctgattttcagacagggaagtctctctattacactggagctgttttatagataagtcaatattgtatcaggcaagataaaccaatgtcataacaggcattgccaacctcactgacacagggtcatagtgtataataatatactgtactatataatatatcatctttagaggtatgattttttcatgaaagataagcttttggtaatattcattttaaagtggacttattaaaattggatgctagagaatcaagtttattttatgtatatatttttctgattataagagtaatatatgttcattgtaaaaatttttaaaacacagaaactatatgcaaagaaaaaataaaaattatctataatctcagaacccagaaatagccactattaacatttcctacgtattttattttacatagatcatattgtatatagttagtatctttattaatttttattatgaaactttcctttgtcattattagtcttcaaaagcatgatttttaatagttgttgagtattccaccacaggaatgtatcacaacttaaccgttcccgtttgttagactagtttcttattaatgttgatgaatgttgtttaaaaataattttgttgctacatttactttaatttccttgactgtaaagagaagtaattttgctccttgataaagtattatattaataataaatctgcctgcaactttttgccttctttcataatcataaaaaaa

SEQ ID NO: 7 - protein, 1012 aa, Homo sapiens
  misc_feature (352)..(352): F352V polymorphism
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDXTESEKKFIKGTADFFALXFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 8 - DNA, 19 bp, Artificial sequence
  Single strand DNA oligonucleotide
gaagaatgaccgaccacag

SEQ ID NO: 9 - DNA, 25 bp, Artificial sequence
  Single strand DNA oligonucleotide
atgaactttttctcagattctttaa

SEQ ID NO: 10 - DNA, 26 bp, Artificial sequence
  Single strand DNA oligonucleotide
gagaaaaagttcatcaaaggaactgc

SEQ ID NO: 11 - DNA, 20 bp, Artificial sequence
  Single strand DNA oligonucleotide
caattggcggaacttcatgt

SEQ ID NO: 12 - DNA, 22 bp, Artificial sequence
  Single strand DNA oligonucleotide
ctctttcctttggacccacctt

SEQ ID NO: 13 - DNA, 21 bp, Artificial sequence
  Single strand DNA oligonucleotide
ctctttgctttggacccacct

SEQ ID NO: 14 - DNA, 20 bp, Artificial sequence
  Single strand DNA oligonucleotide
cctaccattgacactctcag

SEQ ID NO: 15 - DNA, 16 bp, Artificial sequence
  Single strand DNA oligonucleotide
tagggccatccattct

SEQ ID NO: 16 - DNA, 19 bp, Artificial sequence
  Single strand DNA oligonucleotide
agaatcattgccctactta

SEQ ID NO: 17 - DNA, 20 bp, Artificial sequence
  Single strand DNA oligonucleotide
gataacttaccagcatgtga

SEQ ID NO: 18 - DNA, 20 bp, Artificial sequence
  Single strand DNA oligonucleotide
acctgttgtatggcagcagt

SEQ ID NO: 19 - DNA, 20 bp, Artificial sequence
  Single strand DNA oligonucleotide
ggttgactctttccccaact

SEQ ID NO: 20 - DNA, 49712 bp, Homo sapiens
  misc_feature: klotho genomic sequence
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggtgagtgcgaggggccaggcggagggccacgcaggggagacagagggcctccacaggggccagggggaagtgtgggaactgagtctcccccagacgaggcttcacttggacacgtgtatgtggtcaccgggggaaactgagcagttctgacttcccttggaaggcgtggaattaggagagaaatcccttagtgggcacacgagtgagtgccccttggagtccatctgtggaaaggaagcggtgataggtttccgcagtgaggaaagaaactcctttctctgggtgtagaggaattcctagaggtgagggcggggaggatttgctaggagtaactgcaggaagagaatgagcagggtggatgaaagaaacacgtttgttcttaagccgcacagatagcattactctctggagctgtcacgagttcagtgttaatccaataagatctgtcttgcttgtggcacaagttcacactgttgtgaaagtgtcaaaacacaactcccggagagtcagatttaaactgtgtttggaggtcccttctgcggcagggcagggaactgcatccaccaatcttattcaccggggtgtgaagaccgctttatacaatctcaaacaaagctagtcatagaaacacagaaacaactgcagggtgatatagattgttctaccacatctatatttccacagtgctctctacagttgaaagggttcctcacttatgacattgttagcctgataaaattaattgtaacacccttgtgcaaaatattttattaattttgacagcaggaaatattgctatgccatggtaaagttgtctcaagtatttcttcctccttttttctccttagtgttcaaatttaggcaccattgttttggggattcatttataatcatgtcagttgttagtacactttgtacagagttttgtatcacaataaaatattttatgtacatgtataaacattttaataaaagtgtttaaatgtacaacttttggtacccaattctgatcacttgtgggcctactggaagacttatttttaaatttagaattcagccacatattctgaacaaagtcagtactaaaaccatttctaaacacttttaattaaagccatagataatatgggcagagcagtactgggttattgaggtgacttagtgctctgagctgctggtaatgttgtaataatggctaaattagattttatgtcagttccacaaaacctgtttgctgctactggccataaaagaatgttggatttgtttggaaacacattgtgatttttgtgatttcatacttatgtatgtcttttcataaggatgttttagagcggtgattcttatactttttcaatccttttatggatctgaaaaactctaaagaccaccagtgtatggctggtgatagaaaaactatgggaagaatggtctcactttttgaagaaaacataagcaagttatcagttcatactggaagcaaataaatggatcagctggaggatctaatagatcatgcccataaaatctttactttagatttaaatcctacaagttctatttttagagtccctaggataaacttgcatatacaaagtagtcaattgttattatagtgacttataggatcattttattataagataaagacttgcgtgctctctggtgctctggtgttttaaataatcaacatgttttagatctttttgcccactcatggccctctgattaacttctcagttatatttttcaatgcttacacaaaaaatttgccaaacactaaccatccttcctagaaatgcagtggactttgtatttagggaaattagtaacaggtgaatgctccatttgctgatgttgcaaacaaactgttagttggtgtaaagtattaattttgctttcacatttcttttggtttggtgtttagtcattcttgtatcttatttagaagcgagttcccattgtcaaagaaagtatcttgaattaattttgttgtatgagatactttcattctgttttcaatgacctttctcggtgtgtatataggggttgggggaagcgagaatagaaaaatttgagcttacctgaaaaagataaaacattctgcagattttgataaaagggcattttagctggggcttagatattgctgctgtacagatataggggccagttgtgttccgggatctgatgttggtgaaggataaagactggctagcacccttccctcactccctctgtgtttccactaacttacaaatgggctaaggagatcccctgggatgatgggaagcttgacccctcctagagagtcaagaatcattgatggcctggaggacaggaggcagaacaaacaaacagcaggacaaataatggaggccctgctcagaatggaggtcttggtgttctttgtgggttcctcatactggagtgagccttggcaaaaatggtgtgtgtgtgtgtgtgtgtgtgtgagagagagagagagagagagagaggagacagaagagaaggagaagaggggagaggggaggagggacagagagagagaggtctcctacaaagagcttggagctcaggctgctttgctcatttgagtgtaatccaggaggatgcaccttttctctcggacttgaagaccaactgctcagccttcacagctcttgaaacaaagttcttaatgtcccctgaaatcagcactagggactgtagttggggagttcagccccagcagtgcctctgagtctttgttctgtatatgggttctaagaggattagaatcaagttggaaatacacgtgttcccatttcccaccttcaactcccctcaccgcctgagattcttgcaaacatttgtgcaaggaacctgtgaaaaagacttctttcacgtaaggaagtagggctaggagatattaatctgggcattgttgagcattagcatcagtgtgcaggtctcagcccatctgtctgcaatgtttgttgaagccatcacttcttgtcccttgaatccttcctcaacttggatttcaggccctggctctcccttgcttctcctctcagctccctggcgattccctcttagtctcctttgcttgttcctcctcaccttgcctgtaaatagtggagggctcagggctctgtctagattcactccaggccattgctttgaatactgctgcatcccgatgactggagattttgagtctccacccaggtttccccgagcatcagactggcatagacaaccgccttctccacgtggatgtctaatgggcatgtcacatcgaacacatcccaaaccaaacccctgcttccccttctctattatgatgactttttactcttccagttgttggaccccgaatcttaactactcccctttttcatgtcttgtttctaatccatcaacaaatcctgcagctctacctacctccaggatgtgtccagaatcaaaccacttctccccttatctatgactacgatggaggtgcaagacaccatccaaccttgcctgggtaatgggaacagcctcactgctggggttcttatttctgccttgccccctgcagtgtgccatgatggatgctctacagggtgacattatgtgtggacaagacagagtccttgctttttcaaatcccacctgttcttctggagccagctaacacgctcccttgggccccatgcctgaatgccatctcttccttctatggcatcctgcacaggacttcgctttgctcagatgcctcctcccgcgctcttccctttatttaaccatttgtctccattttaccccttcgctatctggggtaatcctgttcccttcatctgtatctctgaaaatggcatcttgccctcccttggtgctcagaaggtggcttctggtttaccgaatagcctgatttcacctttataaactcatgctactagaattttctctccttgtcagtctttctagccactttccaatggaacagagactaccaaatcctaatctgataaggaaaaataggtaaacataacgtggcagactctgacatctgcagtgaaaatttaaggttcatcatcaaatgtatgattccattgtgaaaatgtccagttaaaagctatctgtagtcaccatgcagcattatgaagaagttttcagaataagggcaggtggtaaagtctcctggccagctttaggagtatatattggaaggggctttgttagctgattgaatttttattccccaagtaattagtatgagcgatttgtctactgtatatatgctcaaattacatatactatgcacttttgagaaatactttacaagtttttctttcattaaaatgtatttggggccgggcgcagtggctcacacctgtaatcccagcactttgggaggctgaggcgggtggatcacctgagatcaggagtttgagaacagcctggccaacatggtgaaaccccgtctctactaaaaatatggaaaattcaccagacgtggtggcaggtgcctgtaatcccagctacttgggaggctgaggcaggagaatcgtttgaacatgggaggcagaggttgtagtgagctgagatcgcaccattgcactccatcctgagcaacaagagcgaaactccatctcaaaaaaatgtattttgtacataactagaataatcaactggattgaaagttaatataaattttagaatactactgaattcaatagtgcatttagtcagaaatagttaaaatatctcccaaacagcttgaatcactcctttttgaacacattgttttttgaaggttacagtcaagtccaagaaaaaattttaaaataggaaaagaattaatcaaatcatttcaaaatcatagcagtgttttaacaatgccaattattttgagttgaagaaaagaaggaaaatgtaaactatgcattcaagtaatgtcaatgcaccttctcaggtgcaaaatgaatgaacaattaactgtgccagctgaatatgtagcaatcacggcttttgcacatagaagccttctgaattatcttatattctcaaaaatatcattgctctaaactagaattttattataattaatttgtgcaaaagttgtctttctttcaatgaatgttgtctttttgaggcataaaggaaattttaggtggaggaggagaatagaatacatgcttactgggaggacaagtaataataggtgagtaaaaacacacagcttattgaattgtctcagatccacattttctccagaaatgcaaagttatacttgaaatctatatttaaacagataagcagaatgtgacttttatatgctctattttgatgtattctgagtatgaagataacctgaaggatgcctttttcccctctttatcttggggtaaactcttacttacccttcagagacttgactcaaatattttcattctgcaataaccacaggtagaatggatcatttactcagttgtggctctcttaagagcacagactacctatcatatttatttcaaaatttgtagtgcttatctcagtgcttggcacagaggaaagaaatacaatttttaataaaggcataagtggagaggtaaaatacatatttctggtttcaacgtgtccatattctttcttgtaaatggaatattgcgcttgcagtctgctctcacacaatatctgggaaggagttttaaagagtgaggaagaacagtaaagattttcctaggcagatacgttatgtgacctttgttgatttccagagcacataatatcccaatttcatccagtggacaggaaaagagaggaagagggaaaaggtggcctctcaagtgcctgacctggaagctgcacctatcactgtcccttgggcagaactcagtcacatggtcaaaaggagtctgcaaataattgtcccctgaggagccgggtgctcagtgaatgattctattaagttgaaatcagatgggaacagatcatttaggataaccagcaggctctaccacattgcctaactcccaggcatattgtgagaattaaaagcaccttatatatgtcaacgtgttttgaaaaaatacaaaaagctctacaaaagtgagctataaatttatcattaataataatagtaataatagcaaaatacttgagaaatggtcctcttgagctgtttagaaggaatcatacaaatgcattagacatggtagcctcacttaactacttaatttgcctcttctttgaaattatttcaatagcatttgaccaaaaactatcaaatcatttttgaaataacgtatttttacataaaacacattatcaaatatctttctggatccagcttggtggtaaaaagatacatactaaagtttatgactgatataactttatatattgactaaaccaaagataaatattgattgaacttttgtggccccagtttcaatagatttttttattaacattttttgtttgaagtacagatgtcacgtcatccatgaaccggtatcattatagcttgataaaatactcaaactgaaagcagtgatgtacattaattttaaatataatggttaagcaaatgttatttccatatctataagtgcattttatttgataattagaatgttagaatcagaaggaatttggaaaatcccaggttacacttctctccaggacgaactccttacagtggctctcctggggtcagctagcttttgcttgaccctttgaagcaggaggaactcagttccactcaagttgcccccttgattttctctttcatattgaacctgctgctgcacaggactcctccgtgactgtctctttgccttcctaaatctcaaagaataaagcaagtcactttttgacaagacatctttcacatatttgaaaagacatctttcacatatttgaaaagaccagtacgttttgttcttcctagaattttctccttcatgctaatgtctgctccaagaagacatggaggtgactggacatttagtcaggaacatttcttgagagcttaccctgtggcaggcaggtgcaatgttgaattctgagaatatgaatgtaacctttgaacaggtggctctgtcttctgacgtttcaggataaggtggaggagaggagaggggaaagtcgacctatctccccagcggggagggtttagggttgtctgtgggacctgcctctgtctcatcatccatcaccattgtcctgctgatgggtgcagaggactgaggacgagtggttggagttctccctgtgccggaccctgtagagagtccagagccctggctcccaaaggaggcatatttgtagggctcttttcgtgagggtccagaggacggctgcaaagctgcaggggaggatgggggcatgggaggcaagcccgttgccctgtcagagcctcttggggatgctgctcttccccgcctgtcctctcttctctttgatttctcatcatgtggtcctctttcctctgccttttcctctcttcggtgtcccaagttcttccatagagacctgtgtccccttgttcccatcaggcctttatgccagccctgcacaggtgcggggacagtggtaggggcgtctcactcccgactcaactacattctcccagaaattgtctgcagtcaaacacaactcagcgtggacattgctcaccaaaggtatactgcttgggctgcacgagagattcagttttctcctatctatctggcatgggctgtgagggggctcctaacctagggggcctttttactccttcctggccagagctgccatttccaagtttctgcactgtcagaaaagagggataaggtaagattcctgccctcatgtcacagattagtaggggagaggtaattgtcaaataattacagtaaaatgtataaatgccttaagagaaataagtacttgcttaggaacctatttcaagaaaggagagataggttagcgttttggggaggtagtacgggagcagtcagggaaagcaatacttgtaagatcagaggctggcaatctttttctgtagagggctaagttttggactttgcagatcctacagtgtctgttgcaactattacacagttctgtggttgcagcatgaatgcagccatgggcaatgtgttaatgaatgggcacagctgtgtttcaataaaactttatttacaaaaataggtagtgggccctgctgctgggcttggcttgctgggcacacaaagtgagaagggcattgtgcctttctaggaaacatcccttgcaaagtcagtacaggtgagacagtgtggccattcccaccacagcatgaggtcttttgaatggctgaagcgtcaggtgggaaaggatgaggagctgaagaagaaagcctgggaatttccaaggcctctcttaacatttgctgtgcagaaccaagcagtgccccaggtgttcagagtaggcttcagcgattacatttcttgttagacactgaattttgttcatgccgcccaagattgcattaacaattttggctgtcaccttaccgcggactcaaattagtttgcagtcagctaaaacctctcaggttttttccccccagcttgtgaattgctgtgaagccaaacatccctgtcatctctttggtagtttattttcaaactttcaatcttattaaatttcatctccaatgtgcaaagataagaggaagtttaccgctgatggtggtttattctttaaaactggacatcagctaaatgtgttaaggtaatccgttagaggaatgttggtacgcttatacagtggaatactaattggttattaaaatgttgatgtatgattattaaaaggcagcccatgttatgtctcttctgtatgaataagtatgtaaaaagtatgtaaagattactgtatagttcatgggtgggactgggctctctctagcctctgtccttcattctccctgccagtgccttttcccttgcagtgtcccttccctggctgcatactcaagactggatttgcccagccaaaccccattaactcacagaagcaagaggaaaaaaataaattcttattagttcaattctccaaattgggggcggtctatttcacagcaataggtaactgacgtaaaaggaaaggaaacaatgaagtaattccagaaaatttcctatagctggaggacatgcattttcatattgaatgggaagaatggggtgggggggtcatacctctgtgttattgcagggtcctggctcctagggacctaggcacctcttcagtcagtgaattatagcttggaaaattggctaagtcggggaactgtacaaggggtcatcactttttatgtagccatgctctatgaaccatctttataactccctggcttctggcagttaagcaccactcactatctggcctctgtaatttcaacttctgaccatccccgtgggtggcaacaagcccagctctgtgacacctctgctaccagcagccctggcctggagagcggtgcccccactgaatgtctcagtgagattggcacccccatgagcatattcctgagggtgcagtgaatgctctttctccctacttgggacttctgggaaacactgtcctctggtgggttgtttgacctcacataatatactggttcttgcatgccagcttctctctgcttcttgattcttttatcagctgttagggcaacaatgtcaggcatttccgcctgctcagcattgggcatccctttttacaggcttctcagagccaccctagcagcaactttaccgcgtctcctggggtccttgctggatcgtgaaaacatacttccggagaaagtgttccctagataatgaattatttcttgtcctgtgttaagttccagttccttggtcaaggaccccagcgctactcctcaggatttgcagtgcatgctcaatcttgaagcaatgttgggatatagtatcttttcttctctatcaggcccagctgtctccaggtgggttatactaggatttaatcctcgttagtgccgggtcagctcctgaggggtgcacctgttgctttttagggacgaggcctcttcagcataaagaagtgctatctcttactagggaagaatgggccacctctgtaagctccaaatattcccaggtttccccaaccggggccttgaccttggcataactgatctgccttggctgagcatttaaacatccctaaatctctgcaagtctatcaggtcctaggcctgctgctaagtttctctactcttccactgtaggagagaaggctgtggctcttatattgagcctttaactgtttgttattggccctcagtttctcattagctctctgtagagtctcaatacaatatatcagaaagcattcaattccatctccttgtaggcagggaatgcctaaatcattgcacctacaatgacattctccaccaggatatttcccaggttactgctgttgaaatatttagcaattgaagcaccatttgtgcaaggggactatctgcccctacacaacactcagaatggcattctctttgctttccagaaagtgaatgagctggttccccaacccctcttgtgttagtttgagtcctgaaagaagtaaatgtcaagataggattagatgtacaagaaatttattggggaaagaattgtgaaggataaaggggaaggagctaggggaggcattgaacctttggatcatgatgtgtgtccaacaactatgaaggagagtgggcagggagaaggatgggctgggaagagtttcagttggcactgtgggtctcagaacaacccaggcataggctgatggggacgccttgagccaatgttgcccattggcggagtccacatcttgctgaaatgggcctgcattagttcccctgccatgtttagtcatctgggagtagccgatgagaatcatgatcttaggatcaactgcaatggcagattcaaaggggtagcaactgatggcctcagtcaactgtgctccttatagcaggaacactgagcagtgcatttcttggccatcacaaagactagtgaggagtgcccttcagagaagggaatgaaaattatttccagcctagaatttgatacttatctaaactgtcaatcattcatgagagtcaaagtcccaaaaaataaatcttccataaaccctttctcaggaagttattggagggtaaccgccacataacatgaggaagacaaggaggaagacatgggatctatgaaagggcaggtctaacccaggaaaaggatgatgaactgtagacccatgatgtcagatgtgcagcagactaagatcagccagtgcagaatgggggagattccagaagtgtgtccccccaaaatattggaggctcatgtgacttccctggggaagtttctgctgagaggctattggaaattgagggaagaattagccacagtcccaaagaaaacagagccaatcaaaaagcaatgcaattatgaacttcaaagaaaacaaaaataagaaggaaaacagtcttacttactacattacaaggtccagctgtgaatgatattcatgttggcagaataatgtcaataccaaatgtcggttaaacccaaaactattgtataactaaattgcctgtgtaagagagctaaacccttatctagcataataggaagtcagtggatacttcctgaagtgtggatgtgtggagatgtaaattccaaaagaaaacgtcttaagagtcaaaagtggttgcctctagagagaactgggggaattgtagggcaatgtaggacagggaactgtgatttttcttgtaagttgttctgacattttaaaccatgaacatatattacttgtatttttaattttaaaaagacataaatattttttctatcactttaaatattaattttgctagttatagcacatatttatagcattacactctgtagagctcttttggattcaagttttggagaattttcaaagttttagattaatgcctttgtgagttttcatccctttgatgataacaagttacaaagaaatagggttatgaataaaccttgtgggatattgttagtcaccttctctcaagtcgatctgtcccatgaatcaatgtttcaaacattgaccagcatttgatacttaataagcaacgagtaagtttttgttgaaccaatgaatactcttaaaatatattttttcaagtggcaacagtactatcttatttgcactctacttttctttttgaccctaagaatgtcacaaaaatgtttagcaactgtcaagattattacatacagagatgactattgtgttctcagatatgctgtatgtcttagttcattttgtacagctataagagtagctgagactaggttatttatttatttatttatttattttttgagacggagtcttgctgtgttgcccaggctggagtgcagtggcacaatctcggctcactccaagctccgcctcccaggttcacgccattctcctgcctcagcctcccgagtagctgggactacaggcacctgccgccatgcctggctaattttttgtatttttagtagagacggggtttcaccgtgttagtcgggatggtctcgatctcctgaccttgtgatccactcacctcggcctcccaaagtgctgggattacaggcgtgagccaccgcgcctggccctattttttttttttttaaaggaatttatttcctcacagttctgttggctgggaagtccaagggcaagtctctggcatctggtgagagacttcttgctgctttctcccatggtggaaggtgagagggcaagagagagacaaaaggaggctgaactcatccttttacaaggaacccatgcctgagatattgaacccacttctatgataacagcattaattcattgatgaggacagagacctcattgcctaattggcctaaagtgagacctcttaaaggtctcgcttaatactattacaatggcaattaaatttcaacatgagttttggaggggcaagcattcaaatcatagcaccatataatacaataaaaattttctgagctaagtttggtaaatttattctaggaatccatgttgtctcctagttatcttcccttccatagtactgatgcatttttgttaaccattacctaattttgcttgagtctattatttctgaaattcacattatcttcccttttaaagttgagaaaattttcattcttcaagcgccttattttctataatctctcaaagtaactgatggctgttgcatgatcataagtgcaaattattttgctagaccacacttggagatgatgaatttggaatggcatgcagactcccgacatcaggagtcttgtctcctgcaataatcaggaacccaggcttaaaagggagcaggtacaacagaagggcaaggggtgacaatgctggtgaaagacatttgaggaaagctatcaacatgaaacagaataaaataaacagaaaagcaaaccagggaaaataaattatgcaggaattaaacacatacacaaactgaaacaggaaccatcagaacatataaaaaattcttgaacatcagtaacacaatagttgaaacaaaatatttagtaaaatatttgaaaagtaaggtcaaagcaatgctgtaaaagtagtacaagatgataaagaaatagaacacatttttgaaagggaaaaagatttaaaggatattcataaagatccaacatcagactaatagaagttctggaaagagagaataaggaaatacaggtcaggaaatttgtaaagaaataatataataaaatgccccagaactgaagaacatgagcttttaactttaaagagccgactgagttcctagctcaatgaatgtatagacatgtaagggtattgaaagttcaccttcagtgtcatgtttcctaagaagctaatggaagatatgcaccagcaaaattgtagtaaatacataggaagaaatagtatatagaaaaagtatggtttcaatccataagaaataaaggaaactcccaggacgagagcagctcttgacagcatctagtccaaactggacttggaggctggaaacttctagcagggaaggaagagctctgggtgaaaaagtagactcaacagaatagatacgatcatagaaaacatgatagagaatcactaacacattgaaaaaatcacatatagaatattctgcacgcttaataatgaggtcattatttattcaagggaaaattaaaagctgtttagaaaagggaaatgttatagtgccctatttggctctaaaatgaacatttatataggaatcttcatgtaaatactaacaatgatttaaataagaacagacatttcggaaaataagggaagaaaatggggcatgtaaaagagctaactcctcattatcctaatgaattattaaatttcacaatagcatagtatttagaaatatgatagttattacgagaagaaagagctaaaaggttgccagtggggagcaggagtgagggttagagacgggattgggggagatgcttactgttttcatgataagcctttggtactatttgattttaaactatataaatgcatttattaatttaaagtaatttaaaaaacccataccactggataatgcttgataatttctagagtcctttttttttgtattttggggcaggtaaattcattgagagacccagagagtttagctgactttcctgtgggtaccaagggtcagagctggggtcaaaactcaggttttctgaacccctattcccagtgtacattccatgactccaggctgcctcccgcattgcacaggttacatctaggggtgtgctagcaaatgcctagaccatcctcgtccacatcagcatctgaaatggacaagaatgttagtcatgacttgccactaacgtctttaaccttaattgacatctgagagtgtcatcattacatcattacaaaaacactaacccagatacatctgttcccattactatttctgcgtaattcccccagacttaattgctttaaataaccattttatttggtttacaaacttatgggtcaggattaggggagggctcacccacgcagtttttctctggtccgcagtcatctgaagcttgaacggggtggggcatgcaagacggctcacacatgtgatccgcggttgagcctggctgtggaccagagcatctctgttggcctcgcttacacaagtggtctcagattagtagcctctgtacatggaaactagcttccctcccggcaagcatcccaagagaactaggaggaagtggtatggacttcttttcttcttcttcttcttcttttcacttattcttgatatcatgtagcttcatttctatcagagcagtcacatgcccacagattcaagggggagggtccacagatccgccaatgggaggaacagccaggttatatcgtaaaagatcatggggcatgggagatacccatctattttctgtaaaaaatacattttgccacagcatcactggctttccagctcacagtgatctgcctggaatgcctttttccgtctctccaggatctacatctttcaatataaggtttagaaactacctcctacaggaagacttccttgatttccctcagcaagaaagaatctttccttcctctgaatcgccattgcattaaaaaaaaaatccatcttctggcgttttcactttcagacttatactgtaattattctatgtacctatgtttgacccctcacgatgcccagcataataatttgtctattgagtaaatatttgcttaatgaaataatcattacatgataactcaaatagcagtcctgaaaaagtgcatttcaattcagatctctcttttttcttccttcacaattctcatttccgaaaaatgaaagaagccagaggatcctttatgaggagttacagtataacttatgcgtggctgtttcctgtgtttactgctactcagtgagaaatacgggagatgggagagtgaaaaaccatgtcatttacaatttgattaaaaagctttttatcttttcctttcacgtttaagccttgccgttttaaaaatttccctttcgtcacaggggatcaagcagcagttaacgctgcagttccctgttctggaaacactctcaaaggtgtttcaacacattttgtctcaactctgactcctgccccgctgccccacgccatccagccacactgaaggtcttgcatttgtgccttggggcattatttttttttactcttctccctgcctggaaggttcttcccaacctgccctaccgccatagccacagaaccaacccttcactttccttaagtctatggtcacaagctccttctcaaaggaggcacacctctaaccacccccatttggtgacttcctgtctcctggaggcccccggaactagtcttctctacttccagcaacccactttacgagcagcggagaagactgactacttctgggggcctcgaggagacaggaagtcaccaaatttacaggtgctgtggatgaaactgtgtgataatgaatttattgggcttgtttttttagcttctgaatagaagaaccaataagatcatttttttaaaaaagataaaaacagacaaacacaaaccctctagtataaaagcatttttttttaaaaaagatgaacacacaccctcagattgccttcttttgaaaaggcaatctgagattccttatgaaatccccagacagaagctgtttctttgaatttaatatgctgtacactgtagagccaagaggcttatagagtgttaattaacaccccttgtcaaacatttgtaaatgaatctggctaaagctcaaggaaaccaggtttttctgtgtgataaaatataatctttggagattatttatgatcacaaagggagactgtacagaaaattttcctagacttggaaatgaggcaggattattggtgtccatctggactgaggcctcaggcaggcctgcctagttaatcccattctcctatcctcattctctgggggtgaagaaggcagtgcacctttgttcaatttgctgcttaccatggattagggcattttaaattctgtaagggtagtttttaacttgtagaaaactgatagcgatgggaaggattcttgcctaatagggacacaaacggattttgttctgtagagatgtaaatgaaaagatgaaaatcacaacacatttaatgaaaggaaaataaagactcttgtgagtgccaccaaaaaaaaaaataaataaaatgtatctcctctccgtggcaccccaggccttccttacctcactctatttcttcatagcacttattgccttagaacattctttataatttacatttattatgtttgttgttggttgtcgctcctcctgctaaaatggaatctccgtgagggcacgggtttttctttgatttgttccctgttgtgccccaaagtgctagaatcatgccaggcacacaatagatgctcaataaatacttctggaatgaaaatccccctccacttagaccagtgagttccaaacttttttgatcttgacccatagtaagaaaggcattttccatgttgaatatacaccattgaaacaaaattttcacagaaaaaacttaccattattacaggcagtgcactgtgatatttcctaatctcttctattttgattttcaaaattgctgaggctactcataggttgatttcacaagtggagtttgtgttatgaaaaactttgacttaacaccatgtcgaaactgctaccacaaaaaggcaaatgcgaaagaagggggaaagagcaggccgatgactttccgctatccaccactccataaagcatatttcattttcctgtaattgtatcctgttcaatgatgtagaaatcctcacacactcacatgccactttttctttgggtgaaaagcgttctctactgcaagatgaattgagttatttcaaaagcaaagagctataaatgagcctgttaaagaaagtctcaaggagagtctgttggcatctgctgttgataacttaaagcaggagaattagataaggaggcagaagtagaatgtttagaaaataaaagtgaccattatagaggaaaaactgcttgtctcagttttgttgatattggagagtagctctttcatgagggtattgtggaattattggcaattatactaatagatgtttactgaaaaaatcctatttgactgatgaaccatggaatacttttgctgaccttgtggaaaacatcacttatctgagttccttatcttcttgtctcttttttctcatctagcctatgcctccctacctgttccctcatggttctcattttgttgctattagaaaaacagagatacaaaaaccaggaattggaacccctctgtgtctccagtaataattctgtgattaatgggttagatggattctggtcacaagctggatattttgttaacaacctagcgtcaatgacttggaatgattttcaccgcagcatgatttagtattgaatagaatgatttaactaatgttaattagttctgtacagataaattaatgaagcaagaagctcaatctctgatttattgatgtatttacccagtgtaagttatgaaatcttttttatttcatttgaaggaagtttttatttaaatacaaataaataagccctttattgtcacctactttggaaaagtccagataaaacaatcttaagtaacaaaactccaaaattacaacatgattttcaaaaactaccctgacctttgtcttgcctggttgttacagtgtacttttaactaaacggattcttatagaatctcaagtttggttatatttgtattaaggaactctatatttgcatttgaccagccctaactaaaacaagcttaaggaagaaaagggactttacgacaaggacaaaggattgaagaagaactccagggactttagatgttaacaactgtatttgtcactaaagtaaccattactgtcaaggtgggttctccttctccctcactaaacacatgcacacacaccacacacacatacacataccacatacataccacacacatgcacacacaccacacacacaccacaccacacacacagaaatacacacagacatacacacatgcacttatgcttgggtcagtttggcttttatcatcccagagagtctcttcccatgtcagcaactccagattcacttcctctcctaatataggacagtcacatgtctctattccagcaaaacaatctcagggaaggtatctcattggcctggcttgggtcattcaacttgaagtacaagaagagaaaacagtattcttttatttttatagatttcctgtaaaatcagtggataatggtaaaaatttcatgacagcaggtgttttaccttatttatgtttactatctctagaacccagtatgatagttcataataaatgcttactaaaacaattatcaaactgtacacttaaaaattaaaagagtaaattttaagttgtgtatgttttaccacagtaaaacaaaataaaataaacctattattgatcttattgtctatttctcaaaagtagcatacgtcataatttcatgtgatttctaaaggagatctaatctcaaacttagttcttagagtaaaataaaaggttttggcaatcatatacagcagtagactttaccttgaagatttaacaaaggtttgaaagcaaaatgctatgatactgaatatactaatgtttgagggcttgtgaaaaggtcttaatataggaatcatatatcttcttttagattgtgtcttggggaacattgggggtcagtttttctatcagtgttagtatgtagtaagtaatggcatacatctaagcttgagattttgctcatttctgtattcttagcacttatatatccaacatataataggtgttggataatatttgttgaataaatgactaaataaatgtcttggcatgagaattatgccatctacaaaaccgtcacttttaaaaaaaacaacaaagatttttgaacctgtagatccagccagaaagcaagaaatatcttcacctttccagactgacttattttttggtctagctgtgtactatgcatgagctgtcaactttaatactttattttttaatgcctaggtctagtgagttacaatgtgatgtagactgattgaattaaaggccccaatgttgtcctctcttcattgtatccatgccttttgtcatgtaactttgcagtgtcctctactctaggtgtccattcagcttatcctttgactctggccttcttctcctttgactctggctttatccatgtgacttactttagccaacaatcataatgctggcaaacactgagaaaatgcttattagtgtctgcttagtctcttgcttctctgcaattgccatgagaaaatgcccaagctagtacactggaggatgagacatgtggggcagaagcaacctgtcccatttgtcccagccaacaccatcctagattaaccagatgagaccagagcagaagacatgttcagtggagcccaacccagctcacgaataatatttcttcatgagcatgtgagcaatgtgtaactaatgcaccatgctctttttattctgcacttccaaacatctctcagattgccctgttgttttgcttttcagtagtcatcacctttgtccagaaccttaattgtatctgcattttggcagtatcctgccaattttccacatccagctgttcacattctaatgcattctgcaatggatgattttgagcatatgactgatcatcactgtctcagaaatttatgctaatctattgtgacctattctgtcaaatccaaataactttacattcaaaacttttcatattctggccccatactgtctattgtaatcttattttctaaaagccacaaactattaatcctgtagcctaattaggttgatatcatcattttcacaacacgttgggtaaattcttatatctgttcctttaatcatagtttccatgcttagacactccattgccctccctaagcaccagccagagtccactatggggggatattttaaaggctcttttcccctaacccatctgccccagaggtggcacaggctagaaaagatagtccaattatttggcttaattatttggatcttacagtttttttgggagagttccagatgatttgatgaaatgcaacatggtcaaaaacattttggtggagaataatttaatcaactagaccatttggtgaaaaataaatcccagttactagacttcccagtattaataattttggtatgccaggaatttttaaaacatttctaacaaattttttcagaaatcagaatgactgaaaggtataacaataggaaattaggaaatcaataagtaatcattgaaaagtgagtgattgaattagttggaacaatctatgccatccatcctgtgggggaaaaaatgaaaacagctaaacaagcagagcagacatcatcaacttagaaaaacaatatagtaaataatttgaactagaaaattttccaaaattgaaaattccagttttaaaaaatttgtgttttcttattcaaaaagcaatacatatttcgttacataaaagtcagaaaatcagagaagcagaagaacataataaatgcccactgtcctactcaatagagactataactgctatctctgtacaaatcctttaaaatcctgtcttatgtgtatttattttacatctgtacatatgattccaaaattaggtaatactgtacgtggtctatctatagctaactatagtttgtaatttaataagctatatttgtccatggtgatcatttattgctcataaaattaataaattataatttatttatcaacactgatcatttgttgctaataatttatttcatttaatgtcttattcctgggttattttaactaatccctattgttagccctttaggttgtttcaactaatttaagaattataaatggcccatagtagttacttaataaatatttgagtgaaaggataaatgaatgaataattccacattattggatgttcaagttgtttccattcaactttattgttgtgggggagtataatacaatcttggtgaagagccaccaacttaggaagaactatgaagaagcatgttatttactcctgcttgtgggtggggtgcattgggaaaggaatgcctaaatgcacgtggggagccaaacaagacttcacaggggagatgggaattgagccaaatttggaagaagtgagaattttcagttgataattggggaagatagtactaagagtaagtggggttcattaggaactacttcttagaagaggcaagtccagaacagaaggcagaaagggatatggatatgaaggaaaagtagaatgcaacttcagggctagtaggttggcatccaacgtaggtacagccctggatgaggagtaatgagaaactgacctggctgtagcaaaacagctaggctgggaagtgatgctgtttacatttatatgcgattctactggtcttcctgttactttgtagaagactgtaaggcttggtgctcagagctagaaatgcaacttatttgatttgctaagaagtccaaagcaggtaggggtgccaagctgctgcctgttcacacatattcgtctaactgggtgtcagggaaagcagatatgagcccccttgcattggttaaatcctgagcaacttttaaggaactgcacacagagactactggctattttttgtggataaagttgtagatagttatttttgggaaattatttatgtgttctattaggtggttgttttgtgggaggctgtttgaaattctgtcttaaggaactgcagcttataaaccatagtgctgatagaaataagagaaataatttggtctgcgagcacaaacatcaggctagttggcatgcttatgtaaacaccacacagtagcagtgacttttaaggaagtcctcagacaatgtaatcccaatactcttcttgaagttgaggtaatagggtgccagaaagaaaaagaaaataaatctctagtgccttctaaaattttttcaatcctttgacctttttgaaaacggtcacctttaattcagaactaaagtaacatctggagggcccttttcttagatagggagaagacataaatgagctatttgaattattttctgcttatgggctgcatttcatttcttccaccattggtctcagtccatttaattaagattatttgtaggattaaattaatcctttaagaattattccatttcaatttttaaaaaatctaatgaatgatagataacaaaaagcggtccattcagtattacccctgaaacatatttgggtgattggtgataccatccatgtctctccaacttcagcttcatatactcagctgagtaccaggcatcttcattgaaagctagacttgtgacctaaatagaacttgatcccttcccttccccaactccttattttttgttttctttgagttccttgattcctcttcccctcctcacatgcaacatgtaacatccaattcatcagcaggttctacagattctgccttcaataatagcctaaatctagccacttgttactatcttttttggtccaaaccatcatgatcttttgcctggaccattgcaatagttgtctaactggtatccttgctcccattttgtcctcctagatcctattttccacgtagtagccactgatttttggaaacttatcaatcattacccagacacaatgagtacatttacatagttgtttgattccatttacataaagtcccacaatcactctatgtactgggaatcacaacagtggtctcctatgttgagaattgattaagagtcacaaggaaacaaggagtaatattccattgtattaacagaccacactgttcatacattctcctgtaaatggacatttatgttgttttcagtttttttgctattgtgattaaagctattaggttggtgccaaagtaattgcagtttttgccattactttttaaatggcaaaaactgcaattactttggcaccaacctaatactgtaagtatctttgaacttaccaatcaaacattactacaacacttgaataagaaacacttccatagcatttacaattctaagtgctttccatgtattgacttagttaatcctcacaaggatataaccatgaggtgctattactatccctactttacagatgaggaaatagaggcacagacttcagataatttgcccaaggtcccacagctaataagggggcagaattaggaaagcctggctgagtctatgctctcatcacttgctcaactctgcctggaatgcccttcccgtaccctctaccgcctctgcccaatcctccgggttctctgctccttccacacacccccatctcaggtcccagtcttttttagcattcttcactttttctgttacagatcctcagacaatgcttccttgaagaagacttgcctgtctaaactgccttcccatcccaatccctgttaccttctatcctttctctgctttgttttcatttgttatctctcttcttcccaagaatgtaagctccataagaacaggatcatattttgaatcccctagaaaagtgcctagtgtaatatttgttgaatgaatatgatccctgcaattaaaagctaaatatatatatttttttaactaacagattactgtagtagtttcaaagatgaatttatgtttcggagaaaatcagtattctcttgccacataaattgtaggtaattatatttctatacctgaatagctttgccaatgactaagatattaatctattatatatttattaatctatagatctttaaaattgatgcacatgttttataagcaatttgatgaattttgatgactacatacacctgtatgtgtatggatatctgtaacaaatatccaagtaaagatatagaatattctcattaccccagaaagtttctttgtgacttctaatcaattctcacctccgtaggaaaccgctgttgtgattcccatcaccacagactgattttgggactttatgtaaatggaatcaaacaactctgtgaatgtactctgtgtctgagttctttcaatctacacaatctttttgacattaatccatattactgcctagaagagtagttcactctttgcattaatgaatagtattccattgtataaacaaaccacactgtttatacattctcctgtgaatggacatttgtgttgttttcagttttctgctattgcgatttaagctactataagcatttttttttaattgtgatggggtttcgctcttgttccccaggctggagtgcagtggcagcgatctcagctcactgcagcttctgccacgtgggtccaagcgattctcctgcctcagcctcccgagtacctgggattacaggcatgtgccaccacacctggctaagtttcgtattttcagtagagacggggtttcaccatgttggtcaggctggtctcgaagtcctgacctcaggtgatccacccacctcggcctcccaaagtgctgggattacagtcttgagccactgtgcccggccagcatctttgaacatatcaatttgtaagttttatcttaatattgtacaagtcttttggggggcttatgttgtcatttctcttggtaaatacctaggtatgaacttgctagattatagagaaaatctatctttaattttataagaaactgtcaaatagttttccaaagtggtggtactatttatactcccaccatcaatgtatgaaatttccgttgttttacgtccttgccagaatttgttggtagtctttttttttttttgaggcagcatctcactgtgttgcccaggcatacaatggtgtgatctcggctcactgcaacctctgcctcccaggttcaagggattttcatgcctcagcatcccaagcagctgggactacagaggcgtgccaccacacccagctaatttttgtatttttagtagagatggggtttcaccatgttgcccaagttggtctcgaactcctggcttcaagtgattcgcccgcctcagcctcccaaattgctgggattataggcgtgagtcactgtacccagcctgttgccagtatttttagttgtaggcatcttagtgggtgtgagtgctcgttggggttttaatttgcattttcctgatagtgttgatgttgaggacatttctatgtgtttactgagcattggtgaagattctcttgtgaaatatctattcaaatattttgctcatggtgggaaggggagttatttttcttttactactgataggtaggcttacgtatttatttcggatataattattttgtcaattatatactaatcataaacaaaaactgataaattggacgacgtcaaaattaaaacctgctcatcaaatgttagcgaaatgtaaaggcaaatcacatactgaggggagatattttaatatatgtatatttatatagtgctttctgtgttctaagaagtattttcctactccaagataaagagactattctcttacattttgttctataagttttatagttttagcttttagctttggatctatgatctgtctcaaatttttatgcaagattggggtttaattttttcatacacttttccagttgtcaaggatcatttgttgaaacgtctttcctgttgccacataattgctttgatgcattcgttagaaatcagttggctgtgggtttattttggaattttctgttctgctcctttgatgtatttgtctatccttatgccaatatcaccctatattaaataattatagctttataataagtcttgaaatcaggtaatgtgaatgtttcaactgtgtttttcctttttctagttattttagctgttttatgttcttattgtatatattttagaatcaacttattcatttctacaaaaagtttattgggattctggatgagatggtgttgattcagtaggtcaatctggggaaatctgataacaatattgactcttccaatccatgaaaatggtatctcattctttatttacatattctttaatttctgttagcaatgtgttataattgtagcaaacttgcacatcttttgttaaattattttctaagtattttacgattttggtaccactgtaagtggcattgtatttaaaatttattttctgtttgttttctgttcatatataaatgcaattgattttcttttttttttttttttttttttttttttgagacagaggcttactttgtcacccaggctggagtgcagtggcgtgatcagcactcactgcagacttgaactcctgggctgaagggagcctctcacctcagcctcccaagtagctgggactatgggtgtgagccagtgttcctggccaaatgcagctgatttttgtattgacattgtattctgccaacttgctaaattaacttattcgttttaatagtttttctgtttttttaaaaatcttaggatttctacacagacaatcatgtttttaatgaacaacaaagtttgtttttttgtttgtttgtttttcccttttcaatcaacatgccttttatgtttttatttgccttactgcactggctaggacctccagtacaattttaatagcaatggtgagagtgtttaaatgtactcctgtgtatattttattcttttggaacttattataaatggaattgttttcttaattttctttttggactgttcattgctattgtacagaaatacaactgactattgtgtgttgatcttgtaccttgcaattttgctgaaatcgtttattttttgcaatagatttttgtgaattctttaggattttccatatgtagaatcatgttatctgtgaatagggatagttttacttcttttctaacttggatagttttttccttcctaattgctctggcaagaacttctagtacaatgttagagagcaatagtgaaagcaggcatcttcctttcaatcctgatgttaggggtgaagctctcagcctttcactgtaatgttggctgtggattttcataattttttgtttgtttgtttttttgtttgagacggagtttctcttgttgcctgggctggagtgcagtggcgtgatctcggctcatcacaacctctgcctcctgggttcaagcgattctcctgcctcagcctccagagtagctgggattacaggtgcctgccaccacaccgactaattttgtatttttagtagagacggggtttctcgatgttggtcaggctgctctcgaactcctgacctcagatgatccgcccgcctcggcctcccaaagtgctgggattacaggcatgagccaccacgcccggcccataaatgcttttcttaatcatattaaggaagttcctttctagtcgtagttttctgagtgtttttattatgaaagactttcagatttttgtaaaatgcttttcctgcgttaattgagataatcatatgggttttctccccctttactctattgatgtaatgcattacaacgattttttttaatgtttacccatctttgcattcctggaataaatactagttgattgtgctgtataattcttaaaatatgctgctggatttgttttgttagtatttggttgcattcttttgcatctatattcataagggatattgatctgtaactattattttcttgtggtggctttatctggctttggtatcaagataatgctggccacattggctaagttagaaagtgttctttcttctattttttgaagagcttgaaaagagtcgtgttaattcttctttaaatatttggtacaattcactattaaagccactagtcctgggcttttcttagtttgaaagtttttgattactaattcaatctcttttacacctagattagattttgtattttttccttagccacttttggtaatgtgtgtgcttccgggaatttggccaggtcatctctgttatctaatttgctggcatccaaatgttcataatgttctcttgtaatcctttttattatagaaatgatatacagaaaaggtcagttaccactttcttttatgattgcattaatttgcttcttttctctttttttctctagtcagtcttgctaaaggttggtctgttttgttgatttttttcaaagaaccaacttttgattttgttgattctataatttttctgctctgtattttgtgtatatccattctaatttttattagttcctttattctgttagctgtgagtttagttggctcttcttttttattttcttaaggtggaagactaagttattgagatatatcttgtttttttttttgatgtaggtatttaaagctataaaatttcctctgagcattgcttttgctgcaatttataagtattggtatgttacatattcagttagttttttatatacacatttaagcttttgctaatctaccttgtgatttcttcattgacttattgcttgtttgagtgtgtcaacaatttccacgtatttgtgaatattctagttttccttttgttattgatttctagtttcatttcattgtggtaagaaacaatactttttatgatctcaacagtttaaaatttattaagacttattttctggtctaacatatgatctatcctggaaaatgtatcatgtgcacttgagaaaaatgtatattctgatttttttaggtgggtggcatgttctattaatatatatgtctgttagctctaggtgaattatagtgatgttcaaagcctccattttgttattaatataaccatgccagatttttaatgctgtccatttgcataatatgtctctttccattctttttcttttacatgatcactttatatttatagtatatttcttgcacacagaatgtaattgaaacttacttttctaaaatctactttcttttttttttttttgagatggagtcttgctctgttgcccaggctggagtgcagtggcacaatcttggctcactgcaatctctgcttcccgggttcaagcaattctcctgtctcagcctcccgagtagctgggattacaggcacctgtatttttagtagagacggggtttcaccatgttggtcaggctggtcttgaactcctgacctcgtgatccacctgccttggcctcccaaagtgctgggattacaggcgtgagccactgcacccagcccctaaaatctgctttcatagcatctgtcttgtacttggaacatttagtccatgtgtatttgaatatttattgatatgtttgggtttatgtttacaatcctgctatttgttttctttttgttccatgtgttttttgttttttatttctgcatcttttggaaaaatcagattttttggtaattcatttttcttattttattggctttttagagaaaccttttaatatctttgtgagttttagggattacattatttattcttattattattccatttagaattaatattgttaatcccattcaaaaatttgtttatttcgcttttcagttctagaattttcatgttttcttcagttgttgtttctctgttgttattctccatttgttcattatatctatcttttctttgaaatccttaaaaatatttataatagctattttaaagtcctctgctaatcccaatgtctgggacatcttggcttctgttgatattgaatacttctttttttccccttaattaagggtttcattttcctgctttttcacatatctagtagatttttattatgctctgtgtgctatgaatgagatattataggaagcctggtcaatattgtctctttttaaagggtgttgtatttagttctgccaagaagttaaattaccagatttacttgatgtggctttagtctttgttagagctggtctattcctgctttgttcttacttctcaggtaatgaccttcctgagtttcagctggatgcctgaagtgctcagctgcatttttctactctggctattctgaaatgcaatattttccagacctacccaacctttggtattcatctcccagacctgtggctgcttctctttgctgagcctcacaaaatcttgtcctgtgcatggacagccaaggatccttgggaaatctcatgcagacttctaggtccttcctttgtgtagatttcttctctccagtacgttgacctgcttcagagtcctcatcattgctttcttccttttcagctcagcaatactgctgtgtattgtgtgggcttcatttgcctcagccatacctagaacacaacccaaggcagaaagctggagtggacctgaggctcatcacctgtttccttccacccaccatttaaaagcagcttatttgtgtgaaactttttgttgggttctgtgggggatacgtataggcaaatgacagggagcttataatttagccaaagagaaaatgtctatatgttctgtggaattcagagtattttcctcgacttccaaaattattttcctcgacttccaaaatttggggctactagctagttggtaggaaggaattccagatccatttttcttctagatttttttcagactccatgtttcaaaatctagatggaaggtaagaaggaagcaaggagggcatcacatacgagagagacttataccccaaagtggatcattagcacattatgagataatatggtattattctcagaggcctgccatataaaattccttctaatttattttttaatggtatgtgcctgaaagttttctgtctttcatgatcttgaaagcaaaataagaaccagagtagcttatgagagagttttcctgcttcagcccagaaaaatgttgcttctgtcctagaccctttgatctggttcttagtgccagccttcattcctctagatctgtttattgaatactggctctgttgcagagactttgctagaatctgagagataaaagactcaatgccttcaaaacacctacaatccagacacttaaataaatgcaataatatatggttaaggtcttggatagaggaccatcatagaaacacatagaacagacatttagtccatgctagggagagggaagatgtcaaggaatgcttcctggaggtggcgacatctaagctgagttttaaagtgggtgtaagagtgaaggaactgggaataggcaaaacataccttagagagctgcacctccaaagcatggaaggctagataatcttagaacttcaagtggggttcaatgtgcctgggacttggatttcaggcagagaccatagggcaagttggagaacaggtgggcagggtgagatgaagcagagacttttaataaccatcaaacaggcctgccatccaactgctatcagccattttccctttttgaattttatttttttaatctctagagcaatacatgtttatggtaggaaatgaaaaaaatatatagatgggtaaaatgaagtgaattataccacccacaatcctgctgtaaaacaggttttaacaagttataaatccctccagatcaaagtgctactaaactctacttgctattttataacctttttctgttcagagtattttagaaacgtttttacatgttaattgatattcttttgcattatcctgaataatggtatttctttagttatactatcatgtattaaaacattcatacatggtaaacatatttgaatattagacatttcaattgttttcaatgtttggctattaaaaatagtatttagatgaatgttcttggccatacagttaataacttcactgagtccttgtcaataactttattaaatcccaatcttaagacaacaagcctataagattaacataccaggaataattagaaagcattgcaagactatatgtaaccatctccctgttaatcatccttatttgttgcaacatcctaaatagcgaccttcccacactctgcctcataatatttaatctgtcaaatgtaccatccgcaaagggagtgaactggctcgtgaagaggtggtgaagctggttcaactcagtttaacagacaatgagggcctccgtgtgccagggagggagtgagggaactccacagggagctcattctggggagggatgcccctcattatctgcagtaaatgatattagagaagtatgtgtgagactcttggtgtccagaggaggggcatgcagcacagtatgggggtccatggggttgcagcgatttcttagagacggtgccatgcctgggatgaccaccaagttaggatgctgtaaagagtaatcaaataagtttcaacagggttttggcatgaggtgagcagttaatccaaatgatatctgaaatgctttctaatgctgcaagtgtattcctctgtaaaataatcaactacttaaaagacactctcacttgcattatgtcatttaatcctcacagtgtccctacgggacaatagatgtcattcccacttgtacagagcagtaagtgaggctcacaccggtcatagttagaggagtagggaatctaaacctaagtctcttctactccaaaccccaagttttatcagtatgtcacatcgctactgtgggctgaattccttagcaggggttgtttcctccacctgcaatattcttccttttggtttgttcatcctctagagagagaagggatacgcattcatggagatcctactgttgcaaactctgcattaggagttttacatgaattgtctcatttagtcctcacaacagtcctgtgaggcacaggaaagttagatttacctgcccaaattaaggaatgtggccaactcaagagtgacgaaggccaggtgcggtggctcatgcctgtaatctcagcactttgggaggctgaggtgggaggctgaggatcgcttgagcccaggagtttaagaccagccagggcaacacagcaagatttaatctctactagaaataaaaaagaaattagccaggtgcctgtagtcccagttacttgggaggttgaagtgggacgattgcttgagcctgggaggtagaggctgcagtgagccatgatcccatcacttcactccagtcagggtgacagagtgagacccagtcttaaaacaaaacaaaacaaaaaagagtgatggagcaggaactttatctgccatcagagaccatatatgtctttccttatggcccaggtcatttatcatgtccttgctaaacctcctcctctgagccttgttaaagtcctcaaggttagaagagtggtctataattataaaaaattccattgtatgacttcagctacctgcaaaactgaccgagattaatgcattctttgtttgctttcactctttcattccctgttcagcgcttattctacagaaaggtgcctggtagatttaggacatgactgttcataggcctcaaatgcggtcaaattaggaaagtcccctggtttttggtctcaagaggttaattgctgagtactagcctggactgctcaatggattttatgtttaactgttgtgtttatttgtttgtttgtttttgttttgtgagacatatctttctctgtcacctgggctggaggtcagtggtgtgaacatggctcaccgaagcctccatctcctgggctcaagtgatcctcctgcctcagccttcctagtagctgggactacaggcacacagcaccatgactggttaagttttggattttttggtagaaatggggtctcactctttgcgcaggctagttgtgaactcctggtatcaagggattctcccaatgtgctgggatttcggcctcccaaagtgctgggactgcaggcatgagccatcatgcccagcctctgtttaactgttaacatcatgaggtttcctttcaatgaggaagggaggcctggggaggtgtgatggggaagatggagaagggtaggagacatcataaaactgacagaagtgtggcacgattaagaccctggtttcttagaccacataagcaagtgccactattcttttgatcaacatttgattctctatttccttcttagcatatagatagctgcttcaggtcgtgaaaaaatacatttagtgaaatgaaacatagtagatatgttcaccaagaaactaaaaagaaaagttagccacaaactatcttatttcattgaaatgtttggctgaacccataagaatgttgatgaggccattcttggatgcctgtactgaaatgaaccacgagaggaatatttaggatatgttgaagaggctgtttggcttaatgaacaaaggagtttctgcaggcccagggagtggaatgaaaattggcatgatcattttggagaatatatggagtcaaacagagaatttgaatggagttttcaaagaggatatgttaggggaataaaggctgataaacaacacttgtatttgtaggaaaatagggagagtaaatcaaggaattcaaagagaaagagaaactcacttcaagtaggggagaaaaaacccctataattttcactcttccttgtaaataaaaaggaaacaaatgaaaataagatattagaagtcagtaagaatttatgggagtataaagttggttttatggatgcaaagcccttttcactgctgtacgaaactcctggctgcatgctaacaatggacaactgatttccttgcagctgtattttgctgttttttgctcttggcttggacctagcaccctgggtctgtgggaaaccagaactgtcccagagttctggagggtaggccaaggttagatgctggagtgggttctttaatttattgtactgattcttcttgggaagaaagaagattgcttgttagaattttagctacgagagatgactatgaaacagtaaattaactccaacgacctgagtcattttgaaaactcccagtctcaggataaaaaatataatcctatttagaaattcctggtgtgatcacagatgtagcattggttcttttcatgaaacccgtaaattaaaaagtacataatccaaagtcaattaaatagtaagctattataacaaattcttttatttcattagcttttcaaaatgtggataactacacactcaacccaaggaatctacatttttccactgactgctaaagaccaatggaaataactctagtccccgtagcacctcactgtggggtgacctacctttgaaataatgtattggttctagctgatttttatattgttagtcattaagttaggcttgatgagaaacagatataatctgatttggggattcaagtattatattgcatttctcctcacaactagagataaatttgccatggtttttctcttcataggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaaggtatgattgtgggtaaagttctcatttcctgccaaaatcttctggaaaaaaatctctaagattatctaacataaatgatgtgaatttatatttttaaatcctaatggagacattcattttggcaatagtagaatgcattcatttaacacctttctcatttggagtcttgaggaacttgaattaatttttaaaaacccatttgtaaatgagaaactgggttataatatttgtaattacttaactttcagttattaatctagatttttagattaaattgaacataaaacaaatcccaggatatctagctctctgcacatgtttttcagttcttgttattttggttgaataaaacactttaaagaaaaaggaatgtccatgttttctagagaaaatagtataaatagatcatgcttttaaagccttcatttatttatttattgcatcagacacaaagctgggtgtctaggatggaaagtggtacaagacatctttccagccctgtagaatatctattataaataaggaactattttttcaaggtgctcagaaatccaaaaaacatattagataggccaattttgagggcatttatttgtagagttatataggtttgattagagtctttcgtcaagaagaaaaatcattggcttaccaaacgagaagcattacactttatttatttaagtaggaaacgctcagctgctcttgaaccatgatgcaagtgcccagcgaagggtcatgttgctcttgtcccctcttccctttgcagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtaagtcagctgacaaaaccaatcagcagtctcaccaagccctatcactagtaagtagtgcttccttcctaggctgattgtcatggcacattgtccgttctttgagccaaaaacaattccttatgagtacactaagggcacaatttggaatgctgcacccttctctccaaaactcttccaatcttcatcttgtttaagttagatccaaagataaataaatttaaagcatatcaatatttaagatccgattaagacagtaaaaagataaaacactctcttttcatactgtggtttttgatcctttttaaggcagttgagttttttcatgaacaggatctaacacagaactccaaagcctctgagtttcagtggtgctgctgagactgaggcaggaacattaggcagagtcctccagaggcacaactgtgggctccacaaatgtgcagaaataccctaagaaagtaaaccctagatccaatgattcactggtcagaatgtcttttttagcaatagtcattgaaatgatacgaaatttcttcagaatgatcaaccaatatttattgagcatcttctcagtagtaagcccttaacattctttcagacttcctaaattttgaaggggcttgttttccagcatttgactggatactctagtaagcacttattggatgtctagtgtgtccgaagccttgtgttagttgctcgggtcgcttggttaaggggagtgcaggtagagggtatactgagatgagtaagggtaacctttgctttcaaaggagcaaaggagtctactgagcgaaaacaatgtatgcacaaatgatgcaatggagtgaagcgggcatggtggtaagtaacaagggcggggctgggggattgctgctgatagagtcccaagtgtgaaaatagccctcaagacagagacagagttcagtgtccatagacaagcagttggctttgacatgttgggttatggtagccaattaattggttctgcaaatcacagcttgaaaggaaacacttggaagaatgtgaaatgggttgctgttttcttgtaaatatccaattgaaatcttttatttataaggaaataaattaacaccatccttagtacattttttgctggttgggattattcttctttttcagaccacccagttcattttacaggcagtctcagacttaaaccctcgccttccatttaaaagatgactggctcacgcctgtaatcccagcactttgggaggccgaggcgggcggatcatgaggtcaggagatcaagaccatcctgaataacacggtgaaaccccgtctctactaaaaatacaaaaaaaaaaaaaaaaattatccgggtgtggtggcgggcacctgtagtcccagctactctggaggctggggcaggagaatggcatgaacccaggaggcggagcttgcagtgagccgagattgcgccactgcactccagcctgggtgacagagcaagagtccgtctcttaaaaaaaaaaatgactggatgtgtcatcttttatgccaggatatgtgagcccaggagaaaggcttctgagctccctcctgctcggtgtgcaattttctgccctgccccgactctctccttctctcccagcctcctgctatttgaaatctccttatcctaatttccctcctcagagtggattccactgtggggttcagagaggatctgaggtgggagaagtgaggctggtgaggaagaaggggaggagaaagggaagaagacctccgtagccttccttcctcctcctctttactggggttggggatagatcggatggtccctggtccttgttctatctcttgaccttctgcctgctccctgctgagcacggatctctgatagcagcctgagtctggcaggttcagtcctttgtatgcggcacaatctcccagccagcattgctgtgcagatcatgggaacgaatgcagaacaagagtgggggtgtcggagggagccctacttctcctgttctattcctcatcagggggctgtgcgctggctttgggaattggtaaatagtgagaaagtcttaagggtacatcctatttccttgagggagaagagaaaacgctggtcagaagcaataagtatagcagtgaatagcaagggagatgggagataattccttttcctactacactctagaagctattgttttagaatctgacctaaggtcagccactaattggccccagaggtctctctctcagatcacacggtccttttttcctcatcagcttggggaccccacccctcctcctggcagtctcctcctgtgcagaacccaacaaacacaaaattaagtcactctcaaacccacagcagatgagagcttctctggaagctccctggtggggaaaggctgcaattgctattttcttcttctggttttcacctcaggctttgtgttatattgacagtacccttctcaagctaactccctaactgacctgacgtagtcaaaataagttctttgtatgtcagttctgaggtgtgtgtgttttcacttacaaacagtactctacagctttaagacattatattaaagtcctgagaagtgatttttaaaccactgaacttcatcttttccctcctggctagtatttcagactttcagtgtttgaggcatgcatttcacctgaacaacttgaaaaataatatcctaagaagcacacaacctgactttaggctcattcacatggattgtcactttacttggacccactttctcggctgagaggtttgttttcccataaccacggatgctcatagttaatataaatattgaactcactatgtagtgaggacatagagcctctttaacattggtccctgttaggagaaagtttctcccataacatactaaatacatgttttaatagccgttccttctgaaaggtccaacttcactattttatttttttagtaaaatcttagttaacaaattaatggaggttaggtggaattttgccccaaaagtcctgtattttctttttttttttttctttttttttgacagagtcttgctctgtcgcccaggctggagtgcagtggcgtgatctcggctcactgcaagctccgcctcctaaggtcacgccattctcctgcctcagcctcctgagtagctggggctacaggtgcccgccaccgcacccggctaattttttgtatttttagtagagaccgggtttcactgtgttagccaggatggtctcaatctcctgacctcgggatccacccacctcggcctcccaaagtgctgggattacaggcgtgagccaccatgcccggcctgtcctgtattttcaagaaactttttttttcctccagaaatgataccctagtctttcatatttgttttcagatggactgaataaaagctgttgttttggaacaatcacggttaaaaaaaaaagttatgaatttagtcaactcagagctctataaaaataatccaaaaaattccttcaaactctgaacgcttcaaaagagcgtgcaaatattctgtccttcaaagctaaggaaacatgatttgtggggtgcatcacagtggaaaaatactctgacagcattcccacagcattaggggaagtgcatgtgtgggtgttctgcaagggacaattctccagaaaaggcaatttccctttgacatgctgtttttaatgacttttctttataaacacacttatctctccagagaaatagcagtgcatttgcaacaggcccgtaaaatgcaacaaaacctctgctatggtttctgacccctgcttttatacagagcatcagaccaaggaacctgttctaacaggattatttcagaggggaacacaggcttagggtgcagatcttccagctggatttttcactttgcattccctccacagcagacacatgaaggaatgattttgtgattttgattttataatttgcacacttttcctaaatactttttttaaatttttatttgggaggattttatagcatatgattgagaactataatcatcatcattgttacagaagaataatttagaaaaattttttaactacgttaaaaattccactatgggtggatgacaatattgttctttccttccacattctccctccttagactttcttttctttttttctattttttttttgagatgaagtctcgctctgtcactcaggctggagtgcagtgccatgatcctggctcactgcaacctctgcctcccgggttcaagtgattctcccgcctcagcttcctgagtagctgggattacaggtgtgcaccaccacacctggctactttttgtatttttagtagagatggggtttcaccatgttggtcaggctggtctcaaactcctgatctcatgatctgcccgccttggccccgcaaagtgccgggattacaggcgtgagccactgcgcctggcctctctctcggactttctaccatcagtcagattgaatttgttaaattctgtcactgaccctaaacccaacaaaaggcaagagttatgtttatttagcacttcctctacctatagcaaacctcaatttagagcgtaattttaagcacaatttaattataaatatcttttcattttcttacttaactcactcagttttttaaatcttcttttttgagacaagatcttgctctgtcactgaggccgatgtacagtgatgtgatcatgacttactgcagccttgacctcccaggcttaggtgatcctcatacctcagcctcccgagcaactaggactacaggcccgtgccaccatgccgggccaagacggggtttggacgtgttgccccagctggtctccaactcctggcctcaagtgaccctcccgcctcggcctctcaaagtgctgggattataggcatgagccaccgcacctggccaactcactcacattttaagttttttctttttttcatctagttttttttctttttaaatttgaaagcctcatgacattaatgatttcttacattaaaagaaaaacacccaaaaatactctgcttacataacaccgacaagtagtgtgcaagactcattagcatttgtcatctgaagtgaccaaatccagacttttgggggtcacattaaagaaacagttgaagagttagaactatgggtaaagcgagtgtgcatatcagaaagtggaatattgtcttcctcaggagctgacaatttatgaaaaatagttcacattctcagctagaaaggcttctatttttgctcatattcctggctagttttgctgaaataattgctttgaattacttcctcaggactgcccaggtgacgctaatgtttactctgcccttcacaggtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaaggtaaggagccctagctgcggctatctcctgaaggttatgtcaccagagggcatgacacttgattaaatctccaacatcaacacacactgccacccttggaatggagggctatccattttgtgcctcactgaaacagtccaagagatatctagcatttccccaaggataaaggagtgtagctaaaagtagaagaccagaaatccctagcccctactctggatctatgcaagcctagattcttgtcttccatcttggatggctccacagcagtcttaactgtttcatgtacataaagcagtacataaagatttaaccttgctgggcatggtggctcacacctgtaatcccagcattttggaaggccaaggcaggaggattgcttgagcctagaagtttgagaccagcctgggcaacatagtgagaccttgtctctactaaaaatcacaaaaattagctgggcacggtggcatatacgcctgcagattcagttacttgggaggagaggcgggaggattgcttgagcttgggaggtccagctgcagtgaatcatgatcacagcactgcaatctggcctgggtgacagagcaagacactatttcaaaaaaaaaaagaccaagcatggtggctcatgcctgtaatcccagcactttgggaggctgaggcaggtggatcatctgaggtcagaagttcaagaccagcctgaccaacatggtgaaaccccgtctctactgaaaatacgaaaattatccaggtgtagtgatgcacacctgtaatctcagctactcgggaggctgaggcagaagaatcacttgaactggggacgtggaggctgcagtgagccaagattgcaccattgcactccagcctgggtgacagagcaagactccatctcaaaaaaaaaaaaaaaaaaaaaaggatttaacccaagtatatcatagtagattgaattatgtaaaacacccatttaacaaccaggtccaggtttgttctctctgtgtagtaaatcaatcactgtgacacaggttttgcaaaagagaaaagatttatttgtaaggggaccaagcgagggggtgggagaataacttccaatcctgcctctctgaagacaaggcttaggaatatgtatgggttagggaatgggtggtctaaggcatggtgaagagtgattggcagggggggaaaatgaagtaacaggttagacacatgcacagaaaatggtggtgttagcatgatctgagggcagagttttgggccctctgacgtcaaaagaccacctctcaggcacttgtgcaggcccagtggaagggtcagtggtcttaactagtttgaactggacaggagctgccccaagttcttggaaaaacaactgaagtgaccattgccatggtaacctatgaatgtcatcagtaaagtagccagtgaaggttaagtttcagcatacaatgggacaaccttcagcttcatggaaaaaggaaaaaaaaaaaacacatacacacacgaaaagcaagtgaccaaaagcaagcaggacaggcagacctgatccaattaacccctgggtttcaaccctgctaaatgcagctcaatatttgtcttgataatttgcctatttggctttacataaaataaagccttttctgatgaaatctaattgagtctgaagttgtattaaatggtatcggaaacttcccagcaggaaggctacgtaaaagtggccgggcgtggtgactcacgcctgtaatcccagcactttgggaggctgaggcaggcagatcacaaggtcaagaaatcgagaccatcctggccaacatggcgaaatcccatctctactaaaaaaaaaaatacaaaaatttgccaggtgtggtggtgctcacctgtagtcccagctactcaggaggctgaggcaggagaatctgttgaacctgggaggcggaggttgcagtgagtcaagatggtgccattgcactccagcctgtgtgacagagcaagactccgtctcaaaaaaaaaaaaaagtgatgtgttgtgtgcaaaatacgtaataactactctcctatccttttgtttttccagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttcttctgcacccacttctaaatttaatgtttttctggaagtagtaattgcaagagttcgaatagaaagttatgtaccaagtaaccatttctcagctgccataataatgcctagtggcttcccctctgtcaaatctagtttcctatggaaaagaagatggcagatacaggagagacgacagagggtcctaggctggaatgttcctttcgaaagcaatgcttctatcaaatactagtattaatttatgtatctggttaatgacatacttggagagcaaattatggaaatgtgtattttatatgatttttgaggtcctgtctaaaccctgtgtccctgagggatctgtctcactggcatcttgttgagggccttgcacataggaaacttttgataagtatctgcggaaaaacaaacatgaatcctgtgatattgggctcttcaggaagcataaagcaattgtgaaatacagtataccgcagtggctctaggtggaggaaaggaggaaaaagtgcttattatgtgcaacattatgattaatctgattatacaccatttttgagcagatcttggaatgaatgacatgacctttccctagagaataaggatgaaataatcactcattctatgaacagtgacactactttctattctttagctgtactgtaatttctttgagttgatagttttacaaattcttaataggttcaaaagcaatctggtctgaataacactggatttgtttctgtgatctctgaggtctattttatgtttttgctgctacttctgtggaagtagctttgaactagttttactttgaactttcacgctgaaacatgctagtgatatctagaaagggctaattaggtctcatcctttaatgccccttaaataagtcttgctgattttcagacagggaagtctctctattacactggagctgttttatagataagtcaatattgtatcaggcaagataaaccaatgtcataacaggcattgccaacctcactgacacagggtcatagtgtataataatatactgtactatataatatatcatctttagaggtatgattttttcatgaaagataagcttttggtaatattcattttaaagtggacttattaaaattggatgctagagaatcaagtttattttatgtatatatttttctgattataagagtaatatatgttcattgtaaaaatttttaaaacacagaaactatatgcaaagaaaaaataaaaattatctataatctcagaacccagaaatagccactattaacatttcctacgtattttattttacatagatcatattgtatatagttagtatctttattaatttttattatgaaactttcctttgtcattattagtcttcaaaagcatgatttttaatagttgttgagtattccaccacaggaatgtatcacaacttaaccgttcccgtttgttagactagtttcttattaatgttgatgaatgttgtttaaaaataattttgttgctacatttactttaatttccttgactgtaaagagaagtaattttgctccttgataaagtattatattaataataaatctgcctgcaactttttgccttctttcataatcata

SEQ ID NO: 21 - DNA, 24 bp, Artificial sequence
  Single strand DNA oligonucleotide
gaagttgtcattttataaaccttt

SEQ ID NO: 22 - DNA, 20 bp, Artificial sequence
  Single strand DNA oligonucleotide
tgacttaccagatgggagac

SEQ ID NO: 23 - DNA, 22 bp, Artificial sequence
  Single strand DNA oligonucleotide
ccaaagcgagcaagagaatcac

SEQ ID NO: 24 - DNA, 22 bp, Artificial sequence
  Single strand DNA oligonucleotide
gacgggaatccaaattacacag

SEQ ID NO: 25 - DNA, 21 bp, Artificial sequence
  Single strand DNA oligonucleotide
tgggatttttagcacagcacg

SEQ ID NO: 26 - DNA, 20 bp, Artificial sequence
  Single strand DNA oligonucleotide
ctggtctgaatgttcgttac

SEQ ID NO: 27 - DNA, 5711 bp, Homo sapiens
agctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 28 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 29 - DNA, 10987 bp, Homo sapiens
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 30 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVAHQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETFYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI